(19) United States
(12) Patent Application Publication
(10) Pub. No.: US 2020/0255478 A1
(43) Pub. Date: Aug. 13, 2020

(54) NOVEL COMPOUNDS ACTIVATING THE NRF2 PATHWAY

(71) Applicant: Almirall, S.A., Barcelona (ES)

(72) Inventors: Carlos PUIG DURAN, Barcelona (ES); Fernando ALBERICIO PALOMERA, Barcelona (ES); Miriam GONGORA BENITEZ, Barcelona (ES); Marta PARADIS BAS, Barcelona (ES); Laia MIRET CASALS, Tarragona (ES); Ivan RAMOS TOMILLERO, Ibiza (ES); Stephen FIACCO, Pasadena, CA (US); Andrew DAVIS, Södertälje (SE); Stefan GESCHWINDNER, Södertälje (SE); Omar BRUN CUBERO, Barcelona (ES); Carlos HERAS PANIAGUA, Barcelona (ES); Nuria TRALLERO CANELA, Barcelona (ES)

(21) Appl. No.: 16/636,679

(22) PCT Filed: Aug. 8, 2018
(86) PCT No.: PCT/EP2018/071536

§ 371 Date: Feb. 5, 2020

(30) Foreign Application Priority Data
Aug. 8, 2017 (EP) 17382558.9

(51) Int. Cl. C07K 7/64

(52) U.S. Cl. CPC C07K 7/64

(57) ABSTRACT

The invention relates to peptidic compounds, which peptidic compounds are compounds of formula (I)′, or a pharmaceutically acceptable salt, or solvate, or N-oxide, or stereoisomer thereof:

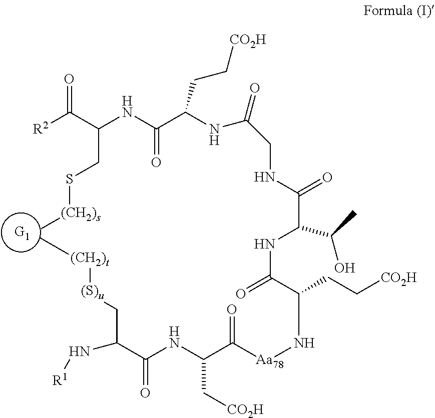

wherein R¹; R²; s; t; u; Aa₇₈ and G₁ are as defined herein.

The peptidic compounds are useful in activating the Nrf2 pathway.

### FIELD OF THE INVENTION

The present invention relates to novel peptides activating the Nrf2 pathway and their use in oxidative stress-dependent pathologies.

### BACKGROUND TO THE INVENTION

Oxidative stress results from the imbalance between reactive oxygen species (ROS) present in a living system and the ability of said system to eliminate them or repair the resulting damage. ROS are also necessary for the immune system of the organism to kill pathogens. In normal conditions the amount of ROS is maintained within a certain limits. When these limits are surpassed the organisms can develop some diseases. Oxidative stress has been linked with the development of several conditions like Parkinson's disease, depression, Alzheimer's disease, atherosclerosis, heart failure, myocardial infarction, diabetes, cancer, COPD, COPD exacerbations, acute lung injury, radiation-induced dermatitis, chemical induced dermatitis, contact induced dermatitis, Epidermolysis bullosa simplex, Pachyonychia congenital, Hailey-Hailey, vitiligo, photoaging and photodamaged skin.

The transcription factor nuclear factor erythroid 2 p45 (NF-E2)-related factor (Nrf2) is a cellular sensor of oxidative stress. Nrf2 is a member of the Cap ‘n’ collar family of basic leucine zipper transcription factors. Under basal conditions Nrf2 levels are tightly controlled by the Kelch-like ECH-associated protein 1 (Keap1), which binds to Nrf2 and targets it for ubiquitination and proteasomal degradation via the cullin 3-dependent ubiquitin E3 ligase complex. The Keap1 dimer binds with its Kelch domains to both the DLG and the ETGE (SEQ ID NO 101) sequence motives of Nrf2. Keap1 contains in the so-called BTB- and IVR-domains highly reactive cysteine residues. These cysteines react with electrophiles in conditions of oxidative stress. As a result, changes of conformation of Keap1 alters Nrf2 binding and promotes its stabilization. Subsequently Nrf2 translocates to the nucleus, where it binds as a heterodimer with small Maf proteins to the so-called anti-oxidant response element (ARE), the promoter region of its target genes. The Nrf2 regulated genes include antioxidants such as γ-glutamyl cysteine synthase catalytic subunit (GCLg), heme oxygensase-1 (HMOX-1), superoxide dismutase, glutathione reductase (GSR), thioredoxin reductase; phase II detoxifying enzymes such as NADP(H) quinone oxyreductase-1 (NQO1), UDP-glucuronosyltransferase; and ATP-dependant drug efflux pumps like MRP1 and MRP2. Furthermore, Nrf2 has been linked to an upregulation of mitochondrial biogenesis and fat oxidation. The inhibition of Nrf2 and subsequent stimulation of Nrf2 also prevents the activation of macrophages by interferon. The keap1/Nrf2 signalling, thus, also controls inflammatory processes. The Nrf2 pathway can be activated by selective inhibition of the protein-protein interaction between Nrf2 and the kelch domain of Keap1. Such interaction contains a high (DEETGE) (SEQ ID NO: 102) and a low (DLG) affinity sequence domain and has been well characterized in mechanistic terms (Lo et al., The EMBO Journal (2006) 25, 3605-361).

Nrf2 activation plays an important role on several respiratory conditions. It has been demonstrated that the Nrf2 pathway is downregulated in pulmonary macrophages of COPD patients (M. Suzuki et al., Am J Respir Cell Mol Biol Vol 39. pp 673-682, 2008) and also in bronchial epithelial cells of such patients (K. Yamada et al., BMC Pulmonary Medicine (2016) 16:27). Nrf2 activators play also a role in animal models of acute lung injury (H.-Y. Cho et al., Arch. Toxicol. 2015 November; 89(11):1931-57; W. Jin et al., Exp Biol Med (Maywood). 2009 February; 234(2):181-9).

There are some dermatological conditions related with Nrf2 activation. Two electrophilic activators of this pathway (sulphoraphane; C. L. Saw et al., Molecular Carcinogenesis 50:479-486 (2011) and RTA-408; S. A. Reisman et al., Radiation Research 181, 000-000 (2014)) have proved to be effective in radiation-induced dermatitis models and are currently in the clinics for the control of this condition. There are also strong evidences of the role of this pathway in skin bullous diseases like Epidermolysis bullosa simplex, Pachyonychia congenital or Hailey-Hailey (M. L. Kerns et al., PNAS, 2007, 104 (36), 14460-14465; M. L. Kerns et al., J. Clin. Inv. 2016, 126 (6), 2356-2366). The role of oxidative stress and Nrf2 activation has been also identified for vitiligo (V. T. Natarajan et al., Journal of Investigative Dermatology (2010) 130, 2781-2789).

Peptidic sequences containing the DXETGE motif (being X whichever aminoacid) (SEQ ID NO: 103), which contains a β-turn region stabilized by the aspartate and threonine residues have been described to bind to Keap1 disrupting its interaction with Nrf2 and thus activating the pathway (see, for example, R. Hancock et al., Free Radical Biology & Medicine 52 (2012) 444-451 or R. Steel et al., Med. Chem. Lett. 2012, 3, 407-410).

It has been found, however, that the peptides containing the DXETGE sequence cannot cross cell membranes easily. In order to improve permeation ability of these compounds, conjugation with a fatty acid (for ex., a stearyl residue) or a cell penetrating peptide (for example, the HIV-TAT sequence) is required.

### SUMMARY OF THE INVENTION

The present inventors have found that a cyclic heterodetic sequence containing an aromatic structure linked to the high affinity sequence through one or two cysteines has an improved binding affinity with respect to similar homodetic cyclic peptides.

Accordingly the present invention provides a peptidic compound, which peptidic compound is a compound of formula (I)′, or a pharmaceutically acceptable salt, or solvate, or N-oxide, or stereoisomer thereof:

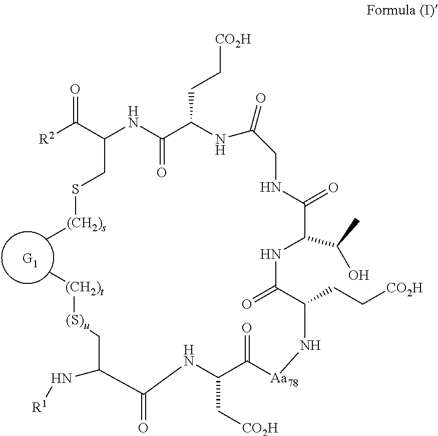

wherein
R¹ represents a hydrogen atom, a —CO(C₁-C₄ alkyl) group, a —CONH(C₁-C₄ alkyl) group or a -Aa₇₅-Aa₇₄-[L₁]ₘ-[Tag₁]ₙ group;
R² represents a —OH group, a —NH₂ group, or a -Aa₈₄-Aa₈₅-[L₂]ₚ-[Tag₂]_{q} group;
Aa₇₄ represents a direct bond, a leucine, a valine, a lysine, an arginine, a phenylalanine, a proline or a N-acetyl-proline residue, wherein when Aa₇₄ is other than a direct bond: (a) Aa₇₄ is optionally linked to Aa₈₅; and/or (b) Aa₇₄ is optionally alkylated with a C₁-C₄ alkyl group on the N at the peptidic bond, and wherein when Aa₇₄ is a proline or a N-acetyl-proline residue it is unsubstituted or substituted by a —NH₂ group or a —NHC(O)CH₃ group;
Aa₇₅ represents a direct bond, a glutamine, a leucine, a lysine, a valine, a phenylalanine or an arginine residue, wherein when Aa₇₅ is other than a direct bond Aa₇₅ is optionally alkylated with a C₁-C₄ alkyl group on the N at the peptidic bond;
m and n each independently represent an integer selected from 0 and 1, wherein when m and n each represent 0 and Aa₇₄ is not linked to Aa₈₅, the or each amino-terminal group of R¹ is a —NH₂ group or a —NHC(O)CH₃ group and provided that when m and n each represent 0, Aa₇₄ and Aa₇₅ cannot simultaneously be a direct bond;
when m is 1 and n is 1 L₁ represents a —C(O)—(CH₂)₍₁₋₄₎—NH— group and when m is 1 and n is 0 L₁ represents a —C(O)—(CH₂)₍₁₋₄₎—NH₂ group;
Tag₁ represents a —C(O)—(CH₂)ᵣ—CH₃ group or a —C(O)—(CH₂)₇—(CH═CH—CH₂)₁₋₃—(CH₂)₀₋₆—CH₃ group, wherein when Aa₇₄ represents a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetyl-proline residue;
r represents an integer selected from 6 to 24;
Aa₈₄ represents a direct bond, a leucine, a valine, a lysine or an arginine residue, wherein, when Aa₈₄ is other than a direct bond, Aa₈₄ is optionally alkylated with a C₁-C₄ alkyl group on the N at the peptidic bond;
Aa₈₅ represents a direct bond, a proline, a leucine, a valine, a lysine, an arginine, or a D-proline residue, wherein when Aa₈₅ is other than a direct bond: (a) Aa₈₅ is optionally linked to Aa₇₄; and/or (b) Aa₈₅ is optionally alkylated with a C₁-C₄ alkyl group on the N at the peptidic bond;
p and q each independently represent an integer selected from 0 and 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond;
when p is 1 and q is 1 L₂ represents a —NH—(CH₂)₍₁₋₄₎—CO— group and when p is 1 and q is 0 L₂ represents a —NH—(CH₂)₍₁₋₄₎—COOH or a —NH—(CH₂)₍₁₋₄₎—CONH₂ group;
Tag₂ represents a peptide containing from 6 to 20 amino acids wherein at least three of these amino acids are selected from the group consisting of lysine and arginine, wherein the or each carboxy-terminal group of Tag₂ is a —COOH group or a —CONH₂ group;
s represents 0 or 1;
t represents 0 or 1;
u represents 0 or 1;
Aa₇₈ represents a proline, a L-thioproline, an alanine, a phenylalanine, an arginine or a glutamic acid residue, wherein the proline, L-thioproline, alanine, phenylalanine, arginine or glutamic acid residue is optionally substituted by one, two or three substituents selected from a halogen atom and amino group and wherein Aa₇₈ is optionally alkylated with a C₁-C₄ alkyl group on the N at the peptidic bond; and
G₁ represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one or more substituents selected from a C₁-C₄ alkyl group and a halogen atom; or a 4-6 membered saturated heterocyclyl group containing one oxygen atom selected from an oxetanyl group, a tetrahydrofuranyl group and a tetrahydro-2H-pyranyl group.

Accordingly the present invention also provides a peptidic compound, which peptidic compound is a compound of formula (I), or a pharmaceutically acceptable salt, or solvate, or N-oxide, or stereoisomer thereof:

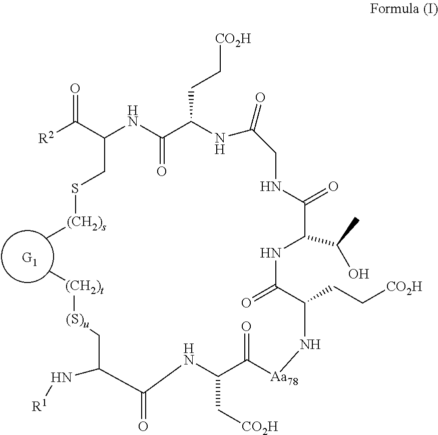

wherein
R¹ represents a hydrogen atom, a —CO(C₁-C₄ alkyl) group, a —CONH(C₁-C₄ alkyl) group or a -Aa₇₅-Aa₇₄-[L₁]ₘ-[Tag₁]ₙ group;
R² represents a —OH group, a —NH₂ group, or a -Aa₈₄-Aa₈₅-[L₂]ₚ-[Tag₂]_{q} group;
Aa₇₄ represents a direct bond, a leucine, a valine, a lysine, an arginine, a phenylalanine, a proline or a N-acetyl-proline residue, wherein when Aa₇₄ is other than a direct bond: (a) Aa₇₄ is optionally linked to Aa₈₅; and/or (b) Aa₇₄ is optionally alkylated with a C₁-C₄ alkyl group on the N at the peptidic bond, and wherein when Aa₇₄ is a proline or a N-acetyl-proline residue it is unsubstituted or substituted by a —NH₂ group or a —NHC(O)CH₃ group;
Aa₇₅ represents a direct bond, a glutamine, a leucine, a lysine, a valine, a phenylalanine or an arginine residue, wherein when Aa₇₅ is other than a direct bond Aa₇₅ is optionally alkylated with a C₁-C₄ alkyl group on the N at the peptidic bond;
m and n each independently represent an integer selected from 0 and 1, wherein when m and n each represent 0 and Aa₇₄ is not linked to Aa₈₅, the or each amino-terminal group of R¹ is a —NH₂ group or a —NHC(O)CH₃ group and provided that when m and n each represent 0, Aa₇₄ and Aa₇₅ cannot simultaneously be a direct bond;
when m is 1 and n is 1 L₁ represents a —C(O)—(CH₂)₍₁₋₄₎—NH— group and when m is 1 and n is 0 L₁ represents a —C(O)—(CH₂)₍₁₋₄₎—NH₂ group;
Tag₁ represents a —C(O)—(CH₂)ᵣ—CH₃ group, wherein when Aa₇₄ represents a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetyl-proline residue;
r represents an integer selected from 6 to 18;
Aa₈₄ represents a direct bond, a leucine, a valine, a lysine or an arginine residue, wherein, when Aa₈₄ is other than a direct bond, Aa₈₄ is optionally alkylated with a C₁-C₄ alkyl group on the N at the peptidic bond;
Aa₈₅ represents a direct bond, a proline, a leucine, a valine, a lysine, an arginine, or a D-proline residue, wherein when Aa₈₅ is other than a direct bond: (a) Aa₈₅ is optionally linked to Aa₇₄; and/or (b) Aa₈₅ is optionally alkylated with a C₁-C₄ alkyl group on the N at the peptidic bond;
p and q each independently represent an integer selected from 0 and 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond;
when p is 1 and q is 1 L₂ represents a —NH—(CH₂)₍₁₋₄₎—CO— group and when p is 1 and q is 0 L₂ represents a —NH—(CH₂)₍₁₋₄₎—COOH or a —NH—(CH₂)₍₁₋₄₎—CONH₂ group;
Tag₂ represents a peptide containing from 6 to 20 amino acids wherein at least three of these amino acids are selected from the group consisting of lysine and arginine, wherein the or each carboxy-terminal group of Tag₂ is a —COOH group or a —CONH₂ group;
s represents 0 or 1;
t represents 0 or 1;
u represents 0 or 1;
Aa₇₈ represents a proline, an alanine, a phenylalanine, an arginine or a glutamic acid residue, wherein the proline, alanine, phenylalanine, arginine or glutamic acid residue is optionally substituted by one, two or three substituents selected from a halogen atom and amino group and wherein Aa₇₈ is optionally alkylated with a C₁-C₄ alkyl group on the N at the peptidic bond; and
G₁ represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one or more substituents selected from a C₁-C₄ alkyl group and a halogen atom.

The invention also provides a pharmaceutical composition comprising a peptidic compound as defined herein together with one or more pharmaceutically acceptable carriers and/or excipients.

Also provided by the invention is a peptidic compound as defined herein or a pharmaceutical composition as defined herein for use in a method of treatment of a human or animal body by therapy.

The invention also provides a peptidic compound as defined herein or a pharmaceutical composition as defined herein for use in treatment of a pathological condition or disease associated with the activation of the Nrf2 pathway.

Also provided by the invention is a method of treating a subject afflicted with a pathological condition or disease as defined herein, which comprises administering to said subject an effective amount of a peptidic compound as defined herein or a pharmaceutical composition as defined herein.

Further provided by the invention is the use of a peptidic compound as defined herein or a pharmaceutical composition as defined herein in the manufacture of a medicament for the treatment of a pathological condition or disease as defined herein.

### DETAILS DESCRIPTION OF THE INVENTION

The term amino acid, as used herein, refers to any one of the twenty standard amino acids, as listed below, or to the equivalent D-amino acid, or to N-acetyl-proline or to L-thioproline (Thz) or to D-thioproline. Preferably, the term amino acid refers to any one of the twenty standard amino acids or D-proline or N-acetyl-proline or L-thioproline.

As used herein, L-thioproline (Thz) refers to (4R)-4-thiazolidinecarboxylic acid.

In another embodiment, the term amino acid, as used herein, refers to any one of the twenty standard amino acids, as listed below, or to the equivalent D-amino acid, or to N-acetyl-proline. Preferably, the term amino acid refers to any one of the twenty standard amino acids or D-proline or N-acetyl-proline.

| | | | |
|---|---|---|---|
| Ala (A) | Met (M) | Gly (G) | Ser (S) |
| Cys (C) | Asn (N) | His (H) | Thr (T) |
| Asp (D) | Pro (P) | Ile (I) | Val (V) |
| Glu (E) | Gln (Q) | Lys (K) | Trp (W) |
| Phe (F) | Arg (R) | Leu (L) | Tyr (Y) |

Each amino acid can be considered as having the general formula NH₂—CHR—COOH, wherein R is the amino acid side chain. As an example, the amino acid alanine has a methyl side chain, i.e., for alanine R is methyl.

The peptidic compounds of the invention comprise amino acid residues. Individual amino acid residues are linked by peptide bonds. When two amino acids join together to form a peptide bond, the two amino acids are linked via a —NH—CO—bond. As used herein, a peptidic bond is a bond having the structure —NH—CO—.

An amino acid residue refers to an amino acid that is lacking either the hydrogen atom of the amino group (i.e., a —NH—CHR—COOH moiety) or the hydroxyl moiety of the carboxyl group (i.e., a NH₂—CHR—CO— moiety) or both (i.e. a —NH—CHR—CO— moiety). When the amino acid side chain R has an amino group or a carboxyl group as substituents, the term amino acid residue also refers to an amino acid that is lacking the hydrogen atom of the amino group or the hydroxyl moiety of the carboxyl group, respectively, within the side chain.

The term amino-terminal group (or N-terminal), as used herein, refers to an amino group within an amino acid (including an amino group within the side chain R) that is not directly linked to another amino acid residue via a peptide bond.

The term carboxy-terminal group (or C-terminal), as used herein, refers to a carboxyl group within an amino acid (including a carboxyl group within the side chain R) that is not directly linked to another amino acid residue via a peptide bond.

As used herein, a C₁-C₄ alkyl group or moiety can be linear, branched or cyclic but is preferably linear. It is preferably a C₁-C₃ alkyl group or a C₁-C₂ alkyl group, more preferably methyl. Suitable such alkyl groups and moieties include methyl, ethyl, n-propyl, i-propyl, n-butyl, sec-butyl and tert-butyl.

As used herein, a halogen is typically chlorine, fluorine, bromine or iodine, and is preferably chlorine or fluorine, more preferably fluorine.

Typically, Aa₇₄ represents a direct bond, a leucine, a valine, a lysine, an arginine, a phenylalanine, a proline or a N-acetyl-proline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond, and wherein when Aa₇₄ is a proline or a N-acetyl-proline residue it is unsubstituted or substituted by a —NH₂ or —NHC(O)CH₃ group.

Preferably, Aa₇₄ represents a direct bond, a leucine, a valine, a lysine, a proline, a 4-aminoproline, a 4-acetaminoproline or a 4-amino-N-acetyl-proline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond.

More preferably, Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond).

As explained above, Aa₇₄ is optionally linked to Aa₈₅ by a peptidic bond. The peptidic bond has the structure —NH—CO—. The —NH— moiety within the peptidic bond is derived from the Aa₇₄ moiety: it can be derived from either a —NH₂ group on the side chain of Aa₇₄ or from the amino group alpha to the carboxyl group of Aa₇₄.

The skilled person will appreciate, therefore, that in some embodiments, R¹ and R² together represent:

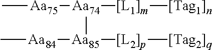

wherein Aa₇₄ is linked to Aa₈₅ by a peptidic bond and wherein Aa₇₅, Aa₇₄, Aa₈₄, Aa₈₅, L₁, L₂, Tag₁, Tag₂, m, n, p and q are as defined herein.

Typically Aa₇₅ represents a direct bond, a glutamine, a leucine, a lysine, a valine, a phenylalanine or an arginine residue, wherein when Aa₇₅ is other than a direct bond, Aa₇₅ is optionally alkylated with a methyl group on the N at the peptidic bond.

Preferably, Aa₇₅ represents a direct bond, a glutamine, a leucine, a lysine or a valine residue, wherein when Aa₇₅ is other than a direct bond, Aa₇₅ is optionally alkylated with a methyl group on the N at the peptidic bond.

More preferably, Aa₇₅ represents a direct bond, a glutamine, a leucine, a lysine or a valine residue.

Preferably, (i) m is 0 and n is 0; or (ii) m is 0 and n is 1; or (iii) m is 1 and n is 1.

When m and n each represent 0, and Aa₇₄ is not linked to Aa₈₅, the or each amino-terminal group of R¹ is typically a —NH₂ group.

Typically, when m is 1 and n is 1 L₁ represents a —C(O)—(CH₂)₍₁₋₃₎—NH— group and when m is 1 and n is 0 L₁ represents a —C(O)—(CH₂)₍₁₋₃₎—NH₂ group. Preferably, when m is 1 and n is 1, L₁ represents a —C(O)—(CH₂)₍₁₋₂₎—NH— group. More preferably, when m is 1 and n is 1, L₁ represents a —C(O)—(CH₂)₂—NH— group.

For the avoidance of doubt, the orientation of the L₁ group is such that the left hand side of the depicted moieties are attached to Aa₇₄ and the right hand side of the depicted moieties are attached to Tag₁ (i.e. the —CO— moiety of the L₁ group is attached to Aa₇₄ and the —NH— moiety is attached to Tag₁).

Typically, r represents an integer from 6 to 24. Preferably, r represents an integer selected from 6 to 22, and more preferably r represents 6 to 20.

In another embodiment, typically, r represents an integer selected from 6 to 17. Preferably, r represents an integer selected from 6 to 17 and more preferably r represents 6 or 16.

For the avoidance of doubt, the orientation of the Tag₁ group is such that the left hand side of the Tag₁ moiety (i.e. the left hand side of the —C(O)—(CH₂)ᵣ—CH₃ group or the left hand side of the —C(O)—(CH₂)₇—(CH═CH—CH₂)₁₋₃—(CH₂)₀₋₆—CH₃ group,) is attached to L₁, i.e. the —CO— moiety is attached to L₁.

For the avoidance of doubt, the orientation of the Tag₁ group is such that the left hand side of the Tag₁ moiety (i.e. the left hand side of the —C(O)—(CH₂)ᵣ—CH₃ group) is attached to L₁, i.e. the —CO— moiety is attached to L₁.

Typically, in the moiety -Aa₇₅-Aa₇₄-[L₁]ₘ-[Tag₁]ₙ, Aa₇₄ represents a direct bond, a leucine, a valine, a lysine, an arginine, a phenylalanine, a proline or a N-acetyl-proline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond, and wherein when Aa₇₄ is a proline or a N-acetyl-proline residue it is unsubstituted or substituted by a —NH₂ or —NHC(O)CH₃ group; Aa₇₅ represents a direct bond, a glutamine, a leucine, a lysine, a valine, a phenylalanine or an arginine residue, wherein when Aa₇₅ is other than a direct bond, Aa₇₅ is optionally alkylated with a methyl group on the N at the peptidic bond; m and n each independently represent an integer selected from 0 and 1, wherein when m and n each represent 0 and Aa₇₄ is not linked to Aa₈₅, the or each amino-terminal group of R¹ is a —NH₂ group or a —NHC(O)CH₃ group and provided that when m and n each represent 0, Aa₇₄ and Aa₇₅ cannot simultaneously be a direct bond; when m is 1 and n is 1 L₁ represents a —C(O)—(CH₂)₍₁₋₃₎—NH— group and when m is 1 and n is 0 L₁ represents a —C(O)—(CH₂)₍₁₋₃₎—NH₂ group; Tag₁ represents a —C(O)—(CH₂)ᵣ—CH₃ group or a —C(O)—(CH₂)₇—(CH═CH—CH₂)₁₋₃—(CH₂)₀₋₆—CH₃ group, wherein when Aa₇₄ represents a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetylproline residue; and r represents an integer selected from 6 to 24.

Preferably, in the moiety -Aa₇₅-Aa₇₄-[L₁]ₘ-[Tag₁]ₙ, Aa₇₄ represents a direct bond, a leucine, a valine, a lysine, a proline, a 4-aminoproline or a 4-acetaminoproline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond; Aa₇₅ represents a direct bond, a glutamine, a leucine, a lysine or a valine residue, wherein when Aa₇₅ is other than a direct bond, Aa₇₅ is optionally alkylated with a methyl group on the N at the peptidic bond; (i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond; L₁ represents a —C(O)—(CH₂)₍₁₋₂₎—NH— group; Tag₁ represents a —C(O)—(CH₂)ᵣ—CH₃ group or a —C(O)—(CH₂)₇—(CH═CH—CH₂)₁—(CH₂)₆—CH₃ group —C(O)—(CH₂)₇—(CH═CH—CH₂)₃—CH₃ group, wherein when Aa₇₄ represents a 4-aminoproline residue and m is 0, the Tag₁ group is linked to Aa₇₄ through the 4-amino substituent of the 4-aminoproline residue; and r represents an integer selected from 6 to 22.

More preferably, in the moiety -Aa₇₅-Aa₇₄-[L₁]ₘ-[Tag₁]ₙ, Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond); Aa₇₅ represents a direct bond, a glutamine, a leucine, a lysine or a valine residue; (i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond; L₁ represents a —C(O)—(CH₂)₂—NH— group; Tag₁ represents a —C(O)—(CH₂)ᵣ—CH₃ group, a —C(O)—(CH₂)₇-((E-CH═CH)—CH₂)₁—(CH₂)₆—CH₃ group, a —C(O)—(CH₂)₇—(Z—CH═CH)—CH₂)₁—(CH₂)₆—CH₃ group or a —C(O)—(CH₂)₇—((Z—CH═CH)—CH₂)₃—CH₃ group, wherein when Aa₇₄ represents a 4-aminoproline residue and m is 0, the Tag₁ group is linked to Aa₇₄ through the 4-amino substituent of the 4-aminoproline residue; and r represents 6 to 20.

Most preferably, in the moiety -Aa₇₅-Aa₇₄-[L₁]ₘ-[Tag₁]ₙ, Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond); Aa₇₅ represents a direct bond, a glutamine, a leucine, a lysine or a valine residue; (i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond; L₁ represents a —C(O)—(CH₂)₂—NH— group; Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue; and r represents 6 to 20.

In another embodiment, typically, in the moiety -Aa₇₅-Aa₇₄-[L₁]ₘ-[Tag₁]ₙ, Aa₇₄ represents a direct bond, a leucine, a valine, a lysine, an arginine, a phenylalanine, a proline or a N-acetyl-proline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond, and wherein when Aa₇₄ is a proline or a N-acetyl-proline residue it is unsubstituted or substituted by a —NH₂ or —NHC(O)CH₃ group; Aa₇₅ represents a direct bond, a glutamine, a leucine, a lysine, a valine, a phenylalanine or an arginine residue, wherein when Aa₇₅ is other than a direct bond, Aa₇₅ is optionally alkylated with a methyl group on the N at the peptidic bond; m and n each independently represent an integer selected from 0 and 1, wherein when m and n each represent 0 and Aa₇₄ is not linked to Aa₈₅, the or each amino-terminal group of R¹ is a —NH₂ group or a —NHC(O)CH₃ group and provided that when m and n each represent 0, Aa₇₄ and Aa₇₅ cannot simultaneously be a direct bond; when m is 1 and n is 1 L₁ represents a —C(O)—(CH₂)₍₁₋₃₎—NH— group and when m is 1 and n is 0 L₁ represents a —C(O)—(CH₂)₍₁₋₃₎—NH₂ group; Tag₁ represents a —C(O)—(CH₂)ᵣ—CH₃ group, wherein when Aa₇₄ represents a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetylproline residue; and r represents an integer selected from 6 to 17.

Still in this embodiment, preferably, in the moiety -Aa₇₅-Aa₇₄-[L₁]ₘ-[Tag₁]ₙ, Aa₇₄ represents a direct bond, a leucine, a valine, a lysine, a proline, a 4-aminoproline or a 4-acetaminoproline residue, wherein when Aa₇₄ is other than a direct bond: (a) Aa₇₄ is optionally linked to Aa₈₅; and/or (b) Aa₇₄ is optionally alkylated with a methyl group on the N at the peptidic bond; Aa₇₅ represents a direct bond, a glutamine, a leucine, a lysine or a valine residue, wherein when Aa₇₅ is other than a direct bond, Aa₇₅ is optionally alkylated with a methyl group on the N at the peptidic bond; (i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond; L₁ represents a —C(O)—(CH₂)₍₁₋₂₎—NH— group; Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue; and r represents an integer selected from 6 to 17.

Still in this embodiment, more preferably, in the moiety -Aa₇₅-Aa₇₄-[L₁]ₘ-[Tag₁]ₙ, Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond); Aa₇₅ represents a direct bond, a glutamine, a leucine, a lysine or a valine residue; (i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond; L₁ represents a —C(O)—(CH₂)₂—NH— group; Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue; and r represents 6 or 16.

Typically, in a preferred embodiment, -Aa₇₅-Aa₇₄-[L₁]ₘ-[Tag₁]ₙ is selected from:
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₂₀—CH₃)—;
-Gln-Pro((4S)—NH—CO—(CH₂)₁₈—CH₃)—;
-Gln-Pro((4S)—NH—CO—(CH₂)₁₇—CH₃)—;
-Gln-Pro((4S)—NH—CO—(CH₂)₁₆—CH₃)—;
-Gln-Pro((4S)—NH—CO—(CH₂)₁₄—CH₃)—;
-Gln-Pro((4S)—NH—CO—(CH₂)₁₂—CH₃)—;
-Gln-Pro((4S)—NH—CO—(CH₂)₁₀—CH₃)—;
-Gln-Pro((4S)—NH—CO—(CH₂)₇-((E-CH═CH)—CH₂)₁—(CH₂)₆—CH₃)—;
-Gln-Pro((4S)—NH—CO—(CH₂)₇—((Z—CH═CH)—CH₂)₁—(CH₂)₆—CH₃)—;
-Gln-Pro((4S)—NH—CO—(CH₂)₇—((Z—CH═CH)—CH₂)₃—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-Lys(N⁶—CO—(CH₂)₁₆—CH₃)—;
-Gln-Lys(-CO—(CH₂)₁₆—CH₃)—;
-Gln-AcPro((4S)—NH—CO—(CH₂)₁₆—CH₃);
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃;
-Lys-Pro((4S)—NH—CO—(CH₂)₁₆—CH₃); and
-Val-Val-CO—(CH₂)₂—NH—CO—(CH₂)₁₆—CH₃.

In another preferred embodiment, -Aa₇₅-Aa₇₄-[L₁]ₘ-[Tag₁]ₙ is selected from:
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₁₆—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-AcPro((4S)—NH—CO—(CH₂)₁₆—CH₃);
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃; and
-Val-Val-CO—(CH₂)₂—NH—CO—(CH₂)₁₆—CH₃.

Typically, R¹ represents a hydrogen atom, a —CO(C₁-C₄ alkyl) group or a -Aa₇₅-Aa₇₄-[L₁]ₘ-[Tag₁]ₙ group, wherein Aa₇₅, Aa₇₄, L₁, Tag₁, m and n are as defined above. R¹ preferably represents a —CO(C₁-C₂ alkyl) group or a -Aa₇₅-Aa₇₄-[L₁]ₘ-[Tag₁]ₙ group, wherein Aa₇₅, Aa₇₄, L₁, Tag₁, m and n are as defined above. More preferably, R¹ represents a —COCH₃ group or a -Aa₇₅-Aa₇₄-[L₁]ₘ-[Tag₁]ₙ group, wherein Aa₇₅, Aa₇₄, L₁, Tag₁, m and n are as defined above.

Most preferably R¹ is selected from:
—COCH₃;
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₂₀—CH₃)—;
-Gln-Pro((4S)—NH—CO—(CH₂)₁₈—CH₃)—;
-Gln-Pro((4S)—NH—CO—(CH₂)₁₇—CH₃)—;
-Gln-Pro((4S)—NH—CO—(CH₂)₁₆—CH₃)—;
-Gln-Pro((4S)—NH—CO—(CH₂)₁₄—CH₃)—;
-Gln-Pro((4S)—NH—CO—(CH₂)₁₂—CH₃)—;
-Gln-Pro((4S)—NH—CO—(CH₂)₁₀—CH₃)—;
-Gln-Pro((4S)—NH—CO—(CH₂)₇-((E-CH═CH)—CH₂)₁—(CH₂)₆—CH₃)—;
-Gln-Pro((4S)—NH—CO—(CH₂)₇—((Z—CH═CH)—CH₂)₁—(CH₂)₆—CH₃)—;
-Gln-Pro((4S)—NH—CO—(CH₂)₇—((Z—CH═CH)—CH₂)₃—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-Lys(N⁶—CO—(CH₂)₁₆—CH₃)—;
-Gln-Lys(-CO—(CH₂)₁₆—CH₃)—;
-Gln-AcPro((4S)—NH—CO—(CH₂)₁₆—CH₃);
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃;
-Lys-Pro((4S)—NH—CO—(CH₂)₁₆—CH₃)—; and
-Val-Val-CO—(CH₂)₂—NH—CO—(CH₂)₁₆—CH₃.

In another embodiment, most preferably R¹ is selected from:
—COCH₃;
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₁₆—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-AcPro((4S)—NH—CO—(CH₂)₁₆—CH₃);
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃; and
-Val-Val-CO—(CH₂)₂—NH—CO—(CH₂)₁₆—CH₃.

Typically, Aa₈₄ represents a direct bond, a leucine, a valine, a lysine or an arginine residue, wherein, when Aa₈₄ is other than a direct bond, Aa₈₄ is optionally alkylated with a methyl group on the N at the peptidic bond.

Preferably, Aa₈₄ represents a direct bond, a leucine, a valine or a lysine residue, wherein, when Aa₈₄ is a leucine residue, Aa₈₄ is optionally alkylated with a methyl group on the N at the peptidic bond (i.e. the leucine residue is optionally N-methylated at the peptidic bond).

Typically, Aa₈₅ represents a direct bond, a proline, a leucine, a valine, a lysine, an arginine, or a D-proline residue, wherein when Aa₈₅ is other than a direct bond: (a) Aa₈₅ is optionally linked to Aa₇₄; and/or (b) Aa₈₅ is optionally alkylated with a methyl group on the N at the peptidic bond.

Preferably, Aa₈₅ represents a direct bond, a proline, a leucine, a valine, a lysine, or a D-proline residue, wherein when Aa₈₅ is other than a direct bond it is optionally linked to Aa₇₄.

Preferably, (i) p is 0 and q is 0; or (ii) p is 0 and q is 1; or (iii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond. More preferably, (i) p is 0 and q is 0; or (ii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond.

Typically, when p is 1 and q is 1 L₂ represents a —NH—(CH₂)₍₁₋₃₎—CO— group and when p is 1 and q is 0 L₂ represents a —NH—(CH₂)₍₁₋₃₎—COOH or a —NH—(CH₂)₍₁₋₃₎—CONH₂ group. Preferably, when p is 1 and q is 1, L₂ represents a —NH—(CH₂)₍₁₋₂₎—CO— group. More preferably, when p is 1 and q is 1, L₂ represents a —NH—(CH₂)₂—CO— group.

For the avoidance of doubt, the orientation of the L₂ group is such that the left hand side of the depicted moieties are attached to Aa₈₅ and the right hand side of the depicted moieties are attached to Tag₂, i.e. the —NH— moiety of the L₂ group is attached to Aa₈₅ and the —CO— moiety is attached to Tag₂.

Typically, Tag₂ is a peptide containing from 6 to 14 amino acids, preferably from 8 to 14 amino acids, more preferably from 8 to 11 amino acids. At least three of these amino acids are selected from the group consisting of lysine and arginine.

Typically, the or each carboxy-terminal group of Tag₂ is a —CONH₂ group.

Typically, Tag₂ is a cell penetrating peptide.

The presence of a cell penetrating peptide in a compound facilitates permeation of that compound across cell and nuclear membranes, and thus assists the compound to reach its target location. This technique is described in, for example, WO2009/147368, WO2013/030569, WO2012/150960 and WO2004/097017, and many commonly used cell penetrating peptides are commercially available. It is also known that the ability of a cell penetrating peptide to perform its function can be assisted by the presence of positively charged amino acids, such as lysine and arginine.

Well-known techniques such as flow cytometric analysis fluorescent microscopy may be used to assess whether a given peptide is a cell penetrating peptide.

The sequences of known cell penetrating peptides include, but are not limited to:

(SEQ ID NO: 104)
YGRKKRRQRRR;
(SEQ ID NO: 105)
GRKKRRQRRRPQ;
(SEQ ID NO: 106)
RRRRRRRR;
(SEQ ID NO: 107)
RQIKIWFQNRRMKWKK;
(SEQ ID NO: 108)
KFHTFPQTAIGVGAP-NH2;
(SEQ ID NO: 109)
KLALKLALKALKAALKLA;
(SEQ ID NO: 110)
RQIKWFQNRRMKWKK;
(SEQ ID NO: 111)
RGGRLSYSRRRFSTSTGR;
(SEQ ID NO: 112)
RRLSYSRRRF;
(SEQ ID NO: 113)
PIRRRKKLRRLK;
(SEQ ID NO: 114)
RRQRRTSKLMKR;
(SEQ ID NO: 115)
RRRRNRTRRNRRRVR;
(SEQ ID NO: 116)
KMTRAQRRAAARRNRWTAR;
(SEQ ID NO: 117)
TRRQRTRRARRNR;
(SEQ ID NO: 118)
GRRRRRRRRRPPQ;
and
(SEQ ID NO: 119)
KLALKLALKLALALKLA.

Specific examples of Tag₂ include:

(SEQ ID NO: 120)
-Arg-Arg-Arg-Arg-Arg-Arg-Arg-Arg-Arg-Arg-NH₂;
(SEQ ID NO: 121)
-Arg-Arg-Arg-Arg-Arg-Arg-Arg-Arg-NH₂;
(SEQ ID NO: 122)
-Arg-Lys-Lys-Arg-Arg-Gln-Arg-Arg-Arg-NH₂;
and
(SEQ ID NO: 123)
-Tyr-Ala-Arg-Ala-Ala-Ala-Arg-Gln-Ala-Arg-Ala-NH₂.

For the avoidance of doubt, in the specific examples of Tag₂ above, the orientation of the Tag₂ group is such that the left hand amino acid in the depicted Tag₂ moiety is attached to L₂ (i.e. such that the —NH— in the left hand amino acid is attached to L₂).

Typically, in the moiety -Aa₈₄-Aa₈₅-[L₂]ₚ-[Tag₂]_{q}, Aa₈₄ represents a direct bond, a leucine, a valine, a lysine or an arginine residue, wherein, when Aa₈₄ is other than a direct bond, Aa₈₄ is optionally alkylated with a methyl group on the N at the peptidic bond; Aa₈₅ represents a direct bond, a proline, a leucine, a valine, a lysine, an arginine, or a D-proline residue, wherein when Aa₈₅ is other than a direct bond: (a) Aa₈₅ is optionally linked to Aa₇₄; and/or (b) Aa₈₅ is optionally alkylated with a methyl group on the N at the peptidic bond; p and q each independently represent an integer selected from 0 and 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond; when p is 1 and q is 1 L₂ represents a —NH—(CH₂)₍₁₋₃₎—CO— group and when p is 1 and q is 0 L₂ represents a —NH—(CH₂)₍₁₋₃₎—COOH or a —NH—(CH₂)₍₁₋₃₎—CONH₂ group; and Tag₂ is a peptide containing from 6 to 14 amino acids, wherein at least three of these amino acids are selected from the group consisting of lysine and arginine and the or each carboxy-terminal group of Tag₂ is a —CONH₂ group.

Preferably, in the moiety -Aa₈₄-Aa₈₅-[L₂]ₚ-[Tag₂]_{q}, Aa₈₄ represents a direct bond, a leucine, a valine or a lysine residue, wherein, when Aa₈₄ is a leucine residue, Aa₈₄ is optionally alkylated with a methyl group on the N at the peptidic bond (i.e. the leucine residue is optionally N-methylated at the peptidic bond); Aa₈₅ represents a direct bond, a proline, a leucine, a valine, a lysine, or a D-proline residue, wherein when Aa₈₅ is other than a direct bond it is optionally linked to Aa₇₄; (i) p is 0 and q is 0; or (ii) p is 0 and q is 1; or (iii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond; L₂ represents a —NH—(CH₂)₍₁₋₂₎—CO— group; and Tag₂ is a peptide containing from 8 to 14 amino acids, wherein at least three of these amino acids are selected from the group consisting of lysine and arginine and the or each carboxy-terminal group of Tag₂ is a —CONH₂ group.

More preferably, in the moiety -Aa₈₄-Aa₈₅-[L₂]ₚ-[Tag₂]_{q}, Aa₈₄ represents a direct bond, a leucine, a valine or a lysine residue, wherein, when Aa₈₄ is a leucine residue, Aa₈₄ is optionally alkylated with a methyl group on the N at the peptidic bond (i.e. the leucine residue is optionally N-methylated at the peptidic bond); Aa₈₅ represents a direct bond, a proline, a leucine, a valine, a lysine, or a D-proline residue, wherein when Aa₈₅ is other than a direct bond it is optionally linked to Aa₇₄; (i) p is 0 and q is 0; or (ii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond; L₂ represents a —NH—(CH₂)₂—CO— group; and Tag₂ is a peptide containing from 8 to 11 amino acids, wherein at least three of these amino acids are selected from the group consisting of lysine and arginine and the or each carboxy-terminal group of Tag₂ is a —CONH₂ group.

In a preferred embodiment, -Aa₈₄-Aa₈₅-[L₂]ₚ-[Tag₂]_{q} is selected from:

-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
-Lys-D-Pro-;
and
-Val-Val-NH₂.

In another preferred embodiment, -Aa₈₄-Aa₈₅-[L₂]^{p}-[Tag₂]_{q} is selected from:

-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
and
-Val-Val-NH₂.

Typically, R² represents a —NH₂ group, or a -Aa₈₄-Aa₈₅-[L₂]ₚ-[Tag₂]_{q} group, wherein Aa₈₄, Aa₈₅, L₂, Tag₂, p and q are as defined above.

Preferably, R² is selected from:

-NH₂;
-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
-Lys-D-Pro-;
and
-Val-Val-NH₂.

In another embodiment, preferably, R² is selected from:

-NH₂;
-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
and
-Val-Val-NH₂.

Preferably either (i) s, t, and u each represent 0; or (ii) s, t, and u each represent 1.

Typically, Aa₇₈ represents a proline, a L-thioproline, an alanine, a phenylalanine, an arginine or a glutamic acid residue, wherein the proline, L-thioproline, alanine, phenylalanine, arginine or glutamic acid residue is optionally substituted by one substituent selected from a halogen atom and amino group and wherein Aa₇₈ is optionally alkylated with a methyl group on the N at the peptidic bond.

Preferably, Aa₇₈ represents a proline, a L-thioproline, an alanine, a phenylalanine, an arginine or a glutamic acid residue, wherein the proline, L-thioproline, alanine, phenylalanine, arginine or glutamic acid residue is optionally substituted by one substituent selected from a fluorine atom and amino group and wherein Aa₇₈ is optionally alkylated with a methyl group on the N at the peptidic bond.

More preferably, Aa₇₈ represents an unsubstituted alanine, arginine, L-thioproline or glutamic acid residue, or a proline or a phenylalanine residue optionally substituted by one substituent selected from a fluorine atom and amino group, and wherein Aa₇₈ is optionally alkylated with a methyl group on the N at the peptidic bond. (i.e., Aa₇₈ is optionally N-methylated at the peptidic bond).

In another embodiment, typically, Aa₇₈ represents a proline, an alanine, a phenylalanine, an arginine or a glutamic acid residue, wherein the proline, alanine, phenylalanine, arginine or glutamic acid residue is optionally substituted by one substituent selected from a halogen atom and amino group and wherein Aa₇₈ is optionally alkylated with a methyl group on the N at the peptidic bond.

Still in this other embodiment, preferably, Aa₇₈ represents a proline, an alanine, a phenylalanine, an arginine or a glutamic acid residue, wherein the proline, alanine, phenylalanine, arginine or glutamic acid residue is optionally substituted by one substituent selected from a fluorine atom and amino group and wherein Aa₇₈ is optionally alkylated with a methyl group on the N at the peptidic bond.

Still in this other embodiment, more preferably, Aa₇₈ represents an unsubstituted alanine, arginine or glutamic acid residue, or a proline or a phenylalanine residue optionally substituted by one substituent selected from a fluorine atom and amino group, and wherein Aa₇₈ is optionally alkylated with a methyl group on the N at the peptidic bond. (i.e., Aa₇₈ is optionally N-methylated at the peptidic bond).

G₁ typically represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one, two, three or four substituents selected from a C₁-C₄ alkyl group and a halogen atom; or a 4-6 membered saturated heterocyclyl group containing one oxygen atom selected from an oxetanyl group, a tetrahydrofuranyl group and a tetrahydro-2H-pyranyl group.

Preferably, G₁ represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one, two, three or four substituents selected from a C₁-C₂ alkyl group and a halogen atom; or a 4-6 membered saturated heterocyclyl group containing one oxygen atom selected from an oxetanyl group, a tetrahydrofuranyl group and a tetrahydro-2H-pyranyl group.

More preferably, G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom; or a 4-6 membered saturated heterocyclyl group containing one oxygen atom selected from an oxetanyl group and a tetrahydro-2H-pyranyl group.

In another embodiment G₁ typically represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one, two, three or four substituents selected from a C₁-C₄ alkyl group and a halogen atom.

Still in this other embodiment preferably, G₁ represents a C₆₋₂₀ aryl group selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one, two, three or four substituents selected from a C₁-C₂ alkyl group and a halogen atom.

Still in this other embodiment more preferably, G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom.

In a preferred embodiment:
R¹ represents a —COCH₃ group or a -Aa₇₅-Aa₇₄-[L₁]ₘ-[Tag₁]ₙ group;
R² represents a —NH₂ group, or a -Aa₈₄-Aa₈₅-[L₂]ₚ-[Tag₂]_{q} group;
Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond);
Aa₇₅ represents a direct bond, a glutamine, a leucine, a lysine or a valine residue;
(i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond;
L₁ represents a —C(O)—(CH₂)₂—NH— group;
Tag₁ represents a —C(O)—(CH₂)ᵣ—CH₃ group, a —C(O)—(CH₂)₇-((E-CH═CH)—CH₂)₁—(CH₂)₆—CH₃ group, a —C(O)—(CH₂)₇—((Z—CH═CH)—CH₂)₁—(CH₂)₆—CH₃ group or a —C(O)—(CH₂)₇—((Z—CH═CH)—CH₂)₃—CH₃ group, wherein when Aa₇₄ represents a 4-aminoproline residue and m is 0, the Tag₁ group is linked to Aa₇₄ through the 4-amino substituent of the 4-aminoproline residue;
r represents 6 to 20;
Aa₈₄ represents a direct bond, a leucine, a valine or a lysine residue, wherein, when Aa₈₄ is a leucine residue, Aa₈₄ is optionally alkylated with a methyl group on the N at the peptidic bond (i.e. the leucine residue is optionally N-methylated at the peptidic bond);
Aa₈₅ represents a direct bond, a proline, a leucine, a valine, a lysine, or a D-proline residue, wherein when Aa₈₅ is other than a direct bond it is optionally linked to Aa₇₄;
(i) p is 0 and q is 0; or (ii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond;
L₂ represents a —NH—(CH₂)₂—CO— group;
Tag₂ is a peptide containing from 8 to 11 amino acids, wherein at least three of these amino acids are selected from the group consisting of lysine and arginine and the or each carboxy-terminal group of Tag₂ is a —CONH₂ group;
(i) s, t, and u each represent 0; or (ii) s, t, and u each represent 1;
Aa₇₈ represents an unsubstituted alanine, arginine, L-thioproline or glutamic acid residue, or a proline or a phenylalanine residue optionally substituted by one substituent selected from a fluorine atom and amino group, and wherein Aa₇₈ is optionally alkylated with a methyl group on the N at the peptidic bond. (i.e., Aa₇₈ is optionally N-methylated at the peptidic bond); and
G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom; or a 4-6 membered saturated heterocyclyl group containing one oxygen atom selected from an oxetanyl group and a tetrahydro-2H-pyranyl group.

In a more preferred embodiment:
R¹ is selected from:
   —COCH₃;
   —CO—(CH₂)₂—NH—CO—(CH₂)₁₆—CH₃;
   —CO—(CH₂)₂—NH—CO—(CH₂)₆—CH₃;
   -Gln-Pro((4S)—NH—CO—(CH₂)₂₀—CH₃)—;
   -Gln-Pro((4S)—NH—CO—(CH₂)₁₈—CH₃)—;
   -Gln-Pro((4S)—NH—CO—(CH₂)₁₇—CH₃)—;
   -Gln-Pro((4S)—NH—CO—(CH₂)₁₆—CH₃)—;
   -Gln-Pro((4S)—NH—CO—(CH₂)₁₄—CH₃)—;
   -Gln-Pro((4S)—NH—CO—(CH₂)₁₂—CH₃)—;
   -Gln-Pro((4S)—NH—CO—(CH₂)₁₀—CH₃)—;
   -Gln-Pro((4S)—NH—CO—(CH₂)₇-((E-CH═CH)—CH₂)₁—(CH₂)₆—CH₃)—;
   -Gln-Pro((4S)—NH—CO—(CH₂)₇—((Z—CH═CH)—CH₂)₁—(CH₂)₆—CH₃)—;
   -Gln-Pro((4S)—NH—CO—(CH₂)₇—((Z—CH═CH)—CH₂)₃—CH₃)—;
   -Gln-Pro((4S)—NHC(O)CH₃)—;
   -Gln-Leu-H;
   -Gln-Leu-;
   -Gln-Leu-CO—(CH₂)₂—NH—CO—(CH₂)₁₆—CH₃;
   -Gln-Leu-CO—(CH₂)₂—NH—CO—(CH₂)₆—CH₃;
   -Gln-MeLeu-CO—(CH₂)₂—NH—CO—(CH₂)₁₆—CH₃;
   -Gln-Lys-CO—(CH₂)₂—NH—CO—(CH₂)₁₆—CH₃;
   -Gln-Lys(-CO—(CH₂)₂—NH—CO—(CH₂)₁₆—CH₃)—;
   -Gln-Lys(N⁶—CO—(CH₂)₁₆—CH₃)—;
   -Gln-Lys(-CO—(CH₂)₁₆—CH₃)—;
   -Gln-AcPro((4S)—NH—CO—(CH₂)₁₆—CH₃);
   -Gln-Pro-CO—(CH₂)₁₆—CH₃;
   -Leu-Leu-CO—(CH₂)₂—NH—CO—(CH₂)₁₆—CH₃;
   -Leu-Leu-H;
   -Lys-Lys-CO—(CH₂)₂—NH—CO—(CH₂)₁₆—CH₃;
   -Lys-Pro((4S)—NH—CO—(CH₂)₁₆—CH₃)—; and
   -Val-Val-CO—(CH₂)₂—NH—CO—(CH₂)₁₆—CH₃.
R² is selected from;

-NH₂;
-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
-Lys-D-Pro-;
and
-Val-Val-NH₂;

either (i) s, t, and u each represent 0; or (ii) s, t, and u each represent 1;
Aa₇₈ represents an unsubstituted alanine, arginine, L-thioproline or glutamic acid residue, or a proline or a phenylalanine residue optionally substituted by one substituent selected from a fluorine atom and amino group, and wherein Aa₇₈ is optionally alkylated with a methyl group on the N at the peptidic bond; and
G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom; or a 4-6 membered saturated heterocyclyl group containing one oxygen atom selected from an oxetanyl group and a tetrahydro-2H-pyranyl group.

In another preferred embodiment:
R¹ represents a —COCH₃ group or a -Aa₇₅-Aa₇₄-[L₁]ₘ-[Tag₁]ₙ group;
R² represents a —NH₂ group, or a -Aa₈₄-Aa₈₅-[L₂]ₚ-[Tag₂]_{q} group;
Aa₇₄ represents a direct bond, a leucine, a valine, a lysine, a proline, a 4-aminoproline or a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N-methylated at the peptidic bond);
Aa₇₅ represents a direct bond, a glutamine, a leucine, a lysine or a valine residue;
(i) m is 0 and n is 0; or (ii) m is 0 and n is 1; or (iii) m is 1 and n is 1, wherein when m and n each represent 0, and Aa₇₄ is not linked to Aa₈₅, the or each amino-terminal group of R¹ is typically a —NH₂ group and provided that when m and n each represent 0, Aa₇₄ and Aa₇₅ cannot simultaneously be a direct bond;
L₁ represents a —C(O)—(CH₂)₂—NH— group;
Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue;
r represents 6 or 16;
Aa₈₄ represents a direct bond, a leucine, a valine or a lysine residue, wherein, when Aa₈₄ is a leucine residue, Aa₈₄ is optionally alkylated with a methyl group on the N at the peptidic bond (i.e. the leucine residue is optionally N-methylated at the peptidic bond);
Aa₈₅ represents a direct bond, a proline, a leucine, a valine, a lysine, or a D-proline residue, wherein when Aa₈₅ is other than a direct bond it is optionally linked to Aa₇₄;
(i) p is 0 and q is 0; or (ii) p is 1 and q is 1, wherein, when p and q each represent 0 and Aa₇₄ is not linked to Aa₈₅, the or each carboxy-terminal group of R² is a —COOH group or a —CONH₂ group and provided that when p and q each represent 0, Aa₈₄ and Aa₈₅ cannot simultaneously be a direct bond;
L₂ represents a —NH—(CH₂)₂—CO— group;
Tag₂ is a peptide containing from 8 to 11 amino acids, wherein at least three of these amino acids are selected from the group consisting of lysine and arginine and the or each carboxy-terminal group of Tag₂ is a —CONH₂ group;
(i) s, t, and u each represent 0; or (ii) s, t, and u each represent 1;
Aa₇₈ represents an unsubstituted alanine, arginine or glutamic acid residue, or a proline or a phenylalanine residue optionally substituted by one substituent selected from a fluorine atom and amino group, and wherein Aa₇₈ is optionally alkylated with a methyl group on the N at the peptidic bond. (i.e., Aa₇₈ is optionally N-methylated at the peptidic bond); and
G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom.

Still in this other preferred embodiment:
R¹ is selected from:
   —COCH₃
   —CO—(CH₂)₂—NH—CO—(CH₂)₁₆—CH₃;
   —CO—(CH₂)₂—NH—CO—(CH₂)₆—CH₃;
   -Gln-Pro((4S)—NH—CO—(CH₂)₁₆—CH₃)—;
   -Gln-Pro((4S)—NHC(O)CH₃)—;
   -Gln-Leu-H;
   -Gln-Leu-;
   -Gln-Leu-CO—(CH₂)₂—NH—CO—(CH₂)₁₆—CH₃;
   -Gln-Leu-CO—(CH₂)₂—NH—CO—(CH₂)₆—CH₃;
   -Gln-MeLeu-CO—(CH₂)₂—NH—CO—(CH₂)₁₆—CH₃;
   -Gln-Lys-CO—(CH₂)₂—NH—CO—(CH₂)₁₆—CH₃;
   -Gln-Lys(-CO—(CH₂)₂—NH—CO—(CH₂)₁₆—CH₃)—;
   -Gln-AcPro((4S)—NH—CO—(CH₂)₁₆—CH₃);
   -Gln-Pro-CO—(CH₂)₁₆—CH₃;
   -Leu-Leu-CO—(CH₂)₂—NH—CO—(CH₂)₁₆—CH₃;
   -Leu-Leu-H;
   -Lys-Lys-CO—(CH₂)₂—NH—CO—(CH₂)₁₆—CH₃; and
   -Val-Val-CO—(CH₂)₂—NH—CO—(CH₂)₁₆—CH₃;
R² is selected from;

-NH₂;
-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
and
-Val-Val-NH₂;

either (i) s, t, and u each represent 0; or (ii) s, t, and u each represent 1;
Aa₇₈ represents an unsubstituted alanine, arginine or glutamic acid residue, or a proline or a phenylalanine residue optionally substituted by one substituent selected from a fluorine atom and amino group, and wherein Aa₇₈ is optionally alkylated with a methyl group on the N at the peptidic bond; and
G₁ represents an unsubstituted 6-10 membered heteroaryl group selected from a pyridine group, an indolyl group and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom.

As used herein, in the case of the R¹ groups -Aa₇₅-Aa₇₄-[L₁]ₘ-[Tag₁]ₙ wherein m and n each represent 0, Aa₇₅ and Aa₇₄ are not a direct bond and Aa₇₄ is not linked to Aa₈₅, the amino-terminal group of R¹ is typically a —NH₂ or a —NHCOCH₃ group and is represented by a —H term or a —COCH₃ term respectively at the end of the sequence. More typically the amino-terminal group of R¹ is a —NH₂ and is represented by a —H term at the end of the sequence.

As used herein, in the case of the R² groups -Aa₈₄-Aa₈₅-[L₂]ₚ-[Tag₂]_{q} wherein p and q each represent 0, Aa₈₄ and Aa₈₅ are not a direct bond and Aa₈₅ is not linked to Aa₇₄, the carboxy-terminal group of R² is typically a —COOH or a —CONH₂ group and is represented by a —OH term or a —NH₂ term respectively at the end of the sequence.

As used herein, in the case of the R¹ groups -Aa₇₅-Aa₇₄-[L₁]ₘ-[Tag₁]ₙ wherein Aa₇₄ residues are linked to Aa₈₅ residues by a peptidic bond, a “-” is depicted at the end of the corresponding sequence. This is the case of:
-Gln-Pro((4S)—NH—CO—(CH₂)₁₆—CH₃)—;
-Gln-Pro((4S)—NHC(O)CH₃)—;
-Gln-Leu-;
-Gln-Lys(-CO—(CH₂)₂—NH—CO—(CH₂)₁₆—CH₃)—;
-Gln-Pro((4S)—NH—CO—(CH₂)₂₀—CH₃)—;
   -Gln-Pro((4S)—NH—CO—(CH₂)₁₈—CH₃)—;
-Gln-Pro((4S)—NH—CO—(CH₂)₁₇—CH₃)—;
-Gln-Pro((4S)—NH—CO—(CH₂)₁₄—CH₃)—;
-Gln-Pro((4S)—NH—CO—(CH₂)₁₂—CH₃)—;
-Gln-Pro((4S)—NH—CO—(CH₂)₁₀—CH₃)—;
   -Gln-Pro((4S)—NH—CO—(CH₂)₇-((E-CH═CH)—CH₂)₁—(CH₂)₆—CH₃)—;
-Gln-Pro((4S)—NH—CO—(CH₂)₇—((Z—CH═CH)—CH₂)₁—(CH₂)₆—CH₃)—;
-Gln-Pro((4S)—NH—CO—(CH₂)₇—((Z—CH═CH)—CH₂)₃—CH₃)—;
-Gln-Lys(N⁶—CO—(CH₂)₁₆—CH₃)—;
-Gln-Lys(-CO—(CH₂)₁₆—CH₃)—;
   -Lys-Pro((4S)—NH—CO—(CH₂)₁₆—CH₃)—;

wherein the corresponding Aa₇₄ residues Pro, Leu, and Lys are linked to Aa₈₅ by a peptidic bond.

As used herein, in the case of the R² groups -Aa₈₄-Aa₈₅-[L₂]ₚ-[Tag₂]_{q} wherein Aa₈₅ residues are linked to Aa₇₄ residues by a peptidic bond, a “-” is depicted at the end of the corresponding sequence. This is the case of:
-Leu-D-Pro-;
-Leu-Pro-;
-MeLeu-D-Pro-;
-Lys-D-Pro-;

wherein the corresponding Aa₈₅ residues D-Pro and Pro are linked to Aa₇₄ by a peptidic bond.

As used herein, wherein the carboxy-terminal group of Tag₂ peptide is a —CONH₂ group it is represented by a —NH₂ term at the end of the sequence.

In a preferred embodiment, the peptidic compound of the invention is a compound of formula (IA)′, or a pharmaceutically acceptable salt, or solvate, or N-oxide, or stereoisomer thereof:

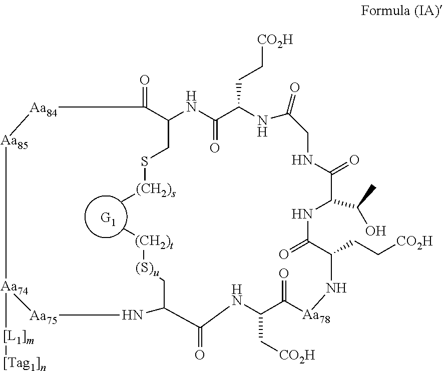

wherein
Aa₇₄ represents a leucine, a lysine, a 4-aminoproline or a 4-acetaminoproline residue;
Aa₇₅ represents a glutamine or a lysine residue;
m and n each independently represent an integer selected from 0 and 1;
when m is 1 and n is 1 L₁ represents a —C(O)—(CH₂)₍₁₋₄₎—NH— group and when m is 1 and n is 0 L₁ represents a —C(O)—(CH₂)₍₁₋₄₎—NH₂ group;
Tag₁ represents a —C(O)—(CH₂)ᵣ—CH₃ group, a —C(O)—(CH₂)₇-((E-CH═CH)—CH₂)₁—(CH₂)₆—CH₃ group, a —C(O)—(CH₂)₇—((Z—CH═CH)—CH₂)₁—(CH₂)₆—CH₃ group or a —C(O)—(CH₂)₇—((Z—CH═CH)—CH₂)₃—CH₃ group, wherein when Aa₇₄ represents a 4-aminoproline residue and m is 0, the Tag₁ group is linked to Aa₇₄ through the 4-amino substituent of the 4-aminoproline residue;
r represents an integer selected from 6 to 20;
Aa₈₄ represents a leucine residue or a lysine residue, wherein the leucine residue is optionally N-methylated at the peptidic bond;
Aa₈₅ represents a proline or a D-proline residue;
s represents 0 or 1;
t represents 0 or 1;
u represents 0 or 1;
Aa₇₈ represents a proline, a L-thioproline, an alanine, an arginine, or a glutamic acid residue, wherein the proline, L-thioproline, alanine, arginine or glutamic acid residue is optionally substituted by one or two substituents selected from a halogen atom and amino group; and

G₁ represents a phenyl group, a pyridine group or an indolyl group; wherein the phenyl, pyridine and indolyl groups are optionally substituted by one, two, three or four substituents selected from a C₁-C₄ alkyl group and a halogen atom; or a 4-6 membered saturated heterocyclyl group containing one oxygen atom selected from an oxetanyl group and a tetrahydro-2H-pyranyl group.

In a more preferred embodiment in the peptidic compound of formula (IA)′:
Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue;
r represents an integer selected from 6 to 20.

In another preferred embodiment, the peptidic compound of the invention is a compound of formula (IA), or a pharmaceutically acceptable salt, or solvate, or N-oxide, or stereoisomer thereof:

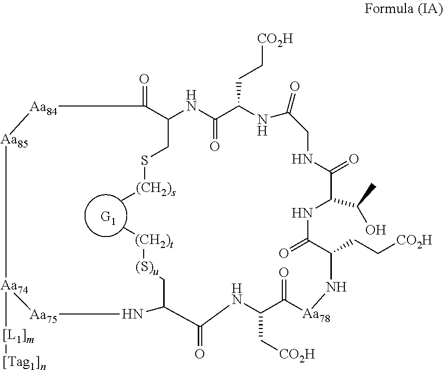

wherein
Aa₇₄ represents a leucine, a lysine, a 4-aminoproline or a 4-acetaminoproline residue;
Aa₇₅ represents a glutamine residue;
m and n each independently represent an integer selected from 0 and 1;
when m is 1 and n is 1 L₁ represents a —C(O)—(CH₂)₍₁₋₄₎—NH— group and when m is 1 and n is 0 L₁ represents a —C(O)—(CH₂)₍₁₋₄₎—NH₂ group;
Tag₁ represents a —C(O)—(CH₂)ᵣ—CH₃ group, wherein when Aa₇₄ represents a 4-aminoproline residue and m is 0, the Tag₁ group is linked to Aa₇₄ through the 4-amino substituent of the 4-aminoproline residue;
r represents an integer selected from 6 to 18;
Aa₈₄ represents a leucine residue, wherein the leucine residue is optionally N-methylated at the peptidic bond;
Aa₈₅ represents a proline or a D-proline residue;
s represents 0 or 1;
t represents 0 or 1;
u represents 0 or 1;
Aa₇₈ represents a proline, an alanine, an arginine or a glutamic acid residue, wherein the proline, alanine, arginine or glutamic acid residue is optionally substituted by one or two substituents selected from a halogen atom and amino group; and
G₁ represents a phenyl group or an indolyl group; wherein the phenyl and indolyl groups are optionally substituted by one, two, three or four substituents selected from a C₁-C₄ alkyl group and a halogen atom.

In a particularly preferred embodiment, in the formula (IA)′, the moiety

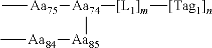

is selected from:

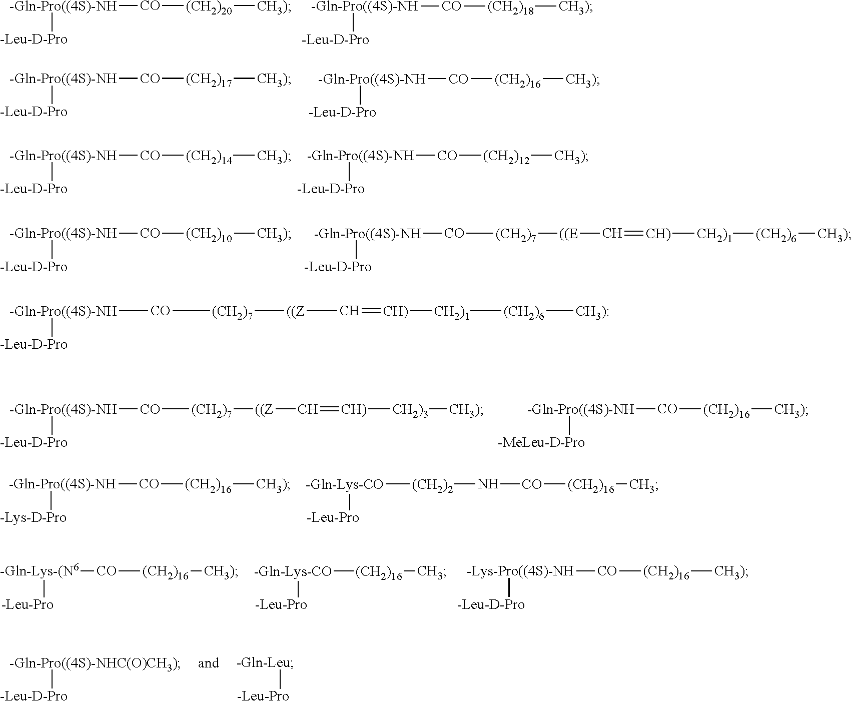

s represents 0 or 1;
t represents 0 or 1;
u represents 0 or 1;
Aa₇₈ represents a proline, a L-thioproline, an alanine, an arginine or a glutamic acid residue, wherein the proline, L-thioproline, alanine, arginine or glutamic acid residue is optionally substituted by one or two substituents selected from a halogen atom and amino group; and
G₁ represents a phenyl group, a pyridine group or an indolyl group; wherein the phenyl, pyridine and indolyl groups are optionally substituted by one, two, three or four substituents selected from a C₁-C₄ alkyl group and a halogen atom; or a 4-6 membered saturated heterocyclyl group containing one oxygen atom selected from an oxetanyl group and a tetrahydro-2H-pyranyl group.

In another particularly preferred embodiment, in the formula (IA), the moiety

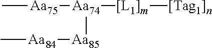

is selected from:

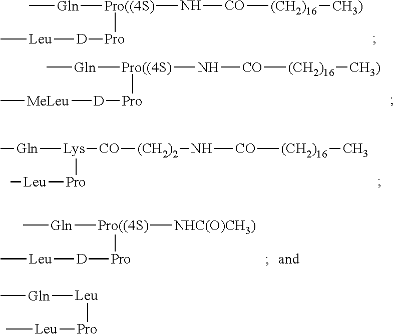

s represents 0 or 1;
t represents 0 or 1;
u represents 0 or 1;
Aa₇₈ represents a proline, an alanine, an arginine or a glutamic acid residue, wherein the proline, alanine, arginine or glutamic acid residue is optionally substituted by one or two substituents selected from a halogen atom and amino group; and
G₁ represents a phenyl group or an indolyl group; wherein the phenyl and indolyl groups are optionally substituted by one, two, three or four substituents selected from a C₁-C₄ alkyl group and a halogen atom.

The peptidic compounds of the invention are cyclic or bicyclic. Particular sequences of cyclic or bicyclic peptidic compounds of the invention include:

(SEQ ID NO: 1)
H-Leu-Gln-Trp(indol-2-yl-&¹)-Asp-Glu-Glu-Thr-Gly-Glu-Cys(&¹)-Leu-Pro-OH
(SEQ ID NO: 2)
{[H-Leu-Gln-Cys(&¹)-Asp-Glu-Glu-Thr-Gly-Glu-Cys(&²)-Leu-Pro-OH][&¹(1,4-
phenylenediyl)dimethylene&²]}
(SEQ ID NO: 3)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]}
(SEQ ID NO: 4)
{[&¹Leu-Gln-Cys(&²)-Asp-Glu-Glu-Thr-Gly-Glu-Cys(&³)-Leu-Pro&¹][&²(1,3-
phenylenediyl)dimethylene&³]}
(SEQ ID NO: 5)
{[&¹Leu-Gln-Cys(&²)-Asp-Glu-Glu-Thr-Gly-Glu-Cys(&³⁾-Leu-Pro&¹][&²(1,4-
phenylenediyl)dimethylene&³]}
(SEQ ID NO: 6)
{[Acetyl-Cys(&¹)-Asp-Glu-Glu-Thr-Gly-Glu-Cys(&²)-NH₂][&¹(1,3-
phenylenediyl)dimethylene&²]}
(SEQ ID NO: 7)
Acetyl-Trp(indol-2-yl-&¹)-Asp-Glu-Glu-Thr-Gly-Glu-Cys(&¹)-NH₂
(SEQ ID NO: 8)
H-Leu-Gln-Trp(indol-2-yl-&¹)-Asp-Glu-Glu-Thr-Gly-Glu-Cys(&¹)-Leu-Pro-βAla-Arg-
Lys-Lys-Arg-Arg-Gln-Arg-Arg-Arg-NH₂
(SEQ ID NO: 9)
{[H-Leu-Gln-Cys(&¹)-Asp-Glu-Glu-Thr-Gly-Glu-Cys(&²)-Leu-Pro-βAla-Arg-Lys-
Lys-Arg-Arg-Gln-Arg-Arg-Arg-NH₂][&¹(1,3-phenylenediyl)dimethylene&²]}
(SEQ ID NO: 10)
Acetyl-Phe(p-&¹)-Asp-Glu-Glu-Thr-Gly-Glu-Cys(&¹)-NH₂
(SEQ ID NO: 11)
&¹Leu-Gln-Trp(indol-2-yl-&²)-Asp-Glu-Glu-Thr-Gly-Glu-Cys(&²)-Leu-Pro&¹
(SEQ ID NO: 12)
{[Acetyl-Cys(&¹)-Asp-Glu-Glu-Thr-Gly-Glu-Cys(&²)-NH₂][&¹(1,2-
phenylenediyl)dimethylene&²]}
(SEQ ID NO: 13)
{[H-Leu-Gln-Cys(&¹)-Asp-Glu-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 14)
{[H-Leu-Gln-Cys(&¹)-Asp-Ala-Glu-Thr-Gly-Glu-Cys(&²)-Leu-Pro-OH][&¹(1,3-
phenylenediyl)dimethylene&²]}
(SEQ ID NO: 15)
{[H-Leu-Gln-Cys(&¹)-Asp-Ala-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 16)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,3-phenylenediyl)dimethylene&²]}
(SEQ ID NO: 17)
{[&¹Leu-Gln-Cys(&²)-Asp-Ala-Glu-Thr-Gly-Glu-Cys(&³)-Leu-Pro&¹][&²(1,3-
phenylenediyl)dimethylene&²]}
(SEQ ID NO: 18)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]}
(SEQ ID NO: 19)
{[H-Leu-Gln-Cys(&¹)-Asp-Ala-Glu-Thr-Gly-Glu-Cys(&²)-Leu-Pro-OH][&¹(1,2-
phenylenediyl)dimethylene&²]}
(SEQ ID NO: 20)
{[Acetyl-Cys(&¹)-Asp-Ala-Glu-Thr-Gly-Glu-Cys(&²)-NH₂]-[&¹(1,2-
phenylenediyl)dimethylene&²]}
(SEQ ID NO: 21)
{[Acetyl-Cys(&¹)-Asp-Ala-Glu-Thr-Gly-Glu-Cys(&²)-βAla-Arg-Lys-Lys-Arg-Arg-
Gln-Arg-Arg-Arg-NH₂][&¹(1,2-phenylenediyl)dimethylene&²]}
(SEQ ID NO: 22)
Acetyl-Phe(m-&¹)-Asp-Glu-Glu-Thr-Gly-Glu-Cys(&¹)-NH₂
(SEQ ID NO: 23)
Acetyl-Phe(o-&¹)-Asp-Glu-Glu-Thr-Gly-Glu-Cys(&¹)-NH₂
(SEQ ID NO: 24)
{[Acetyl-Cys(&¹)-Asp-Glu-Glu-Thr-Gly-Glu-Cys(&²)-NH₂][&¹(1,1′-biphenyl)2,2′-
diyldimethylene&²]}
(SEQ ID NO: 25)
{[Acetyl-Cys(&¹)-Asp-Glu-Glu-Thr-Gly-Glu-Cys(&²)-NH₂][&¹(1,1′-
binaphthalene)2,2′-diyldimethylene&²]}
(SEQ ID NO: 26)
{[Acetyl-Cys(&¹)-Asp-Glu-Glu-Thr-Gly-Glu-Cys(&²)-NH₂][&¹(quinoxaline)2,3-
diyldimethylene&²]}
(SEQ ID NO: 27)
{[Acetyl-Cys(&¹)-Asp-Glu-Glu-Thr-Gly-Glu-Cys(&²)-NH₂][&¹(naphthalene)2,3-
diyldimethylene&²]}
(SEQ ID NO: 28)
{[Stearyl-βAla-Cys(&¹)-Asp-Ala-Glu-Thr-Gly-Glu-Cys(&²)-NH₂][&¹(1,2-
phenylenediyl)dimethylene&²]}
(SEQ ID NO: 29)
{[Stearyl-βAla-Leu-Gln-Cys(&¹)-Asp-Ala-Glu-Thr-Gly-Glu-Cys(&²)-Leu-Pro-
OH][&¹(1,2-phenylenediyl)dimethylene&²]}
(SEQ ID NO: 30)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(naphtalene)2,3-diyldimethylene&²]}
(SEQ ID NO: 31)
{[Stearyl-βAla-Cys(&¹)-Asp-Pro-Glu-Thr-Gly-Glu-Cys(&2)-NH₂][&¹(1,2-
phenylenediyl)dimethylene&²]}
(SEQ ID NO: 32)
{[Stearyl-βAla-Cys(&¹)-Asp-Pro-Glu-Thr-Gly-Glu-Cys(&²)-
NH₂][&¹(naphthalene)2,3-diyldimethylene&²]}
(SEQ ID NO: 33)
{[Stearyl-βAla-Cys(&¹)-Asp-Pro-Glu-Thr-Gly-Glu-Cys(&²)-NH₂][&¹(1,3-
phenylenediyl)dimethylene&²]}
(SEQ ID NO: 34)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]}
(SEQ ID NO: 35)
{[Stearyl-βAla-Leu-Gln-Cys(&¹)-Asp-Pro-Glu-Thr-Gly-Glu-Cys(&²)-Leu-Pro-
OH][&¹(1,2-phenylenediyl)dimethylene&²]}
(SEQ ID NO: 36)
{[Stearyl-βAla-Leu-Gln-Cys(&¹)-Asp-Pro-Glu-Thr-Gly-Glu-Cys(&²)-Leu-Pro-OH]
[&¹(naphthalene)2,3-diyldimethylene&²]}
(SEQ ID NO: 37)
{[Stearyl-βAla-Leu-Gln-Cys(&¹)-Asp-Pro-Glu-Thr-Gly-Glu-Cys(&²)-Leu-Pro-
OH][&¹(1,3-phenylenediyl)dimethylene&²]}
(SEQ ID NO: 38)
{[Stearyl-βAla-Leu-Gln-Cys(&¹)-Asp-Ala-Glu-Thr-Gly-Glu-Cys(&²)-Leu-Pro-OH]
[&¹(naphthalene)2,3-diyldimethylene&²]}
(SEQ ID NO: 39)
{[Stearyl-βAla-Leu-Gln-Cys(&¹)-Asp-Ala-Glu-Thr-Gly-Glu-Cys(&²)-Leu-Pro-
OH][&¹(1,3-phenylenediyl)dimethylene&²]}
(SEQ ID NO: 40)
Stearyl-βAla-Leu-Gln-Phe(m-&¹)-Asp-Pro-Glu-Thr-Gly-Glu-Cys(&¹)-Leu-Pro-OH
(SEQ ID NO: 41)
Stearyl-βAla-Phe(m-&¹)-Asp-Pro-Glu-Thr-Gly-Glu-Cys(&¹)-NH₂
(SEQ ID NO: 42)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(naphthalene)2,3-diyldimethylene&²]}
(SEQ ID NO: 43)
{[Acetyl-Cys(&¹)-Asp-MeAla-Glu-Thr-Gly-Glu-Cys(&²)-NH₂][&¹(1,2-
phenylenediyl)dimethylene&²]}
(SEQ ID NO: 44)
{[Stearyl-βAla-Leu-Leu-Cys(&¹)-Asp-Pro-Glu-Thr-Gly-Glu-Cys(&²)-Leu-Leu-
NH2][&¹(1,2-phenylenediyl)dimethylene&²]}
(SEQ ID NO: 45)
{[Stearyl-βAla-MeLeu-Gln-Cys(&¹)-Asp-Pro-Glu-Thr-Gly-Glu-Cys(&²)-Leu-Pro-
NH2][&¹(1,2-phenylenediyl)dimethylene&²]}
(SEQ ID NO: 46)
{[Stearyl-βAla-Leu-Gln-Cys(&¹)-Asp-Pro-Glu-Thr-Gly-Glu-Cys(&²)-MeLeu-Pro-
NH₂][&¹(1,2-phenylenediyl)dimethylene&²]}
(SEQ ID NO: 47)
{[Stearyl-βAla-Lys-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 48)
{[Stearyl-βAla-Leu-Gln-Cys(&¹)-Asp-Pro-Glu-Thr-Gly-Glu-Cys(&²)-Leu-Pro-
NH₂][&¹(1,2-phenylenediyl)dimethylene&³]}
(SEQ ID NO: 49)
{[Stearyl-βAla-Lys(&¹)-Gln-Cys(&2)-Asp-Pro-Glu-Thr-Gly-Glu-Cys(&³)-Leu-
Pro&¹][&²(1,2-phenylenediyl)dimethylene&³]}
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 51)
{[Stearyl-βAla-Leu-Gln-Cys(&¹)-Asp-Pro-Glu-Thr-Gly-Glu-Cys(&²)-Leu-Pro-
NH₂][&¹(1,3,5-trimethylbenzene)2,4-diyldimethylene&²]}
(SEQ ID NO: 52)
{[Stearyl-βAla-Val-Val-Cys(&¹)-Asp-Pro-Glu-Thr-Gly-Glu-Cys(&²)-Val-Val-
NH₂][&¹(1,2-phenylenediyl)dimethylene&²]}
(SEQ ID NO: 53)
{[Stearyl-βAla-Lys-Lys-Cys(&¹)-Asp-Pro-Glu-Thr-Gly-Glu-Cys(&²)-Lys-Lys-
NH₂][&¹(1,2-phenylenediyl)dimethylene&²]}
(SEQ ID NO: 54)
{[Stearyl-βAla-Leu-Gln-Cys(&¹)-Asp-Pro-Glu-Thr-Gly-Glu-Cys(&²)-Leu-Pro-βAla-
Arg-Lys-Lys-Arg-Arg-Gln-Arg-Arg-Arg-NH₂][&¹(1,2-phenylenediyl)dimethylene&²]}
(SEQ ID NO: 55)
{[Stearyl-βAla-Leu-Gln-Cys(&¹)-Asp-Pro-Glu-Thr-Gly-Glu-Cys(&²)-Leu-Pro-
NH₂][&¹(3,4,5,6-tetrafluorobenzene)1,2-diyldimethylene&²]}
(SEQ ID NO: 56)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(3,4,5,6-tetrafluorobenzene)1,2-diyldimethylene&²]}
(SEQ ID NO: 57)
{[Stearyl-βAla-Lys-Lys-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 58)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(naphthalene)2,3-diyldimethylene&²]}
(SEQ ID NO: 59)
{[H-Leu-Leu-Cys(&¹)-Asp-Pro-Glu-Thr-Gly-Glu-Cys(&²)-Leu-Leu-βAla-Arg-Lys-Lys-
Arg-Arg-Gln-Arg-Arg-Arg-NH₂][&¹(1,3,5-trimethylbenzene)2,4-diyldimethylene&²]}
(SEQ ID NO: 60)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(1,2-phenylenediyl)dimethylene&²]}
(SEQ ID NO: 61)
{[Stearyl-βAla-Leu-Gln-Cys(&¹)-Asp-Pro-Glu-Thr-Gly-Glu-Cys(&²)-Leu-Pro
NH₂][&¹(quinoxaline)2,3-diyldimethylene&²]}
(SEQ ID NO: 62)
{[Stearyl-βAla-Leu-Gln-Cys(&¹)-Asp-Pro-Glu-Thr-Gly-Glu-Cys(&²)-Leu-Pro
NH₂][&¹(pyridine)2,6-diyldimethylene&²]}
(SEQ ID NO: 63)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(1,1′-binaphthalene)2,2′-diyldimethylene&²]}
(SEQ ID NO: 64)
{[&¹Pro((4S)-NH-Acetyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 65)
{[Stearyl-βAla-Leu-Gln-Cys(&¹)-Asp-Pro-Glu-Thr-Gly-Glu-Cys(&²)-Leu-Pro
NH₂][&¹(pyridine)3,5-diyldimethylene&²]}
(SEQ ID NO: 66)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,1′-binaphthalene)2,2′-diyldimethylene&²]}
(SEQ ID NO: 67)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,3,5-trimethylbenzene)2,4-diyldimethylene&²]}
(SEQ ID NO: 68)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]}
(SEQ ID NO: 69)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]}
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&³]}
(SEQ ID NO: 71)
{[H-Leu-Gln-Cys(&¹)-Asp-Phe(4-F)-Glu-Thr-Gly-Glu-Cys(&²)-Leu-Pro-βAla-Arg-
Lys-Lys-Arg-Arg-Gln-Arg-Arg-Arg-NH₂][&¹(1,2-phenylenediyl)dimethylene&²]}
(SEQ ID NO: 72)
{[&¹Pro((4S)-NH-Stearyl)-Gln-Cys(&²)-Asp-Pro((4S)-F)-Glu-Thr-Gly-Glu-Cys(&³)-
Leu-D-Pro&¹][&²(1,2-phenylenediyl)dimethylene&³]}
(SEQ ID NO: 73)
{[&¹Pro((4S)-NH-Stearyl)-Gln-Cys(&²)-Asp-Pro((4S)-NH₂)-Glu-Thr-Gly-Glu-
Cys(&³)-Leu-D-Pro&¹][&²(1,2-phenylenediyl)dimethylene&³]}
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]}
(SEQ ID NO: 75)
{[Acetyl-Pro((4S)-NH-Stearyl)-Gln-Cys(&¹)-Asp-Pro-Glu-Thr-Gly-Glu-Cys(&²)-Leu-
D-Pro-NH₂][&¹(1,2-phenylenediyl)dimethylene²]}
(SEQ ID NO: 76)
{[Stearyl-Pro-Gln-Cys(&¹)-Asp-Pro-Glu-Thr-Gly-Glu-Cys(&²)-Leu-D-Pro-
NH2][&¹(1,2-phenylenediyl)dimethylene²]}
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&³]}
(SEQ ID NO: 78)
{[&¹Pro((4S)-NH-Stearyl)-Gln-Cys(&²)-Asp-Thz-Glu-Thr-Gly-Glu-Cys(&³)-Leu-D-
Pro&¹][&²(1,2-phenylenediyl)dimethylene&³]}
(SEQ ID NO: 79)
{[&¹Pro((4S)-NH-Stearyl)-Gln-Cys(&²)-Asp-Pro-Glu-Thr-Gly-Glu-Cys(&³)-Leu-D-
Pro&¹][&²(3,3-oxetanediyl)dimethylene&³]}
(SEQ ID NO: 80)
{[&¹Pro((4S)-NH-Miristoyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 81)
{[&¹Pro((4S)-NH-Stearyl)-Lys-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 82)
{[&¹Pro((4S)-NH-Stearyl)-Gln-Cys(&²)-Asp-Pro-Glu-Thr-Gly-Glu-Cys(&³)-Lys-D-
Pro&¹][&²(1,2-phenylenediyl)dimethylene&³]}
(SEQ ID NO: 83)
{[&¹Pro((4S)-NH-Palmitoyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 84)
{[&¹Pro((4S)-NH-Stearyl)-Gln-Cys(&²)-Asp-Pro-Glu-Thr-Gly-Glu-Cys(&³)-Leu-D-
Pro&¹][&¹(pyridine)3,5-diyldimethylene&²]}
(SEQ ID NO: 85)
{[&¹Pro((4S)-NH-Lauryl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 86)
{[&¹Pro((4S)-NH-α-linolenyl)-Gln-Cys(&²)-Asp-Pro-Glu-Thr-Gly-Glu-Cys(&³)-Leu-
D-Pro&¹][&²(1,2-phenylenediyl)dimethylene&³]}
(SEQ ID NO: 87)
{[&¹Pro((4S)-NH-elaidyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 88)
{[&¹Pro((4S)-NH-oleyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 89)
{[&¹Pro((4S)-NH-behenyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 90)
{[&¹Pro((4S)-NH-arachidyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 91)
{[&¹Lys(N6-Stearyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 92)
{[Stearyl-Lys(&¹)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 93)
{[&¹Pro((4S)-NH-Arachidyl)-Gln-Cys(&²)-Asp-Thz-Glu-Thr-Gly-Glu-Cys(&³)-Leu-D-
Pro&¹][&²(1,2-phenylenediyl)dimethylene&³]}
(SEQ ID NO: 94)
{[&¹Pro((4S)-NH-Arachidyl)-Gln-Cys(&²)-Asp-Thz-Glu-Thr-Gly-Glu-Cys(&³)-Leu-D-
Pro&¹][&²(3,3-oxetanediyl)dimethylene&³]}
(SEQ ID NO: 95)
{[&¹Pro((4S)-NH-Arachidyl)-Gln-Cys(&²)-Asp-Pro-Glu-Thr-Gly-Glu-Cys(&³)-Leu-D-
Pro&¹][&²(3,3-oxetanediyl)dimethylene&³]}
(SEQ ID NO: 96)
{[&¹Pro((4S)-NH-Stearyl)-Gln-Cys(&²)-Asp-Thz-Glu-Thr-Gly-Glu-Cys(&³)-Leu-D-
Pro&¹][&²(1,3-phenylenediyl)dimethylene&³]}
(SEQ ID NO: 97)
{[&¹Pro((4S)-NH-Stearyl)-Gln-Cys(&²)-Asp-Thz-Glu-Thr-Gly-Glu-Cys(&³)-Leu-D-
Pro&1][&²(3,3-oxetanediyl)dimethylene&³]}
(SEQ ID NO: 98)
{[&¹Pro((4S)-NH-Nonadecanoyl)-Gln-Cys(&²)-Asp-Pro-Glu-Thr-Gly-Glu-Cys(&³)-
Leu-D-Pro&¹][&²(1,2-phenylenediyl)dimethylene&³]}
(SEQ ID NO: 99)
{[Stearyl-βAla-Leu-Leu-Cys(&¹)-Asp-Pro-Glu-Thr-Gly-Glu-Cys(&²)-Leu-Leu-
NH₂][&¹(3,3-oxetanediyl)dimethylene&²]}
(SEQ ID NO: 100)
{[&¹Pro((4S)-NH-Stearyl)-Gln-Cys(&²)-Asp-Pro-Glu-Thr-Gly-Glu-Cys(&³)-Leu-D-
Pro&¹][&²(tetrahydro-2H-pyran-4,4-diyl)dimethylene&³]}

or a pharmaceutically acceptable salt, or solvate, or N-oxide, or stereoisomer thereof.

Particular preferred sequences of cyclic and bicyclic peptides of the compound of the present invention include:

(SEQ ID NO: 37)
{[Stearyl-βAla-Leu-Gln-Cys(&¹)-Asp-Pro-Glu-Thr-Gly-Glu-Cys(&²)-Leu-Pro-
OH][&¹(1,3-phenylenediyl)dimethylene&²]}
(SEQ ID NO: 47)
{[Stearyl-βAla-Lys-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 49)
{[Stearyl-βAla-Lys(&¹)-Gln-Cys(&²)-Asp-Pro-Glu-Thr-Gly-Glu-Cys(&³)-Leu-
Pro&¹][&²(1,2-phenylenediyl)dimethylene&³]}
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 62)
{[Stearyl-βAla-Leu-Gln-Cys(&¹)-Asp-Pro-Glu-Thr-Gly-Glu-Cys(&²)-Leu-Pro-
NH₂][&¹(pyridine)2,6-diyldimethylene&²]}
(SEQ ID NO: 65)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]}
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&³]}
(SEQ ID NO: 72)
{[&¹Pro((4S)-NH-Stearyl)-Gln-Cys(&²)-Asp-Pro((4S)-F)-Glu-Thr-Gly-Glu-Cys(&³)-
Leu-D-Pro&¹][&²(1,2-phenylenediyl)dimethylene&³]}
(SEQ ID NO: 73)
{[&¹Pro((4S)-NH-Stearyl)-Gln-Cys(&²)-Asp-Pro((4S)-NH₂)-Glu-Thr-Gly-Glu-
Cys(&³)-Leu-D-Pro&¹][&²(1,2-phenylenediyl)dimethylene&³]}
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]}
(SEQ ID NO: 76)
{[Stearyl-Pro-Gln-Cys(&¹)-Asp-Pro-Glu-Thr-Gly-Glu-Cys(&²)-Leu-D-Pro-
NH₂][&¹(1,2-phenylenediyl)dimethylene&²]}
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&²]}
(SEQ ID NO: 78)
{[&¹Pro((4S)-NH-Stearyl)-Gln-Cys(&²)-Asp-Thz-Glu-Thr-Gly-Glu-Cys(&³)-Leu-D-
Pro&¹][&²(1,2-phenylenediyl)dimethylene&³]}
(SEQ ID NO: 79)
{[&¹Pro((4S)-NH-Stearyl)-Gln-Cys(&²)-Asp-Pro-Glu-Thr-Gly-Glu-Cys(&³)-Leu-D-
Pro&¹][&²(3,3-oxetanediyl)dimethylene&³]}
(SEQ ID NO: 89)
{[&¹Pro((4S)-NH-behenyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 90)
{[&¹Pro((4S)-NH-arachidyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 93)
{[&¹Pro((4S)-NH-Arachidyl)-Gln-Cys(&²)-Asp-Thz-Glu-Thr-Gly-Glu-Cys(&³)-Leu-D-
Pro&¹][&²(1,2-phenylenediyl)dimethylene&³]}
(SEQ ID NO: 94)
{[&¹Pro((4S)-NH-Arachidyl)-Gln-Cys(&²)-Asp-Thz-Glu-Thr-Gly-Glu-Cys(&³)-Leu-D-
Pro&¹][&²(3,3-oxetanediyl)dimethylene&³]}
(SEQ ID NO: 95)
{[&¹Pro((4S)-NH-Arachidyl)-Gln-Cys(&²)-Asp-Pro-Glu-Thr-Gly-Glu-Cys(&³)-Leu-D-
Pro&¹][&²(3,3-oxetanediyl)dimethylene&³]}
(SEQ ID NO: 96)
{[&¹Pro((4S)-NH-Stearyl)-Gln-Cys(&²)-Asp-Thz-Glu-Thr-Gly-Glu-Cys(&³)-Leu-D-
Pro&¹][&²(1,3-phenylenediyl)dimethylene&³]}
(SEQ ID NO: 97)
{[&¹Pro((4S)-NH-Stearyl)-Gln-Cys(&²)-Asp-Thz-Glu-Thr-Gly-Glu-Cys(&³)-Leu-D-
Pro&¹][&²(3,3-oxetanediyl)dimethylene&³]}

or a pharmaceutically acceptable salt, or solvate, or N-oxide, or stereoisomer thereof.

In another embodiment particular sequences of cyclic or bicyclic peptidic compounds of the invention include:

(SEQ ID NO: 1)
H-Leu-Gln-Trp(indol-2-yl-&¹)-Asp-Glu-Glu-Thr-Gly-Glu-Cys(&¹)-Leu-Pro-OH
(SEQ ID NO: 2)
{[H-Leu-Gln-Cys(&¹)-Asp-Glu-Glu-Thr-Gly-Glu-Cys(&²)-Leu-Pro-OH][&¹(1,4-
phenylenediyl)dimethylene&²]}
(SEQ ID NO: 3)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]}
(SEQ ID NO: 4)
{[H-Leu-Gln-Cys(&²)-Asp-Glu-Glu-Thr-Gly-Glu-Cys(&³)-Leu-Pro&¹][&²(1,3-
phenylenediyl)dimethylene&³]}
(SEQ ID NO: 5)
{[&¹Leu-Gln-Cys(&²)-Asp-Glu-Glu-Thr-Gly-Glu-Cys(&³)-Leu-Pro&¹][&²(1,4-
phenylenediyl)dimethylene&³]}
(SEQ ID NO: 6)
{[Acetyl-Cys(&¹)-Asp-Glu-Glu-Thr-Gly-Glu-Cys(&²)-NH_{2]}[&¹(1,3-
phenylenediyl)dimethylene&²]}
(SEQ ID NO: 7)
Acetyl-Trp(indol-2-yl-&¹)-Asp-Glu-Glu-Thr-Gly-Glu-Cys(&¹)-NH₂
(SEQ ID NO: 8)
H-Leu-Gln-Trp(indol-2-yl-&¹)-Asp-Glu-Glu-Thr-Gly-Glu-Cys(&¹)-Leu-Pro-βAla-Arg-
Lys-Lys-Arg-Arg-Gln-Arg-Arg-Arg-NH₂
(SEQ ID NO: 9)
{[H-Leu-Gln-Cys(&¹)-Asp-Glu-Glu-Thr-Gly-Glu-Cys(&²)-Leu-Pro-βAla-Arg-Lys-
Lys-Arg-Arg-Gln-Arg-Arg-Arg-NH₂][&¹(1,3-phenylenediyl)dimethylene&²]}
(SEQ ID NO: 10)
Acetyl-Phe(p-&¹)-Asp-Glu-Glu-Thr-Gly-Glu-Cys(&¹)-NH₂
(SEQ ID NO: 11)
&¹Leu-Gln-Trp(indol-2-yl-&²)-Asp-Glu-Glu-Thr-Gly-Glu-Cys(&²)-Leu-Pro&¹
(SEQ ID NO: 12)
{[Acetyl-Cys(&¹)-Asp-Glu-Glu-Thr-Gly-Glu-Cys(&2)-NH₂][&¹(1,2-
phenylenediyl)dimethylene&²]}
(SEQ ID NO: 13)
{[H-Leu-Gln-Cys(&¹)-Asp-Glu-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 14)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]}
(SEQ ID NO: 15)
{[H-Leu-Gln-Cys(&¹)-Asp-Ala-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 16)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,3-phenylenediyl)dimethylene&²]}
(SEQ ID NO: 17)
{[&¹Leu-Gln-Cys(&²)-Asp-Ala-Glu-Thr-Gly-Glu-Cys(&³)-Leu-Pro&¹][&²(1,3-
phenylenediyl)dimethylene&³]}
(SEQ ID NO: 18)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]}
(SEQ ID NO: 19)
{[H-Leu-Gln-Cys(&¹)-Asp-Glu-Glu-Thr-Gly-Glu-Cys(&²)-Leu-Pro-OH][&¹(1,2-
phenylenediyl)dimethylene&²]}
(SEQ ID NO: 20)
{[Acetyl-Cys(&¹)-Asp-Ala-Glu-Thr-Gly-Glu-Cys(&²)-NH₂]-[&¹(1,2-
phenylenediyl)dimethylene&²]}
(SEQ ID NO: 21)
{[Acetyl-Cys(&¹)-Asp-Ala-Glu-Thr-Gly-Glu-Cys(&²)-βAla-Arg-Lys-Lys-Arg-Arg-
Gln-Arg-Arg-Arg-NH₂][&¹(1,2-phenylenediyl)dimethylene&²]}
(SEQ ID NO: 22)
Acetyl-Phe(m-&¹)-Asp-Glu-Glu-Thr-Gly-Glu-Cys(&¹)-NH₂
(SEQ ID NO: 23)
Acetyl-Phe(o-&¹)-Asp-Glu-Glu-Thr-Gly-Glu-Cys(&¹)-NH₂
(SEQ ID NO: 24)
{[Acetyl-Cys(&¹)-Asp-Glu-Glu-Thr-Gly-Glu-Cys(&²)-NH₂][&¹(1,1′-biphenyl)2,2′-
diyldimethylene&²]}
(SEQ ID NO: 25)
{[Acetyl-Cys(&¹)-Asp-Glu-Glu-Thr-Gly-Glu-Cys(&²)-NH₂][&¹(1,1′-
binaphthalene)2,2′-diyldimethylene&²]}
(SEQ ID NO: 26)
{[Acetyl-Cys(&¹)-Asp-Glu-Glu-Thr-Gly-Glu-Cys(&²)-NH₂][&¹(quinoxaline)2,3-
diyldimethylene&²]}
(SEQ ID NO: 27)
{[Acetyl-Cys(&¹)-Asp-Glu-Glu-Thr-Gly-Glu-Cys(&²)-NH₂][&¹(naphthalene)2,3-
diyldimethylene&²]}
(SEQ ID NO: 28)
{[Stearyl-βAla-Cys(&¹)-Asp-Ala-Glu-Thr-Gly-Glu-Cys(&²)-NH₂][&¹(1,2-
phenylenediyl)dimethylene&²]}
(SEQ ID NO: 29)
{[Stearyl-βAla-Leu-Gln-Cys(&¹)-Asp-Ala-Glu-Thr-Gly-Glu-Cys(&²)-Leu-Pro-
OH][&¹(1,2-phenylenediyl)dimethylene&²]}
(SEQ ID NO: 30)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(naphtalene)2,3-diyldimethylene&²]}
(SEQ ID NO: 31)
{[Stearyl-βAla-Cys(&¹)-Asp-Pro-Glu-Thr-Gly-Glu-Cys(&²)-NH₂][&¹(1,2-
phenylenediyl)dimethylene&²]}
(SEQ ID NO: 32)
{[Stearyl-βAla-Cys(&¹)-Asp-Pro-Glu-Thr-Gly-Glu-Cys(&²)-
NH₂][&¹(naphthalene)2,3-diyldimethylene&²]}
(SEQ ID NO: 33)
{[Stearyl-βAla-Cys(&¹)-Asp-Pro-Glu-Thr-Gly-Glu-Cys(&²)-NH₂][&¹(1,3-
phenylenediyl)dimethylene&²]}
(SEQ ID NO: 34)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]}
(SEQ ID NO: 35)
{[Stearyl-βAla-Leu-Gln-Cys(&¹)-Asp-Pro-Glu-Thr-Gly-Glu-Cys(&²)-Leu-Pro-
OH][&¹(1,2-phenylenediyl)dimethylene&²]}
(SEQ ID NO: 36)
{[Stearyl-βAla-Leu-Gln-Cys(&¹)-Asp-Pro-Glu-Thr-Gly-Glu-Cys(&²)-Leu-Pro-OH]
[&¹(naphthalene)2,3-diyldimethylene&²]}
(SEQ ID NO: 37)
{[Stearyl-βAla-Leu-Gln-Cys(&¹)-Asp-Pro-Glu-Thr-Gly-Glu-Cys(&²)-Leu-Pro-
OH][&¹(1,3-phenylenediyl)dimethylene&²]}
(SEQ ID NO: 38)
{[Stearyl-βAla-Leu-Gln-Cys(&¹)-Asp-Ala-Glu-Thr-Gly-Glu-Cys(&²)-Leu-Pro-OH]
[&¹(naphthalene)2,3-diyldimethylene&²]}
(SEQ ID NO: 39)
{[Stearyl-βAla-Leu-Gln-Cys(&¹)-Asp-Ala-Glu-Thr-Gly-Glu-Cys(&²⁾-Leu-Pro-
OH][&¹(1,3-phenylenediyl)dimethylene&²]}
(SEQ ID NO: 40)
Stearyl-βAla-Leu-Gln-Phe(m-&¹)-Asp-Pro-Glu-Thr-Gly-Glu-Cys(&¹)-Leu-Pro-OH
(SEQ ID NO: 41)
Stearyl-βAla-Phe(m-&¹)-Asp-Pro-Glu-Thr-Gly-Glu-Cys(&¹)-NH₂
(SEQ ID NO: 42)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(naphthalene)2,3-diyldimethylene&²]}
(SEQ ID NO: 43)
{[Acetyl-Cys(&¹)-Asp-MeAla-Glu-Thr-Gly-Glu-Cys(&²)-NH₂][&¹(1,2-
phenylenediyl)dimethylene&²]}
(SEQ ID NO: 44)
{[Stearyl-βAla-Leu-Leu-Cys(&¹)-Asp-Pro-Glu-Thr-Gly-Glu-Cys(&²)-Leu-Leu-
NH₂][&¹(1,2-phenylenediyl)dimethylene&²]}
(SEQ ID NO: 45)
{[Stearyl-βAla-MeLeu-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 46)
{[Stearyl-βAla-Leu-Gln-Cys(&¹)-Asp-Pro-Glu-Thr-Gly-Glu-Cys(&²)-MeLeu-Pro-
NH₂][&¹(1,2-phenylenediyl)dimethylene&²]}
(SEQ ID NO: 47)
{[Stearyl-βAla-Lys-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 48)
{[Stearyl-βAla-Leu-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 49)
{[Stearyl-βAla-Lys(&1)-Gln-Cys(&²)-Asp-Pro-Glu-Thr-Gly-Glu-Cys(&³)-Leu-
Pro&1][&²(1,2-phenylenediyl)dimethylene&³]}
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 51)
{[Stearyl-βAla-Leu-Gln-Cys(&¹)-Asp-Pro-Glu-Thr-Gly-Glu-Cys(&²)-Leu-Pro-
NH₂][&¹(¹,3,5-nimethylbenzene)2,4-diyldimethylene&²]}
(SEQ ID NO: 52)
{[Stearyl-βAla-Val-Val-Cys(&¹)-Asp-Pro-Glu-Thr-Gly-Glu-Cys(&²)-Val-Val-
NH₂][&¹(1,2-phenylenediyl)dimethylene&²]}
(SEQ ID NO: 53)
{[Stearyl-βAla-Lys-Lys-Cys(&¹)-Asp-Pro-Glu-Thr-Gly-Glu-Cys(&²)-Lys-Lys-
NH₂][&¹(1,2-phenylenediyl)dimethylene&²]}
(SEQ ID NO: 54)
{[Stearyl-βAla-Leu-Gln-Cys(&¹)-Asp-Pro-Glu-Thr-Gly-Glu-Cys(&²)-Leu-Pro-βAla-
Arg-Lys-Lys-Arg-Arg-Gln-Arg-Arg-Arg-NH₂][&¹(1,2-phenylenediyl)dimethylene&²]}
(SEQ ID NO: 55)
{[Stearyl-βAla-Leu-Gln-Cys(&¹)-Asp-Pro-Glu-Thr-Gly-Glu-Cys(&²)-Leu-Pro-
NH₂][&¹(3,4,5,6-tenafluorobenzene)1,2-diyldimethylene&²]}
(SEQ ID NO: 56)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(3,4,5,6-tetrafluorobenzene)1,2-
diyldimethylene&²}
(SEQ ID NO: 57)
{[Stearyl-βAla-Lys-Lys-Cys(&¹)-Asp-Pro-Glu-Thr-Gly-Glu-Cys(&²)-Leu-Pro-
NH₂][&¹(1,2-phenylenediyl)dimethylene&²}
(SEQ ID NO: 58)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(naphthalene)2,3-diyldimethylene&²]}
(SEQ ID NO: 59)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(1,3,5-trimethylbenzene)2,4-diyldimethylene&²]}
(SEQ ID NO: 60)
{[H-Leu-Leu-Cys(&¹)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(1,2-phenylenediyl)dimethylene&²]}
(SEQ ID NO: 61)
{[Stearyl-βAla-Leu-Gln-Cys(&¹)-Asp-Pro-Glu-Thr-Gly-Glu-Cys(&²)-Leu-Pro-
NH₂][&¹(quinoxaline)2,3-diyldimethylene&²]}
(SEQ ID NO: 62)
{[Stearyl-βAla-Leu-Gln-Cys(&¹)-Asp-Pro-Glu-Thr-Gly-Glu-Cys(&²)-Leu-Pro-
NH₂][&¹(pyridine)2,6-diyldimethylene&²]}
(SEQ ID NO: 63)
{[H-Leu-Leu-Cys(&1)-Asp-Pro-Glu-Thr-Gly-Glu-Cys(&²)-Leu-Leu-βAla-Arg-Lys-
Lys-Arg-Arg-Gln-Arg-Arg-Arg-NH₂][&¹(1,1′-binaphthalene)2,2′-diyldimethylene&²]}
(SEQ ID NO: 64)
{[&¹Pro((4S)-NH-Acetyl)-Gln-Cys(&²)-Asp-Pro-Glu-Thr-Gly-Glu-Cys(&³)-Leu-D-
Pro&¹][&²(1,2-phenylenediyl)dimethylene&³]}
(SEQ ID NO: 65)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]}
(SEQ ID NO: 66)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,1′-binaphthalene)2,2′-diyldimethylene&²]}
(SEQ ID NO: 67)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,3,5-trimethylbenzene)2,4-diyldimethylene&²]}
(SEQ ID NO: 68)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]}
(SEQ ID NO: 69)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]}
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&³]}
(SEQ ID NO: 71)
{[H-Leu-Gln-Cys(&¹)-Asp-Phe(4-F)-Glu-Thr-Gly-Glu-Cys(&²)-Leu-Pro-βAla-Arg-
Lys-Lys-Arg-Arg-Gln-Arg-Arg-Arg-NH2][&¹(1,2-phenylenediyl)dimethylene&²]}
(SEQ ID NO: 72)
{[&¹Pro((4S)-NH-Stearyl)-Gln-Cys(&²)-Asp-Pro((4S)-F)-Glu-Thr-Gly-Glu-Cys(&³)-
Leu-D-Pro&¹][&²(1,2-phenylenediyl)dimethylene&³]}
(SEQ ID NO: 73)
{[&¹Pro((4S)-NH-Stearyl)-Gln-Cys(&²)-Asp-Pro((4S)-NH2)-Glu-Thr-Gly-Glu-
Cys(&³)-Leu-D-Pro&¹][&²(1,2-phenylenediyl)dimethylene&³]}
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]}
(SEQ ID NO: 75)
{[Acetyl-Pro((4S)-NH-Stearyl)-Gln-Cys(&¹)-Asp-Pro-Glu-Thr-Gly-Glu-Cys(&²)-Leu-
D-Pro-NH₂][&¹(1,2-phenylenediyl)dimethylene&²]}
(SEQ ID NO: 76)
{[Stearyl-Pro-Gln-Cys(&¹)-Asp-Pro-Glu-Thr-Gly-Glu-Cys(&²)-Leu-D-Pro-
NH₂][&¹(1,2-phenylenediyl)dimethylene&²]}
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&³]}

or a pharmaceutically acceptable salt, or solvate, or N-oxide, or stereoisomer thereof.

Still in this embodiment, particular preferred sequences of cyclic and bicyclic peptides of the compound of the present invention include:

(SEQ ID NO: 37)
{[Stearyl-βAla-Leu-Gln-Cys(&¹)-Asp-Pro-Glu-Thr-Gly-Glu-Cys(&²)-Leu-Pro-
OH][&¹(1,3-phenylenediyl)dimethylene&²]}
(SEQ ID NO: 47)
{[Stearyl-βAla-Lys-Gln-Cys(&¹)-Asp-Pro-Glu-Thr-Gly-Glu-Cys(&²)-Leu-Pro-
NH₂][&¹(1,2-phenylenediyl)dimethylene&²]}
(SEQ ID NO: 49)
{[Stearyl-βAla-Lys(&¹)-Gln-Cys(&²)-Asp-Pro-Glu-Thr-Gly-Glu-Cys(&³)-Leu-
Pro&¹[&²(1,2-phenylenediyl)dimethylene&²]}
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹[&²(1,2-phenylenediyl)dimethylene&²]}
(SEQ ID NO: 62)
{[Stearyl-βAla-Leu-Gln-Cys(&¹⁾-Asp-Pro-Glu-Thr-Gly-Glu-Cys(&²)-Leu-Pro-
NH₂][&¹(pyridine)2,6-diyldimethylene&²]}
(SEQ ID NO: 65)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]}
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&²]}
(SEQ ID NO: 72)
{[&¹Pro((4S)-NH-Stearyl)-Gln-Cys(&²)-Asp-Pro((4S)-F)-Glu-Thr-Gly-Glu-Cys(&³)-
Leu-D-Pro&¹][&²(1,2-phenylenediyl)dimethylene&²]}
(SEQ ID NO: 73)
{[&¹Pro((4S)-NH-Stearyl)-Gln-Cys(&²)-Asp-Pro((4S)-NH₂)-Glu-Thr-Gly-Glu-
Cys(&³)-Leu-D-Pro&¹][&²(1,2-phenylenediyl)dimethylene&²]}
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&2]}
(SEQ ID NO: 76)
{[Stearyl-Pro-Gln-Cys(&¹)-Asp-Pro-Glu-Thr-Gly-Glu-Cys(&²)-Leu-D-Pro-
NH₂][&¹(1,2-phenylenediyl)dimethylene&²]}
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&²]}

or a pharmaceutically acceptable salt, or solvate, or N-oxide, or stereoisomer thereof.

Peptidic compounds of the invention containing one or more chiral centre may be used in enantiomerically or diastereoisomerically pure form, in the form of racemic mixtures and in the form of mixtures enriched in one or more stereoisomer. The peptidic compounds of the present invention as described and claimed encompass the racemic forms of the compounds as well as the individual enantiomers, diastereomers, and stereoisomer-enriched mixtures.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomer conglomerates may be separated by conventional techniques known to those skilled in the art. See, e.g. “Stereochemistry of Organic Compounds” by Ernest L. Eliel (Wiley, New York, 1994).

The peptidic compounds of the present invention may exist in different physical forms, i.e. amorphous and crystalline forms.

Moreover, the peptidic compounds of the invention may have the ability to crystallize in more than one form, a characteristic which is known as polymorphism. Polymorphs can be distinguished by various physical properties well known in the art such as X-ray diffraction pattern, melting point or solubility. All physical forms of the peptidic compounds of the present invention, including all polymorphic forms (“polymorphs”) thereof, are included within the scope of the invention.

As used herein, the term pharmaceutically acceptable salt refers to a salt prepared from a base or acid which is acceptable for administration to a patient, such as a mammal. Such salts can be derived from pharmaceutically-acceptable inorganic or organic bases and from pharmaceutically-acceptable inorganic or organic acids.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid; and organic acids, for example citric, formic, fumaric, gluconic, glutamic, lactic, maleic, malic, mandelic, mucic, ascorbic, oxalic, pantothenic, succinic, tartaric, benzoic, acetic, methanesulphonic, ethanesulphonic, benzenesulphonic, p-toluenesulphonic acid, xinafoic (1-hydroxy-2-naphthoic acid), napadisilic (1,5-naphthalenedisulfonic acid) and the like. Further examples of pharmaceutically acceptable inorganic or organic acid addition salts include the pharmaceutically acceptable salts listed in Journal of Pharmaceutical Science, 66, 2 (1977) which are known to the skilled artisan. Particularly preferred are salts derived from fumaric, hydrobromic, hydrochloric, acetic, sulfuric, methanesulfonic, xinafoic, and tartaric acids.

Salts derived from pharmaceutically-acceptable inorganic bases include aluminum, ammonium, calcium, copper, ferric, ferrous, lithium, magnesium, manganic, manganous, potassium, sodium, zinc and the like. Particularly preferred are ammonium, calcium, magnesium, potassium and sodium salts.

Salts derived from pharmaceutically-acceptable organic bases include salts of primary, secondary and tertiary amines, including alkyl amines, arylalkyl amines, heterocyclyl amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Other preferred salts according to the invention are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methanesulphonate.

As used herein, an N-oxide is formed from the tertiary basic amines or imines present in the molecule, using a convenient oxidising agent.

The invention also includes isotopically-labeled peptidic compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ^{N}C, chlorine, such as ³⁶C₁, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as 3²P, and sulfur, such as ³⁵S. Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, 2H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled peptidic compounds of the invention can generally be prepared by conventional techniques known to those skilled in the art or by processes analogous to those described herein, using an appropriate isotopically-labeled reagent in place of the non-labeled reagent otherwise employed.

Preferred isotopically-labeled peptidic compounds include deuterated derivatives of the compounds of the invention. As used herein, the term deuterated derivative embraces compounds of the invention where in a particular position at least one hydrogen atom is replaced by deuterium. Deuterium (D or ²H) is present at a natural abundance of 0.015 molar %.

The peptidic compounds of the invention may exist in both unsolvated and solvated forms. The term solvate is used herein to describe a molecular complex comprising a compound of the invention and an amount of one or more pharmaceutically acceptable solvent molecules. The term hydrate is employed when said solvent is water. Examples of solvate forms include, but are not limited to, peptidic compounds of the invention in association with water, acetone, dichloromethane, 2-propanol, ethanol, methanol, dimethylsulfoxide (DMSO), ethyl acetate, acetic acid, ethanolamine, or mixtures thereof. It is specifically contemplated that in the present invention one solvent molecule can be associated with one molecule of the peptidic compounds of the present invention, such as a hydrate.

Furthermore, it is specifically contemplated that in the present invention, more than one solvent molecule may be associated with one molecule of the peptidic compounds of the present invention, such as a dihydrate. Additionally, it is specifically contemplated that in the present invention less than one solvent molecule may be associated with one molecule of the peptidic compounds of the present invention, such as a hemihydrate. Furthermore, solvates of the present invention are contemplated as solvates of compounds of the present invention that retain the biological effectiveness of the non-solvate form of the peptidic compounds.

Prodrugs of the peptidic compounds described herein are also within the scope of the invention. Thus certain derivatives of the peptidic compounds of the present invention, which derivatives may have little or no pharmacological activity themselves, when administered into or onto the body may be converted into peptidic compounds of the present invention having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the peptidic compounds of the present invention with certain moieties known to those skilled in the art as ‘pro-moieties’ as described, for example, in Design of Prodrugs by H. Bundgaard (Elsevier, 1985).

Peptidic compounds of the invention intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

The present invention includes a pharmaceutical composition comprising a peptidic compound according to the invention and a pharmaceutically acceptable carrier or diluent. Said pharmaceutical composition typically contains up to 85 wt % of a compound of the invention. More typically, it contains up to 50 wt % of a compound of the invention. Preferred pharmaceutical compositions are sterile and pyrogen free. Where a peptidic compound of the invention can exist as optical isomers, the pharmaceutical compositions provided by the invention typically contain a substantially pure optical isomer.

As used herein, the term pharmaceutical composition refers to a mixture of one or more of the compounds described herein, or physiologically/pharmaceutically acceptable salts, solvates, N-oxides, isomers, isotopes, polymorphs or prodrugs thereof, with other chemical components, such as physiologically/pharmaceutically acceptable carriers and excipients. The purpose of a pharmaceutical composition is to facilitate administration of a compound to an organism.

As used herein, a physiologically/pharmaceutically acceptable diluent or carrier refers to a carrier or diluent that does not cause significant irritation to an organism and does not abrogate the biological activity and properties of the administered compound.

A pharmaceutically acceptable excipient refers to an inert substance added to a pharmaceutical composition to further facilitate administration of a compound.

Preferably the compositions of the invention are made up in a form suitable for oral, inhalation, topical, nasal, rectal, percutaneous or injectable administration.

Pharmaceutical compositions suitable for the delivery of peptidic compounds of the invention and methods for their preparation will be readily apparent to those skilled in the art. Such compositions and methods for their preparation can be found, for example, in Remington: The Science and Practice of Pharmacy, 21st Edition, Lippincott Williams & Wilkins, Philadelphia, Pa., 2001.

The pharmaceutically acceptable excipients which are admixed with the active compound or salts of such compound, to form the compositions of this invention are well-known per se and the actual excipients used depend inter alia on the intended method of administering the compositions. Examples, without limitation, of excipients include calcium carbonate, calcium phosphate, various sugars and types of starch, cellulose derivatives, gelatin, vegetable oils and polyethylene glycols.

Additional suitable carriers for formulations of the compounds of the present invention can be found in Remington: The Science and Practice of Pharmacy, 21st Edition, Lippincott Williams & Wilkins, Philadelphia, Pa., 2001; or in Handbook of Pharmaceutical Excipients, 6th ed., published by Pharmaceutical Press and American Pharmacists Association, 2009.

The peptidic compounds of the invention may be administered orally (peroral administration; per os (latin)). Oral administration involve swallowing, so that the compound is absorbed from the gut and delivered to the liver via the portal circulation (hepatic first pass metabolism) and finally enters the gastrointestinal (GI) tract.

Compositions for oral administration may take the form of tablets, retard tablets, sublingual tablets, capsules, inhalation aerosols, inhalation solutions, dry powder inhalation, or liquid preparations, such as mixtures, solutions, elixirs, syrups or suspensions, all containing the compound of the invention; such preparations may be made by methods well-known in the art. The active ingredient may also be presented as a bolus, electuary or paste.

Where the composition is in the form of a tablet, any pharmaceutical carrier routinely used for preparing solid formulations may be used. Examples of such carriers include magnesium stearate, talc, gelatine, acacia, stearic acid, starch, lactose and sucrose.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent.

Moulded tablets may be made by moulding in a suitable machine a mixture of the powdered compound moistened with an inert liquid diluent. The tablets may optionally be coated or scored and may be formulated so as to provide slow or controlled release of the active ingredient therein.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include anti-oxidants, colorants, flavoring agents, preservatives and taste-masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant. Tablet blends may be compressed directly or by roller to form tablets. Tablet blends or portions of blends may alternatively be wet-, dry-, or melt-granulated, melt congealed, or extruded before tabletting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., 1980.

Where the composition is in the form of a capsule, any routine encapsulation is suitable, for example using the aforementioned carriers in a hard gelatine capsule. Where the composition is in the form of a soft gelatine capsule any pharmaceutical carrier routinely used for preparing dispersions or suspensions may be considered, for example aqueous gums, celluloses, silicates or oils, and are incorporated in a soft gelatine capsule.

Solid formulations for oral administration may be formulated to be immediate and/or modified release. Modified release formulations include delayed-, sustained-, pulsed-, controlled-, targeted and programmed release.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent. Liquid formulations may also be prepared by the reconstitution of a solid, for example, from a sachet.

The peptidic compounds of the invention can also be administered via the oral mucosal. Within the oral mucosal cavity, delivery of drugs is classified into three categories: (a) sublingual delivery, which is systemic delivery of drugs through the mucosal membranes lining the floor of the mouth, (b) buccal delivery, which is drug administration through the mucosal membranes lining the cheeks (buccal mucosa), and (c) local delivery, which is drug delivery into the oral cavity.

Pharmaceutical products to be administered via the oral mucosal can be designed using mucoadhesive, quick dissolve tablets and solid lozenge formulations, which are formulated with one or more mucoadhesive (bioadhesive) polymers (such as hydroxy propyl cellulose, polyvinyl pyrrolidone, sodium carboxymethyl cellulose, hydroxy propyl methyl cellulose, hydroxy ethyl cellulose, polyvinyl alcohol, polyisobutylene or polyisoprene); and oral mucosal permeation enhancers (such as butanol, butyric acid, propranolol, sodium lauryl sulphate and others)

The peptidic compounds of the invention can also be administered by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

Dry powder compositions for topical delivery to the lung by inhalation may, for example, be presented in capsules and cartridges of for example gelatine or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 0.0001-10 mg, more preferably 0.001-2 mg of active ingredient or the equivalent amount of a pharmaceutically acceptable salt thereof. Alternatively, the active ingredient (s) may be presented without excipients.

Packaging of the formulation may be suitable for unit dose or multi-dose delivery. In the case of multi-dose delivery, the formulation can be pre-metered or metered in use. Dry powder inhalers are thus classified into three groups: (a) single dose, (b) multiple unit dose and (c) multi dose devices.

For inhalers of the first type, single doses have been weighed by the manufacturer into small containers, which are mostly hard gelatine capsules. A capsule has to be taken from a separate box or container and inserted into a receptacle area of the inhaler. Next, the capsule has to be opened or perforated with pins or cutting blades in order to allow part of the inspiratory air stream to pass through the capsule for powder entrainment or to discharge the powder from the capsule through these perforations by means of centrifugal force during inhalation. After inhalation, the emptied capsule has to be removed from the inhaler again. Mostly, disassembling of the inhaler is necessary for inserting and removing the capsule, which is an operation that can be difficult and burdensome for some patients.

Other drawbacks related to the use of hard gelatine capsules for inhalation powders are (a) poor protection against moisture uptake from the ambient air, (b) problems with opening or perforation after the capsules have been exposed previously to extreme relative humidity, which causes fragmentation or indenture, and (c) possible inhalation of capsule fragments. Moreover, for a number of capsule inhalers, incomplete expulsion has been reported (e. g. Nielsen et al, 1997).

Some capsule inhalers have a magazine from which individual capsules can be transferred to a receiving chamber, in which perforation and emptying takes place, as described in WO 92/03175. Other capsule inhalers have revolving magazines with capsule chambers that can be brought in line with the air conduit for dose discharge (e. g. WO91/02558 and GB 2242134). They comprise the type of multiple unit dose inhalers together with blister inhalers, which have a limited number of unit doses in supply on a disk or on a strip.

Blister inhalers provide better moisture protection of the medicament than capsule inhalers. Access to the powder is obtained by perforating the cover as well as the blister foil, or by peeling off the cover foil. When a blister strip is used instead of a disk, the number of doses can be increased, but it is inconvenient for the patient to replace an empty strip. Therefore, such devices are often disposable with the incorporated dose system, including the technique used to transport the strip and open the blister pockets.

Multi-dose inhalers do not contain pre-measured quantities of the powder formulation. They consist of a relatively large container and a dose measuring principle that has to be operated by the patient. The container bears multiple doses that are isolated individually from the bulk of powder by volumetric displacement. Various dose measuring principles exist, including rotatable membranes (Ex. EP0069715) or disks (Ex. GB 2041763; EP 0424790; DE 4239402 and EP 0674533), rotatable cylinders (Ex. EP 0166294; GB 2165159 and WO 92/09322) and rotatable frustums (Ex. WO 92/00771), all having cavities which have to be filled with powder from the container. Other multi dose devices have measuring slides (Ex. U.S. Pat. No. 5,201,308 and WO 97/00703) or measuring plungers with a local or circumferential recess to displace a certain volume of powder from the container to a delivery chamber or an air conduit (Ex. EP 0505321, WO 92/04068 and WO 92/04928), or measuring slides such as the Genuair® (formerly known as Novolizer SD2FL), which is described the following patent applications Nos: WO97/000703, WO03/000325 and WO2006/008027.

Reproducible dose measuring is one of the major concerns for multi dose inhaler devices.

The powder formulation has to exhibit good and stable flow properties, because filling of the dose measuring cups or cavities is mostly under the influence of the force of gravity.

For reloaded single dose and multiple unit dose inhalers, the dose measuring accuracy and reproducibility can be guaranteed by the manufacturer. Multi dose inhalers on the other hand, can contain a much higher number of doses, whereas the number of handlings to prime a dose is generally lower.

Because the inspiratory air stream in multi-dose devices is often straight across the dose measuring cavity, and because the massive and rigid dose measuring systems of multi dose inhalers can not be agitated by this inspiratory air stream, the powder mass is simply entrained from the cavity and little de-agglomeration is obtained during discharge.

Consequently, separate disintegration means are necessary. However in practice, they are not always part of the inhaler design. Because of the high number of doses in multi-dose devices, powder adhesion onto the inner walls of the air conduits and the de-agglomeration means must be minimized and/or regular cleaning of these parts must be possible, without affecting the residual doses in the device. Some multi dose inhalers have disposable drug containers that can be replaced after the prescribed number of doses has been taken (Ex. WO 97/000703). For such semi-permanent multi dose inhalers with disposable drug containers, the requirements to prevent drug accumulation are even more strict.

Apart from applications through dry powder inhalers the peptidic compositions of the invention can be administered in aerosols which operate via propellant gases or by means of so-called atomisers, via which solutions of pharmacologically-active substances can be sprayed under high pressure so that a mist of inhalable particles results. The advantage of these atomisers is that the use of propellant gases can be completely dispensed with. Such atomiser is the Respimat® which is described, for example, in PCT Patent Applications Nos. WO 91/14468 and WO 97/12687, reference here is being made to the contents thereof.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the active ingredient (s) and a suitable propellant such as a fluorocarbon or hydrogen-containing chlorofluorocarbon or mixtures thereof, particularly hydrofluoroalkanes, e. g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant.

The aerosol composition may be excipient free or may optionally contain additional formulation excipients well known in the art such as surfactants (eg oleic acid or lecithin) and cosolvens (eg ethanol). Pressurised formulations will generally be retained in a canister (eg an aluminium canister) closed with a valve (eg a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means eg by micronisation. The desired fraction may be separated out by air classification or sieving. Preferably, the particles will be crystalline.

Achieving high dose reproducibility with micronised powders is difficult because of their poor flowability and extreme agglomeration tendency. To improve the efficiency of dry powder compositions, the particles should be large while in the inhaler, but small when discharged into the respiratory tract. Thus, an excipient such as lactose or glucose is generally employed. The particle size of the excipient will usually be much greater than the inhaled medicament within the present invention. When the excipient is lactose it will typically be present as milled lactose, preferably crystalline alpha lactose monohydrate.

Pressurized aerosol compositions will generally be filled into canisters fitted with a valve, especially a metering valve. Canisters may optionally be coated with a plastics material e. g. a fluorocarbon polymer as described in WO96/32150. Canisters will be fitted into an actuator adapted for buccal delivery.

The peptidic compounds of the invention may also be administered via the nasal mucosal.

Typical compositions for nasal mucosa administration are typically applied by a metering, atomizing spray pump and are in the form of a solution or suspension in an inert vehicle such as water optionally in combination with conventional excipients such as buffers, anti-microbials, tonicity modifying agents and viscosity modifying agents.

The peptidic compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art. The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The peptidic compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and microneedle or needle-free injection.

Formulations for topical administration may be formulated to be immediate and/or modified release. Modified release formulations include delayed-, sustained-, pulsed-, controlled-, targeted and programmed release.

Peptidic compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

Peptidic compounds of the invention may also be administered directly to the eye or ear, typically in the form of drops of a micronized suspension or solution in isotonic, pH-adjusted, sterile saline. Other formulations suitable for ocular and aural administration include ointments, biodegradable {e.g. absorbable gel sponges, collagen) and nonbiodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

Peptidic compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

The amount of the active compound administered will be dependent on the subject being treated, the severity of the disorder or condition, the rate of administration, the disposition of the compound and the discretion of the prescribing physician. However, an effective dosage is typically in the range of 0.01-3000 μg, more preferably 0.5-1000 μg of active ingredient or the equivalent amount of a pharmaceutically acceptable salt thereof per day. Daily dosage may be administered in one or more treatments, preferably from 1 to 4 treatments, per day.

The pharmaceutical formulations may conveniently be presented in unit dosage form and may be prepared by any of the methods well known in the art of pharmacy. Preferably the composition is in unit dosage form, for example a tablet, capsule or metered aerosol dose, so that the patient may administer a single dose.

The peptidic compound of the invention and compositions of the invention are suitable for use in treatment of a pathological condition or disease associated with the activation of the Nrf2 pathway.

The pathological condition or disease may be selected from Parkinson's disease, depression, Alzheimer's disease, atherosclerosis, heart failure, myocardial infarction, diabetes, cancer, COPD, COPD exacerbations, acute lung injury, radiation-induced dermatitis, chemical induced dermatitis, contact induced dermatitis, Epidermolysis bullosa simplex, Pachyonychia congenital, Hailey-Hailey, vitiligo, photoaging and photodamaged skin.

### EXAMPLES

Specific examples according to the present invention are represented by the following sequences:

Peptide list (SEQ ID NO)

H-Leu-Gln-Trp(indol-2-yl-&¹)-Asp-Glu-Glu-Thr-Gly-Glu-Cys(&¹)-Leu-Pro-OH (SEQ
ID NO: 1)
{[H-Leu-Gln-Cys(&¹)-Asp-Glu-Glu-Thr-Gly-Glu-Cys(&²)-Leu-Pro-OH][&¹(1,4-
phenylenediyl)dimethylene&²]} (SEQ ID NO: 2)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]} (SEQ ID NO: 3)
{[&¹Leu-Gln-Cys(&²)-Asp-Glu-Glu-Thr-Gly-Glu-Cys(&³)-Leu-Pro&¹][&²(1,3-
phenylenediyl)dimethylene&³]} (SEQ ID NO: 4)
{[&¹Leu-Gln-Cys(&²)-Asp-Glu-Glu-Thr-Gly-Glu-Cys(&³)-Leu-Pro&¹][&²(1,4-
phenylenediyl)dimethylene&³]} (SEQ ID NO: 5)
{[Acetyl-Cys(&¹)-Asp-Glu-Glu-Thr-Gly-Glu-Cys(&²)-NH₂][&¹(1,3-
phenylenediyl)dimethylene&²]} (SEQ ID NO: 6)
Acetyl-Trp(indol-2-yl-&¹)-Asp-Glu-Glu-Thr-Gly-Glu-Cys(&¹)-NH₂ (SEQ ID NO: 7)
H-Leu-Gln-Trp(indol-2-yl-&¹)-Asp-Glu-Glu-Thr-Gly-Glu-Cys(&¹)-Leu-Pro-βAla-Arg-
Lys-Lys-Arg-Arg-Gln-Arg-Arg-Arg-NH₂ (SEQ ID NO: 8)
{[H-Leu-Gln-Cys(&¹)-Asp-Glu-Glu-Thr-Gly-Glu-Cys(&²)-Leu-Pro-βAla-Arg-Lys-
Lys-Arg-Arg-Gln-Arg-Arg-Arg-NH₂] [&¹(1,3-phenylenediyl)dimethylene&²]} (SEQ
ID NO: 9)
Acetyl-Phe(p-&¹)-Asp-Glu-Glu-Thr-Gly-Glu-Cys(&¹)-NH₂ (SEQ ID NO: 10)
&¹Leu-Gln-Trp(indol-2-yl-&²)-Asp-Glu-Glu-Thr-Gly-Glu-Cys(&²)-Leu-Pro&¹ (SEQ
ID NO: 11)
{[Acetyl-Cys(&¹)-Asp-Glu-Glu-Thr-Gly-Glu-Cys(&²)-NH₂][&¹(1,2-
phenylenediyl)dimethylene&²]} (SEQ ID NO: 12)
{[H-Leu-Gln-Cys(&¹)-Asp-Glu-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 13)
{[H-Leu-Gln-Cys(&¹)-Asp-Ala-Glu-Thr-Gly-Glu-Cys(&²)-Leu-Pro-OH][&¹(1,3-
phenylenediyl)dimethylene&²]} (SEQ ID NO: 14)
{[H-Leu-Gln-Cys(&¹)-Asp-Ala-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]}
(SEQ ID NO: 15)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,3-phenylenediyl)dimethylene&²]} (SEQ ID
NO: 16)
{[&1Leu-Gln-Cys(&²)-Asp-Ala-Glu-Thr-Gly-Glu-Cys(&³)-Leu-Pro&¹][&²(1,3-
phenylenediyl)dimethylene&³]]} (SEQ ID NO: 17)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]} (SEQ ID
NO: 18)
{[H-Leu-Gln-Cys(&¹)-Asp-Ala-Glu-Thr-Gly-Glu-Cys(&²)-Leu-Pro-OH][&¹(1,2-
phenylenediyl)dimethylene&²]} (SEQ ID NO: 19)
{[Acetyl-Cys(&¹)-Asp-Ala-Glu-Thr-Gly-Glu-Cys(&²)-NH₂][&¹(1,2-
phenylenediyl)dimethylene&²]} (SEQ ID NO: 20)
{[Acetyl-Cys(&¹)-Asp-Ala-Glu-Thr-Gly-Glu-Cys(&²)-βAla-Arg-Lys-Lys-Arg-Arg-
Gln-Arg-Arg-Arg-NH₂][&¹(1,2-phenylenediyl)dimethylene&²]} (SEQ ID NO: 21)
Acetyl-Phe(m-&¹)-Asp-Glu-Glu-Thr-Gly-Glu-Cys(&¹)-NH₂ (SEQ ID NO: 22)
Acetyl-Phe(o-&¹)-Asp-Glu-Glu-Thr-Gly-Glu-Cys(&¹)-NH₂ (SEQ ID NO: 23)
{[Acetyl-Cys(&¹)-Asp-Glu-Glu-Thr-Gly-Glu-Cys(&²)-NH₂][&¹(1,1′-biphenyl)2,2′-
diyldimethylene&²]} (SEQ ID NO: 24)
{[Acetyl-Cys(&¹)-Asp-Glu-Glu-Thr-Gly-Glu-Cys(&²)-NH₂][&¹(1,1′-binaphthalene)2,2′-
diyldimethylene&²]}(SEQ ID NO: 25)
{[Acetyl-Cys(&¹)-Asp-Glu-Glu-Thr-Gly-Glu-Cys(&²)-NH₂][&¹(quinoxaline)2,3-
diyldimethylene&²]} (SEQ ID NO: 26)
{[Acetyl-Cys(&¹)-Asp-Glu-Glu-Thr-Gly-Glu-Cys(&²)-NH₂][&¹(naphthalene)2,3-
diyldimethylene&²]} (SEQ ID NO: 27)
{[Stearyl-βAla-Cys(&¹)-Asp-Ala-Glu-Thr-Gly-Glu-Cys(&²)-NH₂][&¹(1,2-
phenylenediyl)dimethylene&²]} (SEQ ID NO: 28)
{[Stearyl-Ala-Leu-Gln-Cys(&¹)-Asp-Ala-G1u-Thr-Gly-Glu-Cys(&²)-Leu-Pro-OH][&¹(1,2-
phenylenediyl)dimethylene&²]} (SEQ ID NO: 29)
{[H-Leu-Gln-Cys(&¹)-Asp-Ala-Glu-Thr-Gly-Glu-Cys(&²)-Leu-Pro-βAla-Arg-Lys-Lys-Arg-
Arg-Gln-Arg-Arg-Arg-NH₂][&¹(naphtalene)2,3-diyldimethylene&²]} (SEQ ID
NO: 30)
{[Stearyl-βAla-Cys(&¹)-Asp-Pro-Glu-Thr-Gly-Glu-Cys(&²)-NH₂][&¹(1,2-
phenylenediyl)dimethylene&²]} (SEQ ID NO: 31)
{[Stearyl-βAla-Cys(&¹)-Asp-Pro-Glu-Thr-Gly-Glu-Cys(&²)-NH₂][&¹(naphthalene)2,3-
diyldimethylene&²]} (SEQ ID NO: 32)
{[Stearyl-βAla-Cys(&¹)-Asp-Pro-Glu-Thr-Gly-Glu-Cys(&²)-NH₂][&¹(1,3-
phenylenediyl)dimethylene&²]} (SEQ ID NO: 33)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]} (SEQ ID
NO: 34)
{[Stearyl-βAla-Leu-Gln-Cys(&¹)-Asp-Pro-Glu-Thr-Gly-G1u-Cys(&²)-Leu-Pro-OH][&¹(1,2-
phenylenediyl)dimethylene&²]} (SEQ ID NO: 35)
{[Stearyl-βAla-Leu-Gln-Cys(&¹)-Asp-Pro-Glu-Thr-Gly-Glu-Cys(&²)-Leu-Pro-OH]
[&¹(naphthalene)2,3-diyldimethylene&²]} (SEQ ID NO: 36)
{[Stearyl-βA1a-Leu-Gln-Cys(&¹)-Asp-Pro-Glu-Thr-Gly-Glu-Cys(&²)-Leu-Pro-OH][&¹(1,3-
phenylenediyl)dimethylene&²]} (SEQ ID NO: 37)
{[Stearyl-βAla-Leu-Gln-Cys(&¹)-Asp-Ala-Glu-Thr-Gly-Glu-Cys(&²)-Leu-Pro-OH]
[&¹(naphthalene)2,3-diyldimethylene&²]} (SEQ ID NO: 38)
{[Stearyl-βAla-Leu-Gln-Cys(&¹)-Asp-Ala-Glu-Thr-Gly-Glu-Cys(&²)-Leu-Pro-OH][&¹(1,3-
phenylenediyl)dimethylene&²]} (SEQ ID NO: 39)
Stearyl-βAla-Leu-Gln-Phe(m-&¹)-Asp-Pro-Glu-Thr-Gly-Glu-Cys(&¹)-Leu-Pro-OH (SEQ
ID NO: 40)
Stearyl-βAla-Phe(m-&¹)-Asp-Pro-Glu-Thr-Gly-Glu-Cys(&¹)-NH₂ (SEQ ID NO: 41)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(naphthalene)2,3-diyldimethylene&²]} (SEQ ID
NO: 42)
{[Acetyl-Cys(&¹)-Asp-MeAla-Glu-Thr-Gly-Glu-Cys(&²)-NH₂][&¹(1,2-
phenylenediyl)dimethylene&²]} (SEQ ID NO: 43)
{[Stearyl-βAla-Leu-Leu-Cys(&¹)-Asp-Pro-Glu-Thr-Gly-Glu-Cys(&²)-Leu-Leu-
NH₂][&¹(1,2-phenylenediyl)dimethylene&²]} (SEQ ID NO: 44)
{[Stearyl-βAla-MeLeu-Gln-Cys(&¹)-Asp-Pro-Glu-Thr-Gly-Glu-Cys(&²)-Leu-Pro-
NH₂][&¹(1,2-phenylenediyl)dimethylene&²]} (SEQ ID NO: 45)
{[Stearyl-Ala-Leu-Gln-Cys(&¹)-Asp-Pro-Glu-Thr-Gly-Glu-Cys(&²)-MeLeu-Pro-
NH₂][&¹(1,2-phenylenediyl)dimethylene&²]} (SEQ ID NO: 46)
{[Stearyl-βAla-Lys-Gln-Cys(&¹)-Asp-Pro-Glu-Thr-Gly-Glu-Cys(&²)-Leu-Pro-NH₂][&¹(1,2-
phenylenediyl)dimethylene&²]} (SEQ ID NO: 47)
{[Stearyl-βAla-Leu-Gln-Cys(&¹)-Asp-Pro-Glu-Thr-Gly-Glu-Cys(&²)-Leu-Pro-NH₂][&¹(1,2-
phenylenediyl)dimethylene&²]} (SEQ ID NO: 48)
{[Stearyl-βAla-Lys(&¹)-Gln-Cys(&²)-Asp-Pro-Glu-Thr-Gly-Glu-Cys(&³)-Leu-
Pro&¹][&²(1,2-phenylenediyl)dimethylene&³]} (SEQ ID NO: 49)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 50)
{[Stearyl-Ala-Leu-Gln-Cys(&¹)-Asp-Pro-Glu-Thr-Gly-Glu-Cys(&²)-Leu-Pro-
NH₂][&¹(1,3,5-nimethylbenzene)2,4-diyldimethylene&²]} (SEQ ID NO: 51)
{[Stearyl-βAla-Val-Val-Cys(&¹)-Asp-Pro-Glu-Thr-Gly-Glu-Cys(&²)-Val-Val-NH₂][&¹(1,2-
phenylenediyl)dimethylene&²]} (SEQ ID NO: 52)
{[Stearyl-βAla-Lys-Lys-Cys(&¹)-Asp-Pro-Glu-Thr-Gly-Glu-Cys(&²)-Lys-Lys-NH₂][&¹(1,2-
phenylenediyl)dimethylene&²]} (SEQ ID NO: 53)
{[Stearyl-βAla-Leu-Gln-Cys(&¹)-Asp-Pro-Glu-Thr-Gly-Glu-Cys(&²)-Leu-Pro-βAla-Arg-
Lys-Lys-Arg-Arg-Gln-Arg-Arg-Arg-NH₂][&¹(1,2-phenylenediyl)dimethylene&²]}
(SEQ ID NO: 54)
{[Stearyl-Ala-Leu-Gln-Cys(&¹)-Asp-Pro-Glu-Thr-Gly-Glu-Cys(&²)-Leu-Pro-
NH₂][&¹(3,4,5,6-tenafluorobenzene)1,2-diyldimethylene&²]} (SEQ ID NO: 55)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(3,4,5,6-tetrafluorobenzene)1,2-diyldimethylene&²]} (SEQ
ID NO: 56)
{[Stearyl-βAla-Lys-Lys-Cys(&¹)-Asp-Pro-Glu-Thr-Gly-Glu-Cys(&²)-Leu-Pro-NH₂][&¹(1,2-
phenylenediyl)dimethylene&²]} (SEQ ID NO: 57)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(naphthalene)2,3-diyldimethylene&²]} (SEQ
ID NO: 58)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,3,5-trimethylbenzene)2,4-diyldimethylene&²]}(SEQ ID
NO: 59)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,2-phenylenediy1)dimethylene&²]} (SEQ ID
NO: 60)
{[Stearyl-βAla-Leu-Gln-Cys(&¹)-Asp-Pro-Glu-Thr-Gly-Glu-Cys(&²)-Leu-Pro-
NH₂][&¹(quinoxaline)2,3-diyldimethylene&²]} (SEQ ID NO: 61)
{[Stearyl-βAla-Leu-Gln-Cys(&¹)-Asp-Pro-Glu-Thr-Gly-Glu-Cys(&²)-Leu-Pro-
NH₂][&¹(pyridine)2,6-diyldimethylene&²]} (SEQ ID NO: 62)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,1′-binaphthalene)2,2′-diyldimethylene&²]}
(SEQ ID NO: 63)
{[&¹Pro((4S)-NH-Acetyl)-Gln-Cys(&²)-Asp-Pro-Glu-Thr-Gly-Glu-Cys(&³)-Leu-D-
Pro&¹][&²(1,2-phenylenediy1)dimethylene&³]} (SEQ ID NO: 64)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]} (SEQ ID NO: 65)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,1′-binaphthalene)2,2′-diyldimethylene&²]}
(SEQ ID NO: 66)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,3,5-trimethylbenzene)2,4-diyldimethylene&²]}
(SEQ ID NO: 67)
{[H-Leu-G1n-Cys(&¹)-Asp-Pro-Glu-Thr-G1y-Glu-Cys(&²)-Leu-Pro-βAla-Arg₈-
NH₂][&¹(naphthalene)2,3-diyldimethylene&²]} (SEQ ID NO: 68)
{[H-Leu-G1n-Cys(&¹)-Asp-Pro-Glu-Thr-Gly-Glu-Cys(&²)-Leu-Pro-βAla-Arg₁₀-
NH₂][&¹(naphthalene)2,3-diyldimethylene&²]} (SEQ ID NO: 69)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&²]} (SEQ ID NO: 70)
{[H-Leu-Gln-Cys(&¹)-Asp-Phe(4-F)-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]}
(SEQ ID NO: 71)
{[&¹Pro((4S)-NH-Stearyl)-Gln-Cys(&²)-Asp-Pro((4S)-F)-Glu-Thr-Gly-Glu-Cys(&³)-Leu-D-
Pro&¹][&²(1,2-phenylenediyl)dimethylene&²]} (SEQ ID NO: 72)
{[&¹Pro((4S)-NH-Stearyl)-Gln-Cys(&²)-Asp-Pro((4S)-NH₂)-Glu-Thr-Gly-Glu-Cys(&³)-Leu-
D-Pro&¹][&²(1,2-phenylenediyl)dimethylene&³]} (SEQ ID NO: 73)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]} (SEQ ID NO: 74)
{[Acetyl-Pro((4S)-NH-Stearyl)-Gln-Cys(&¹)-Asp-Pro-Glu-Thr-Gly-Glu-Cys(&²)-Leu-D-
Pro-NH₂][&¹(1,2-phenylenediyl)dimethylene²]} (SEQ ID NO: 75)
{[Stearyl-Pro-Gln-Cys(&¹)-Asp-Pro-Glu-Thr-Gly-Glu-Cys(&²)-Leu-D-Pro-NH₂][&¹(1,2-
phenylenediyl)dimethylene²]} (SEQ ID NO: 76)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&³]} (SEQ ID NO: 77)
{[&¹Pro((4S)-NH-Stearyl)-Gln-Cys(&²)-Asp-Thz-Glu-Thr-Gly-Glu-Cys(&³)-Leu-D-
Pro&¹][&²(1,2-phenylenediyl)dimethylene&³]} (SEQ ID NO: 78)
{[&¹Pro((4S)-NH-Stearyl)-Gln-Cys(&²)-Asp-Pro-Glu-Thr-Gly-Glu-Cys(&³)-Leu-D-
Pro&¹][&²(3,3-oxetanediyl)dimethylene&³]} (SEQ ID NO: 79)
{[&¹Pro((4S)-NH-Miristoyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 80)
{[&¹Pro((4S)-NH-Stearyl)-Lys-Cys(&²)-Asp-Pro-Glu-Thr-Gly-Glu-Cys(&³)-Leu-D-
Pro&¹][&²(1,2-phenylenediy1)dimethylene&³]} (SEQ ID NO: 81)
{[&¹Pro((4S)-NH-Stearyl)-Gln-Cys(&²)-Asp-Pro-Glu-Thr-Gly-Glu-Cys(&³)-Lys-D-
Pro&¹][&²(1,2-phenylenediyl)dimethylene&³]} (SEQ ID NO: 82)
{[&¹Pro((4S)-NH-Palmitoyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 83)
{[&¹Pro((4S)-NH-Stearyl)-Gln-Cys(&²)-Asp-Pro-Glu-Thr-Gly-Glu-Cys(&³)-Leu-D-
Pro&¹][&¹(pyridine)3,5-diyldimethylene&²]} (SEQ ID NO: 84)
{[&¹Pro((4S)-NH-Lauryl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 85)
{[&¹Pro((4S)-NH-α-linolenyl)-Gln-Cys(&²)-Asp-Pro-Glu-Thr-Gly-Glu-Cys(&³)-Leu-
D-Pro&¹][&²(1,2-phenylenediyl)dimethylene&³]} (SEQ ID NO: 86)
{[&¹Pro((4S)-NH-elaidyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 87)
{[&¹Pro((4S)-NH-oleyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 88)
{[&¹Pro((4S)-NH-behenyl)-Gln-Cys(&²)-Asp-Pro-Glu-Thr-Gly-Glu-Cys(&3)-Leu-D-
Pro&¹][&²(1,2-phenylenediyl)dimethylene&³]} (SEQ ID NO: 89)
{[&¹Pro((4S)-NH-arachidyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 90)
{[&¹Lys(N⁶-Stearyl)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 91)
{[Stearyl-Lys(&¹)-Gln-Cys(&²)-Asp-Pro-Glu-Thr-Gly-Glu-Cys(&³)-Leu-D-
Pro&¹][&²(1,2-phenylenediyl)dimethylene&³]} (SEQ ID NO: 92)
{[&¹Pro((4S)-NH-Arachidyl)-Gln-Cys(&²)-Asp-Thz-Glu-Thr-Gly-Glu-Cys(&³)-Leu-D-
Pro&¹][&²(1,2-phenylenediyl)dimethylene&³]} (SEQ ID NO: 93)
{[&¹Pro((4S)-NH-Arachidyl)-Gln-Cys(&²)-Asp-Thz-Glu-Thr-Gly-Glu-Cys(&³)-Leu-D-
Pro&¹][&²(3,3-oxetanediyl)dimethylene&³]} (SEQ ID NO: 94)
{[&¹Pro((4S)-NH-Arachidyl)-Gln-Cys(&²)-Asp-Pro-Glu-Thr-Gly-Glu-Cys(&³)-Leu-D-
Pro&¹][&²(3,3-oxetanediyl)dimethylene&³]} (SEQ ID NO: 95)
{[&¹Pro((4S)-NH-Stearyl)-Gln-Cys(&²)-Asp-Thz-Glu-Thr-Gly-Glu-Cys(&³)-Leu-D-
Pro&¹][&²(1,3-phenylenediyl)dimethylene&³]} (SEQ ID NO: 96)
{[&¹Pro((4S)-NH-Stearyl)-Gln-Cys(&²)-Asp-Thz-Glu-Thr-Gly-Glu-Cys(&³)-Leu-D-
Pro&¹][&²(3,3-oxetanediyl)dimethylene&³]} (SEQ ID NO: 97)
{[&¹Pro((4S)-NH-Nonadecanoyl)-Gln-Cys(&²)-Asp-Pro-Glu-Thr-Gly-Glu-Cys(&³)-
Leu-D-Pro&¹][&²(1,2-phenylenediyl)dimethylene&³]} (SEQ ID NO: 98)
{[Stearyl-βAla-Leu-Leu-Cys(&¹)-Asp-Pro-Glu-Thr-Gly-Glu-Cys(&²)-Leu-Leu-
NH₂][&¹(3,3-oxetanediyl)dimethylene&²]} (SEQ ID NO: 99)
{[&¹Pro((4S)-NH-Stearyl)-Gln-Cys(&²)-Asp-Pro-Glu-Thr-Gly-Glu-Cys(&³)-Leu-D-
Pro&¹][&²(tetrahydro-2H-pyran-4,4-diyl)dimethylene&³]} (SEQ ID NO: 100)
¹(a) Merrifield, R.B. J. Am. Chem. Soc. 85, 2149, 1963. (b) Merrifield, R.B. Angew. Chem. Int. Ed. 24, 799, 1985.
²Vazquez J, Qushair G, Albericio F. Methods Enzymol. 369:21-35, 2003.

or a pharmaceutically acceptable salt, or solvate, or N-oxide, or stereoisomer thereof.

### Synthesis of Peptide Analogs

The peptides of the invention were synthesized manually by the solid phase peptide synthesis (SPPS)′ methodology using standard Fmoc/tBu chemistry. All work carried out in solid-phase was performed in polypropylene syringes fitted with a polyethylene porous disk facilitating the removal of solvents and soluble reagents by suction under reduced pressure.

Two type of solid supports were selected depending on the sequence required, when the peptide had a C-terminal amide group, the Rink amide resin was selected; and when the peptide had a C-terminal acid group or when the peptide was a bicyclic analog, the 2-chlorotrityl (2-CTC) resin was preferred. The nature of the polymer support of those resins was polystyrene (PS) or polyethylene glycol (PEG), depending on the difficulty of the elongation of the peptide sequence, at is indicated in each example. The 2-CTC resin afforded also lineal C-terminal acid peptide sequences without removing the side-chain protecting groups, which allow the synthesis of the bicyclic analogs.

For incorporation of amino acids, the coupling agents based in neutral conditions such as DIPCDI with OxymaPure® were used as a first attempt. In some cases, coupling conditions that required basic media were also selected, such as HBTU in DIEA. The standard defined colorimetric² tests were performed to evaluate the completed incorporation of amino acids in each elongation. General methods for Fmoc/tBu strategy to remove the Fmoc group with piperidine and the DMF/DCM washes to remove byproducts from the peptidyl-resin were carried out as it is specified below. The cleavage from the resin was performed in acidic media and the percentage of the TFA used was concentrated (95%) when the peptide was cleaved with concomitant global deprotection (removal of all side-chain protecting groups); and the percentage of the TFA used was diluted (2%) when the peptide was cleaved without removing the side-chain protecting groups.

In this invention there are described cyclic and bicyclic peptides, some of them conjugated to a fatty acid, conjugated to a cell penetrating peptide (CPP) or conjugated to both of them. The structural characteristics of the peptides are summarized in TABLE 1.

The synthetic steps to afford the cyclic and bicyclic peptide analogs are defined in scheme 1 and 2, respectively. The elongation of sequences was performed on solid phase. Mostly of the cases, the peptides were cleaved from the resin and cyclizations were carried out in solution using different protocols to afford cyclic and byclic peptides. For cyclizations of lineal sequences different protocols were performed in solution to afford cyclic and bicyclic peptides. Different type of connections and linkers were explored in this invention to obtain cyclic sequences with specific structural characteristics. The method selected for each peptide depends on the structural characteristics of the peptide analog and also depends on the kind of connection which cyclize the sequence, being specified in each example described in this invention.

Alternatively, a solid-phase cyclization protocol (scheme 3) has been used to obtain some cyclic peptide analogs. This method allowed to afford cyclic peptide sequences totally on solid phase, without the necessity to perform reactions in solution. The last step of those syntheses consists of the cleavage from the resin which afford the final peptide crudes prepared to be purified.

In all cases described in this invention the peptides were purified by RP-HPLC semi-preparative equipment to afford peptides pure enough to be tested. Two acidic eluent systems were selected to purify the peptides, one of them based on trifluoroacetic and other in formic acid solutions. For those peptide sequences with basic net charge, the corresponding peptides were obtained as trifluoroacetate or formate salts, respectively. Different purification conditions and gradients were carried out also depending on the sequence analog, being specified in each example described in this invention.

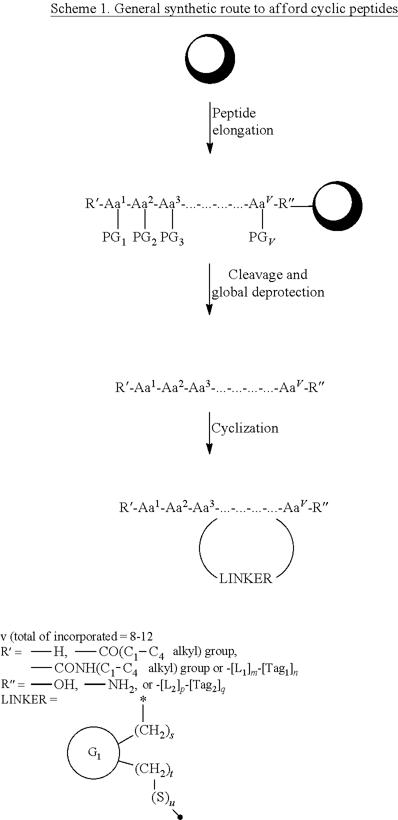

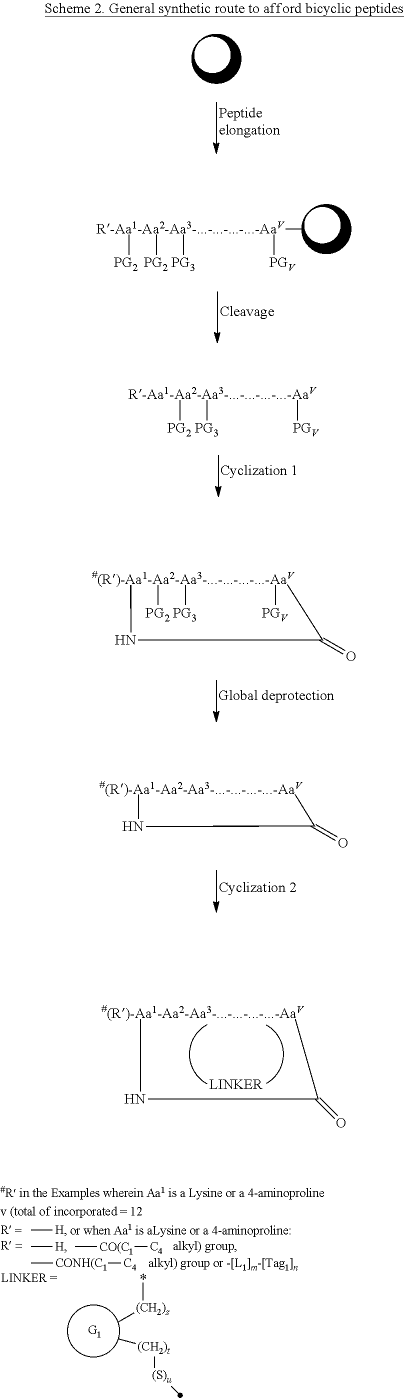

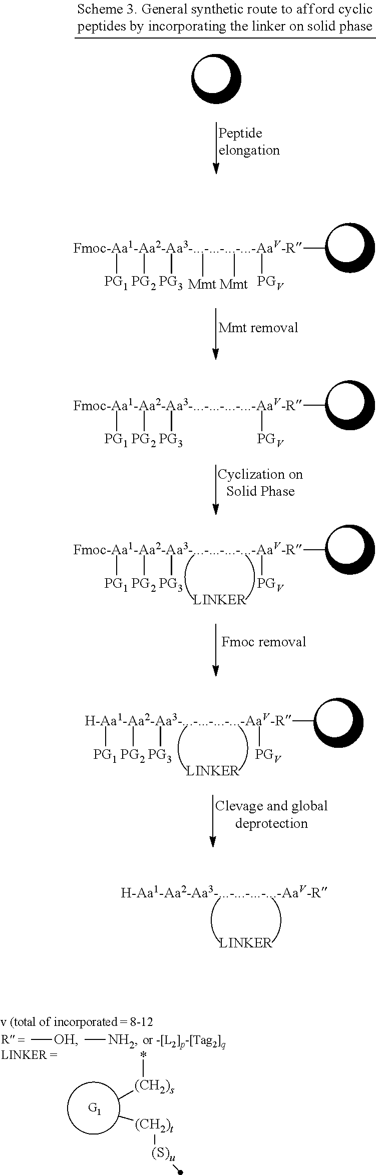

The structural characteristics of the synthesized peptides are summarized in TABLE 1.

TABLE 1 shows for each synthesized peptide: R¹ and R² substituents, the Aa in position 78, and the different LINKERS represented by:

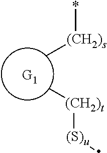

wherein
* represents the point of attachment with the S atom of the Cys⁸³
● represents the point of attachment with the carbon atom of the side chain of the Aa residue in position 76 as defined in Formula (I)′, Formula (I), Formula (IA)′ and Formula (IA).

TABLE 1
Structural characteristics of synthesized peptides

| SEQ ID NO | Total No Aa | Peptide Structure | —R¹ | —R² | -Aa₇₈- | 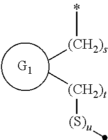 |
|---|---|---|---|---|---|---|
| 1 | 12 | 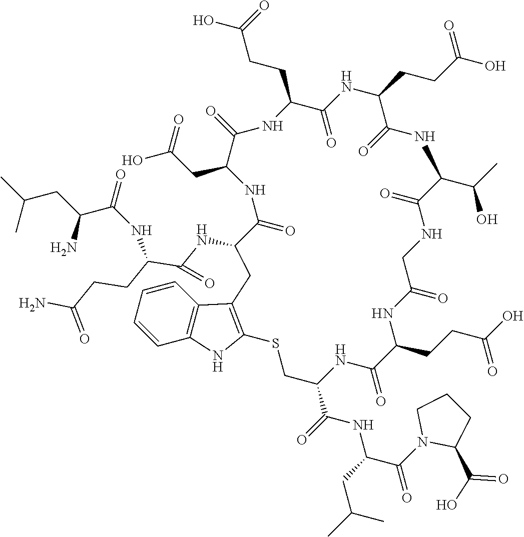 [SMILES: CC(C)C[C@H](NC(=O)[C@@H]1CSc2[nH]c3ccccc3c2C[C@H](NC(=O)[C@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)O] | -Gln-Leu-H | -Leu-Pro-OH | -Glu- | 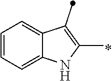 |
| 2 | 12 | 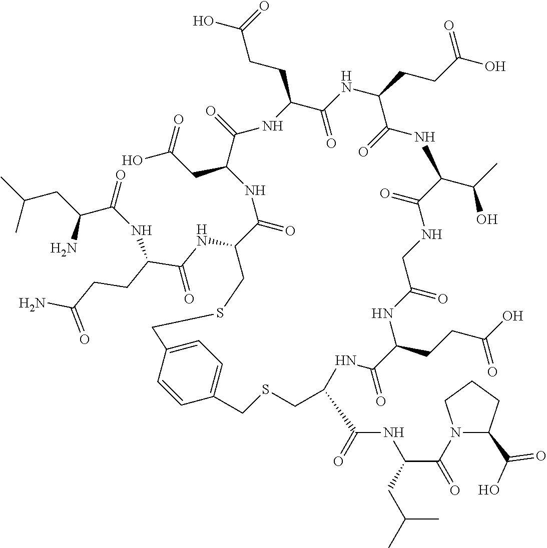 [SMILES: CC(C)C[C@H](NC(=O)[C@@H]1CSCc2ccc(cc2)CSC[C@H](NC(=O)[C@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)O] | -Gln-Leu-H | -Leu-Pro-OH | -Glu- | 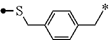 |
| 3 | 12 | 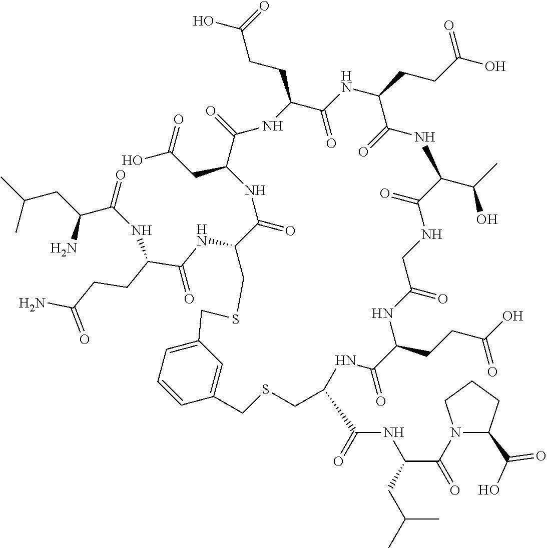 [SMILES: CC(C)C[C@H](NC(=O)[C@@H]1CSCc2cccc(c2)CSC[C@H](NC(=O)[C@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)O] | -Gln-Leu-H | -Leu-Pro-OH | -Glu- | 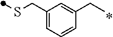 |
| 4 | 12 | 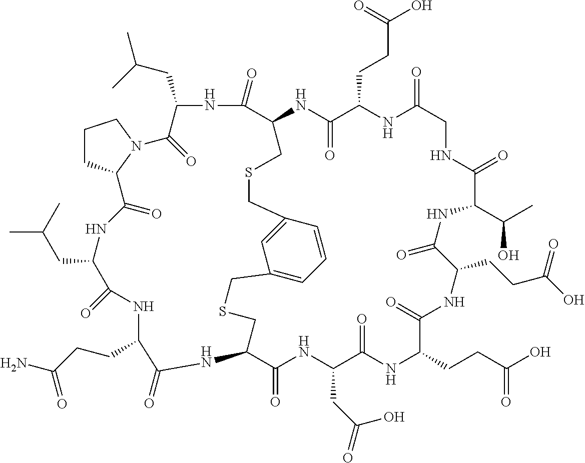 [SMILES: CC(C)C[C@@H]1NC(=O)[C@@H]2CCCN2C(=O)[C@H](CC(C)C)NC(=O)[C@@H]2CSCc3cccc(c3)CSC[C@H](NC(=O)[C@H](CCC(N)=O)NC1=O)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N2] | -Gln-Leu- | -Leu-Pro- | -Glu- | 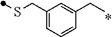 |
| 5 | 12 | 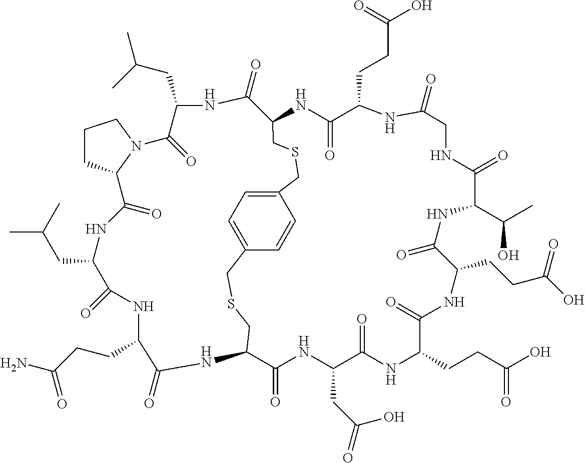 [SMILES: CC(C)C[C@@H]1NC(=O)[C@@H]2CCCN2C(=O)[C@H](CC(C)C)NC(=O)[C@@H]2CSCc3ccc(cc3)CSC[C@H](NC(=O)[C@H](CCC(N)=O)NC1=O)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N2] | -Gln-Leu- | -Leu-Pro- | -Glu- | 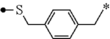 |
| 6 | 8 | 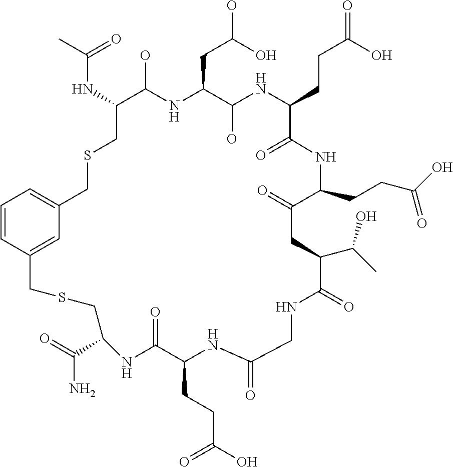 [SMILES: CC(=O)N[C@H]1CSCc2cccc(c2)CSC[C@@H](C(N)=O)NC(=O)[C@H](CCC(=O)O)NC(=O)CNC(=O)[C@H]([C@@H](C)O)CC(=O)[C@H](CCC(=O)O)NC(=O)[C@H](CCC(=O)O)NC(O)[C@H](CC(O)O)NC1O] | —COCH₃ | —NH₂ | -Glu- | 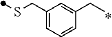 |
| 7 | 8 | 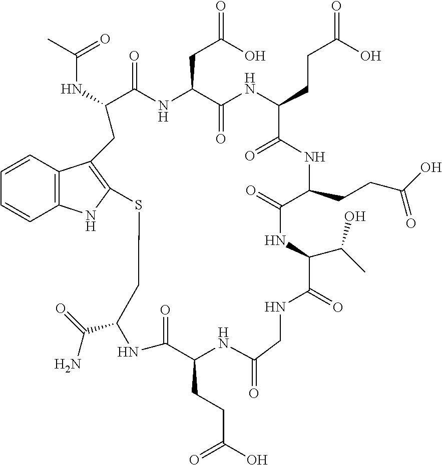 [SMILES: CC(=O)N[C@H]1Cc2c([nH]c3ccccc23)SC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 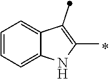 |
| 8 | 12 | 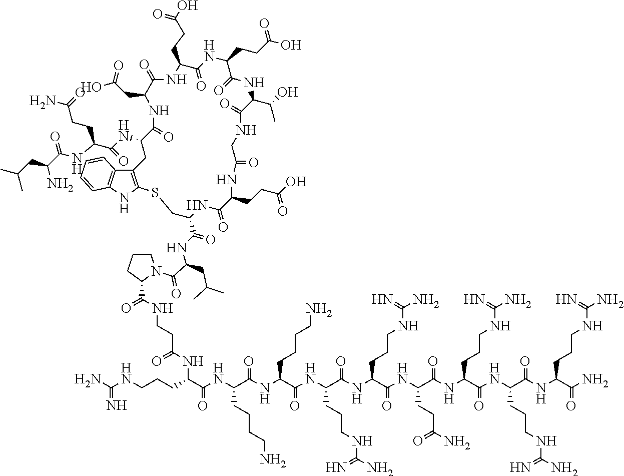 [SMILES: CC(C)C[C@H](NC(=O)[C@@H]1CSc2[nH]c3ccccc3c2C[C@H](NC(=O)[C@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Glu- | 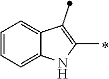 |
| 9 | 12 | 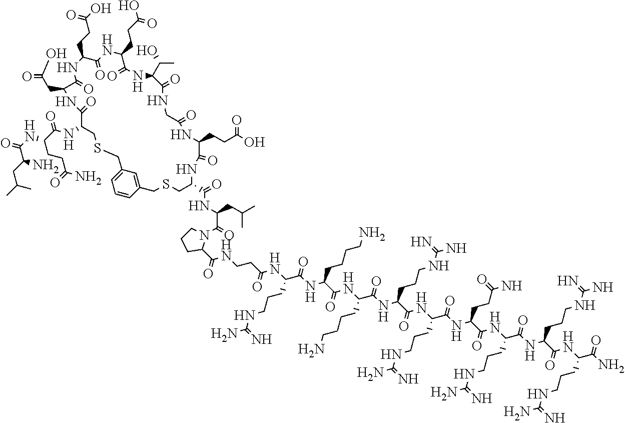 [SMILES: CC(C)C[C@H](NC(=O)[C@@H]1CSCc2cccc(c2)CSC[C@H](NC(=O)[C@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCCC1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Glu- | 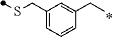 |
| 10 | 8 | 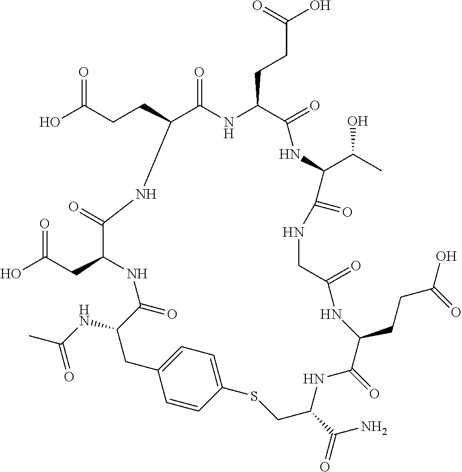 [SMILES: CC(=O)N[C@H]1Cc2ccc(cc2)SC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 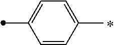 |
| 11 | 12 | 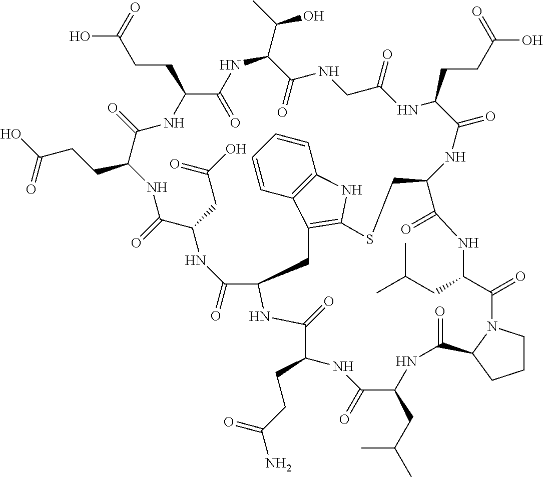 [SMILES: CC(C)C[C@@H]1NC(=O)[C@@H]2CCCN2C(=O)[C@H](CC(C)C)NC(=O)[C@H]2CSc3[nH]c4ccccc4c3C[C@@H](NC(=O)[C@H](CCC(N)=O)NC1=O)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N2] | -Gln-Leu- | -Leu-Pro- | -Glu- | 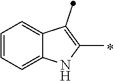 |
| 12 | 8 | 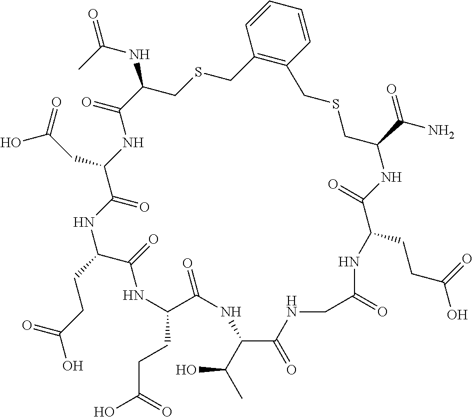 [SMILES: CC(=O)N[C@H]1CSCc2ccccc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 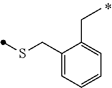 |
| 13 | 12 | 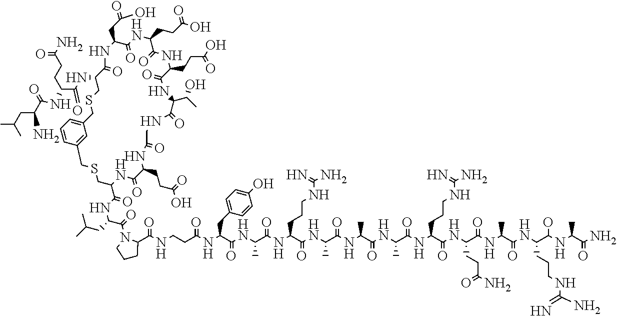 [SMILES: CC(C)C[C@H](NC(=O)C1CSCc2cccc(c2)CSC[C@H](NC(=O)[C@@H](CCC(N)=O)NC(=O)[C@@H](N)CC(C)C)C(=O)N[C@@H](CC(=O)O)C(=O)N[C@@H](CCC(=O)O)C(=O)N[C@@H](CCC(=O)O)C(=O)N[C@@H]([C@@H](C)O)C(=O)NCC(=O)N[C@@H](CCC(=O)O)C(=O)N1)C(=O)N1CCCC1C(=O)NCCC(=O)N[C@@H](Cc1ccc(O)cc1)C(=O)N[C@@H](C)C(=O)N[C@@H](CCCNC(=N)N)C(=O)N[C@@H](C)C(=O)N[C@@H](C)C(=O)N[C@@H](C)C(=O)N[C@@H](CCCNC(=N)N)C(=O)N[C@@H](CCC(N)=O)C(=O)N[C@@H](C)C(=O)N[C@@H](CCCNC(=N)N)C(O)N[C@@H](C)C(N)=O] | -Gln-Leu-H | -Leu-Pro-NH—(CH₂)₂— CO-Tyr-Ala-Arg-Ala- Ala-Ala-Arg-Gln-Ala- Arg-Ala-NH₂ | -Glu- | 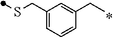 |
| 14 | 12 | 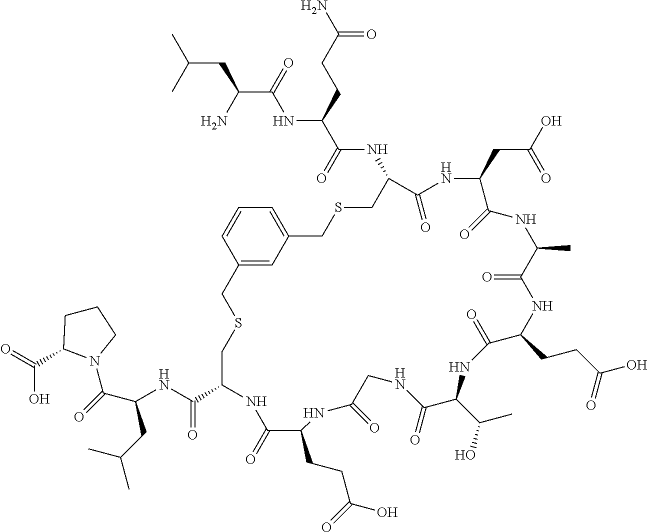 [SMILES: CC(C)C[C@H](NC(=O)[C@@H]1CSCc2cccc(c2)CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)O] | -Gln-Leu-H | -Leu-Pro-OH | -Ala- | 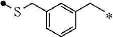 |
| 15 | 12 | 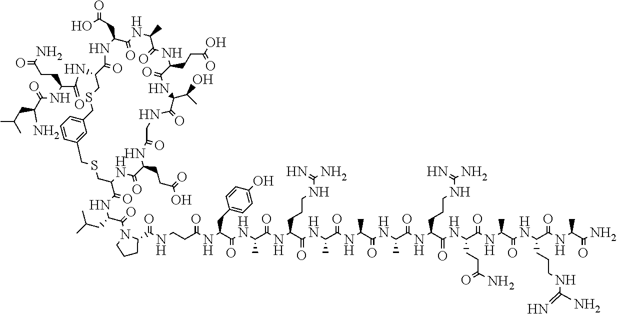 [SMILES: CC(C)C[C@H](NC(=O)C1CSCc2cccc(c2)CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)NCCC(=O)N[C@@H](Cc1ccc(O)cc1)C(=O)N[C@@H](C)C(=O)N[C@@H](CCCNC(=N)N)C(=O)N[C@@H](C)C(=O)N[C@@H](C)C(=O)N[C@@H](C)C(=O)N[C@@H](CCCNC(=N)N)C(=O)N[C@@H](CCC(N)=O)C(=O)N[C@@H](C)C(=O)N[C@@H](CCCNC(=N)N)C(=O)N[C@@H](C)C(N)=O] | -Gln-Leu-H | -Leu-Pro-NH—(CH₂)₂— CO-Tyr-Ala-Arg-Ala- Ala-Ala-Arg-Gln-Ala- Arg-Ala-NH₂ | -Ala- | 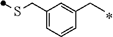 |
| 16 | 12 | 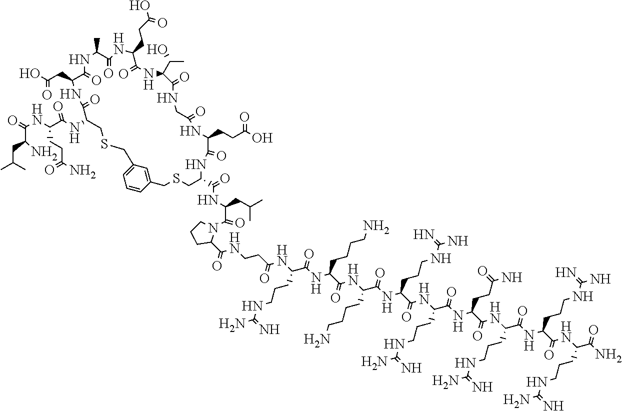 [SMILES: CC(C)C[C@H](NC(=O)[C@@H]1CSCc2cccc(c2)CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@@H](C)O)C(=O)NCC(=O)N[C@@H](CCC(=O)O)C(=O)N1)C(=O)N1CCCC1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Ala- | 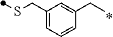 |
| 17 | 12 | 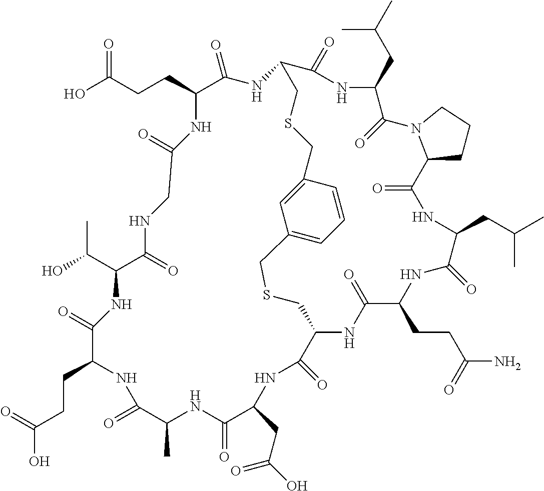 [SMILES: CC(C)C[C@@H]1NC(=O)[C@@H]2CCCN2C(=O)[C@H](CC(C)C)NC(=O)[C@@H]2CSCc3cccc(c3)CSC[C@H](NC(=O)[C@H](CCC(N)=O)NC1=O)C(=O)N[C@@H](CC(=O)O)C(=O)N[C@@H](C)C(=O)N[C@@H](CCC(=O)O)C(=O)N[C@@H]([C@@H](C)O)C(=O)NCC(=O)N[C@@H](CCC(=O)O)C(=O)N2] | -Gln-Leu- | -Leu-Pro- | -Ala- | 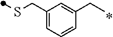 |
| 18 | 12 | 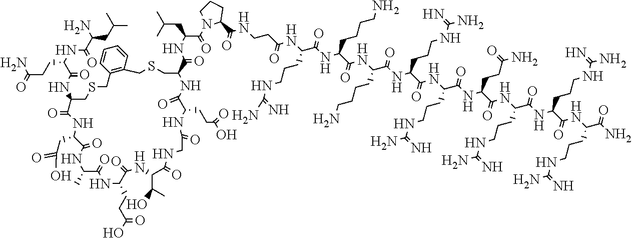 [SMILES: CC(C)C[C@H](NC(=O)[C@@H]1CSCc2ccccc2CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Ala- | 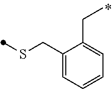 |
| 19 | 12 | 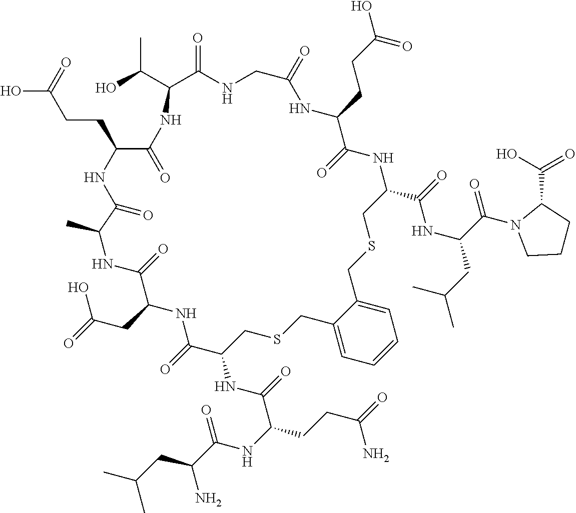 [SMILES: CC(C)C[C@H](NC(=O)[C@@H]1CSCc2ccccc2CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)O] | -Gln-Leu-H | -Leu-Pro-OH | -Ala- | 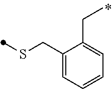 |
| 20 | 8 | 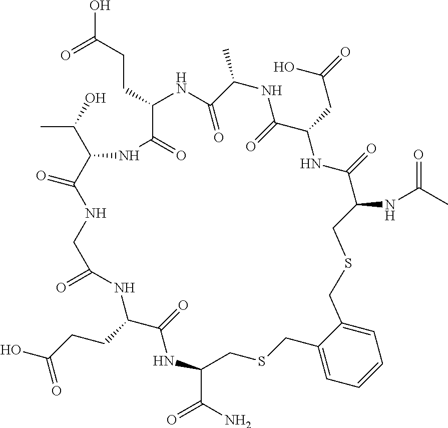 [SMILES: CC(=O)N[C@H]1CSCc2ccccc2CSC[C@@H](C(N)=O)NC(=O)[C@H](CCC(=O)O)NC(=O)CNC(=O)[C@H]([C@H](C)O)NC(=O)[C@H](CCC(=O)O)NC(=O)[C@H](C)NC(=O)[C@H](CC(=O)O)NC1=O] | —COCH₃ | —NH₂ | -Ala- | 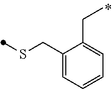 |
| 21 | 8 | 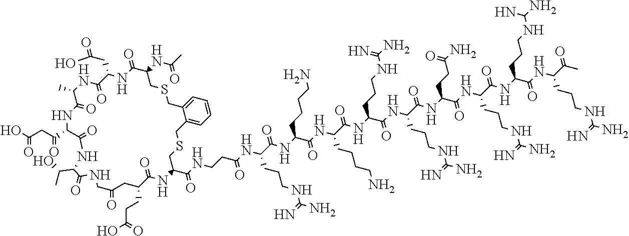 [SMILES: CC(=O)N[C@H]1CSCc2ccccc2CSC[C@@H](C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(N)N)C(=O)N[C@@H](CCCNC(=N)N)C(C)=O)NC(=O)[C@H](CCC(=O)O)CC(=O)CNC(=O)[C@H]([C@H](C)O)NC(=O)[C@H](C(=O)CC(=O)O)NC(=O)[C@H](C)NC(=O)[C@H](CC(=O)O)NC1=O] | —COCH₃ | —NH—(CH₂)₂—CO- Arg-Lys-Lys-Arg-Arg- Gln-Arg-Arg-Arg-NH₂ | -Ala- | 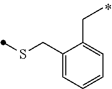 |
| 22 | 8 | 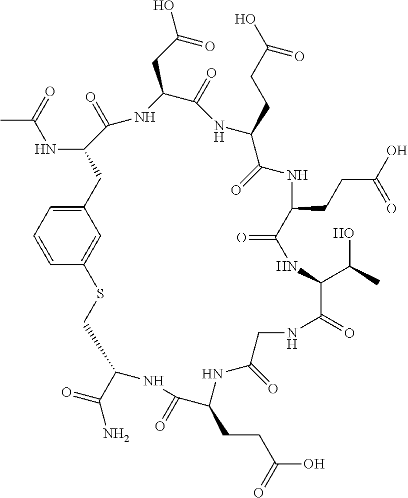 [SMILES: CC(=O)N[C@H]1Cc2cccc(c2)SC[C@@H](C(N)=O)NC(=O)[C@H](CCC(=O)O)NC(=O)CNC(=O)[C@H]([C@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 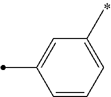 |
| 23 | 8 | 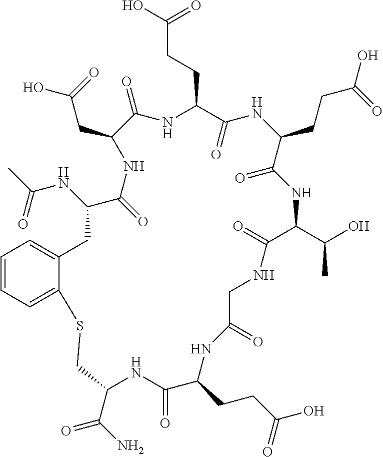 [SMILES: CC(=O)N[C@H]1Cc2ccccc2SC[C@@H](C(N)=O)NC(=O)[C@H](CCC(=O)O)NC(=O)CNC(=O)[C@H]([C@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 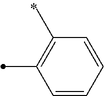 |
| 24 | 8 | 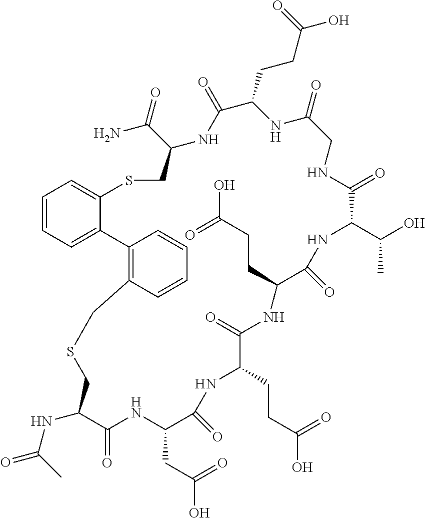 [SMILES: CC(=O)N[C@H]1CSCc2ccccc2-c2ccccc2SC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 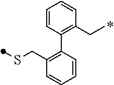 |
| 25 | 8 | 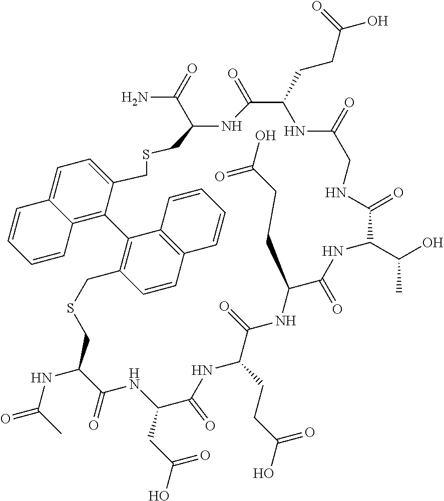 | —COCH₃ | —NH₂ | -Glu- | 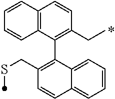 |
| 26 | 8 | 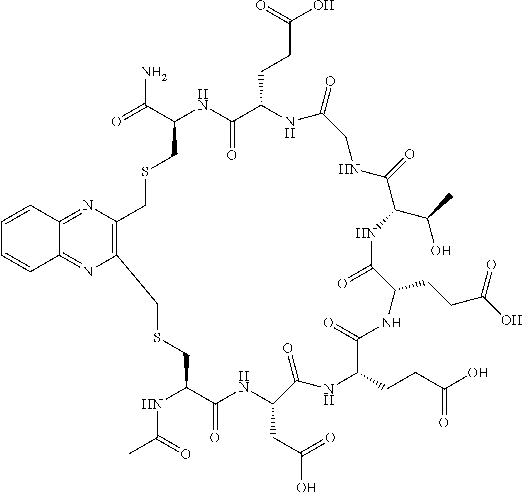 [SMILES: CC(=O)N[C@H]1CSCc2nc3ccccc3nc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 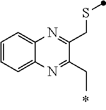 |
| 27 | 8 | 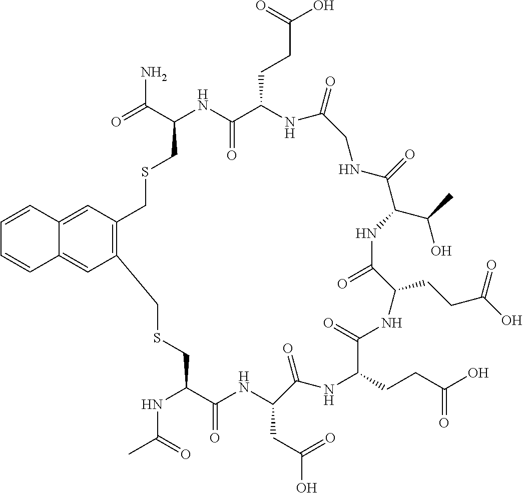 [SMILES: CC(=O)N[C@H]1CSCc2cc3ccccc3cc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](CCC(=O)O)NC(=O)[C@H](CC(=O)O)NC1=O] | —COCH₃ | —NH₂ | -Glu- | 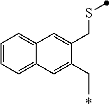 |
| 28 | 8 | 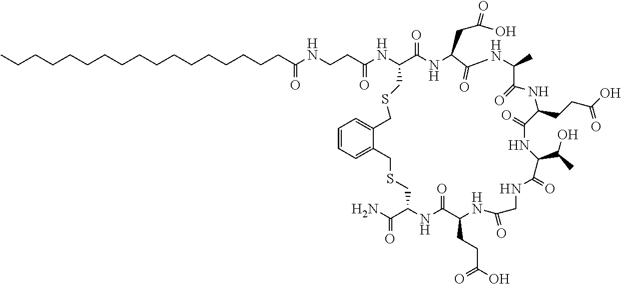 [SMILES: CCCCCCCCCCCCCCCCCC(=O)NCCC(=O)N[C@H]1CSCc2ccccc2CSC[C@@H](C(N)=O)NC(=O)[C@H](CCC(=O)O)NC(=O)CNC(=O)[C@H]([C@H](C)O)NC(=O)[C@H](CCC(=O)O)NC(=O)[C@H](C)NC(=O)[C@H](CC(=O)O)NC1=O] | —CO—(CH₂)₂—NH— CO—(CH₂)₁₆—CH₃ | —NH₂ | -Ala- | 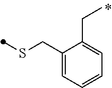 |
| 29 | 12 | 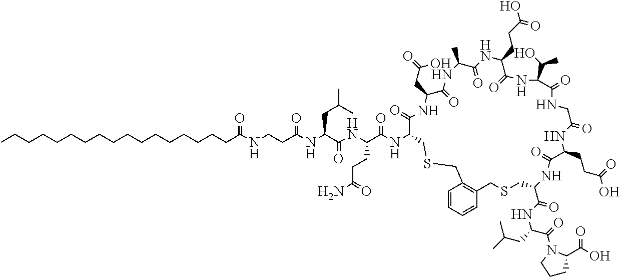 [SMILES: CCCCCCCCCCCCCCCCCC(=O)NCCC(=O)N[C@@H](CC(C)C)C(=O)N[C@@H](CCC(N)=O)C(=O)N[C@H]1CSCc2ccccc2CSC[C@@H](C(=O)N[C@@H](CC(C)C)C(=O)N2CCC[C@H]2C(=O)O)NC(=O)[C@H](CCC(=O)O)NC(=O)CNC(=O)[C@H]([C@H](C)O)NC(=O)[C@H](CCC(=O)O)NC(=O)[C@H](C)NC(=O)[C@H](CC(=O)O)NC1=O] | -Gln-Leu-CO— (CH₂)₂—NH—CO— (CH₂)₁₆—CH₃ | -Leu-Pro-OH | -Ala- | 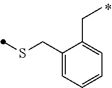 |
| 30 | 12 | 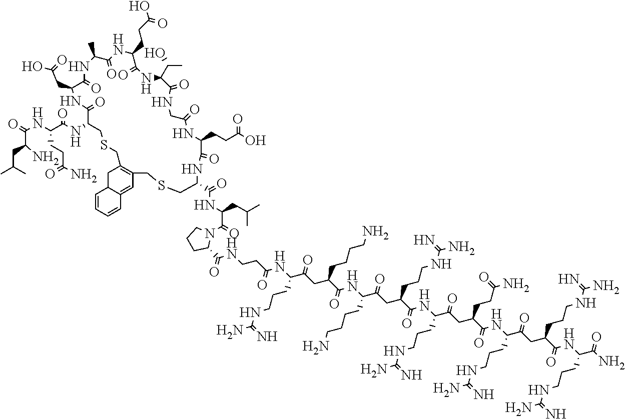 [SMILES: CC(C)C[C@H](NC(=O)[C@@H]1CSCC2=C(CSC[C@H](NC(=O)[C@H](CCC(N)=O)NC(=O)[C@@H](N)CC(C)C)C(=O)N[C@@H](CC(=O)O)C(=O)N[C@@H](C)C(=O)N[C@@H](CCC(=O)O)C(=O)N[C@@H]([C@@H](C)O)C(=O)NCC(=O)N[C@@H](CCC(=O)O)C(=O)N1)CC1C=CC=CC1=C2)C(=O)N1CCC[C@H]1C(=O)NCCC(=O)N[C@@H](CCCNC(=N)N)C(=O)C[C@@H](CCCCN)C(=O)N[C@@H](CCCCN)C(=O)C[C@@H](CCCNC(=N)N)C(=O)N[C@@H](CCCNC(=N)N)C(=O)C[C@@H](CCC(N)=O)C(=O)N[C@@H](CCCNC(=N)N)C(=O)C[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Ala- | 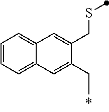 |
| 31 | 8 | 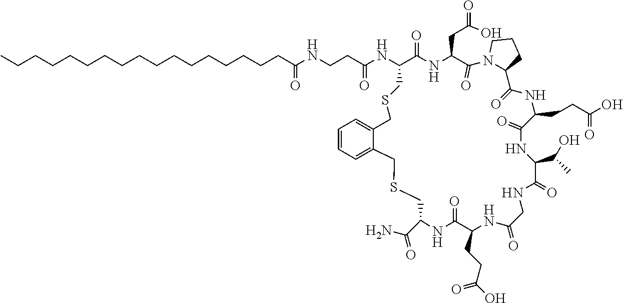 [SMILES: CCCCCCCCCCCCCCCCCC(=O)NCCC(=O)N[C@H]1CSCc2ccccc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | —CO—(CH₂)₂—NH— CO—(CH₂)₁₆—CH₃ | NH₂ | -Pro- | 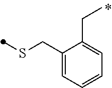 |
| 32 | 8 | 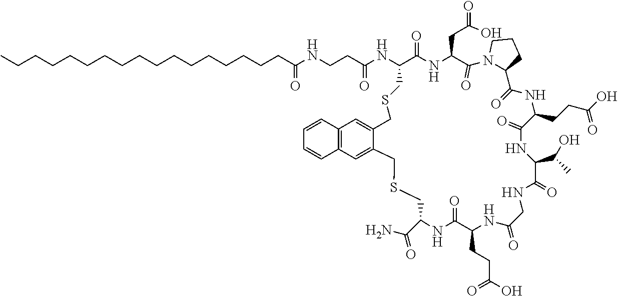 [SMILES: CCCCCCCCCCCCCCCCCC(=O)NCCC(=O)N[C@H]1CSCc2cc3ccccc3cc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | —CO—(CH₂)₂—NH— CO—(CH₂)₁₆—CH₃ | NH₂ | -Pro- | 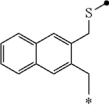 |
| 33 | 8 | 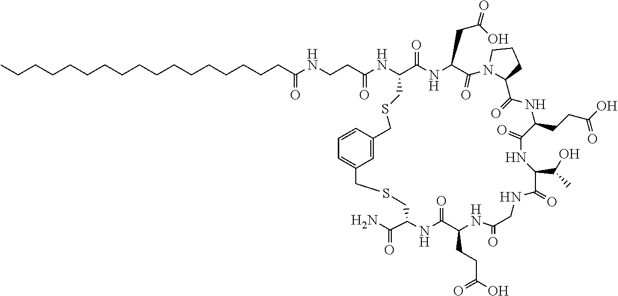 [SMILES: CCCCCCCCCCCCCCCCCC(=O)NCCC(=O)N[C@H]1CSCc2cccc(c2)CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | —CO—(CH₂)₂—NH— CO—(CH₂)₁₆—CH₃ | NH₂ | -Pro- | 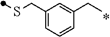 |
| 34 | 12 | 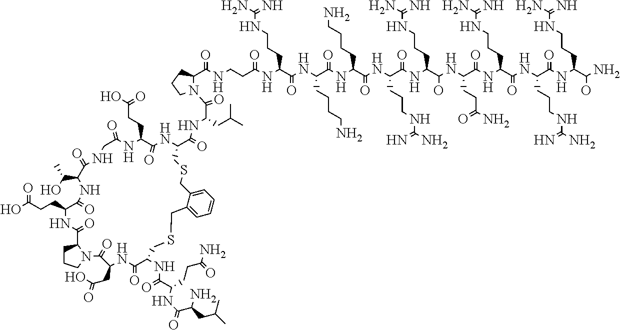 [SMILES: CC(C)C[C@H](NC(=O)[C@@H]1CSCc2ccccc2CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)O] | -Gln-Leu-H | -Leu-Pro-NH—(CH₂)₂— CO-Arg-Lys-Lys- Arg-Arg-Gln-Arg-- Arg-Arg-NH₂ | -Pro- | 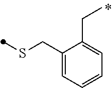 |
| 35 | 12 | 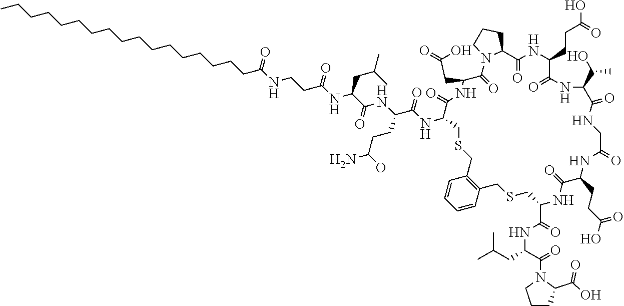 [SMILES: CCCCCCCCCCCCCCCCCC(=O)NCCC(=O)N[C@@H](CC(C)C)C(=O)N[C@@H](CCC(N)O)C(=O)N[C@H]1CSCc2ccccc2CSC[C@@H](C(=O)N[C@@H](CC(C)C)C(=O)N2CCC[C@H]2C(=O)O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-OH | -Pro- | 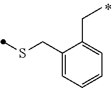 |
| 36 | 12 | 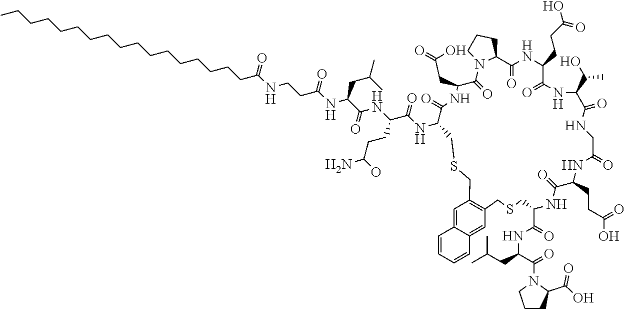 [SMILES: CCCCCCCCCCCCCCCCCC(=O)NCCC(=O)N[C@@H](CC(C)C)C(=O)N[C@@H](CCC(N)O)C(=O)N[C@H]1CSCc2cc3ccccc3cc2CSC[C@@H](C(=O)N[C@H](CC(C)C)C(=O)N2CCC[C@@H]2C(=O)O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-OH | -Pro- | 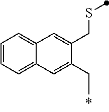 |
| 37 | 12 | 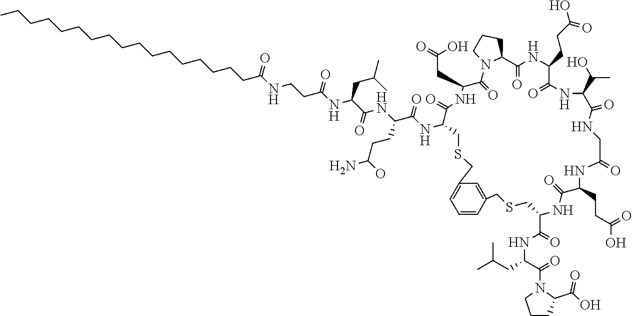 [SMILES: CCCCCCCCCCCCCCCCCC(=O)NCCC(=O)N[C@@H](CC(C)C)C(=O)N[C@@H](CCC(N)O)C(=O)N[C@H]1CSCc2cccc(c2)CSC[C@@H](C(=O)N[C@@H](CC(C)C)C(=O)N2CCC[C@H]2C(=O)O)NC(=O)[C@H](CCC(=O)O)NC(=O)CNC(=O)[C@H](C(C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-OH | -Pro- | 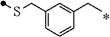 |
| 38 | 12 | 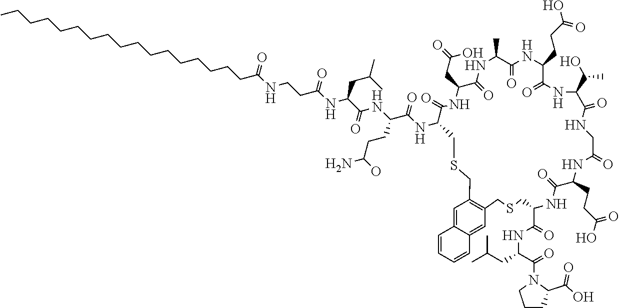 [SMILES: CCCCCCCCCCCCCCCCCC(=O)NCCC(=O)N[C@@H](CC(C)C)C(=O)N[C@@H](CCC(N)O)C(=O)N[C@H]1CSCc2cc3ccccc3cc2CSC[C@@H](C(=O)N[C@@H](CC(C)C)C(=O)N2CCC[C@H]2C(=O)O)NC(=O)[C@H](CCC(=O)O)NC(=O)CNC(=O)[C@H]([C@@H](C)O)NC(=O)[C@H](CCC(=O)O)NC(=O)[C@H](C)NC(=O)[C@H](CC(=O)O)NC1=O] | -Gln-Leu-CO— (CH₂)₂—NH—CO— (CH₂)₁₆—CH₃ | -Leu-Pro-OH | -Ala- | 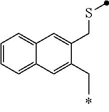 |
| 39 | 12 | 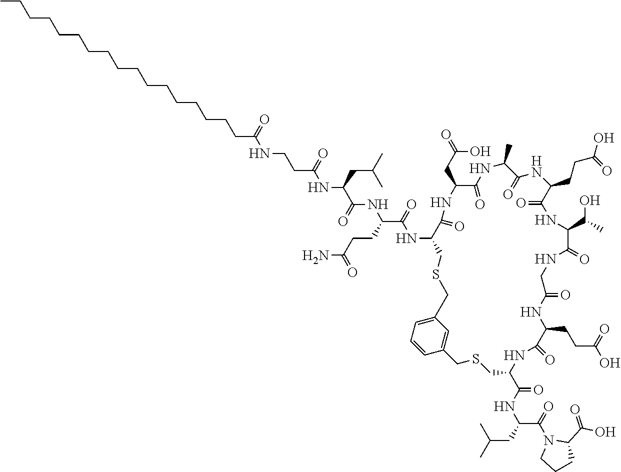 [SMILES: CCCCCCCCCCCCCCCCCC(=O)NCCC(=O)N[C@@H](CC(C)C)C(=O)N[C@@H](CCC(N)=O)C(=O)N[C@H]1CSCc2cccc(c2)CSC[C@@H](C(=O)N[C@@H](CC(C)C)C(=O)N2CCC[C@H]2C(=O)O)NC(=O)[C@H](CCC(=O)O)NC(=O)CNC(=O)[C@H]([C@@H](C)O)NC(=O)[C@H](CCC(=O)O)NC(=O)[C@H](C)NC(=O)[C@H](CC(=O)O)NC1=O] | -Gln-Leu-CO— (CH₂)₂—NH—CO— (CH₂)₁₆—CH₃ | -Leu-Pro-OH | -Ala- | 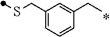 |
| 40 | 12 | 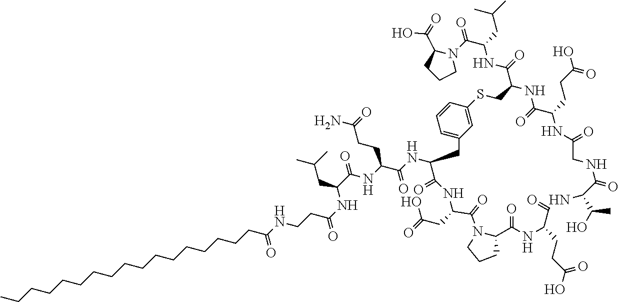 [SMILES: CCCCCCCCCCCCCCCCCC(=O)NCCC(=O)N[C@@H](CC(C)C)C(=O)N[C@@H](CCC(N)=O)C(=O)N[C@H]1Cc2cccc(c2)SC[C@@H](C(=O)N[C@@H](CC(C)C)C(=O)N2CCC[C@H]2C(=O)O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-OH | -Pro- | 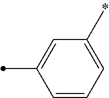 |
| 41 | 8 | 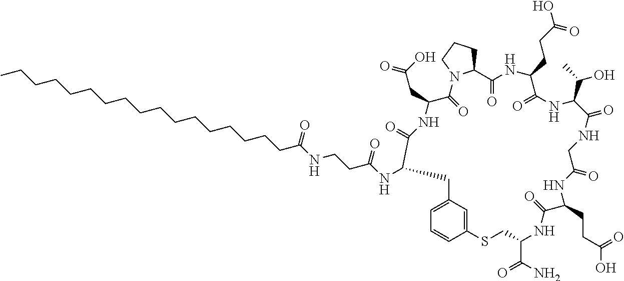 [SMILES: CCCCCCCCCCCCCCCCCC(=O)NCCC(=O)N[C@H]1Cc2cccc(c2)SC[C@@H](C(N)=O)NC(=O)[C@H](CCC(=O)O)NC(=O)CNC(=O)[C@H]([C@H](C)O)NC(=O)[C@H](CCC(=O)O)NC(=O)[C@@H]2CCCN2C(=O)[C@H](CC(=O)O)NC1=O] | —CO—(CH₂)₂—NH— CO—(CH₂)₁₆—CH₃ | —NH₂ | -Pro- | 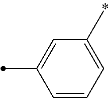 |
| 42 | 12 | 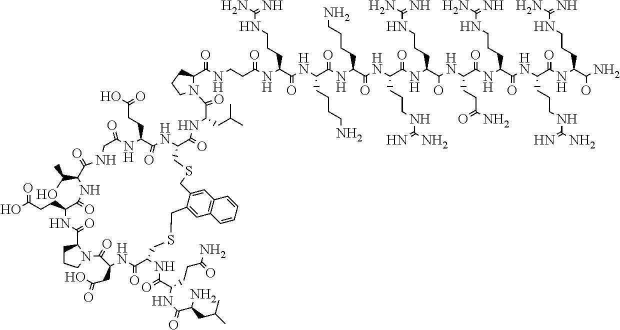 [SMILES: CC(C)C[C@H](NC(=O)[C@@H]1CSCc2cc3ccccc3cc2CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)O] | -Gln-Leu-H | -Leu-Pro-NH—(CH₂)₂— CO-Arg-Lys-Lys- Arg-Arg-Gln-Arg-- Arg-Arg-NH₂ | -Pro- | 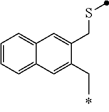 |
| 43 | 8 | 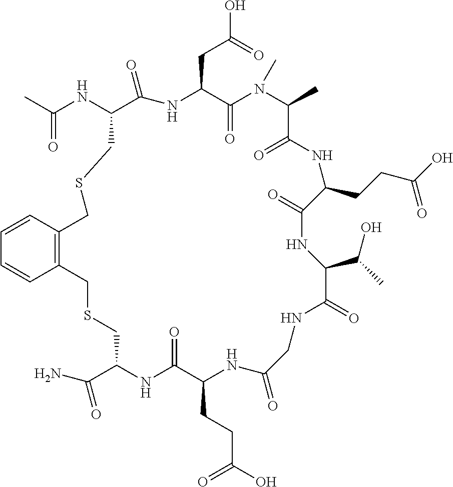 [SMILES: CC(=O)N[C@H]1CSCc2ccccc2CSC[C@@H](C(N)=O)NC(=O)[C@H](CCC(=O)O)NC(=O)CNC(=O)[C@H]([C@@H](C)O)NC(=O)[C@H](CCC(=O)O)NC(=O)[C@H](C)N(C)C(=O)[C@H](CC(=O)O)NC1=O] | —COCH₃ | —NH₂ | -MeAla- | 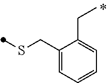 |
| 44 | 12 | 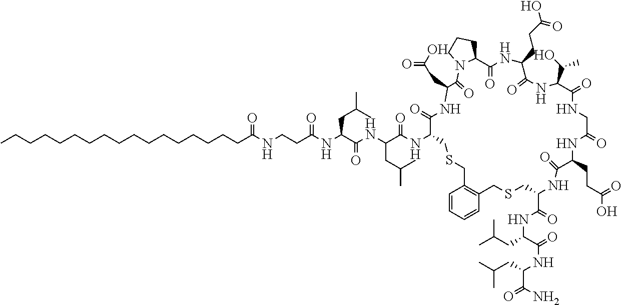 [SMILES: CCCCCCCCCCCCCCCCCC(=O)NCCC(=O)N[C@@H](CC(C)C)C(=O)NC(CC(C)C)C(=O)N[C@H]1CSCc2ccccc2CSC[C@@H](C(=O)N[C@@H](CC(C)C)C(=O)N[C@@H](CC(C)C)C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Leu-Leu-CO— (CH₂)₂—NH—CO— (CH₂)₁₆—CH₃ | -Leu-Leu-NH₂ | -Pro- | 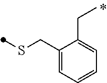 |
| 45 | 12 | 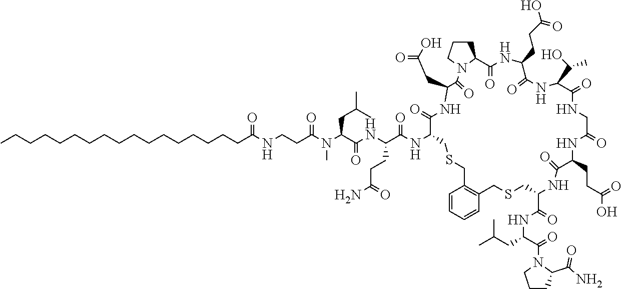 [SMILES: CCCCCCCCCCCCCCCCCC(=O)NCCC(=O)N(C)[C@@H](CC(C)C)C(=O)N[C@@H](CCC(N)=O)C(=O)N[C@H]1CSCc2ccccc2CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-MeLeu-CO— (CH₂)₂—NH—CO— (CH₂)₁₆—CH₃ | -Leu-Pro-NH₂ | -Pro- | 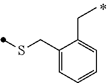 |
| 46 | 12 | 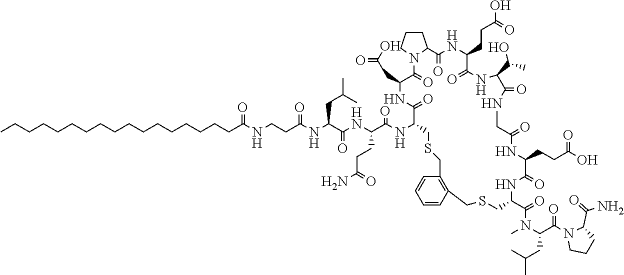 [SMILES: CCCCCCCCCCCCCCCCCC(=O)NCCC(=O)N[C@@H](CC(C)C)C(=O)N[C@@H](CCC(N)=O)C(=O)N[C@H]1CSCc2ccccc2CSC[C@@H](C(=O)N(C)[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)C2CCCN2C(=O)[C@H](CC(=O)O)NC1=O] | -Gln-Leu-CO— (CH₂)₂—NH—CO— (CH₂)₁₆—CH₃ | -MeLeu-Pro-NH₂ | -Pro- | 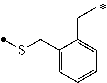 |
| 47 | 12 | 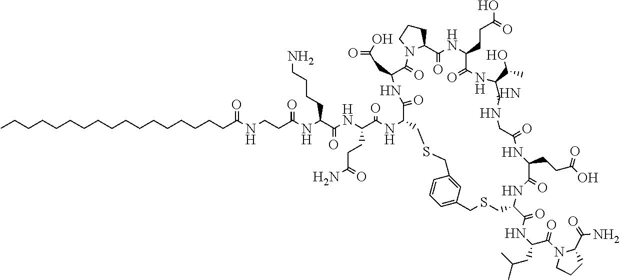 [SMILES: CCCCCCCCCCCCCCCCCC(=O)NCCC(=O)N[C@@H](CCCCN)C(=O)N[C@@H](CCC(N)=O)C(=O)N[C@H]1CSCc2cccc(c2)CSC[C@@H](C(=O)N[C@@H](CC(C)C)C(=O)N2CCC[C@H]2C(N)=O)NC(=O)[C@H](CCC(=O)O)NC(=O)CNC(=N)[C@H]([C@@H](C)O)NC(=O)[C@H](CCC(=O)O)NC(=O)[C@@H]2CCCN2C(=O)[C@H](CC(=O)O)NC1=O] | -Gln-Lys-CO— (CH₂)₂—NH—CO— (CH₂)₁₆—CH₃ | -Leu-Pro-NH₂ | -Pro- | 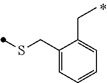 |
| 48 | 12 | 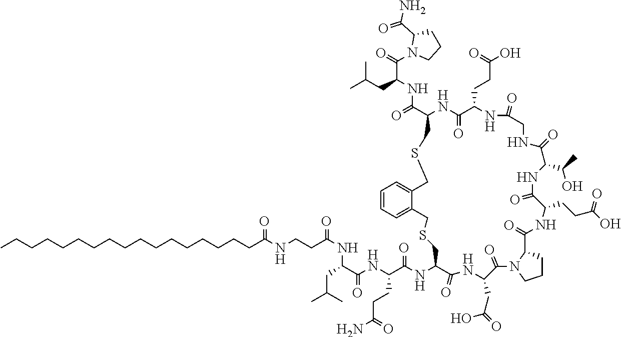 [SMILES: CCCCCCCCCCCCCCCCCC(=O)NCCC(=O)N[C@@H](CC(C)C)C(=O)N[C@@H](CCC(N)=O)C(=O)N[C@H]1CSCc2ccccc2CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH₂ | -Pro- | 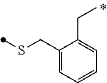 |
| 49 | 12 | 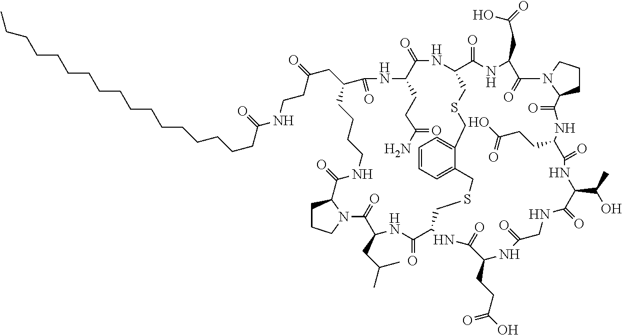 [SMILES: CCCCCCCCCCCCCCCCCC(=O)NCCC(=O)C[C@H]1CCCCNC(=O)[C@@H]2CCCN2C(=O)[C@H](CC(C)C)NC(=O)[C@@H]2CSCc3ccccc3CSC[C@H](NC(=O)[C@H](CCC(N)=O)NC1=O)C(=O)N[C@@H](CC(=O)O)C(=O)N1CCC[C@H]1C(=O)N[C@@H](CCC(=O)O)C(=O)N[C@@H]([C@@H](C)O)C(=O)NCC(=O)N[C@@H](CCC(=O)O)C(=O)N2] | -Gln-Lys(—CO— (CH₂)₂—NH—CO— (CH₂)₁₆—CH₃)— | -Leu-Pro- | -Pro- | 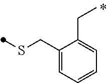 |
| 50 | 12 | 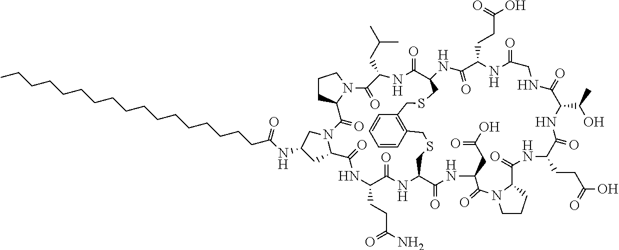 [SMILES: CCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro-((4S)-NH— CO—(CH₂)₁₆—CH₃)— | -Leu-D-Pro- | -Pro- | 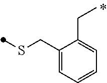 |
| 51 | 12 | 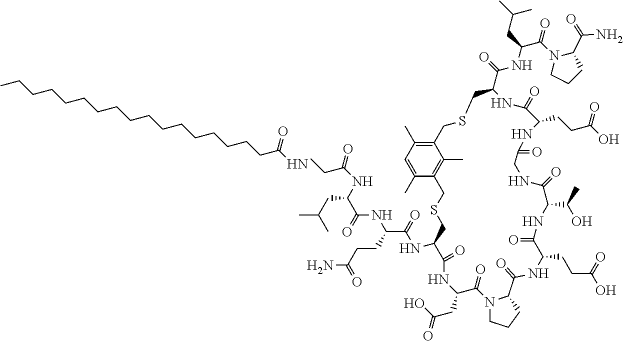 [SMILES: CCCCCCCCCCCCCCCCCC(=O)NCCC(=O)N[C@@H](CC(C)C)C(=O)N[C@@H](CCC(N)=O)C(=O)N[C@H]1CSCc2c(C)cc(C)c(c2C)CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH₂ | -Pro- | 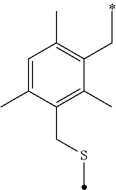 |
| 52 | 12 | 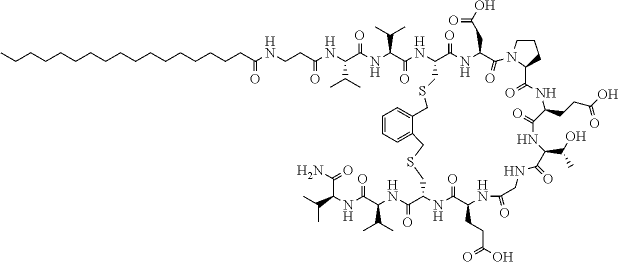 [SMILES: CCCCCCCCCCCCCCCCCC(=O)NCCC(=O)N[C@H](C(=O)N[C@H](C(=O)N[C@H]1CSCc2ccccc2CSC[C@@H](C(=O)N[C@H](C(=O)N[C@H](C(N)=O)C(C)C)C(C)C)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O)C(C)C)C(C)C] | -Val-Val-CO— (CH₂)₂—NH—CO— (CH₂)₁₆—CH₃ | -Val-Val-NH₂ | -Pro- | 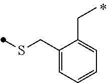 |
| 53 | 12 | 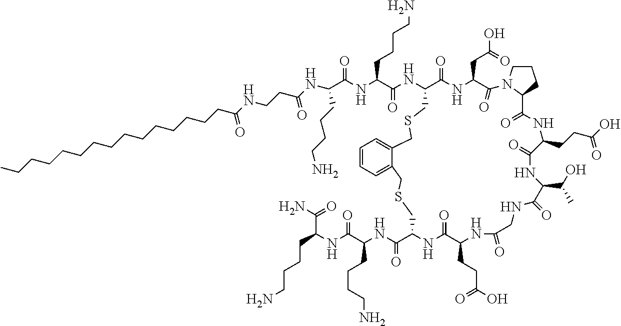 [SMILES: CCCCCCCCCCCCCCCC(=O)NCCC(=O)N[C@@H](CCCCN)C(=O)N[C@@H](CCCCN)C(=O)N[C@H]1CSCc2ccccc2CSC[C@@H](C(=O)N[C@@H](CCCCN)C(=O)N[C@@H](CCCCN)C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Lys-Lys-CO— (CH₂)₂—NH—CO— (CH₂)₁₆—CH₃ | -Lys-Lys-NH₂ | -Pro- | 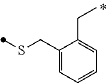 |
| 54 | 12 | 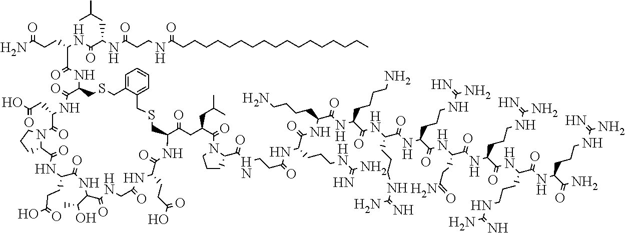 [SMILES: CCCCCCCCCCCCCCCCCC(=O)NCCC(=O)N[C@@H](CC(C)C)C(=O)N[C@@H](CCC(N)=O)C(=O)N[C@H]1CSCc2ccccc2CSC[C@@H](C(=O)C[C@@H](CC(C)C)C(=O)N2CCC[C@H]2C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O)NC(=O)[C@H](CCC(=O)O)NC(=O)CNC(=O)C([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH—(CH₂)₂— CO-Arg-Lys-Lys- Arg-Arg-Gln-Arg- Arg-Arg-NH₂; | -Pro- | 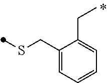 |
| 55 | 12 | 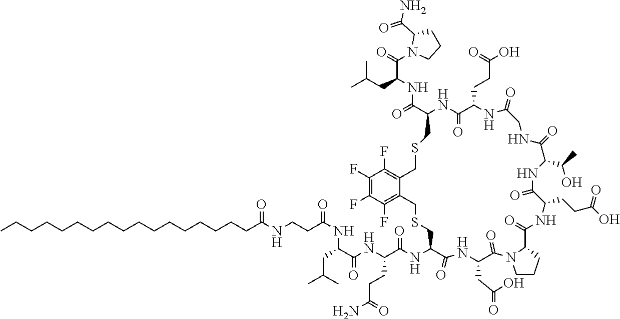 [SMILES: CCCCCCCCCCCCCCCCCC(=O)NCCC(=O)N[C@@H](CC(C)C)C(=O)N[C@@H](CCC(N)=O)C(=O)N[C@H]1CSCc2c(F)c(F)c(F)c(F)c2CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH₂ | -Pro- | 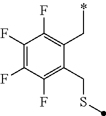 |
| 56 | 12 | 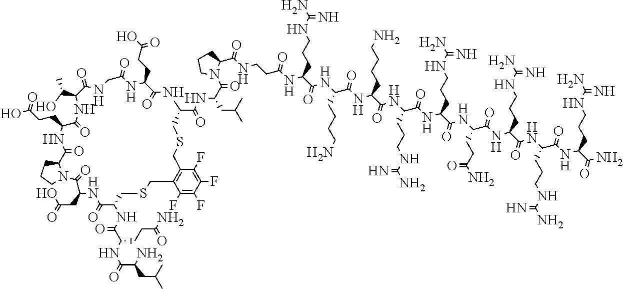 [SMILES: CC(C)C[C@H](NC(=O)[C@@H]1CSCc2c(F)c(F)c(F)c(F)c2CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂; | -Pro- | 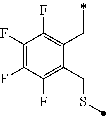 |
| 57 | 12 | 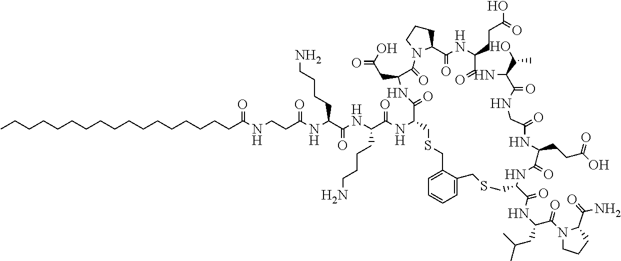 [SMILES: CCCCCCCCCCCCCCCCCC(=O)NCCC(=O)N[C@@H](CCCCN)C(=O)N[C@@H](CCCCN)C(=O)N[C@H]1CSCc2ccccc2CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Lys-Leu-CO— (CH₂)₂—NH—CO— (CH₂)₁₆—CH₃ | -Leu-Pro-NH₂ | -Pro- | 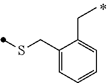 |
| 58 | 12 | 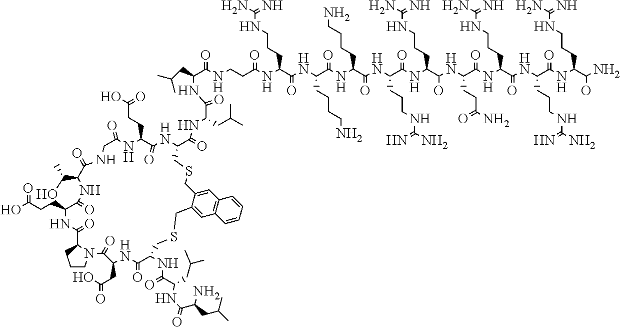 [SMILES: CC(C)C[C@H](NC(=O)[C@H](CC(C)C)NC(=O)[C@@H]1CSCc2cc3ccccc3cc2CSC[C@H](NC(=O)[C@H](CC(C)C)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@@H](C)O)C(=O)NCC(=O)N[C@@H](CCC(=O)O)C(=O)N1)C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)O] | -Leu-Leu-H | -Leu-Leu-NH—(CH₂)₂— CO-Arg-Lys-Lys- Arg-Arg-Gln-Arg- Arg-Arg-NH₂ | -Pro- | 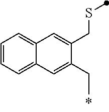 |
| 59 | 12 | 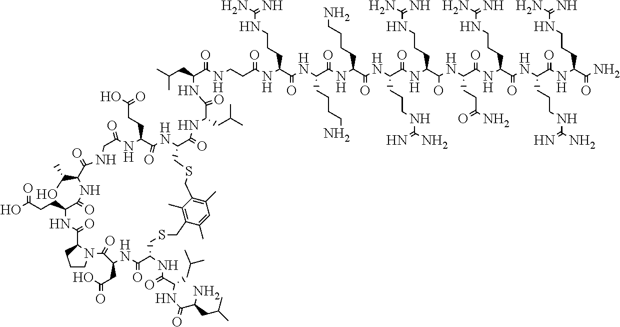 [SMILES: Cc1cc(C)c2c(C)c1CSC[C@@H](C(=O)N[C@@H](CC(C)C)C(=O)N[C@@H](CC(C)C)C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O)NC(=O)[C@H](CCC(=O)O)NC(=O)CNC(=O)[C@H]([C@@H](C)O)NC(=O)[C@H](CCC(=O)O)NC(=O)[C@@H]1CCCN1C(=O)[C@H](CC(=O)O)NC(=O)[C@@H](NC(=O)[C@H](CC(C)C)NC(=O)[C@@H](N)CC(C)C)CSC2] | -Leu-Leu-H | -Leu-Leu-NH—(CH₂)₂— CO-Arg-Lys-Lys- Arg-Arg-Gln-Arg- Arg-Arg-NH₂ | -Pro- | 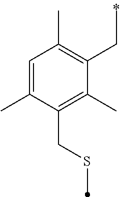 |
| 60 | 12 | 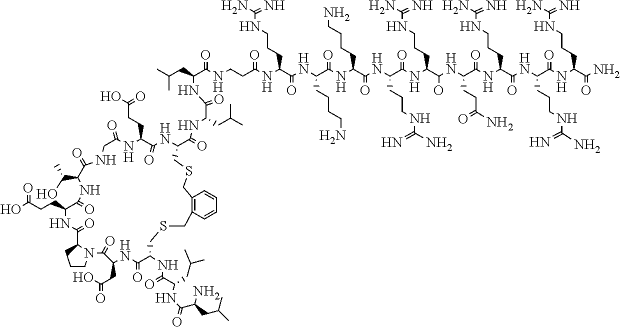 [SMILES: CC(C)C[C@H](NC(=O)[C@H](CC(C)C)NC(=O)[C@@H]1CSCc2ccccc2CSC[C@H](NC(=O)[C@H](CC(C)C)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@@H](C)O)C(=O)NCC(=O)N[C@@H](CCC(=O)O)C(=O)N1)C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Leu-Leu-H | -Leu-Leu-NH—(CH₂)₂— CO-Arg-Lys-Lys- Arg-Arg-Gln-Arg- Arg-Arg-NH₂ | -Pro- | 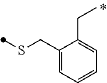 |
| 61 | 12 | 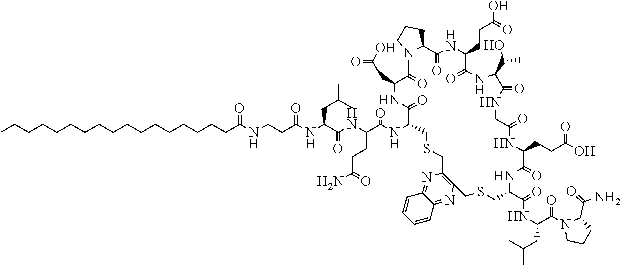 [SMILES: CCCCCCCCCCCCCCCCCC(=O)NCCC(=O)N[C@@H](CC(C)C)C(=O)NC(CCC(N)=O)C(=O)N[C@H]1CSCc2nc3ccccc3nc2CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH₂ | -Pro- | 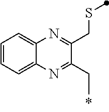 |
| 62 | 12 | 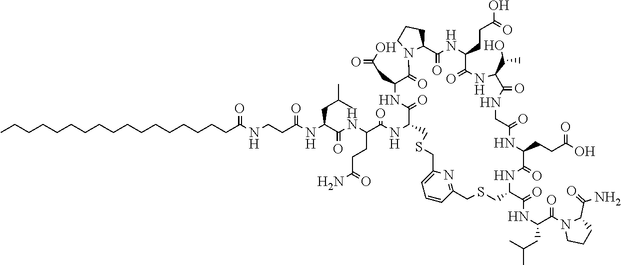 [SMILES: CCCCCCCCCCCCCCCCCC(=O)NCCC(=O)N[C@@H](CC(C)C)C(=O)NC(CCC(N)=O)C(=O)N[C@H]1CSCc2cccc(n2)CSC[C@@H](C(=O)N[C@@H](CC(C)C)C(=O)N2CCC[C@H]2C(N)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Gln-Leu-CO— (CH₂)₂—NH—CO— (CH₂)₁₆—CH₃ | -Leu-Pro-NH₂ | -Pro- | 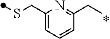 |
| 63 | 12 | 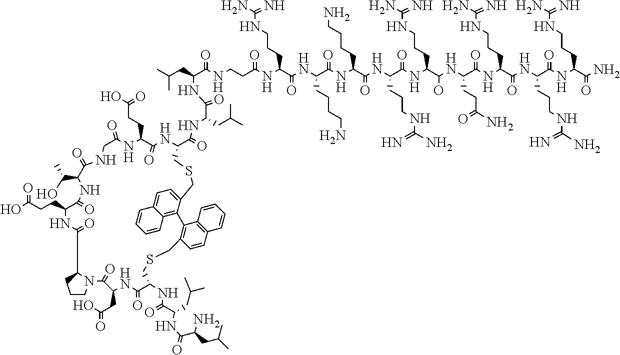 [SMILES: CC(C)C[C@H](NC(=O)[C@H](CC(C)C)NC(=O)[C@@H]1CSCc2ccc3ccccc3c2-c2c(ccc3ccccc23)CSC[C@H](NC(=O)[C@H](CC(C)C)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@@H](C)O)C(=O)NCC(=O)N[C@@H](CCC(=O)O)C(=O)N1)C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Leu-Leu-H | -Leu-Leu-NH—(CH₂)₂— CO-Arg-Lys-Lys- Arg-Arg-Gln-Arg- Arg-Arg-NH₂ | -Pro- | 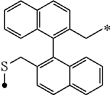 |
| 64 | 12 | 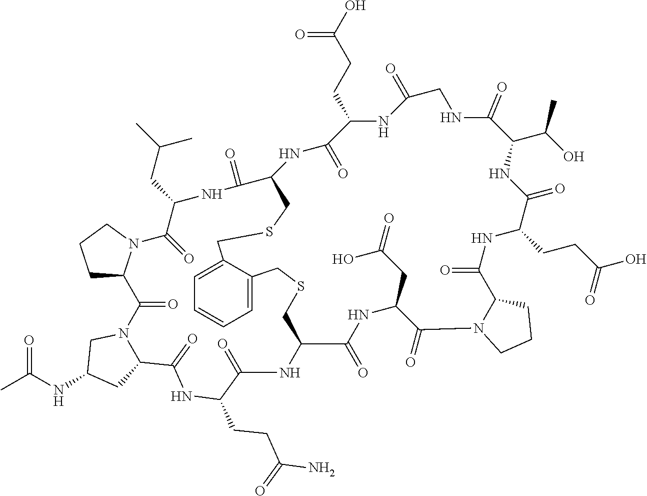 [SMILES: CC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)- NHC(O)CH₃)— | -Leu-D-Pro | -Pro- | 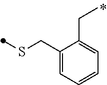 |
| 65 | 12 | 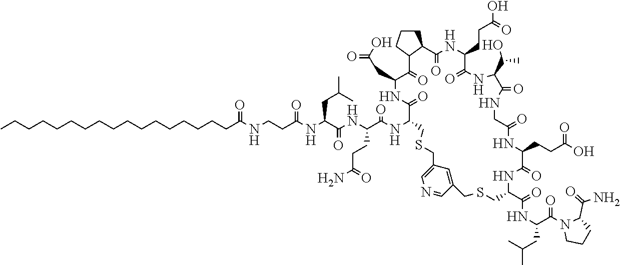 | -Gln-Leu-CO— (CH₂)₂—NH—CO— (CH₂)₁₆—CH₃ | -Leu-Pro-NH₂ | -Pro- | 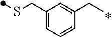 |
| 66 | 12 | 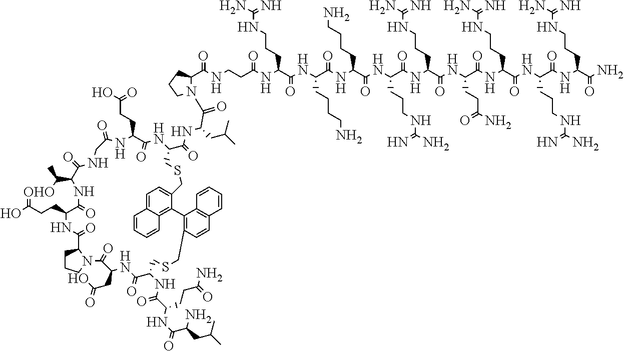 [SMILES: CC(C)C[C@H](NC(=O)[C@@H]1CSCc2ccc3ccccc3c2-c2c(ccc3ccccc23)CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Lys-Lys- Arg-Arg-Gln-Arg- Arg-Arg-NH₂ | -Pro- | 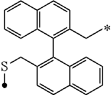 |
| 67 | 12 | 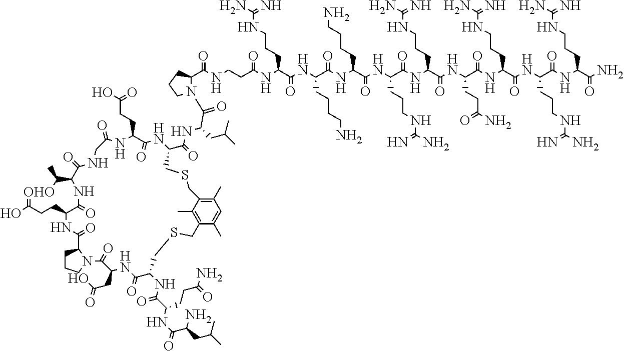 [SMILES: Cc1cc(C)c2c(C)c1CSC[C@@H](C(=O)N[C@@H](CC(C)C)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O)NC(=O)[C@H](CCC(=O)O)NC(=O)CNC(=O)[C@H]([C@H](C)O)NC(=O)[C@H](CCC(=O)O)NC(=O)[C@@H]1CCCN1C(=O)[C@H](CC(=O)O)NC(=O)[C@@H](NC(=O)[C@H](CCC(N)=O)NC(=O)[C@@H](N)CC(C)C)CSC2] | -Gln-Leu-H | -Leu-Pro-NH—(CH₂)₂— CO-Arg-Lys-Lys- Arg-Arg-Gln-Arg- Arg-Arg-NH₂ | -Pro- | 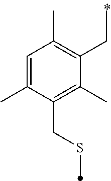 |
| 68 | 12 | 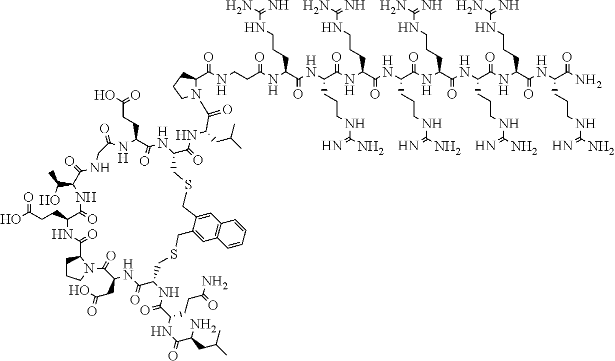 [SMILES: CC(C)C[C@H](NC(=O)[C@@H]1CSCc2cc3ccccc3cc2CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Arg-Arg- Arg-Arg-Arg-Arg- Arg-NH₂ | -Pro- | 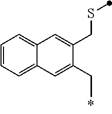 |
| 69 | 12 | 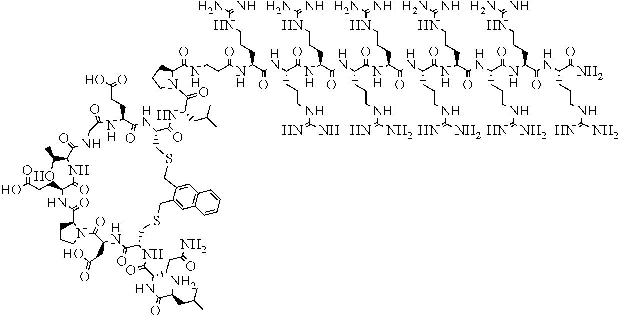 [SMILES: CC(C)C[C@H](NC(=O)[C@@H]1CSCc2cc3ccccc3cc2CSC[C@H](NC(=O)[C@H](CCC(N)=O)NC(=O)[C@@H](N)CC(C)C)C(=O)N[C@@H](CC(=O)O)C(=O)N2CCC[C@H]2C(=O)N[C@@H](CCC(=O)O)C(=O)N[C@@H]([C@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH—(CH₂)₂— CO-Arg-Arg-Arg- Arg-Arg-Arg-Arg- Arg-Arg-Arg-NH₂ | -Pro- | 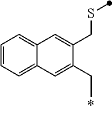 |
| 70 | 12 | 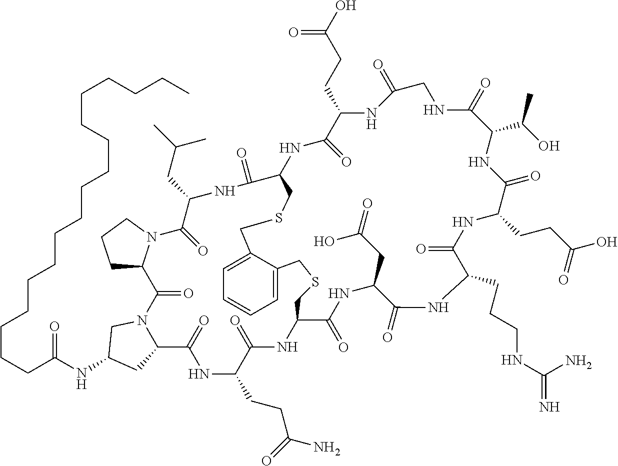 [SMILES: CCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@H](CCCNC(=N)N)NC(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₆— CH₃)— | -Leu-D-Pro- | -Arg- | 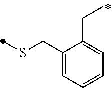 |
| 71 | 12 | 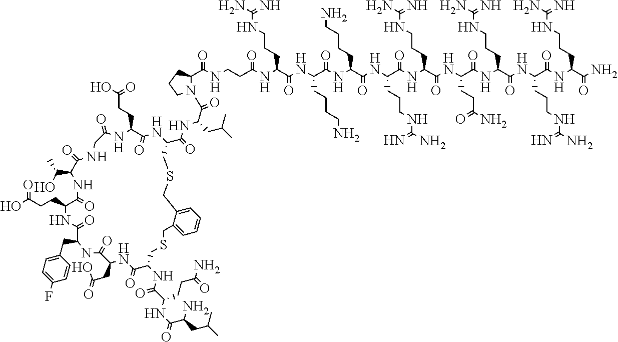 [SMILES: CC(C)C[C@H](NC(=O)[C@@H]1CSCc2ccccc2CSC[C@H](NC(=O)[C@H](CCC(N)=O)NC(=O)[C@@H](N)CC(C)C)C(=O)N[C@@H](CC(=O)O)C(=O)N[C@@H](Cc2ccc(F)cc2)C(=O)N[C@@H](CCC(=O)O)C(=O)N[C@@H]([C@@H](C)O)C(=O)NCC(=O)N[C@@H](CCC(=O)O)C(=O)N1)C(=O)N1CCC[C@H]1C(=O)NCCC(=O)N[C@@H](CCCNC(=N)N)C(=O)N[C@@H](CCCCN)C(=O)N[C@@H](CCCCN)C(=O)N[C@@H](CCCNC(=N)N)C(=O)N[C@@H](CCCNC(=N)N)C(=O)N[C@@H](CCC(N)=O)C(=O)N[C@@H](CCCNC(=N)N)C(=O)N[C@@H](CCCNC(=N)N)C(=O)N[C@@H](CCCNC(=N)N)C(N)=O] | -Gln-Leu-H | -Leu-Pro-NH-(CH₂)₂— CO-Arg-Lys-Lys- Arg-Arg-Gln-Arg- Arg-Arg-NH₂ | -Pro- | 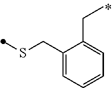 |
| 72 | 12 | 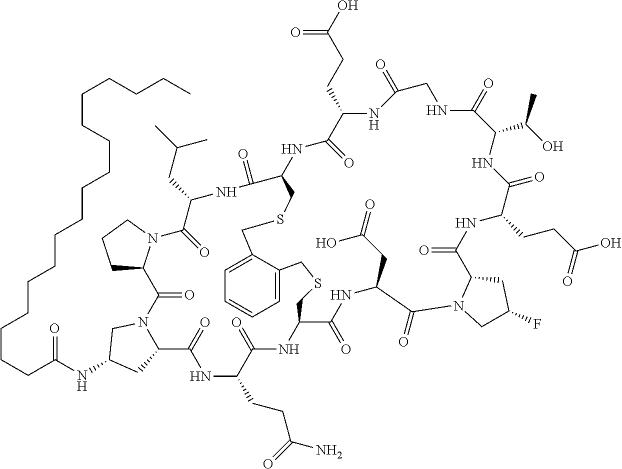 [SMILES: CCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4C[C@H](F)CN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₆— CH₃)— | -Leu-D-Pro- | -(4- F)Pro- | 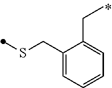 |
| 73 | 12 | 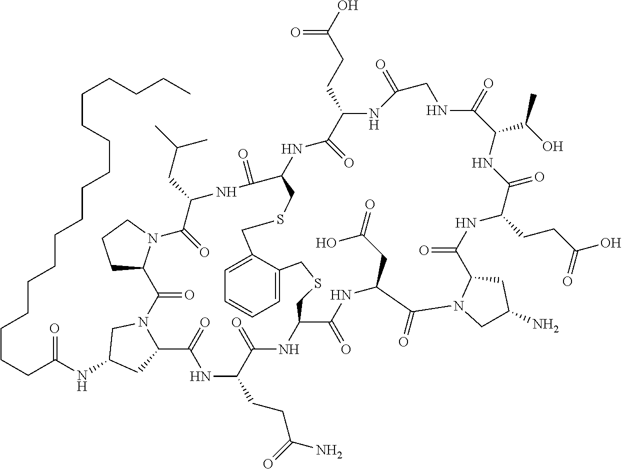 [SMILES: CCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4C[C@H](N)CN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₆— CH₃)— | -Leu-D-Pro- | -(4- NH₂) Pro- | 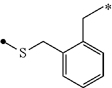 |
| 74 | 12 | 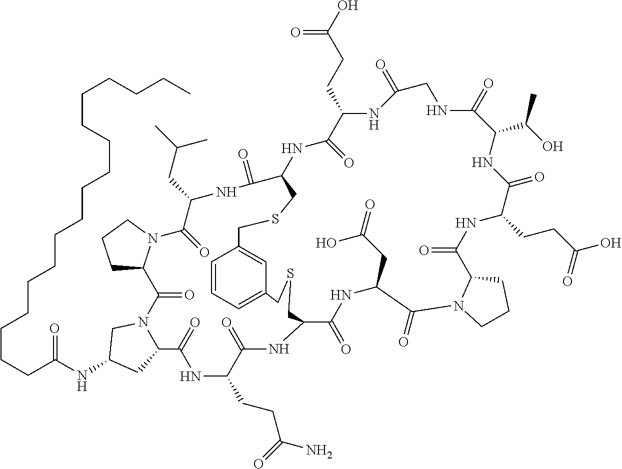 [SMILES: CCCCCCCCCCCCCCCCCC(=O)N[C@H]1C[C@H]2C(=O)N[C@@H](CCC(N)=O)C(=O)N[C@H]3CSCc4cccc(c4)CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₆— CH₃)— | -Leu-D-Pro- | -Pro- | 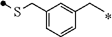 |
| 75 | 12 | 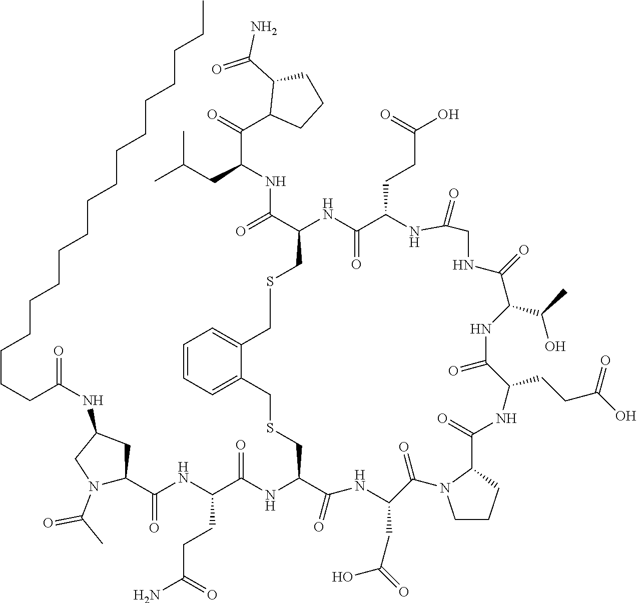 | -Gln-AcPro((4S)- NH—CO—(CH₂)₁₆— CH₃)— | -Leu-D-Pro-NH₂ | -Pro- | 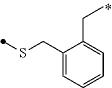 |
| 76 | 12 | 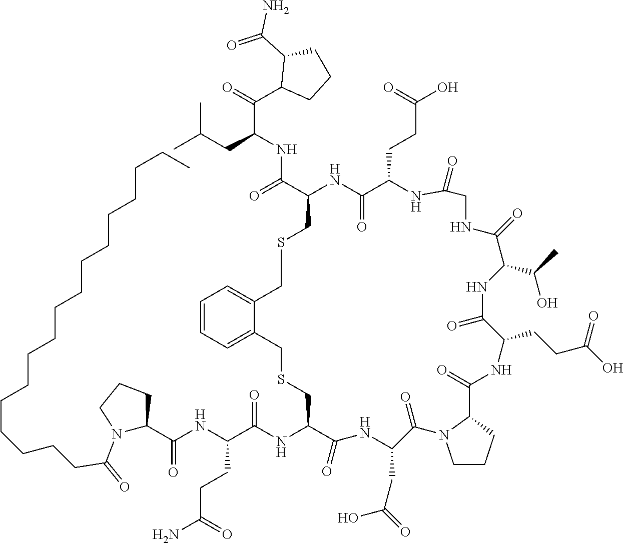 | -Gln-Pro-CO— (CH₂)₁₆—CH₃ | -Leu-D-Pro-NH₂ | -Pro- | 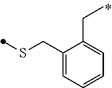 |
| 77 | 12 | 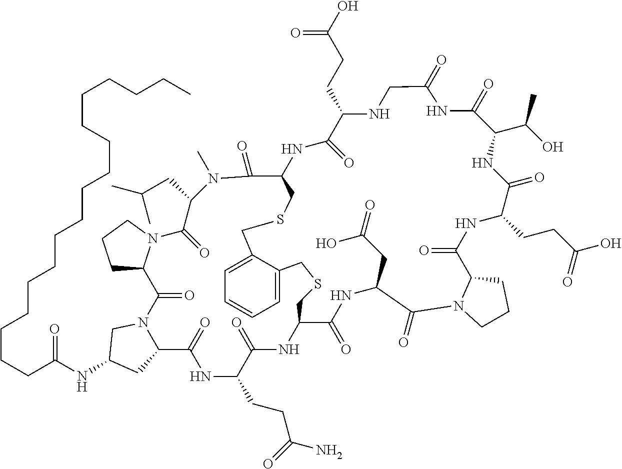 [SMILES: CCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NCC(=O)NC(=O)[C@H]([C@@H](C)O)NC(=O)[C@H](CCC(=O)O)NC(=O)[C@@H]4CCCN4C(=O)[C@H](CC(=O)O)NC3=O)C(=O)N(C)[C@@H](CC(C)C)C(=O)N3CCC[C@@H]3C(=O)N2C1] | -Gln-Pro((4S)—NH— CO—(CH₂)₁₆—CH₃)— | -MeLeu-D-Pro- | -Pro- | 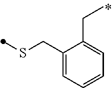 |
| 78 | 12 | 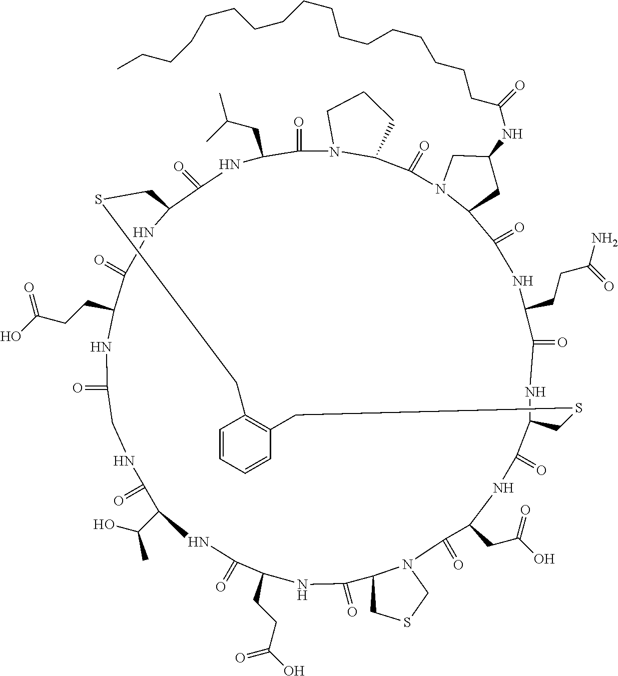 | -Gln-Pro((4S)—NH— CO—(CH₂)₁₆—CH₃)— | -Leu-D-Pro- | -Thz- | 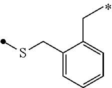 |
| 79 | 12 | 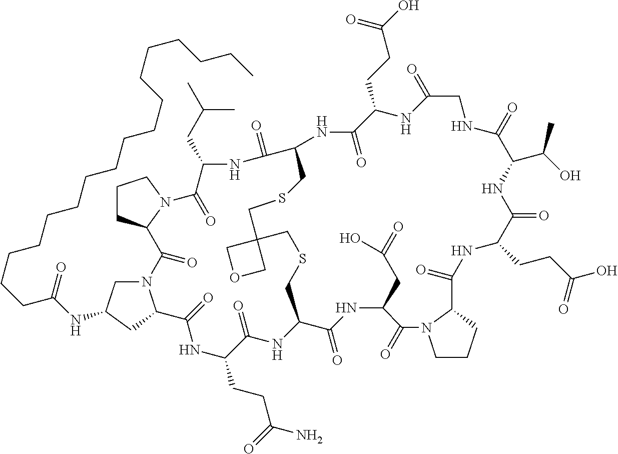 [SMILES: CCCCCCCCCCCCCCCCCC(=O)N[C@H]1C[C@H]2C(=O)N[C@@H](CCC(N)=O)C(=O)N[C@H]3CSCC4(COC4)CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)—NH— CO—(CH₂)₁₆—CH₃)— | -Leu-D-Pro- | -Pro- | 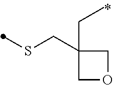 |
| 80 | 12 | 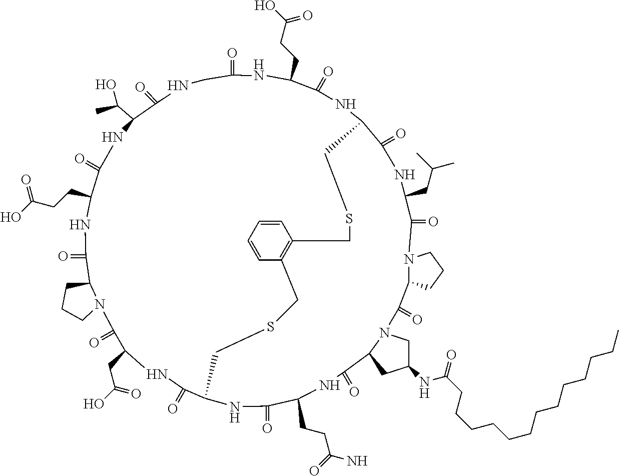 | -Gln-Pro((4S)-NH— CO—(CH₂)₁₂—CH₃)— | -Leu-D-Pro- | -Pro- | 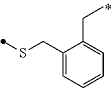 |
| 81 | 12 | 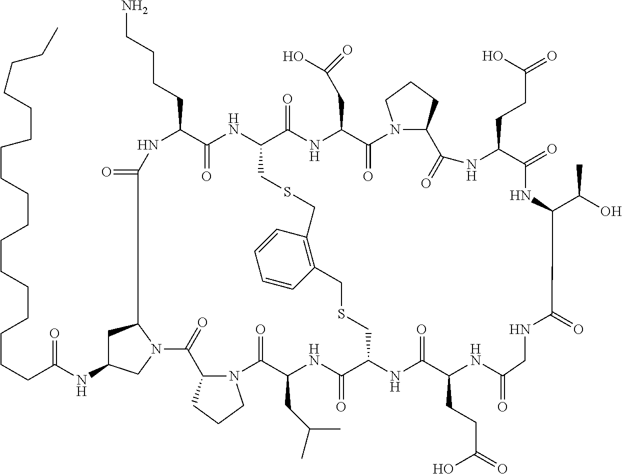 [SMILES: CCCCCCCCCCCCCCCCCC(=O)N[C@H]1C[C@H]2C(=O)N[C@@H](CCCCN)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Lys-Pro((4S)-NH— CO—(CH₂)₁₆—CH₃)— | -Leu-D-Pro- | -Pro- | 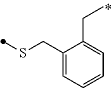 |
| 82 | 12 | 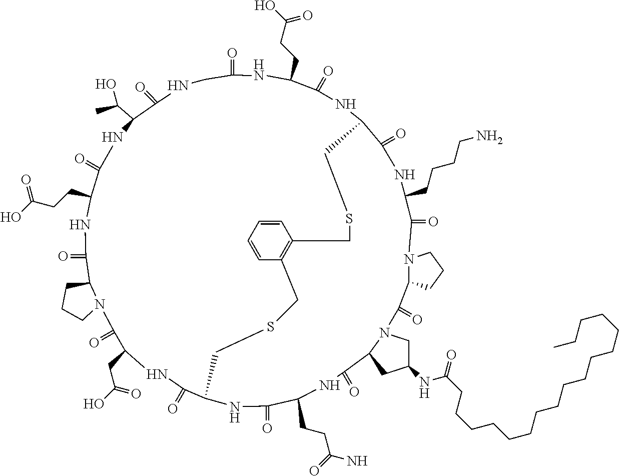 | -Gln-Pro((4S)-NH— CO—(CH₂)₁₆—CH₃)— | -Leu-D-Pro- | -Pro- | 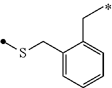 |
| 83 | 12 | 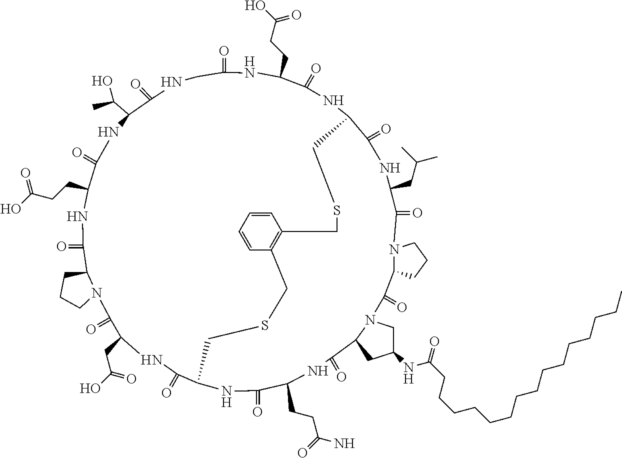 | -Gln-Pro((4S)-NH— CO—(CH₂)₁₄—CH₃)— | -Leu-D-Pro- | -Pro- | 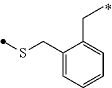 |
| 84 | 12 | 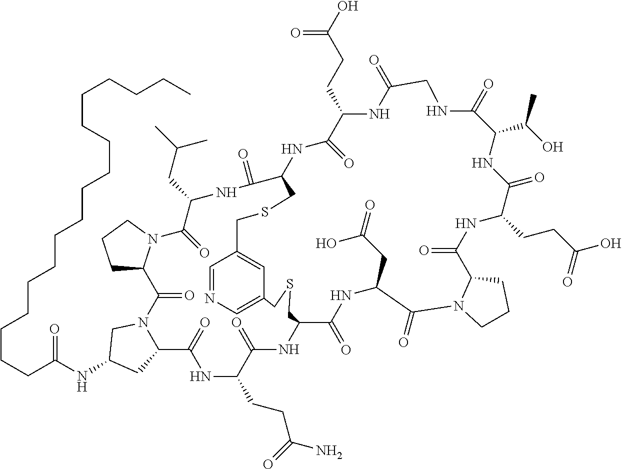 [SMILES: CCCCCCCCCCCCCCCCCC(=O)N[C@H]1C[C@H]2C(=O)N[C@@H](CCC(N)=O)C(=O)N[C@H]3CSCc4cncc(c4)CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₆—CH₃)— | -Leu-D-Pro- | -Pro- | 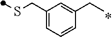 |
| 85 | 12 | 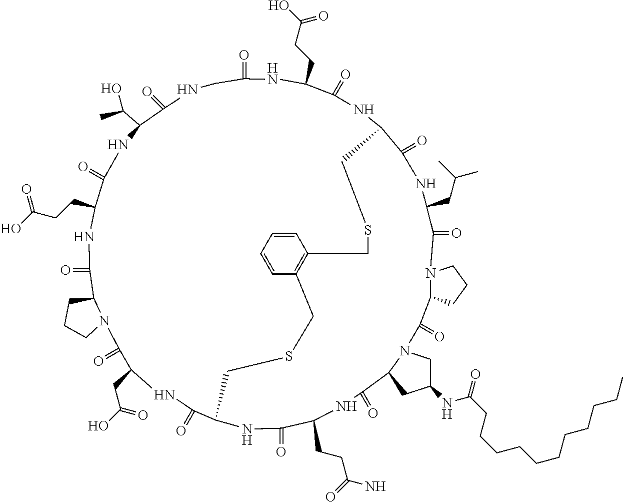 | -Gln-Pro((4S)-NH— CO—(CH₂)₁₀—CH₃)— | -Leu-D-Pro- | -Pro- | 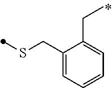 |
| 86 | 12 | 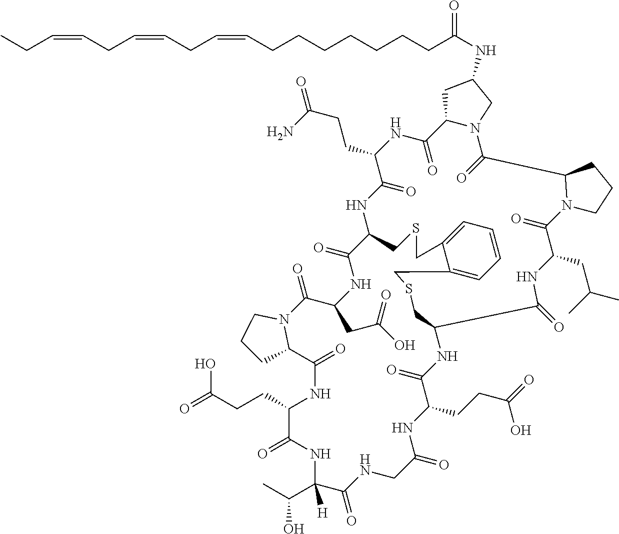 [SMILES: CC/C=C\C/C=C\C/C=C\CCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₇—((Z- CH═CH)—CH₂)₃— CH₃)— | -Leu-D-Pro- | -Pro- | 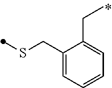 |
| 87 | 12 | 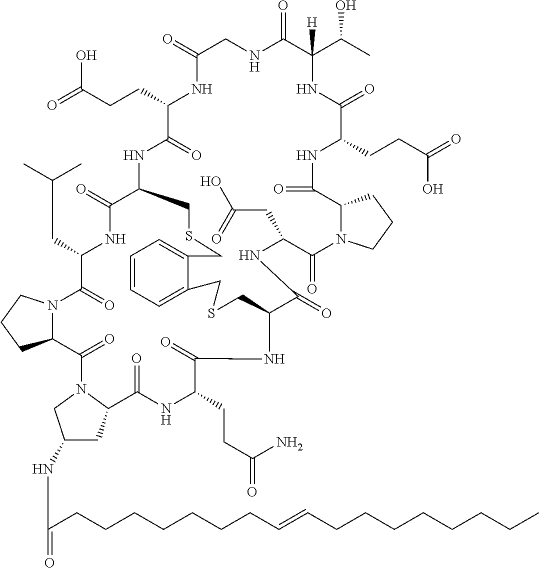 [SMILES: CCCCCCCC/C=C/CCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₇—((E- CH═CH)—CH₂)₁— (CH₂)₆—CH₃)— | -Leu-D-Pro- | -Pro- | 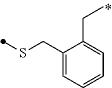 |
| 88 | 12 | 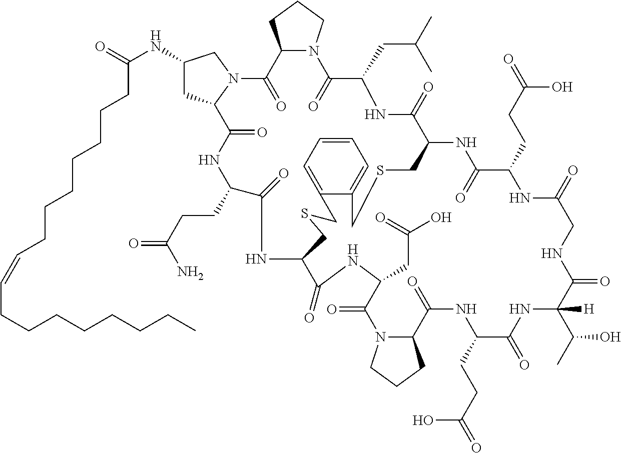 [SMILES: CCCCCCCC/C=C\CCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@H]4CCCN4C(=O)[C@@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₇—((Z- CH═CH)—CH₂)₁— (CH₂)₆—CH₃)— | -Leu-D-Pro- | -Pro- | 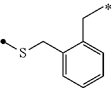 |
| 89 | 12 | 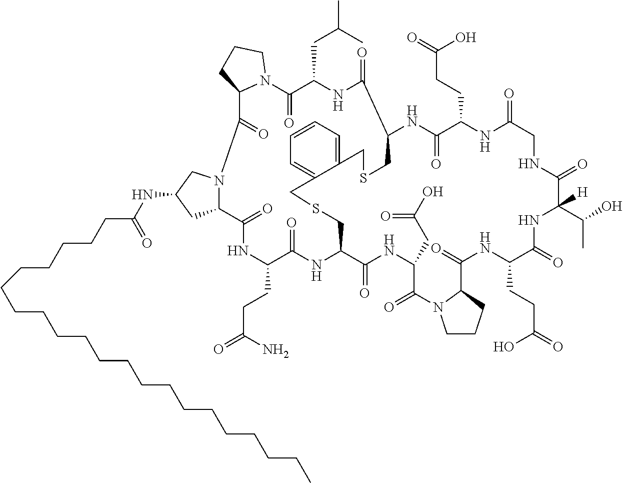 [SMILES: CCCCCCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@H]4CCCN4C(=O)[C@@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₂₀—CH₃)— | -Leu-D-Pro- | -Pro- | 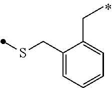 |
| 90 | 12 | 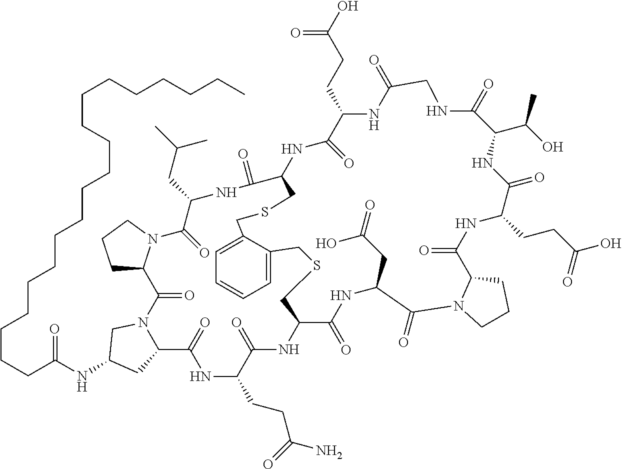 [SMILES: CCCCCCCCCCCCCCCCCCCC(=O)N[C@H]1C[C@H]2C(=O)N[C@@H](CCC(N)=O)C(=O)N[C@H]3CSCc4ccccc4CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₈—CH₃)— | -Leu-D-Pro- | -Pro- | 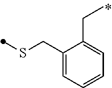 |
| 91 | 12 | 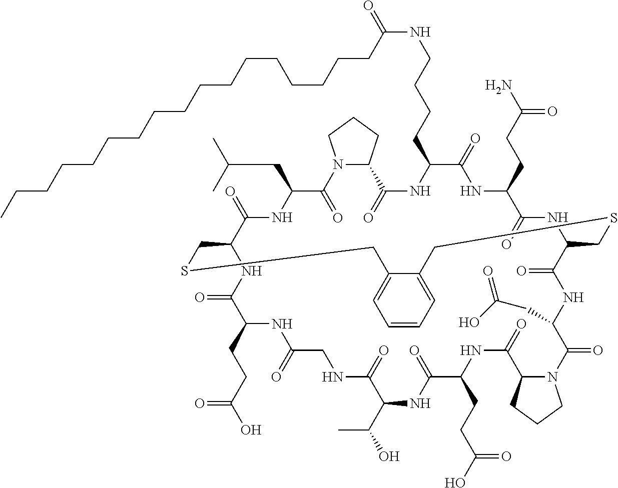 | -Gln-Lys((N⁶—CO— (CH₂)₁₆—CH₃)— | -Leu-D-Pro- | -Pro- | 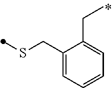 |
| 92 | 12 | 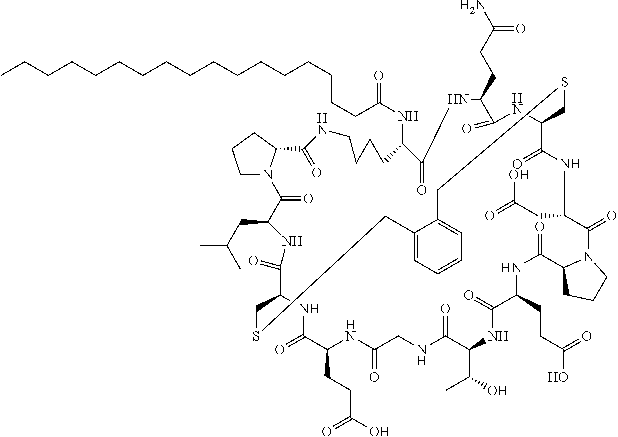 [SMILES: CCCCCCCCCCCCCCCCCC(=O)N[C@H]1CCCCNC(=O)[C@H]2CCCN2C(=O)[C@H](CC(C)C)NC(=O)[C@@H]2CSCc3ccccc3CSC[C@H](NC(=O)[C@H](CCC(N)=O)NC1=O)C(=O)N[C@@H](CC(=O)O)C(=O)N1CCC[C@H]1C(=O)N[C@@H](CCC(=O)O)C(=O)N[C@@H]([C@@H](C)O)C(=O)NCC(=O)N[C@@H](CCC(=O)O)C(=O)N2] | -Gln-Lys((—CO— (CH₂)₁₆—CH₃)— | -Leu-D-Pro- | -Pro- | 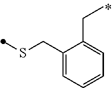 |
| 93 | 12 | 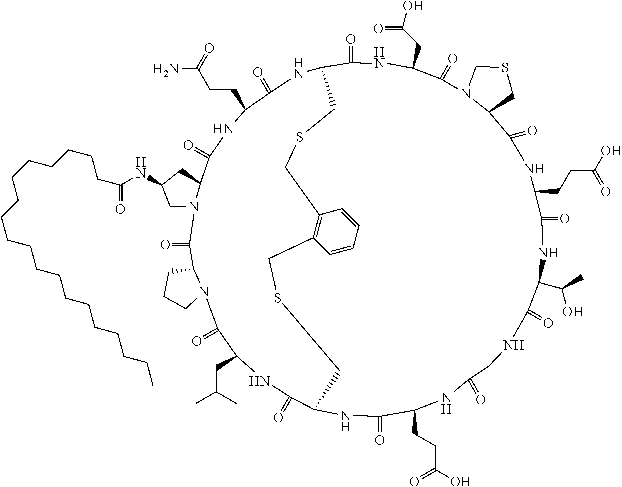 | -Gln-Pro((4S)-NH— CO—(CH₂)₁₈—CH₃)— | -Leu-D-Pro- | -Thz- | 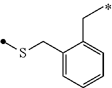 |
| 94 | 12 | 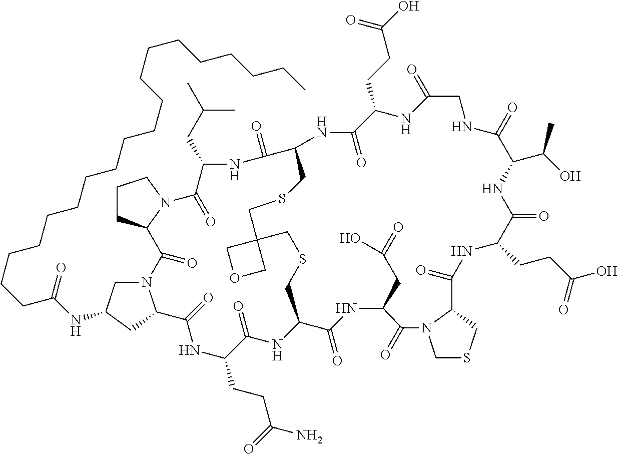 [SMILES: CCCCCCCCCCCCCCCCCCCC(=O)N[C@H]1C[C@H]2C(=O)N[C@@H](CCC(N)=O)C(=O)N[C@H]3CSCC4(COC4)CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CSCN4C(=O)[C@H](CC(=O)O)NC3=O)C(=O)N[C@@H](CC(C)C)C(=O)N3CCC[C@@H]3C(=O)N2C1] | -Gln-Pro((4S)-NH— CO—(CH₂)₁₈—CH₃)— | -Leu-D-Pro- | -Thz- | 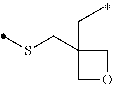 |
| 95 | 12 | 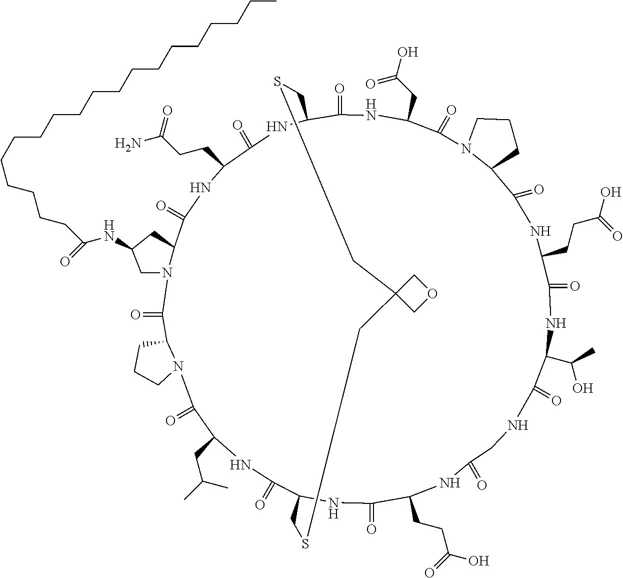 | -Gln-Pro((4S)-NH— CO—(CH₂)₁₈—CH₃)— | -Leu-D-Pro- | -Pro- | 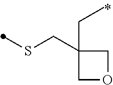 |
| 96 | 12 | 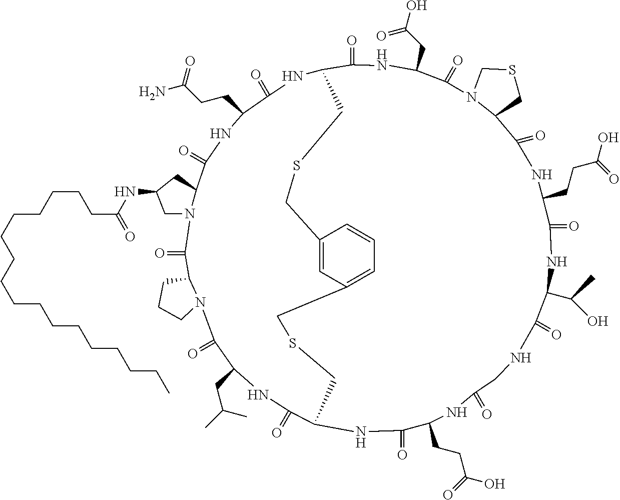 | -Gln-Pro((4S)-NH— CO—(CH₂)₁₆—CH₃)— | -Leu-D-Pro- | -Thz- | 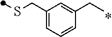 |
| 97 | 12 | 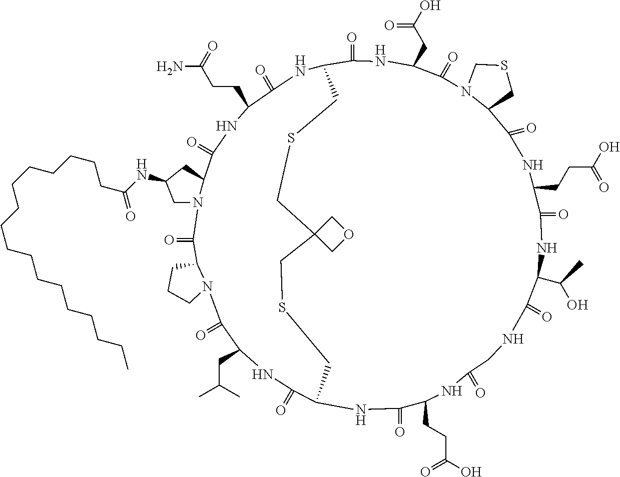 | -Gln-Pro((4S)-NH— CO—(CH₂)₁₆—CH₃)— | -Leu-D-Pro- | -Thz- | 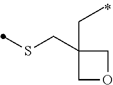 |
| 98 | 12 | 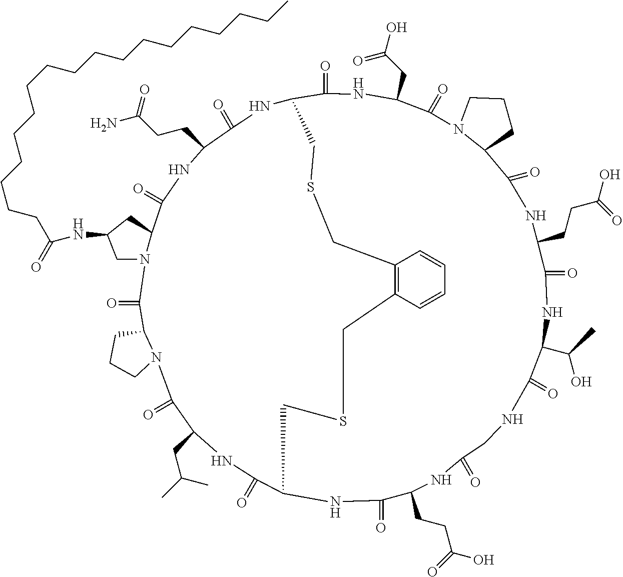 | -Gln-Pro((4S)-NH— CO—(CH₂)₁₇—CH₃)— | -Leu-D-Pro- | -Pro- | 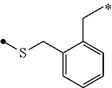 |
| 99 | 12 | 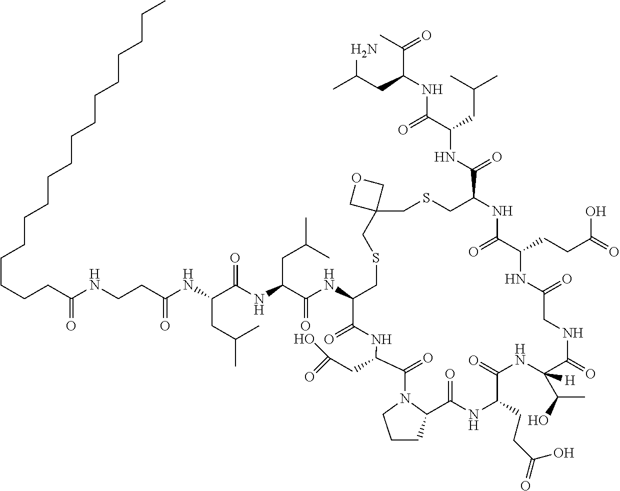 [SMILES: CCCCCCCCCCCCCCCCCC(=O)NCCC(=O)N[C@@H](CC(C)C)C(=O)N[C@@H](CC(C)C)C(=O)N[C@H]1CSCC2(COC2)CSC[C@@H](C(=O)N[C@@H](CC(C)C)C(=O)N[C@@H](CC(C)N)C(C)=O)NC(=O)[C@H](CCC(=O)O)NC(=O)CNC(=O)[C@H]([C@@H](C)O)NC(=O)[C@H](CCC(=O)O)NC(=O)[C@@H]2CCCN2C(=O)[C@H](CC(=O)O)NC1=O] | -Leu-Leu-CO— (CH₂)₂—NH—CO— (CH₂)₁₆—CH₃ | -Leu-Leu-NH₂ | -Pro- | 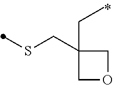 |
| 100 | 12 | 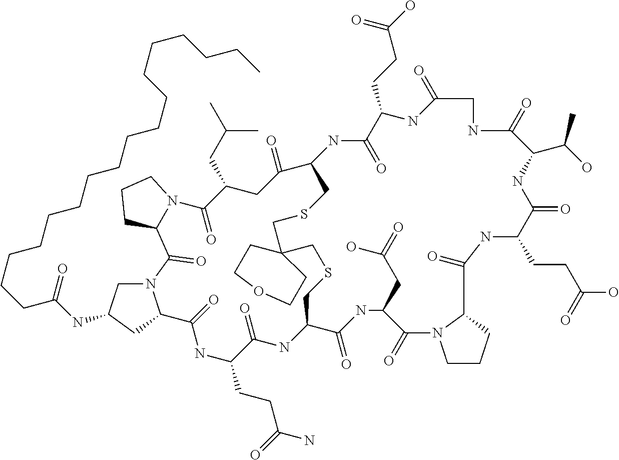 [SMILES: CCCCCCCCCCCCCCCCCC(=O)N[C@H]1C[C@H]2C(=O)N[C@@H](CCC(N)=O)C(=O)N[C@H]3CSCC4(CCOCC4)CSC[C@H](NC(=O)[C@H](CCC(=O)O)NC(=O)CNC(=O)[C@H]([C@@H](C)O)NC(=O)[C@H](CCC(=O)O)NC(=O)[C@@H]4CCCN4C(=O)[C@H](CC(=O)O)NC3=O)C(=O)C[C@@H](CC(C)C)C(=O)N3CCC[C@@H]3C(=O)N2C1] | -Gln-Pro((4S)-NH— CO—(CH₂)₁₆—CH₃)— | -Leu-D-Pro- | -Pro- | 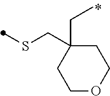 |

### Abbreviations

Aa: amino acid
Ac: acetyl
ACN: acetonitrile
AM: aminomethyl
Alloc: allyloxycarbonyl
βAla: —NH—(CH₂)₂—CO— or —CO—(CH₂)₂—NH—, see TABLE 1.
Boc: tert-butoxycarbonyl
CPP: cell-penetrating peptide
eq: equivalent
Fmoc: 9-fluorenylmethyloxycarbonyl
DCM: dichloromethane
DIEA: N,N′-diisopropylethylamine
DIPCDI: N,N′-diisopropylcarbodiimide
DMF: N,N′-dimethylformamide
DMSO: dimethyl sulfoxide
HBTU: N-[(6-chloro-1H-benzotriazol-1-yl)-(dimethylamino)methylene]N-methylmethanaminium hexafluorophosphate N-oxide
Mmt: 4-methoxytrityl
MW: molecular weight
m/z: mass-to-charge ratio
Oxyma Pure®: ethyl 2-cyano-2-(hydroxyimino)acetate
Pbf: 2,2,4,6,7-pentamethyldihydrobenzofurane-5-sulfonyl
PEG: polyethylene glycol
PG: protecting group
PS: polystyrene
PyBOP: 1-benzotriazole-1-yloxytris(pyrrolidino)phosphonium hexafluorophosphate
PTD4-NH₂: -Tyr-Ala-Arg-Ala-Ala-Ala-Arg-Gln-Ala-Arg-Ala-NH₂
rt: room temperature
RP-HPLC: reverse-phase high-performance liquid chromatography
RP-HPLC-ESI-MS: reverse-phase high-performance liquid chromatography coupled to an electrospray ionization mass spectrometer
RP-UPLC: reverse phase ultra-performance liquid chromatography
RP-UPLC-ESI-MS: reverse phase ultra-performance liquid chromatography-electrospray mass spectrometry
SPPS: solid phase peptide synthesis
TAT-NH₂: -Arg-Lys-Lys-Arg-Arg-Gln-Arg-Arg-Arg-NH₂
tBu: tert-butyl
tBuOH: tert-butanol
TCEP: tris(2-carboxyethyl)phosphine
TFA: trifluoroacetic acid
Thz: L-Thioproline ((4R)-4-Thiazolidinecarboxylic acid)
TIS: triisopropylsilane
t_{R}: retention time
Trt: trityl
UV: ultraviolet

### Materials and Methods

Fmoc-L-Aa-OH derivatives, Fmoc-β-Ala-OH, Fmoc-D-Pro-OH, 2-chlorotrityl chloride PS and Fmoc-Rink amide AM PS resins and HBTU were obtained from IRIS Biotech (Marktredwitz, Germany). Fmoc-Rink amide AM ChemMatrix resin (0.49 mmol/g) and H-Rink amide AM ChemMatrix resin (0.47 mmol/g) were obtained from PCAS (Quebec, Canada). Fmoc-L-Phe(4-I)—OH, Fmoc-L-Phe(34)-OH and Fmoc-L-Phe(2-I)—OH were supplied from Chem-Impex Int. (Illinois, USA). Fmoc-L-NMe-Aa-OH derivatives, stearic acid, caprylic acid, 2,3-bis(bromomethyl)naphthalene, 2,6-bis(bromomethyl)pyridine, PyBOP, Oxyma Pure®, DIEA, DIPCDI, tBuOH, TCEP, I₂, CuI, ethylene glycol, NH₄HCO₃, cis-ciclohexane-1,2-diol, Ac₂O, phenylsilane, Pd(Ph₃P)₄, TIS, 3,3-bis(bromomethyl)oxetane and formic acid were obtained from Aldrich (Schnelldorf, Germany). 3,5-bis(chloromethyl)pyridine.HCl and tetrahydro-2H-pyran-4,4-dicarboxylic acid dimethyl ester were supplied from Fluorochem (Derbyshire, UK). 1,2-bis(bromomethyl)benzene, 1,3-bis(bromomethyl)benzene, 1,4-bis(bromomethyl)benzene, 2,2′-bis(bromomethyl)-1,1′-biphenyl, (R)-2,2′-bis(bromomethyl)-1,1′-binaphthyl, 2,4-bis(chloromethyl)mesitylene and 2,3-bis(bromomethyl)quinoxaline were purchased from Alpha Aesar (Karlsruhe, Germany). 1,2-bis(bromomethyl)-3,4,5,6-tetrafluorobenzene was prepared according to Coe, P. L. et al, Tetrahedron (1967), 23(1), 505-8. MgSO₄ salt was supplied from Acros Organics-Thermo Fisher Scientific (New Jersey, US) and K₂CO₃ salt was purchased from Panreac (Castellar del Valles, Spain). TFA was obtained from Scharlau (Barcelona, Spain). DMF, DCM, DMSO, MeOH, Et₂O, piperidine and ACN (HPLC grade) were purchased from SDS (Peypin, France). All commercial reagents and solvents were used as received.

Tetrahydro-2H-pyran-4,4-diyl)bis(methylene) bis(trifluoromethanesulfonate) was prepared from tetrahydro-2H-pyran-4,4-diyl)bis(hydroxymethylene) (prepared from tetrahydro-2H-pyran-4,4-dicarboxylic acid dimethyl ester according to US2010/0099688 preparation 17) by conventional methods

### 1-Analytical Methods

### Analytical RP-HPLC

Analytical RP-HPLC was performed on a Waters instrument comprising a separation module (Waters 2695), an automatic injector (Waters 717 autosampler), a photodiode array detector (Waters 2998), and a software system controller (Empower). UV detection was at 220 nm, and linear gradients of eluent B (ACN+0.036% TFA) into A (water+0.045% TFA) were run at a flow rate of 1.0 mL/min over 8 min. The analytical RP-HPLC gradients used to determine the retention time (t_{R}) for the herein described peptides (TABLE 2) are expressed by indicating the variation of eluent B into eluent A.

Example of a gradient by using column 1, Gradient (% B)=0-100:

| Time (min) | Eluent A (%) | Eluent B (%) |
|---|---|---|
| 0 | 100 | 0 |
| 8 | 0 | 100 |

Column 1: reverse-phase C₁₈, XBridge™ BEH130, 4.6×100 mm, 3.5 μm from WATERS.

In a few cases it was used the analytical HPLC column 2, and those gradients required 30 min instead of 8 min of total gradient time.

Column 2: reverse-phase C₁₈, XBridge™, 4.6×150 mm, 5 μm from WATERS.

### Analytical RP-UPLC

Analytical RP-UPLC was performed on a Waters Acquity system equipped with a PDA eλ detector, a sample manager FNT, a Quaternary solvent manager and a software system controller (Empower). Linear gradients of eluent B (ACN+0.036% TFA) into A (water+0.045% TFA) were run at a flow rate of 0.6 mL/min over 2 min.

The analytical RP-UPLC gradients used to determine the retention time (t_{R}) for the herein described peptides (TABLE 2) are expressed by indicating the variation of eluent B into eluent A.

Column 1: reverse-phase C₁₈, Acquity BEH, 2.1×50 mm, 1.7 μm, Waters.

### Analytical RP-HPLC-ESI-MS

Analytical RP-HPLC-ESMS was performed on a Waters Micromass ZQ spectrometer comprising a separation module (Waters 2695), an automatic injector (Waters 717 autosampler), a photodiode array detector (Waters 2998), and a software system controller MassLynx v. 4.1). UV detection was at 220 nm, mass scans were acquired in positive ion mode, and linear gradients of B (ACN+0.07% formic acid) into A (water+0.1% formic acid) were run at a flow rate of 0.3 mL/min over 8 min.

Column: reverse-phase C₁₈, SunFire™, 2.1×100 mm, 5 μm from WATERS.

### Analytical RP-UPLC-ESI-MS

Analytical RP-UPLC-ESI-MS was performed on a Waters Acquity system equipped with a PDA eλ detector, a sample manager FNT, Quaternary solvent manager, a ZSpray MS detector and a MassLynx v4.1 system controller. Linear gradients of eluent B (ACN+0.7% FA) into A (water+0.1% FA) were run at a flow rate of 0.6 mL/min over 2 min, and mass spectra were acquired in positive ion mode.

Column 1: reverse-phase C₁₈, Acquity BEH, 2.1×50 mm, 1.7 μm, Waters.

### Semi-Preparative RP-HPLC

Two semi-preparative RP-HPLC systems and three different reverse-phase columns were used to purify the peptide analogs:

Equipment A: Semi-preparative RP-HPLC was performed on a Waters Delta 600 system comprising an automatic injector (Waters 2747 autosampler), a controller (Waters 600), a dual λ UV/Visible absorbance detector (Waters 2487), a fraction collector II, and a software system controller (MassLynx). UV detection was at 220 and 254 nm.

Equipment B: Semi-preparative RP-HPLC was performed on a Waters 2545 system comprising an automatic injector (Waters 2707 autosampler), a controller (Waters 2545 quaternary gradient module), a dual λ UV/Visible absorbance detector (Waters 2489), a fraction collector III, and a software system controller (Waters Chromscope). UV detection was at 220 and 254 nm.

Column 1: reverse-phase C₁₈ XBridge™ Prep OBD, 130 Å, 19×100 mm, 5 μm from WATERS.

Column 2: reverse-phase C₁₂ Jupiter Proteo 90 Å, AXI 21.2×100 mm, 10 μm from Phenomenex.

Column 3: reverse-phase C₁₈ XBridge™ Prep BEH130, 19×100 mm, 5 μm from WATERS.

Column 4: reverse-phase C₈ XBridge™ Prep OBD, 19×100 mm, 5 μm from WATERS

TABLE 2
Characterization of final synthesized peptides

| | RP-HPLC Analysis | | | | | | |
|---|---|---|---|---|---|---|---|
| SEQ | t_{R} | Gradient | | | Mass Spectrometry | | Purification |
| ID NO | (min) | (% B) | Column | % | MS calculated | MS observed | Method |
| 1 | 6.5 | 18-25 | 1 (HPLC) | 93 | 1416.6 | 1418 [M + H]⁺ | G |
| 2 | 4.2 | 20-40 | 1 (HPLC) | 98 | 1437.6 | 1439 [M + H]⁺ | G |
| 3 | 4.2 | 20-40 | 1 (HPLC) | 99.3 | 1437.6 | 1439 [M + H]⁺ | G |
| 4 | 3.4 | 30-40 | 1 (HPLC) | 94.5 | 1419.6 | 1421 [M + H]⁺ | G |
| 5 | 6.6 | 25-30 | 1 (HPLC) | 97.7 | 1419.6 | 1421 [M + H]⁺ | G |
| 6 | 4.5 | 15-30 | 1 (HPLC) | 99.7 | 1027.3 | 1028 [M + H]⁺ | G |
| 7 | 6.4 | 10-25 | 1 (HPLC) | 99.3 | 1006.3 | 1007 [M + H]⁺ | G |
| 8 | 17.5 | 10-20 | 2 | 97.8 | 2807.5 | 1405 [M + 2H]⁺/2 | H |
| | | | | | | 937 [M + 3H]⁺/3 | |
| 9 | 21.2 | 10-20 | 2 | 93.6 | 2828.5 | 1416 [M + 2H]⁺/2 | H |
| | | | | | | 944 [M + 3H]⁺/3 | |
| 10 | 4.0 | 10-30 | 1 (HPLC) | 99.9 | 967.3 | 968 [M + H]⁺ | G |
| 11 | 6.0 | 25-30 | 1 (HPLC) | 94.9 | 1398.6 | 1400 [M + H]⁺ | G |
| 12 | 5.5 | 15-30 | 1 (HPLC) | 99.2 | 1027.3 | 1028 [M + H]⁺ | G |
| 13 | 4.8 | 20-30 | 1 (HPLC) | 97.3 | 2693.3 | 1348 [M + 2H]⁺/2 | H |
| | | | | | | 899 [M + 3H]⁺/3 | |
| 14 | 5.1 | 20-40 | 1 (HPLC) | 99.1 | 1379.6 | 1381 [M + H]⁺ | G |
| 15 | 4.0 | 20-40 | 1 (HPLC) | 99.9 | 2635.3 | 1319 [M + 2H]⁺/2 | H |
| | | | | | | 880 [M + 3H]⁺/3 | |
| 16 | 5.9 | 10-30 | 1 (HPLC) | 99.9 | 2770.5 | 1388 [M + 2H]⁺/2 | H |
| | | | | | | 925 [M + 3H]⁺/3 | |
| 17 | 3.8 | 30-40 | 1 (HPLC) | 99.9 | 1361.6 | 1363 [M + H]⁺ | G |
| 18 | 5.9 | 10-30 | 1 (HPLC) | 99.9 | 2770.5 | 1388 [M + 2H]⁺/2 | H |
| | | | | | | 925 [M + 3H]⁺/3 | |
| 19 | 6.1 | 20-30 | 1 (HPLC) | 94.5 | 1379.6 | 1381 [M + H]⁺ | G |
| 20 | 3.8 | 20-40 | 1 (HPLC) | 99.9 | 969.3 | 970 [M + H]⁺ | G |
| 21 | 6.0 | 5-30 | 1 (HPLC) | 99.9 | 2361.2 | 1182 [M + 2H]⁺/2 | H |
| | | | | | | 788 [M + 3H]⁺/3 | |
| 22 | 4.8 | 5-50 | 1 (HPLC) | 99.9 | 967.3 | 968 [M + H]⁺ | G |
| 23 | 4.5 | 5-50 | 1 (HPLC) | 99.9 | 967.3 | 968 [M + H]⁺ | G |
| 24 | 4.2 | 20-40 | 1 (HPLC) | 99.9 | 1103.4 | 1105 [M + 2H]⁺ | G |
| 25 | 4.7 | 30-50 | 1 (HPLC) | 99.9 | 1203.4 | 1205 [M + 2H]⁺ | G |
| 26 | 5.5 | 10-30 | 1 (HPLC) | 98.0 | 1079.3 | 1081 [M + 2H]⁺ | G |
| 27 | 3.2 | 25-40 | 1 (HPLC) | 96.1 | 1077.3 | 1079 [M + 2H]⁺ | G |
| 28 | 5.4 | 70-100 | 1 (HPLC) | 95.6 | 1264.6 | 1267 [M + 2H]⁺ | I |
| 29 | 4.1 | 80-100 | 1 (HPLC) | 92.5 | 1716.9 | 1719 [M + 2H]⁺ | I |
| 30 | 5.9 | 0-50 | 1 (HPLC) | 99.7 | 2820.5 | 942 [M + 3H]⁺/3 | H |
| | | | | | | 707 [M + 4H]⁺/4 | |
| 31 | 5.6 | 70-100 | 1 (HPLC) | 97.7 | 1290.6 | 1292.0 [M + H]⁺ | I |
| | | | | | | 646.0 [M + 2H]⁺/2 | |
| 32 | 6.4 | 70-100 | 1 (HPLC) | 97.0 | 1340.6 | 1341.9 [M + H]⁺ | I |
| | | | | | | 670.8 [M + 2H]⁺/2 | |
| 33 | 5.2 | 70-100 | 1 (HPLC) | 98.7 | 1290.6 | 1291.8 [M + H]⁺ | I |
| | | | | | | 645.9 [M + 2H]⁺/2 | |
| 34 | 5.6 | 0-50 | 1 (HPLC) | 97.4 | 2796.5 | 700.0 [M + 4H]⁺/4 | H |
| | | | | | | 560.1 [M + 5H]⁺/5 | |
| 35 | 4.0 | 80-100 | 1 (HPLC) | 98.5 | 1742.9 | 872.2 [M + 2H]⁺/2 | I |
| | | | | | | 581.8 [M + 3H]⁺/3 | |
| 36 | 4.4 | 80-100 | 1 (HPLC) | 94.2 | 1792.9 | 897.7 [M + 2H]⁺/2 | I |
| | | | | | | 598.8 [M + 3H]⁺/3 | |
| 37 | 4.0 | 80-100 | 1 (HPLC) | 94.8 | 1742.9 | 872.6 [M + 2H]⁺/2 | I |
| | | | | | | 582.1 [M + 3H]⁺/3 | |
| 38 | 4.3 | 80-100 | 1 (HPLC) | 93.2 | 1766.9 | 1769 [M + 2H]⁺ | |
| 39 | 5.2 | 70-100 | 1 (HPLC) | 97.6 | 1716.9 | 1717 [M + H]⁺ | I |
| 40 | 5.3 | 70-100 | 1 (HPLC) | 96.3 | 1682.9 | 1683 [M + H]⁺ | I |
| 41 | 6.4 | 60-100 | 1 (HPLC) | 99.9 | 1230.6 | 1231 [M + H]⁺ | I |
| 42 | 6.3 | 0-50 | 1 (HPLC) | 95.1 | 2846.5 | 712.4 [M + 4H]⁺/4 | H |
| | | | | | | 750.1 [M + 5H]⁺/5 | |
| 43 | 2.9 | 10-60 | 1 (HPLC) | 99.9 | 983.3 | 985 [M + 2H]⁺ | G |
| 44 | 6.2 | 80-100 | 1 (HPLC) | 95.3 | 1742.9 | 872.4 [M + 2H]⁺/2 | I |
| | | | | | | 582.0 [M + 3H]⁺/3 | |
| 45 | 4.3 | 80-100 | 1 (HPLC) | 96.3 | 1755.9 | 1756.1 [M + H]⁺ | I |
| | | | | | | 879.1 [M + 2H]⁺/2 | |
| 46 | 5.2 | 70-90 | 1 (HPLC) | 99.5 | 1755.9 | 1756.0 [M + H]⁺ | I |
| | | | | | | 879.0 [M + 2H]⁺/2 | |
| 47 | 6.9 | 50-70 | 1 (HPLC) | 98.7 | 1756.9 | 879.5 [M + 2H]⁺/2 | J |
| | | | | | | 586.6 [M + 3H]⁺/3 | |
| 48 | 3.9 | 80-100 | 1 (HPLC) | 97.1 | 1741.9 | 1745 [M + 3H]⁺ | I |
| 49 | 4.9 | 70-100 | 1 (HPLC) | 95.0 | 1739.9 | 1743 [M + 3H]⁺ | I |
| 50 | 5.3 | 70-100 | 1 (HPLC) | 98.6 | 1652.8 | 1654.1 [M + H]⁺ | I |
| | | | | | | 827.0 [M + 2H]⁺/2 | |
| 51 | 4.9 | 70-100 | 1 (HPLC) | 94.5 | 1784.0 | 1788 [M + 4H]⁺ | I |
| 52 | 6.3 | 70-100 | 1 (HPLC) | 91.7 | 1686.9 | 844.6 [M + 2H]⁺/2 | I |
| | | | | | | 563.5 [M + 3H]⁺/3 | |
| 53 | 5.9 | 30-80 | 1 (HPLC) | 97.3 | 1803.0 | 902.3 [M + 2H]⁺/2 | J |
| | | | | | | 602.2 [M + 3H]⁺/3 | |
| 54 | 4.7 | 45-55 | 1 (HPLC) | 95.3 | 3133.8 | 1047 [M + 3H]⁺/3 | H |
| | | | | | | 786 [M + 4H]⁺/4 | |
| 55 | 6.4 | 75-80 | 1 (HPLC) | 91.7 | 1813.9 | 1817 [M + 3H]⁺ | I |
| 56 | 3.8 | 20-40 | 1 (HPLC) | 95.5 | 2868.5 | 959 [M + 3H]⁺/3 | H |
| | | | | | | 719 [M + 4H]⁺/4 | |
| 57 | 5.2 | 50-70 | 1 (HPLC) | 95.0 | 1756.9 | 879.6 [M + 2H]⁺/2 | H |
| | | | | | | 586.8 [M + 3H]⁺/3 | |
| 58 | 5.8 | 10-50 | 1 (HPLC) | 96.0 | 2848.6 | 712.8 [M + 4H]⁺/4 | H |
| | | | | | | 570.6 [M + 5H]⁺/5 | |
| 59 | 3.8 | 20-60 | 1 (HPLC) | 97.0 | 2839.6 | 948.1 [M + 3H]⁺/3 | H |
| | | | | | | 711.3 [M + 4H]⁺/4 | |
| 60 | 3.3 | 20-60 | 1 (HPLC) | 96.0 | 2797.5 | 700.5 [M + 4H]⁺/4 | H |
| | | | | | | 560.8 [M + 5H]⁺/5 | |
| 61 | 4.8 | 70-100 | 1 (HPLC) | 99.5 | 1793.9 | 898.3 [M + 2H]⁺/2 | J |
| | | | | | | 599.2 [M + 3H]⁺/3 | |
| 62 | 4.6 | 70-100 | 1 (HPLC) | 98.0 | 1742.9 | 872.7 [M + 2H]⁺/2 | J |
| | | | | | | 582.0 [M + 3H]⁺/3 | |
| 63 | 5.3 | 20-60 | 1 (HPLC) | 99.5 | 2973.6 | 992.2 [M + 3H]⁺/3 | H |
| | | | | | | 744.7 [M + 4H]⁺/4 | |
| 64 | 3.8 | 20-60 | 1 (HPLC) | 99.0 | 1428.6 | 1430.0 [M + H]⁺ | G |
| | | | | | | 715.6 [M + 2H]⁺/2 | |
| 65 | 3.2 | 70-100 | 1 (HPLC) | 98.0 | 1742.9 | 1744.7 [M + H]⁺ | J |
| | | | | | | 872.6 [M + 2H]⁺/2 | |
| 66 | 3.9 | 25-35 | 1 (HPLC) | 95.6 | 2972.6 | 744.3 [M + 4H]⁺/4 | H |
| | | | | | | 595.8 [M + 5H]⁺/5 | |
| 67 | 6.1 | 10-40 | 1 (HPLC) | 97.9 | 2838.5 | 710.8 [M + 4H]⁺/4 | H |
| | | | | | | 569.2 [M + 5H]⁺/5 | |
| 68 | 5.7 | 0-60 | 1 (HPLC) | 97.3 | 2774.5 | 1388.4 [M + 2H]⁺/2 | H |
| | | | | | | 925.9 [M + 3H]⁺/3 | |
| 69 | 6.5 | 0-50 | 1 (HPLC) | 98.3 | 3086.7 | 773.2 [M + 4H]⁺/4 | H |
| | | | | | | 618.9 [M + 5H]⁺/5 | |
| | | | | | | 515.9 [M + 6H]⁺/6 | |
| 70 | 5.3 | 70-100 | 1 (HPLC) | 95.1 | 1711.8 | 1712.8 [M + H]⁺ | J |
| | | | | | | 857.2 [M + 2H]⁺/2 | |
| | | | | | | 572.0 [M + 3H]⁺/3 | |
| 71 | 12.7 | 10-25 | 2 (HPLC) | 90.2 | 2864.5 | 1433.6 [M + 2H]⁺/2 | H |
| | | | | | | 956.2 [M + 3H]⁺/3 | |
| | | | | | | 717.4 [M + 4H]⁺/4 | |
| | | | | | | 574.2 [M + 5H]⁺/5 | |
| 72 | 5.2 | 70-100 | 1 (HPLC) | 91.7 | 1670.8 | 1694.1 [M + Na]⁺ | I |
| | | | | | | 1671.2 [M + H]⁺ | |
| | | | | | | 836.6 [M + 2H]⁺/2 | |
| 73 | 3.7 | 70-100 | 1 (HPLC) | 98.0 | 1667.8 | 1668.8 [M + H]⁺ | I |
| 74 | 5.3 | 70-100 | 1 (HPLC) | 97.6 | 1652.8 | 1654.6 [M + H]⁺ | I |
| | | | | | | 827.6 [M + 2H]⁺/2 | |
| 75 | 5.2 | 60-100 | 1 (HPLC) | 98.3 | 1711.8 | 1713.9 [M + H]⁺ | I |
| | | | | | | 857.1 [M + 2H]⁺/2 | |
| 76 | 5.2 | 60-100 | 1 (HPLC) | 94.5 | 1654.8 | 1655.6 [M + H]⁺ | I |
| | | | | | | 828.7 [M + 2H]⁺/2 | |
| 77 | 1.2 | 70-100 | 1 (HPLC) | 92.2 | 1666.8 | 1689.6 [M + Na]⁺ | I |
| | | | | | | 1667.0 [M + H]⁺ | |
| 78 | 4.9 | 70-100 | 3 | 93.5 | 1670.8 | 1671.9 [M + H]⁺ | I |
| | | | | | | 836.6 [M + 2H]⁺/2 | |
| 79 | 4.7 | 70-100 | 1 (HPLC) | 93.4 | 1634.0 | 1634.3 [M + H]⁺ | I |
| | | | | | | 817.7 [M + 2H]⁺/2 | |
| 80 | 5.1 | 50-100 | 3 | 99.3 | 1596.8 | 1598.5 [M + H]⁺ | I |
| | | | | | | 799.8 [M + 2H]⁺/2 | |
| 81 | 3.6 | 60-90 | 3 | 97.6 | 1652.9 | 1654.0 [M + H]⁺ | I |
| | | | | | | 827.8 [M + 2H]²⁺/2 | |
| 82 | 3.1 | 60-90 | 3 | 94.8 | 1667.8 | 1669.0 [M + H]⁺ | I |
| | | | | | | 835.1 [M + 2H]⁺/2 | |
| 83 | 6.4 | 50-100 | 3 | 98.6 | 1624.8 | 1625.8 [M + H]⁺ | I |
| | | | | | | 813.7 [M + 2H]⁺/2 | |
| 84 | 1.22 | 50-100 | 1 (UPLC) | 93.6 | 1653.8 | 1654.8 [M + H]⁺ | K |
| | | | | | | 828.2 [M + 2H]⁺/2 | |
| 85 | 6.5 | 40-70 | 1 (HPLC) | 99.6 | 1568.7 | 1569.7 [M + H]⁺ | I |
| | | | | | | 785.6 [M + 2H]⁺/2 | |
| 86 | 5.5 | 50-80 | 3 | 94.0 | 1646.8 | 1647.9 [M + H]⁺ | K |
| | | | | | | 824.5 [M + 2H]²⁺/2 | |
| 87 | 4.8 | 65-85 | 3 | 95.6 | 1650.8 | 1651.9 [M + H]⁺ | K |
| | | | | | | 826.5 [M + 2H]²⁺/2 | |
| 88 | 4.7 | 70-100 | 3 | 96.1 | 1650.8 | 1651.4 [M + H]⁺ | K |
| | | | | | | 826.6 [M + 2H]²⁺/2 | |
| 89 | 8.9 | 45-60 | 4 | 95.1 | 1708.9 | 855.4 [M + 2H]²⁺/2 | K |
| 90 | 7.9 | 45-55 | 4 | 97.3 | 1680.9 | 841.6 [M + 2H]²⁺/2 | K |
| 91 | 6.0 | 70-100 | 3 | 97.0 | 1668.9 | 835.7 [M + 2H]²⁺/2 | I |
| | | | | | | 846.6 [M + H + Na]²⁺/2 | |
| 92 | 6.0 | 70-100 | 3 | 98.3 | 1668.9 | 846.6 [M + H + Na]²⁺/2 | I |
| 93 | 0.58 | 70-100 | 1 (HPLC) | 90.8 | 1652.8 | 1653.8 [M + H]⁺ | I |
| | | | | | | 846.7 [M + 2H]⁺/2 | |
| 94 | 5.9 | 80-100 | 1 (HPLC) | 95.1 | 1698.8 | 1699.5 [M + H]⁺ | I |
| | | | | | | 850.3 [M + 2H]⁺/2 | |
| 95 | 0.76 | 70-100 | 1 (UPLC) | 99.3 | 1662.0 | 831.7 [M + 2H]²⁺/2 | I |
| 96 | 0.76 | 70-100 | 1 (HPLC) | 99.3 | 1662.0 | 831.7 [M + 2H]⁺/2 | I |
| 97 | 5.2 | 70-100 | 1 (HPLC) | 98.8 | 1650.8 | 1651.5 [M + H]⁺ | I |
| | | | | | | 826.5 [M + 2H]⁺/2 | |
| 98 | 6.9 | 70-100 | 1 (HPLC) | 94.9 | 1666.8 | 834.5 [M + 2H]²⁺/2 | I |
| 99 | 0.82 | 70-100 | 1 (UPLC) | 95.1 | 1723.0 | 1746.4 [M + Na]⁺ | K |
| | | | | | | 1762.4 [M + K]⁺ | |
| 100 | 4.48 | 70-100 | 1 (HPLC) | 100.0 | 1662.0 | 1662.7 [M + H]⁺¹ | I |
| | | | | | | 1680.1 [M + H₂O]⁺¹ | |
| | | | | | | 831.6 [M + 2H/2]⁺¹ | |

### General Synthetic Procedures

### 1-Solid Phase Peptide Synthesis

### 1a) C-terminal Amide Peptide Sequences

Linear C-terminal amide sequences were synthesized on a Fmoc-Rink amide AM PS resin. Initially, the resin (0.2 mmol; 1 eq.; 0.69 or 0.71 mmol/g) was washed with DCM and DMF (3×1 min; 1 mL per 100 mg of resin, each solvent).

Alternatively, some peptides were synthesized on a PEG based resin, Fmoc-Rink amide AM ChemMatrix resin. In those cases, initially, the resin (0.2 mmol; 1 eq.; 0.49 mmol/g) was washed with DCM and DMF (3×1 min; 1 mL per 100 mg of resin, each solvent) and subsequently, it was treated with a mixture of TFA-DCM (1:99) (6×30 s, 1 mL per 100 mg of resin) at rt with constant stirring. The resin was washed with DCM (3×1 min) and neutralized by treatment with a mixture of DIEA-DCM (5:95) (6×30 s, 1 mL per 100 mg of resin) at rt with constant stirring, and finally washed with DCM and DMF (3×1 min; 1 mL per 100 mg of resin, each solvent).

Those peptide analogs with a linker incorporated on solid phase were synthesized on a PEG based resin, H-Rink amide AM ChemMatrix resin (0.1 mmol; 1 eq.; 0.47 mmol/g) according to the same initial treatments performed for other ChemMatrix resins. In this case the initial loading was decreased by assessing the equivalents of first AA coupled, as it is described in examples 68 and 69. The Fmoc group from the resin was removed by treatment with piperidine-DMF (1:4) (1×1 min, 2×5 min, 1 mL per 100 mg of resin). After Fmoc cleavage, the peptidyl-resin was thoroughly washed sequentially with DMF/DCM/DMF.

The incorporation of the Fmoc-Aas was performed under neutral conditions by adding to the resin the solution of Fmoc-Aa-OH (3 eq.), Oxyma Pure® (3 eq.) and DIPCDI (3 eq.) in DMF (0.2 M), previously activated for 5 min. Each Aa coupling was carried out at rt for 40 min with constant shaking. In all cases, after the Aa incorporation, the resin was washed with DCM/DMF cycle and the colorimetric ninhydrin test was performed to evaluate the extension of the reaction. When the test showed the presence of free amino groups (positive result), another attempt to introduce the Aa was performed by using neutral or basic conditions. In the latter case, a solution of Fmoc-Aa (3 eq.), HBTU (3 eq.) and DIEA (3 eq.) in DMF (0.2 M) was added to the peptidyl-resin and left the mixture react for 40 min at rt with constant shaking. When the Aa incorporations were completed, the peptidyl-resin was washed and Fmoc was removed following the same protocol described before.

Those peptide analogs with its N-terminus acetylated required an extra step after the last Fmoc removal. The N-terminal acetylation was performed by adding to the peptidyl-resin a mixture of Ac₂O (10 eq.) and DIEA (10 eq.) in DMF (0.2 M), leaving the mixture react for 30 min at rt. The sequential DMF/DCM washes were required and again the ninhydrin test confirmed the completion of the acetylation.

When Alloc was contained into the sequence its removal was performed by adding to the peptidyl-resin a solution of Pd(Ph₃P)₄ (0.1 eq.) in DCM (0.2 M) followed by the addition of phenylsilane (10 eq.) for 3×15 min at rt with constant shaking. The cleavage mixture was filtered and, without any washes, two more consecutive Alloc removal treatments were performed. Subsequently, in order to remove the residues generated from the palladium reagent, the peptidyl-resin was washed thoroughly and sequentially with DCM/DMF/DCM.

Those peptide analogs conjugated to a fatty acid required longer reaction time of 120 min to couple it, by using the same neutral conditions and equivalents described for the incorporation of the Fmoc-Aas.

After the elongation of the linear C-terminal amide sequences, the peptidyl-resins were prepared to perform the cleavage (section 2 described below).

### 1b) C-Terminal Acid Peptide Sequences

Linear C-terminal acid sequences were synthesized on a 2-chlorotrityl chloride PS resin. Initially, the resin (0.2 mmol; 0.8 eq.; 1.6 mmol/g) was washed with DCM and DMF (3×1 min, 1 mL per 100 mg of resin, each solvent) and subsequently, the incorporation of the first Fmoc-Aa (0.5-0.8 eq.) in DCM (0.2 M) was performed by adding to the resin DIEA in two separated portions, first 3 eq. and after 10 min, 7 eq. which were left to react 45 min at rt. Subsequently, MeOH (0.8 μL per 1 mg of resin) was added to the previous mixture for capping the free active sides from the resin. After 10 min, the peptidyl-resin was washed sequentially with DCM/DMF and the removal of the Fmoc group of the first Aa allowed determining the new loading of the resin. Fmoc cleavage was carried out by treatment with piperidine-DMF (1:4) (1×1 min, 2×5 min; 1 mL per 100 mg of resin) and all the residual solutions removed by suction were collected in a volumetric flask. After Fmoc cleavage, the peptidyl-resin required again to be thoroughly washed with DMF and the residual washing solution was also combined with the previous piperidine treatments. This final solution contained in a volumetric flask was diluted with DMF to a total volume (V) of 100 mL. A 1 mL aliquot (V₁) of this solution was diluted with DMF to a volume of 10 mL (V₂). The UV absorption (A) was measured at 290 nm against the DMF as a reference. The loading of the resin was calculated according to the following:

New Loading (mmol/g)=(A×V₂×V)/(1×ε×V₁×m)

(where 1 corresponds to the length of the cuvette=1 cm; ε corresponds to the extinction coefficient=5800 Lmol⁻¹ cm⁻¹; and m corresponds to the total grams of resin)

Taking into account the new calculated loading, the following Fmoc-Aas were coupled to the peptidyl-resin following the same conditions described for the C-terminal amide sequences (section 1a). Fmoc/Alloc removals, the acetylation step and the fatty acid incorporation were carried out by using the same methodologies detailed in section 1a.

After the elongation of the linear C-terminal acid sequences, the peptidyl-resins were prepared to perform the cleavage (step 2 described below). Specifically, those peptide analogs may be cleaved from the resin through two different protocols (section 2a or 2b), depending on the sequence required.

2-Cleavage from the Resin with/without Global Deprotection
2a) Bicyclic Peptides: Cleavage without Global Deprotection: Acyclic Peptides with Protected Side-Chains

The preparation of byclic peptides requires an acyclic precursor with the side-chains protected and the N- and C-termini free (cyclication 1, scheme 2).

After the elongation of the linear sequence, the peptidyl-resin (0.2 mmol) was treated with a mixture of TFA-DCM (2:98) (7×30 s, 1 mL per 100 mg of resin) at rt with constant stirring. All the acidic washes were filtered and collected in a flask with some water (1 mL per 100 mg of resin) and the organic solvents were removed with nitrogen sparge. The solution was diluted with a mixture of H₂O-ACN to a volume of 20 mL, which was lyophilized to afford the acyclic crude peptide as a solid, which was used without purification for the following step (section 3, cyclization 1, Method E).

2b) Cleavage with Global Deprotection: Acyclic Peptides with Unprotected Side-Chains

Monocyclic peptides, whose cycle is formed through a linker, are best prepared form the acyclic precursor with unprotected side-chains (Scheme 1).

After the elongation of the linear sequence, the peptidyl-resin (0.2 mmol) was treated with a mixture of TFA-TIS-H₂O (95:2.5:2.5, v/v/v) (10 mL) for 1 h at rt. The cleavage mixture was filtered and collected in a flask. The resulted peptidyl-resin was washed using the same cleavage mixture (3×1 min, 1 mL per 100 mg of resin) and the combined solutions were added to the previous one. The solution was concentrated under vacuum until ca. 5 mL and the crude peptide was precipitated with cold Et₂₀ (40 mL). The mixture was centrifuged and the solid was washed twice with Et₂₀ (40 mL). Alternatively, the final acidic solution was directly poured onto cold Et₂₀ (30 mL). The mixture was centrifuged and the solid was washed twice with Et₂O (30 mL). The final peptide intermediate was left to dryness at rt before cyclization.

### 3-Cyclization

### 3a) Cyclization Method A (Side-Chain-to-Side-Chain Via a Linker in Solution in H₂O-ACN)

This cyclization method is applied to peptides soluble in aqueous medium (unconjugated peptides and those conjugated to a CPP)

The fully unprotected acyclic crude peptide (1 eq.) obtained from cleavage 2b was dissolved in a mixture of H₂O-ACN (1:1 or 4:1; 0.5-1 mM) in a round-bottom flask, at 35-40° C. with constant stirring. For those peptide analogs conjugated to a CPP, 3,3,3-phosphanetriyltripropanoic acid (TCEP) (1-5 eq.) was added to the solution and subsequently, the corresponding bis(bromomethyl)aryl or bis(chloromomethyl)aryl linker (2 eq.) was added followed by the addition of NH₄HCO₃ (20-40 mM) (or DIEA in those cases where the bicarbonate was not effective). The reaction was stirred at 35-40° C. until it was completed as shown by analytical RP-HPLC. The mixture was directly lyophilized and the solid crude peptide was directly purified according to the protocol described in section 5a.

### 3b) Cyclization Method B (Side-Chain-to-Side-Chain Via a Linker in Solution in DMF)

This cyclization method is applied to peptides only soluble in DMF (conjugated to a fatty acid)

The fully unprotected acyclic crude peptide (1 eq.) obtained from cleavage 2b was dissolved in DMF (0.5-1 mM) in a round-bottom flask, at rt with constant stirring. The corresponding bis(bromomethyl)aryl or bis(chloromomethyl)aryl linker (2 eq.; 0.5-2 mM) was added followed by the dropwise addition of NH₄HCO₃ (5, 20 or 40 mM), previously dissolved in H₂O (2% v/v). The reaction was stirred at rt until t was completed, as shown by analytical RP-HPLC. The solution was concentrated to near dryness under vacuum to afford the crude peptide which was directly purified according to the protocol described in section 5b.

### 3c) Cyclization Method C (Trp-Cys Cross-Bridge)

In a round-bottom flask, the fully deprotected acyclic peptide (1 eq.) was dissolved in mixtures of H₂O-ACN (0.5 mM). A solution of I₂ (2-5 eq.) in ACN was added dropwise to the round-bottom flask and the mixture was stirred at rt. The reaction progress was monitored by analytical RP-HPLC to completion. Finally, the reaction mixture was washed up to 5 times with DCM to remove the excess of I₂, and the solvent was removed by lyophilization.

### 3d) Cyclization Method D (Phe-Cys Cross-Bridge)

This cyclization method is applied to those unconjugated peptides.

In a round-bottom flask, the fully deprotected acyclic peptide (1 eq.; 0.5 mM), CuI (0.5 eq.) and K₂CO₃ (2 eq.) were added and the system was purged with N₂ (g) for 10 min. The ethylene glycol (2 eq.) dissolved in water was added to the round-bottom flask and the mixture was stirred at 50° C. under N₂ (g) atmosphere. The reaction progress was monitored by analytical RP-HPLC to completion. Finally, the solvent was removed by lyophilization.

### 3e) Cyclization Method E (Phe-Cys Cross-Bridge)

This cyclization method is applied to those peptide analogs conjugated to a fatty acid.

In a round-bottom flask, the fully deprotected linear peptide (1 eq.; 0.5 mM), the CuI (0.5 eq.) and the cis-ciclohexane-1,2-diol (2 eq.) were added and the system was purged with N₂ (g) for 10 min. The DIEA (2 eq.) dissolved in a mixture of water-tBuOH (1:1) was added to the round-bottom flask and the mixture was stirred at 70-75° C. under N₂ (g) atmosphere. The reaction progress was monitored by analytical RP-HPLC to completion. Finally, the solvent was removed by lyophilization.

### 3f) Double Cyclization Method F ((1) Lactam Formation and (2) Via Linker Incorporation or Trp-Cys Cross-Bridge)

This cyclization method is used for those bicyclic unconjugated peptide analogs (scheme 2). The two cyclization protocols are not performed consecutively because after cyclization 1, a global deprotection (as shown in section 4) is required before performing cyclization 2.

### Cyclization 1 (Lactam Formation Between C- and N-Termini or Between C-Terminus and Side-Chain of Aa):

In a round-bottom flask, the fully protected acyclic peptide (1 eq.) and HBTU (2 eq.) were dissolved in ACN or mixtures of ACN-DMF (0.5-1 mM). DIEA (0.5-1% v/v) was added to adjust the pH to 8 and the reaction mixture was stirred at rt. The reaction progress was monitored by analytical RP-HPLC to completion. The solvent was removed under vacuum and the resulting crude was dissolved in DCM. The DCM phase was washed up to 3 times with an aqueous NaHCO₃ (sat.) solution and dried over anhydrous MgSO₄. The MgSO₄ was removed by filtration and the DCM was evaporated at reduced pressure to afford the fully protected cyclic intermediate. After the treatment described in section 4, the crude peptide was globally deprotected and the cyclization 2 was performed.

### Cyclization 2a (Side-Chain-to-Side-Chain Trough a Linker):

The fully unprotected cyclic crude peptide obtained after the global deprotection (section 4) was cyclized according to the protocol detailed in cyclization Method A.

### Cyclization 2b (Trp-Cys Cross-Bridge):

The fully unprotected cyclic crude peptide obtained after the global deprotection (section 4) was cyclized according to the protocol detailed in cyclization Method C.

### 3g) Double Cyclization Method G ((1) Lactam Formation and (2) Trough a Linker)

This cyclization method is applied for those bicyclic peptide analogs (scheme 2) conjugated to a fatty acid. The two cyclization protocols are not performed consecutively because after cyclization 1, a global deprotection is required before performing cyclization 2.

### Cyclization 1 (Lactam Formation):

The fully protected acyclic crude peptide obtained from cleavage 2a was dissolved in DMF (1 mM) in a round-bottom flask, at rt with constant stirring. HBTU or PyBOP (2 eq.) was added to the solution followed by the addition of DIEA (1% v/v) to adjust to pH=8-9. The reaction was stirred at rt until it was completed, as shown by analytical RP-HPLC. The solution was concentrated to near dryness under vacuum to afford the cyclic intermediate which was treated as it is described in Section 4 to perform the global deprotection of the cyclized sequence.

### Cyclization 2 (Side-Chain-to-Side-Chain Trough a Linker):

The fully unprotected cyclic crude peptide obtained after the global deprotection (Section 4) was cyclized according to a method similar to that described in cyclization Method A (for those sequences not containing a fatty acid) or Method B (for those sequences containing a fatty acid).

### 3h) Cyclization Method H (Through Linker on Solid-Phase)

After peptide elongation, the Mmt groups, which were used to protect the side-chain the two Cys, were selectively removed by treating the peptidyl-resin with a mixture of TFA-TIS-DCM (2:2.5:95.5, v/v/v) (3×10 min, 1 mL per 100 mg of resin) and washed with DCM and DMF (3×1 min, 1 mL per 100 mg of resin, each solvent). Subsequently, the peptidyl-resin was treated with a mixture of bis(bromomethyl)aryl or bis(chloromethyl)aryl derivative (3 eq.) and DIEA (6 eq.) in DMF (1 mL per 50-100 mg of resin) for 3 h at rt to afford the fully protected cyclic peptide anchored on the resin. The peptidyl-resin was washed with DMF and the Fmoc group from the N-terminus was removed by using piperidine-DMF (1:4) (1:4) (1×1 min, 2×5 min, 1 mL per 100 mg of resin). Finally, the cyclic peptide is simultaneously deprotected and cleaved from the resin using a mixture of TFA-TIS-H₂O (95:2.5:2.5, v/v/v) (1 mL per 100 mg of resin) for 2-16 h at rt (depending of the number of Arg in the peptide, which require more time to remove their protecting group). The acidic mixture was concentrated under vacuum to 5 mL, and the peptide was precipitated with cold Et₂O (10 mL per 100 mg of peptide). The crude peptide was centrifuged and the residue washed twice with cold Et₂O (10 mL per 100 mg of peptide). The cyclic peptide was left to dryness at rt.

### 3i) Cyclization Method I (Side Chain-to-Side-Chain Through Oxetane Linker).

The fully unprotected cyclic crude peptide obtained after the global deprotection (Section 4) was dissolved in DMF at a 15 mM concentration and stirred at 37° C. in the presence of TCEP (0.5-1 eq.) and K₂CO₃ (5 eq.) for 30 min under N₂. After this time, the corresponding 3,3-bis(bromomethyl)oxetane linker (1.1 eq.) previously dissolved in a solution of KI (1.1 eq) in DMF (1 mL) was added dropwise to the solution. The reaction was stirred at 37° C. until it was completed, as shown by analytical RP-HPLC. When the reaction still uncompleted, an extra addition of the 3,3-bis(bromomethyl)oxetane (1.1 eq.) was performed. The solution was concentrated to near dryness under vacuum to afford the crude peptide which was directly purified according to the protocol described in section 5b.

### 4-Global Deprotection for Bicyclic Peptides

In a round-bottom flask, the fully protected cyclic peptide was treated with a mixture of TFA-water-TIS (95:2.5:2.5, v/v/v) (5 mL per 100 mg of peptide) for 1-2 h at rt. The acidic mixture was evaporated under vacuum to 5 mL and the peptide was precipitated with cold Et₂O (10 mL per 100 mg of peptide). The crude peptide was centrifuged and the residue washed twice with cold Et₂O (10 mL per 100 mg of peptide). The cyclic peptide intermediate was left to dryness at rt.

Alternatively, the acidic mixture was directly poured onto cold Et₂O (10 mL per 100 mg of peptide). The suspension was centrifuged and the residue was washed twice with cold Et₂O (10 mL per 100 mg of peptide). The cyclic peptide intermediate was left to dryness at rt.

For those compounds containing an unsatured fatty acid the procedure was analogous to the previously described but a 3 h TFA-ethanethiol-water-TIS (70:25:2.5:2.5, v/v/v/v) treatment was used instead of the previously described 1-2 h reaction with a TFA-water-TIS (95:2.5:2.5, v/v/v) mixture.

### 5-Purification

Final cyclic and bicyclic crude peptides were purified by standard semi-preparative RP-HPLC. Eluents and linear gradients were optimized for each crude peptide.

Fractions were collected and analyzed by analytical RP-HPLC and RP-HPLC-ESI-MS. Pure product fractions were combined and lyophilized to afford the pure final peptides. All peptides were obtained as white powders with a purity >90%.

### 5a) Method G

The crude peptides were dissolved in a minimum amount of water or a mixture of water-ACN, filtered and purified by semi-preparative RP-HPLC performing multiple injections. For each injection, the crude peptide solutions were loaded onto the RP-HPLC column and eluted with linear gradients of B (ACN+0.05% formic acid) into A (water+0.1% formic acid) run at a flow rate of 16 or 20 mL/min over 20 min. The elution was monitored at 220 nm and 254 nm. For those peptides with basic net charge, the final lyophilized products were obtained as formate salts.

### 5b) Method H

The crude peptides were dissolved in a minimum amount of water or mixture of water-ACN, filtered, and purified by semi-preparative RP-HPLC performing multiple injections. For each injection, the crude peptide solutions were loaded onto the RP-HPLC column and eluted with linear gradients of B (ACN+0.05% TFA) into A (water+0.1% TFA) run at a flow rate of 16 mL/min over 20 min. The elution was monitored at 220 nm and 254 nm. For those peptides with basic net charge, the final lyophilized products were obtained as trifluoroacetate salts.

### 5c) Method I

The crude peptides were dissolved in a minimum amount of MeOH, DMF or DMSO, filtered, and purified by semi-preparative RP-HPLC performing multiple injections. For each injection, the crude peptide solutions were loaded onto the RP-HPLC column and eluted with linear gradients of B (ACN+0.05% formic acid) into A (water+0.1% formic acid) run at a flow rate of 16 mL/min over 20 min. The elution was monitored at 220 nm and 254 nm. For peptides with basic net charge, the final lyophilized products were obtained as formate salts.

### 5d) Method J

The crude peptides were dissolved in a minimum amount of MeOH, DMF, filtered, and purified by semi-preparative RP-HPLC performing multiple injections. For each injection, the crude peptide solutions were loaded onto the RP-HPLC column and eluted with linear gradients of B (ACN+0.05% TFA) into A (water+0.1% TFA) run at a flow rate of 16 mL/min over 20 min. The elution was monitored at 220 nm and 254 nm. For peptides with basic net charge, the final lyophilized products were obtained as trifluoroacetate salts.

### 5e) Method K

Crude peptides were purified as described in Method J (5d) but they were lyophilized twice from 5% formic acid in H₂O/CAN (1:1, v/v) to obtain the peptide as a formate salt in case it had a basic net charge.

The nomenclature rules followed for the name assignation of the peptides have been extracted from Spengler, J., Jiménez, J.-C., Burger, K., Giralt, E., Albericio, F. Abbreviated nomenclature for cyclic and branched homo- and hetero-detic peptides. J. Peptide Res., 2005, 65, 550-555.

A methylene group as used herein (methylene bridge, methylene spacer, or methanediyl group) is any part of a molecule with the formula “—CH₂-”; namely, a carbon atom bound to two hydrogen atoms and connected by single bonds to two other distinct atoms in the rest of the molecule.

### Example 1

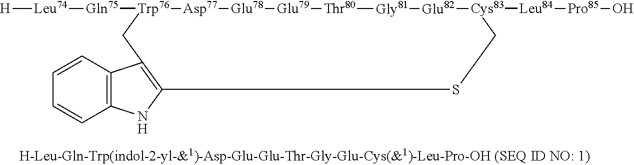

This 12 mer peptide analog was synthesized according to the general scheme 1. The linear sequence was obtained according to the procedure described in section 1b for those C-terminal acid peptide sequences. The cleavage from the resin with global deprotection was carried out as described in section 2b. The cyclization to form the Trp-Cys cross-bridge was done by the Method C. Finally, the peptide crude was purified with equipment A and column 1 according to the Method G to afford a white solid as a formate salt with a purity of 93%.

This pure peptide was analyzed by analytical RP-HPLC with column 1 (gradient 18-25; t_{R}=6.5 min) and RP-HPLC-ESI-MS (calculated: 1416.6 (M); observed: 1418 [M+H]⁺).

### Example 2

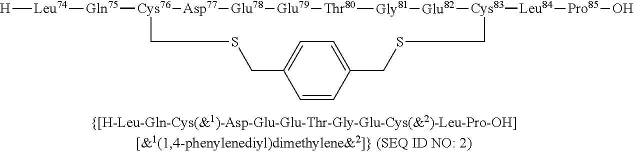

This 12 mer peptide analog was synthesized according to the general scheme 1. The linear sequence was obtained following the procedure described in section 1b for those C-terminal acid peptide sequences. The cleavage from the resin with global deprotection was carried out as described in section 2b. The cyclization through incorporation of 1,4-bis(bromomethyl)benzene linker between the cysteines was done by the Method A. Finally, the peptide crude was purified with equipment A and column 1 according to the Method G to afford a white solid as a formate salt with a purity of 98%.

This pure peptide was analyzed by analytical RP-HPLC with column 1 (gradient 20-40; t_{R}=4.2 min) and RP-HPLC-ESI-MS (calculated: 1437.6 (M); observed: 1439 [M+H]⁺).

### Example 3

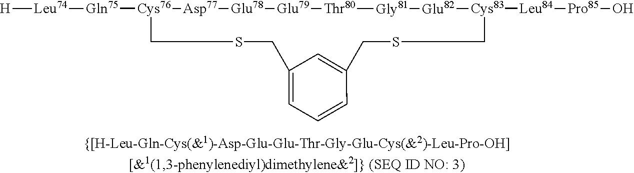

This 12 mer peptide analog was synthesized according to the general scheme 1. The linear sequence was obtained following the procedure described in section 1b for those C-terminal acid peptide sequences. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method G to afford a white solid as a formate salt with a purity of 99.3%.

This pure peptide was analyzed by analytical RP-HPLC with column 1 (gradient 20-40; t_{R}=4.2 min) and RP-HPLC-ESI-MS (calculated: 1437.6 (M); observed: 1439 [M+H]⁺).

### Example 4

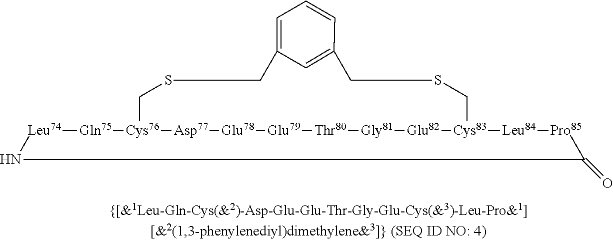

This 12 mer bicyclic peptide analog was synthesized according to the general scheme 2. The linear sequence was obtained following the procedure described in section 1b for those C-terminal acid peptide sequences. The cleavage from the resin without global deprotection was carried out as described in section 2a. The cyclization 1 (see scheme 2), to form the amide bond between the C-terminal and the N-terminal, was done by the Method F, cyclization 1. The global deprotection was performed according to the protocol described in section 4. The cyclization 2 (see scheme 2), to form the incorporation of 1,3-bis(bromomethyl)benzene linker between the cysteines, was done by the Method F, cyclization 2a. Finally, the peptide crude was purified with equipment A and column 1 according to the Method G to afford a white solid with a purity of 94.5%.

This pure peptide was analyzed by analytical RP-HPLC with column 1 (gradient 30-40; t_{R}=3.4 min) and RP-HPLC-ESI-MS (calculated: 1419.6 (M); observed: 1421 [M+H]⁺).

### Example 5

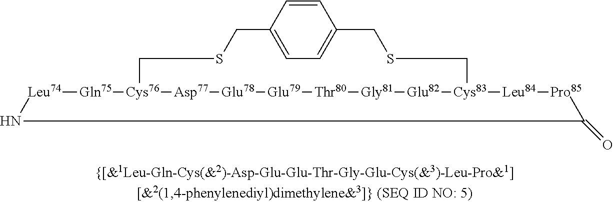

This 12 mer bicyclic peptide analog was synthesized according to the general scheme 2. The linear sequence was obtained following the procedure described in section 1b for those C-terminal acid peptide sequences. The cleavage from the resin without global deprotection was carried out as described in section 2a. The cyclization 1 (see scheme 2), to form the amide bond between the C-terminal and the N-terminal, was done by the Method F, cyclization 1. The global deprotection was performed according to the protocol described in section 4. The cyclization 2 (see scheme 2), to form the incorporation of 1,4-bis(bromomethyl)benzene linker between the cysteines, was done by the Method F, cyclization 2a. Finally, the peptide crude was purified with equipment A and column 1 according to the Method G to afford a white solid with a purity of 97.7%.

This pure peptide was analyzed by analytical RP-HPLC with column 1 (gradient 25-30; t_{R}=6.6 min) and RP-HPLC-ESI-MS (calculated: 1419.6 (M); observed: 1421 [M+H]⁺).

### Example 6

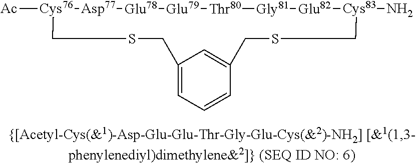

This 8 mer peptide analog was synthesized following the general scheme 1. The linear sequence was obtained according to the procedure described in section 1a for those C-terminal amide peptide sequences and using the Fmoc-Rink amide AM PS resin. The N-terminus was acetylated according to the protocol detailed in section 1a. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method G to afford a white solid with a purity of 99.7%.

This pure peptide was analyzed by analytical RP-HPLC with column 1 (gradient 15-30; t_{R}=4.5 min) and RP-HPLC-ESI-MS (calculated: 1027.3 (M); observed: 1028 [M+H]⁺).

### Example 7

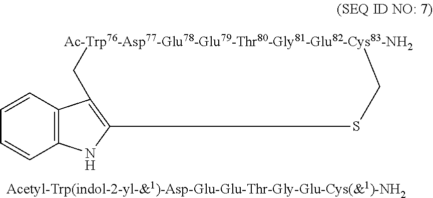

This 8 mer peptide analog was synthesized according to the general scheme 1. The linear sequence was obtained according to the procedure described in section 1a for those C-terminal amide peptide sequences and using the Fmoc-Rink amide AM PS resin. The N-terminus was acetylated following the protocol detailed in section 1a. The cleavage from the resin with global deprotection was carried out as described in section 2b. The cyclization to form the Trp-Cys cross-bridge was done by the Method C. Finally, the peptide crude was purified with equipment A and column 1 according to the Method G to afford a white solid with a purity of 99.3%.

This pure peptide was analyzed by analytical RP-HPLC with column 1 (gradient 10-25; t_{R}=6.4 min) and RP-HPLC-ESI-MS (calculated: 1006.3 (M); observed: 1007 [M+H]⁺).

### Example 8

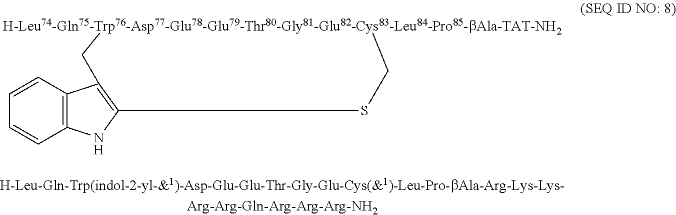

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. The cleavage from the resin with global deprotection was carried out as described in section 2b. The cyclization to form the Trp-Cys cross-bridge was done by the Method C. Finally, the peptide crude was purified with equipment A and column 1 according to the Method H to afford a white solid as a trifluoroacetate salt with a purity of 97.8%.

This pure peptide was analyzed by analytical RP-HPLC with column 2 (gradient 10-20; t_{R}=17.5 min) and RP-HPLC-ESI-MS (calculated: 2807.5 (M); observed: 1405 [M+2H]⁺/2 and 937 [M+3H]⁺/3).

### Example 9

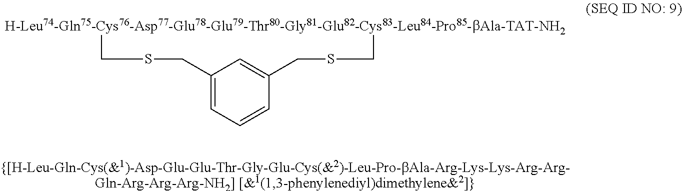

This 12 mer peptide analog conjugated to the CPP TAT was synthesized according to the general scheme 1. The linear sequence was obtained according to the procedure described in section 1a for those C-terminal amide peptide sequences and using the Fmoc-Rink amide AM PS resin. The incorporation of the CPP TAT was carried out on solid phase stepwise until complete its nine amino acid sequence. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method H to afford a white solid as a trifluoroacetate salt with a purity of 93.6%.

This pure peptide was analyzed by analytical RP-HPLC with column 2 (gradient 10-20; t_{R}=21.2 min) and RP-HPLC-ESI-MS (calculated: 2828.5 (M); observed: 1416 [M+2H]⁺/2 and 944 [M+3H]⁺/3).

### Example 10

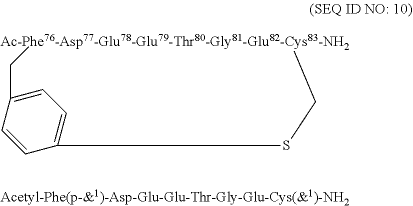

This 8 mer peptide analog was synthesized according to the general scheme 1. The linear sequence was obtained following the procedure described in section 1a for those C-terminal amide peptide sequences and using the Fmoc-Rink amide AM PS resin. This analog was synthesized by incorporating the Fmoc-L-Phe(4-I)—OH in position 76 to allow the cyclization with Cys from position 83. The N-terminus was acetylated following the protocol detailed in section 1a. The cleavage from the resin with global deprotection was carried out as described in section 2b. The cyclization was done by the Method D. Finally, the peptide crude was purified with equipment A and column 1 according to the Method G to afford a white solid with a purity of 99.9%.

This pure peptide was analyzed by analytical RP-HPLC with column 1 (gradient 10-30; t_{R}=4.0 min) and RP-HPLC-ESI-MS (calculated: 967.3 (M); observed: 968 [M+H]⁺).

### Example 11

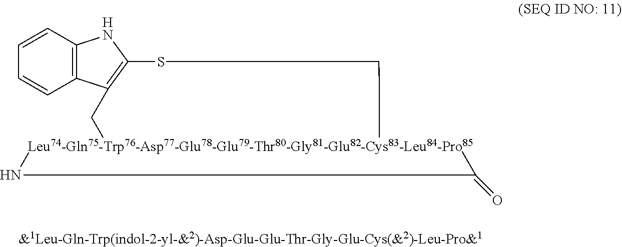

This 12 mer bicyclic peptide analog was synthesized according to the general scheme 2. The linear sequence was obtained following the procedure described in section 1b for those C-terminal acid peptide sequences. The cleavage from the resin without global deprotection was carried out as described in section 2a. The cyclization 1 (see scheme 2), to form the amide bond between the C-terminal and the N-terminal, was done by the Method F, cyclization 1. The global deprotection was performed according to the protocol described in section 4. The cyclization 2 (see scheme 2), to form the Trp-Cys cross-bridge between the cysteines, was done by the Method F, cyclization 2b. Finally, the peptide crude was purified with equipment A and column 1 according to the Method G to afford a white solid with a purity of 94.9%.

This pure peptide was analyzed by analytical RP-HPLC with column 1 (gradient 25-30; t_{R}=6.0 min) and RP-HPLC-ESI-MS (calculated: 1398.6 (M); observed: 1400 [M+H]⁺).

### Example 12

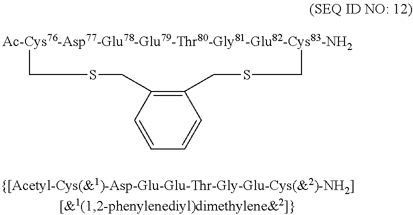

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1 a. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment A and column 1 according to the Method G to afford a white solid with a purity of 99.9%.

This pure peptide was analyzed by analytical RP-HPLC with column 1 (gradient 15-30; t_{R}=5.5 min) and RP-HPLC-ESI-MS (calculated: 1027.3 (M); observed: 1028 [M+H]⁺).

### Example 13

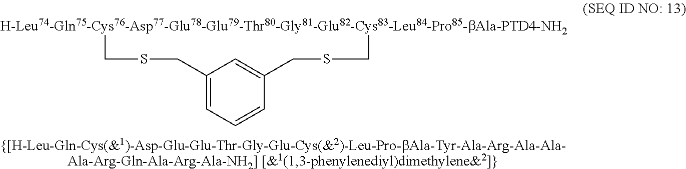

This 12 mer peptide analog conjugated to the CPP PTD4 was synthesized according to the general scheme 1. The linear sequence was obtained following the procedure described in section 1a for those C-terminal amide peptide sequences and using the Fmoc-Rink amide AM PS resin. The incorporation of the CPP PTD4 was carried out on solid phase stepwise until complete its eleven amino acid sequence. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method H to afford a white solid as a trifluoroacetate salt with a purity of 97.3%.

This pure peptide was analyzed by analytical RP-HPLC with column 1 (gradient 20-30; t_{R}=4.8 min) and RP-HPLC-ESI-MS (calculated: 2693.3 (M); observed: 1348 [M+2H]⁺/2 and 899 [M+3H]⁺/3).

### Example 14

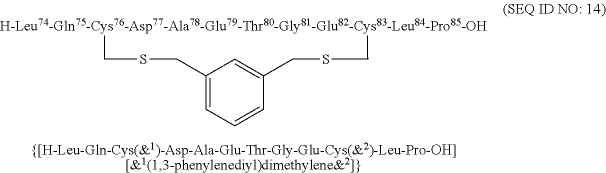

This 12 mer peptide analog was synthesized according to the general scheme 1. The linear sequence was obtained following the procedure described in section 1b for those C-terminal acid peptide sequences. This analog contained an alanine in position 78 instead of a glutamic acid. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method G to afford a white solid as a formate salt with a purity of 99.1%.

This pure peptide was analyzed by analytical RP-HPLC with column 1 (gradient 20-40; t_{R}=5.1 min) and RP-HPLC-ESI-MS (calculated: 1379.6 (M); observed: 1381 [M+H]⁺).

### Example 15

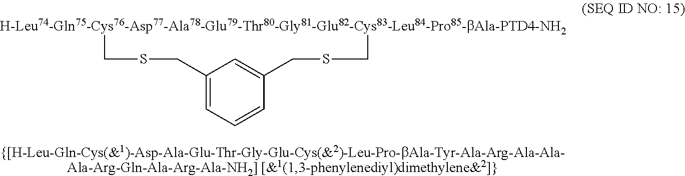

This 12 mer peptide analog conjugated to the CPP PTD4 was synthesized according to the general scheme 1. The linear sequence was obtained following the procedure described in section 1a for those C-terminal amide peptide sequences and using the Fmoc-Rink amide AM PS resin. The incorporation of the CPP PTD4 was carried out on solid phase stepwise until complete its eleven amino acid sequence. This analog contained an alanine in position 78 instead of a glutamic acid. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 by according to the Method H to afford a white solid as a trifluoroacetate salt with a purity of 99.9%.

This pure peptide was analyzed by analytical RP-HPLC with column 1 (gradient 20-40; t_{R}=4.0 min) and RP-HPLC-ESI-MS (calculated: 2635.3 (M); observed: 1319 [M+2H]⁺/2 and 880 [M+3H]⁺/3).

### Example 16

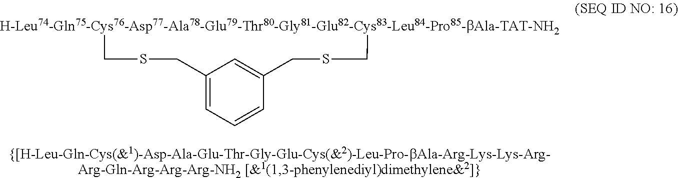

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained an alanine in position 78 instead of a glutamic acid. The cleavage from the resin with global deprotection was carried out as described in section 2b. The cyclization through incorporation of 1,3-bis(bromomethyl)benzene linker between the cysteines was done by the Method A. Finally, the peptide crude was purified with equipment A and column 1 according to the Method H to afford a white solid as a trifluoroacetate salt with a purity of 99.9%.

This pure peptide was analyzed by analytical RP-HPLC with column 1 (gradient 10-30; t_{R}=5.9 min) and RP-HPLC-ESI-MS (calculated: 2770.5 (M); observed: 1388 [M+2H]⁺/2 and 925 [M+3H]⁺/3).

### Example 17

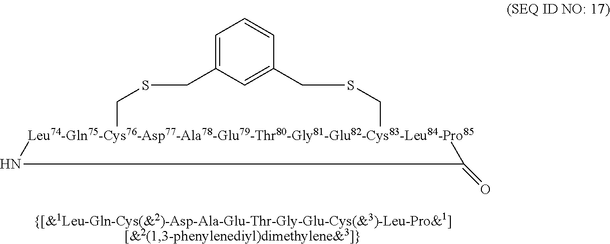

This 12 mer bicyclic peptide analog was synthesized according to the general scheme 2. The linear sequence was obtained following the procedure described in section 1b for those C-terminal acid peptide sequences. This analog contained an alanine in position 78 instead of a glutamic acid. The cleavage from the resin without global deprotection was carried out as described in section 2a. The cyclization 1 (see scheme 2), to form the amide bond between the C-terminal and the N-terminal, was done by the Method F, cyclization 1. The global deprotection was performed by according to the protocol described in section 4. The cyclization 2 (see scheme 2), to form the incorporation of 1,3-bis(bromomethyl)benzene linker between the cysteines, was carried on according to the Method F, cyclization 2a. Finally, the peptide crude was purified with equipment A and column 1 by according to the Method G to afford a white solid with a purity of 99.9%.

This pure peptide was analyzed by analytical RP-HPLC with column 1 (gradient 30-40; t_{R}=3.8 min) and RP-HPLC-ESI-MS (calculated: 1361.6 (M); observed: 1363 [M+H]⁺).

### Example 18

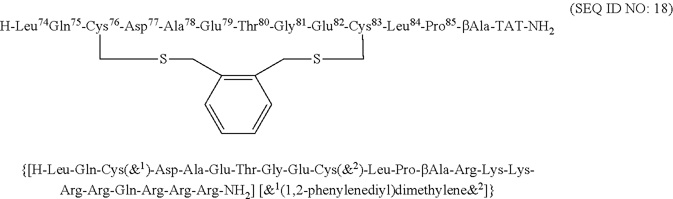

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase stepwise until complete its nine amino acid sequence. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment A and column 1 according to the Method H to afford a white solid as a trifluoroacetate salt with a purity of 99.9%.

This pure peptide was analyzed by analytical RP-HPLC with column 1 (gradient 10-30; t_{R}=5.9 min) and RP-HPLC-ESI-MS (calculated: 2770.5 (M); observed: 1388 [M+2H]⁺/2 and 925 [M+3H]⁺/3).

### Example 19

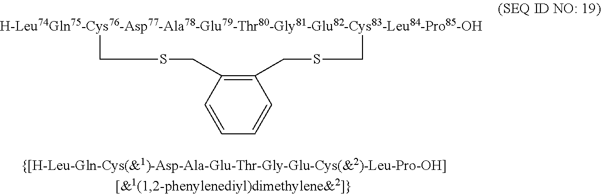

This 12 mer peptide analog was synthesized according to the general scheme 1. The linear sequence was obtained following the procedure described in section 1b for those C-terminal acid peptide sequences. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment A and column 1 according to the Method G to afford a white solid as a formate salt with a purity of 94.5%. This pure peptide was analyzed by analytical RP-HPLC with column 1 (gradient 20-30; t_{R}=6.1 min) and RP-HPLC-ESI-MS (calculated: 1379.6 (M); observed: 1381 [M+H]⁺).

### Example 20

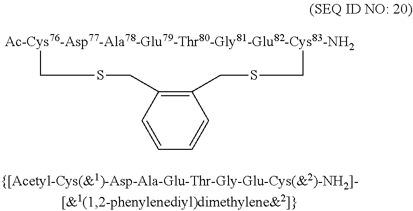

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment A and column 1 according to the Method G to afford a white solid with a purity of 99.9%.

This pure peptide was analyzed by analytical RP-HPLC with column 1 (gradient 20-40; t_{R}=3.8 min) and RP-HPLC-ESI-MS (calculated: 969.3 (M); observed: 970 [M+H]⁺).

### Example 21

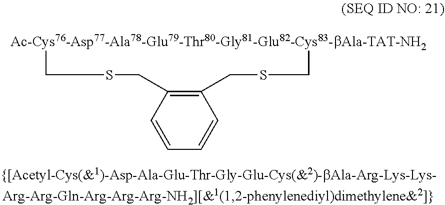

This 8 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. The N-terminus was acetylated following the protocol detailed in section 1a. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment A and column 1 according to the Method H to afford a white solid as a trifluoroacetate salt with a purity of 99.9%.

This pure peptide was analyzed by analytical RP-HPLC with column 1 (gradient 5-30; t_{R}=6.0 min) and RP-HPLC-ESI-MS (calculated: 2361.2 (M); observed: 1182 [M+2H]⁺/2 and 788 [M+3H]⁺/3).

### Example 22

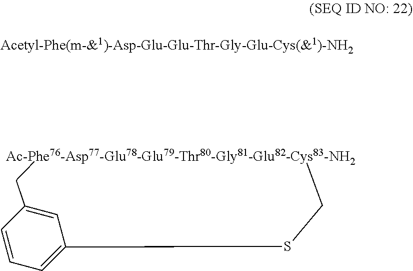

This 8 mer peptide analog was synthesized according to the general scheme 1. The linear sequence was obtained following the procedure described in section 1a for those C-terminal amide peptide sequences and using the Fmoc-Rink amide AM PS resin. This analog was synthesized by incorporating the Fmoc-L-Phe(3-I)—OH in position 76 to allow the cyclization with Cys from position 83. The N-terminus was acetylated following the protocol detailed in section 1a. The cleavage from the resin with global deprotection was carried out as described in section 2b. The cyclization was done by the Method D. Finally, the peptide crude was purified with equipment A and column 1 according to the Method G to afford a white solid as a formate salt with a purity of 99.9%.

This pure peptide was analyzed by analytical RP-HPLC with column 1 (gradient 5-50; t_{R}=4.8 min) and RP-HPLC-ESI-MS (calculated: 967.3 (M); observed: 968 [M+H]⁺).

### Example 23

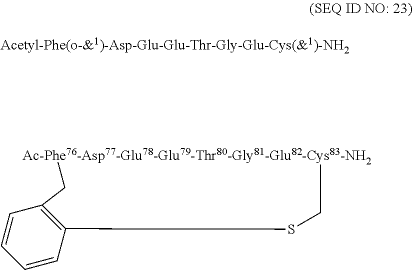

This 8 mer peptide analog was synthesized according to the general scheme 1. The linear sequence was obtained following the procedure described in section 1a for those C-terminal amide peptide sequences and using the Fmoc-Rink amide AM PS resin. This analog was synthesized by incorporating the Fmoc-L-Phe(2-I)—OH in position 76 to allow the cyclization with Cys from position 83. The N-terminus was acetylated following the protocol detailed in section 1a. The cleavage from the resin with global deprotection was carried out as described in section 2b. The cyclization was done by the Method D. Finally, the peptide crude was purified with equipment A and column 1 according to the Method G to afford a white solid with a purity of 99.9%.

This pure peptide was analyzed by analytical RP-HPLC with column 1 (gradient 5-50; t_{R}=4.5 min) and RP-HPLC-ESI-MS (calculated: 967.3 (M); observed: 968 [M+H]⁺).

### Example 24

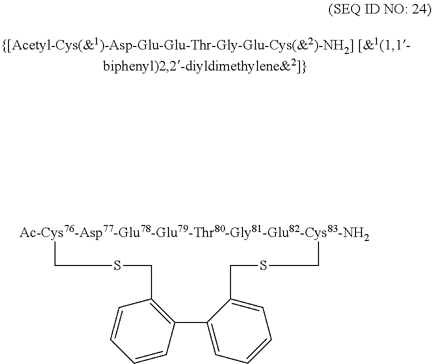

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. The cleavage from the resin with global deprotection was carried out as described in section 2b. The cyclization through incorporation of 2,2′-bis(bromomethyl)-1,1′-biphenyl linker between the cysteines was done by the Method A. Finally, the peptide crude was purified with equipment A and column 1 according to the Method G to afford a white solid with a purity of 99.9%.

This pure peptide was analyzed by analytical RP-HPLC with column 1 (gradient 20-40; t_{R}=4.2 min) and RP-HPLC-ESI-MS (calculated: 1103.4 (M); observed: 1105 [M+2H]⁺).

### Example 25

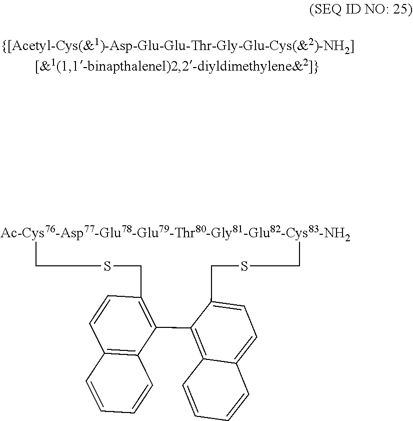

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. The cleavage from the resin with global deprotection was carried out as described in section 2b. The cyclization through incorporation of (R)-2,2′-bis(bromomethyl)-1,1′-binaphthyl linker between the cysteines was done by the Method A. Finally, the peptide crude was purified with equipment A and column 1 according to the Method G to afford a white solid with a purity of 99.9%.

This pure peptide was analyzed by analytical RP-HPLC with column 1 (gradient 30-50; t_{R}=4.7 min) and RP-HPLC-ESI-MS (calculated: 1203.4 (M); observed: 1205 [M+2H]⁺).

### Example 26

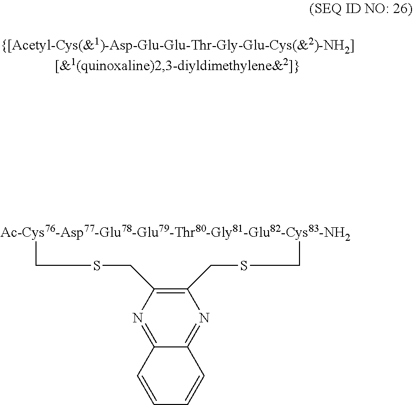

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. The cleavage from the resin with global deprotection was carried out as described in section 2b. The cyclization through incorporation of 2,3-bis(bromomethyl)quinoxaline linker between the cysteines was done by the Method A. Finally, the peptide crude was purified with equipment A and column 1 according to the Method G to afford a white solid with a purity of 98%.

This pure peptide was analyzed by analytical RP-HPLC with column 1 (gradient 10-20; t_{R}=5.5 min) and RP-HPLC-ESI-MS (calculated: 1079.3 (M); observed: 1081 [M+2H]⁺).

### Example 27

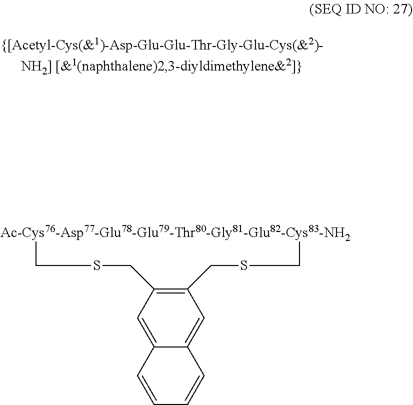

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. The cleavage from the resin with global deprotection was carried out as described in section 2b. The cyclization through incorporation of 2,3-bis(bromomethyl)naphthalene linker between the cysteines was done the Method A. Finally, the peptide crude was purified with equipment A and column 1 according to the Method G to afford a white solid with a purity of 96.1%.

This pure peptide was analyzed by analytical RP-HPLC with column 1 (gradient 25-40; t_{R}=3.2 min) and RP-HPLC-ESI-MS (calculated: 1077.3 (M); observed: 1079 [M+2H]⁺).

### Example 28

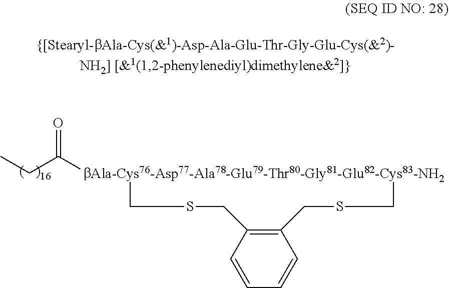

This 8 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 1 according to the Method I to afford a white solid with a purity of 99.9%.

This pure peptide was analyzed by analytical RP-HPLC with column 1 (gradient 70-100; t_{R}=5.4 min) and RP-HPLC-ESI-MS (calculated: 1264.6 (M); observed: 1267 [M+2H]⁺).

### Example 29

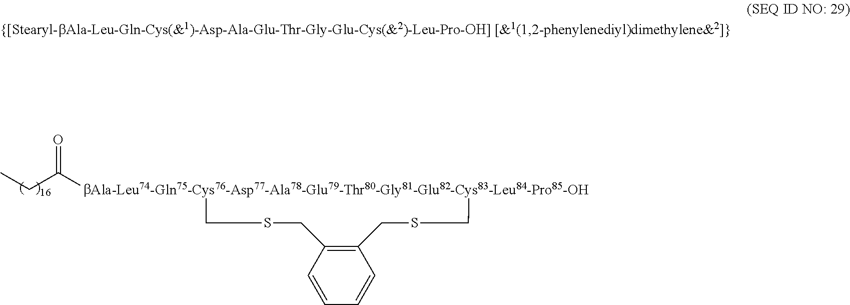

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained an alanine in position 78 instead of a glutamic acid. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 1 according to the Method I to afford a white solid with a purity of 92.5%.

This pure peptide was analyzed by analytical RP-HPLC with column 1 (gradient 80-100; t_{R}=4.1 min) and RP-HPLC-ESI-MS (calculated: 1716.9 (M); observed: 1719 [M+2H]⁺).

### Example 30

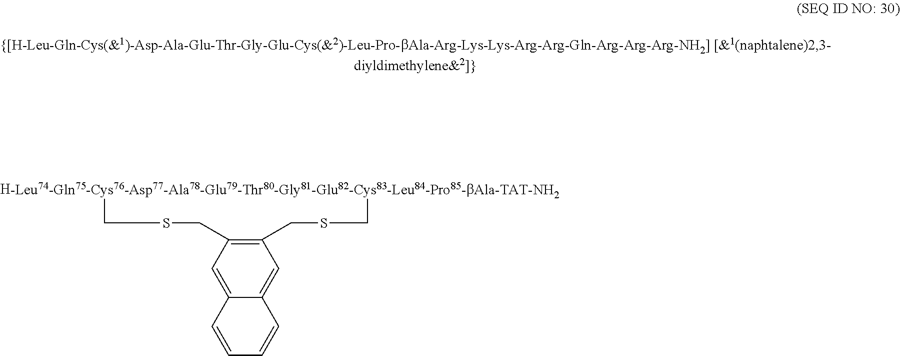

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained an alanine in position 78 instead of a glutamic acid. The cleavage from the resin with global deprotection was carried out as described in section 2b. The cyclization through incorporation of 2,3-bis(bromomethyl)naphthalene linker between the cysteines was done by the Method A. Finally, the peptide crude was purified with equipment B and column 1 according to the Method H to afford a white solid as a trifluoroacetate salt with a purity of 99.9%.

This pure peptide was analyzed by analytical RP-HPLC with column 1 (gradient 0-50; t_{R}=5.9 min) and RP-HPLC-ESI-MS (calculated: 2820.5 (M); observed: 942 [M+3H]⁺/3 and 707 [M+4H]⁺/4).

### Example 31

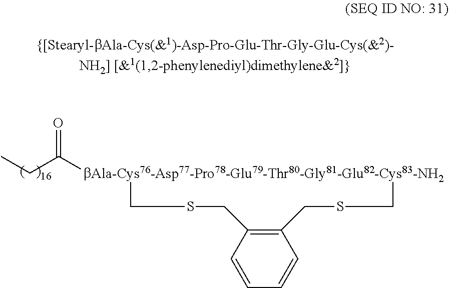

This 8 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 97.7%.

This pure peptide was analyzed by analytical RP-HPLC with column 1 (gradient 70-100; t_{R}=5.6 min) and RP-HPLC-ESI-MS (calculated: 1290.6 (M); observed: 1292.0 [M+H]⁺ and 646.0 [M+2H]⁺/2).

### Example 32

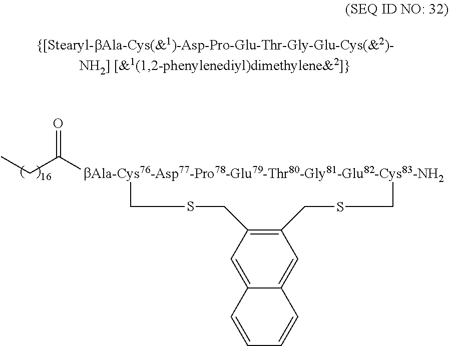

This 8 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 2,3-bis(bromomethyl)naphthalene linker between the cysteines was done by the Method B. Finally, the peptide crude was purified with equipment B and column 3 according to the Method I to afford a white solid with a purity of 97.0%.

This pure peptide was analyzed by analytical RP-HPLC with column 1 (gradient 70-100; t_{R}=6.4 min) and RP-HPLC-ESI-MS (calculated: 1340.6 (M); observed: 1341.0 [M+H]⁺ and 670.8 [M+2H]⁺/2).

### Example 33

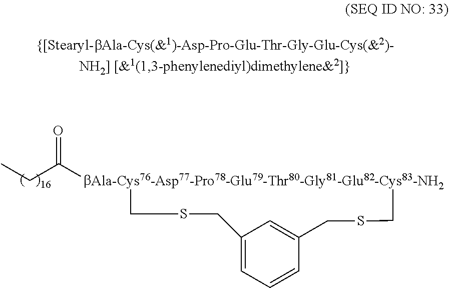

This 8 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,3-bis(bromomethyl)benzene linker between the cysteines was done the Method B. Finally, the peptide crude was purified with equipment B and column 3 according to the Method I to afford a white solid with a purity of 98.7%.

This pure peptide was analyzed by analytical RP-HPLC with column 1 (gradient 70-100; t_{R}=5.2 min) and RP-HPLC-ESI-MS (calculated: 1290.6 (M); observed: 1291.8 [M+H]⁺ and 645.9 [M+2H]⁺/2).

### Example 34

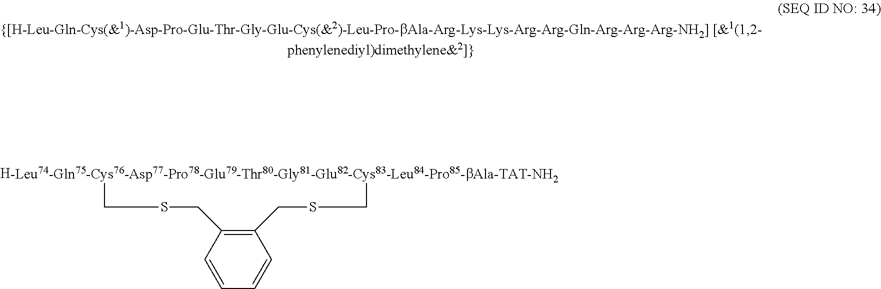

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment B and column 3 according to the Method H to afford a white solid as a trifluoroacetate salt with a purity of 97.4%.

This pure peptide was analyzed by analytical RP-HPLC with column 1 (gradient 0-50; t_{R}=5.6 min) and RP-HPLC-ESI-MS (calculated: 2796.5 (M); observed: 700.0 [M+4H]⁺/4 and 560.1 [M+5H]⁺/5).

### Example 35

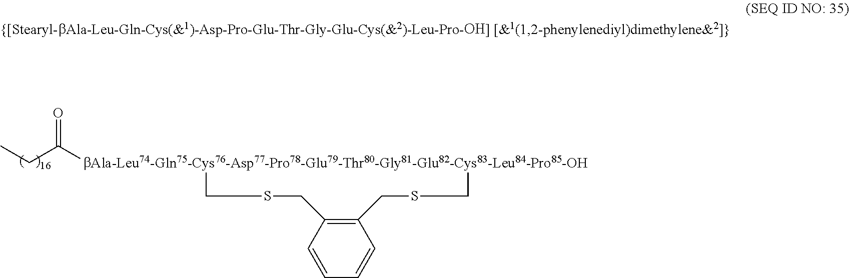

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 98.5%.

This pure peptide was analyzed by analytical RP-HPLC with column 1 (gradient 80-100; t_{R}=4.0 min) and RP-HPLC-ESI-MS (calculated: 1742.9 (M); observed: 872.2 [M+2H]⁺/2 and 581.8 [M+3H]⁺/3).

### Example 36

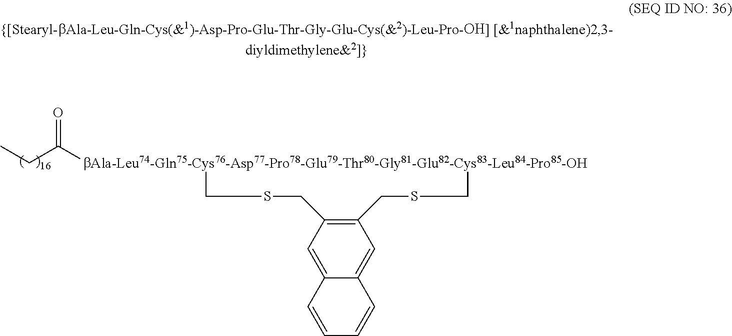

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 2,3-bis(bromomethyl)naphthalene linker between the cysteines was done by the Method B. Finally, the peptide crude was purified with equipment B and column 3 according to the Method I to afford a white solid with a purity of 94.2%.

This pure peptide was analyzed by analytical RP-HPLC with column 1 (gradient 80-100; t_{R}=4.4 min) and RP-HPLC-ESI-MS (calculated: 1792.9 (M); observed: 897.7 [M+2H]⁺/2 and 598.8 [M+3H]⁺/3).

### Example 37

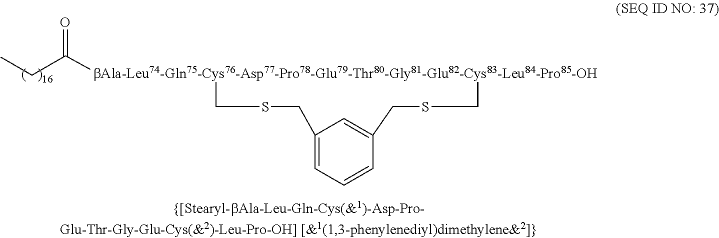

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,3-bis(bromomethyl)benzene linker between the cysteines was done by the Method B. Finally, the peptide crude was purified with equipment B and column 3 according to the Method I to afford a white solid with a purity of 94.8%.

This pure peptide was analyzed by analytical RP-HPLC with column 1 (gradient 80-100; t_{R}=4.0 min) and RP-HPLC-ESI-MS (calculated: 1742.9 (M); observed: 872.6 [M+2H]⁺/2 and 582.1 [M+3H]⁺/3).

### Example 38

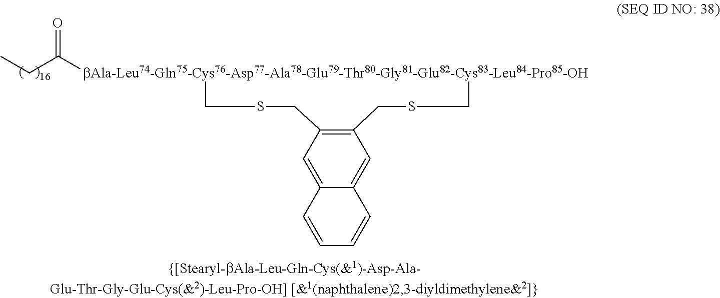

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained an alanine in position 78 instead of a glutamic acid. The cleavage from the resin with global deprotection was carried out as described in section 2b. The cyclization through incorporation of 2,3-bis(bromomethyl)naphthalene linker between the cysteines was done by the Method B. Finally, the peptide crude was purified with equipment B and column 3 according to the Method I to afford a white solid with a purity of 93.2%.

This pure peptide was analyzed by analytical RP-HPLC with column 1 (gradient 80-100; t_{R}=4.3 min) and RP-HPLC-ESI-MS (calculated: 1766.9 (M); observed: 1769 [M+2H]⁺).

### Example 39

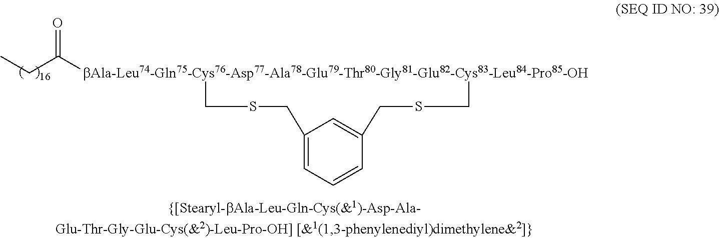

This 12 mer peptide analog conjugated to the stearic fatty acid was synthesized according to the general scheme 1. The linear sequence was obtained following the procedure described in section 1b for those C-terminal acid peptide. The incorporation of the stearic acid was carried out on solid phase at the N-terminal position following the protocol detailed in section 1a. This analog contained an alanine in position 78 instead of a glutamic acid. The cleavage from the resin with global deprotection was carried out as described in section 2b. The cyclization through incorporation of 1,3-bis(bromomethyl)benzene linker between the cysteines was done by the Method B. Finally, the peptide crude was purified with equipment B and column 3 according to the Method I to afford a white solid with a purity of 97.6%.

This pure peptide was analyzed by analytical RP-HPLC with column 1 (gradient 70-100; t_{R}=5.2 min) and RP-HPLC-ESI-MS (calculated: 1716.9 (M); observed: 1717 [M+H]⁺).

### Example 40

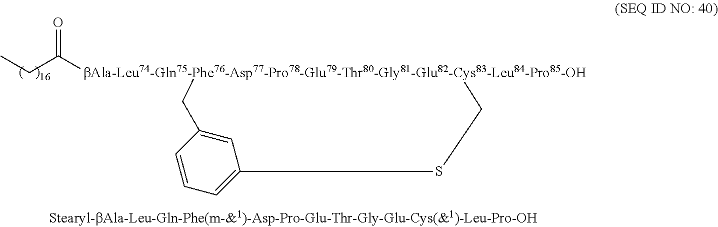

This 12 mer peptide analog conjugated to the stearic fatty acid was synthesized according to the general scheme 1. The linear sequence was obtained following the procedure described in section 1b for those C-terminal acid peptide sequences. The incorporation of the stearic acid was carried out on solid phase at the N-terminal position following the protocol detailed in section 1a. This analog contained a proline in position 78 instead of a glutamic acid. This analog was synthesized by incorporating the Fmoc-L-Phe(3-I)—OH in position 76 to allow the cyclization with Cys from position 83. The cleavage from the resin with global deprotection was carried out as described in section 2b. The cyclization was done by the Method E. Finally, the peptide crude was purified with equipment B and column 3 according to the Method I to afford a white solid with a purity of 96.3%.

This pure peptide was analyzed by analytical RP-HPLC with column 1 (gradient 70-100; t_{R}=5.3 min) and RP-HPLC-ESI-MS (calculated: 1682.9 (M); observed: 1683 [M+H]⁺).

### Example 41

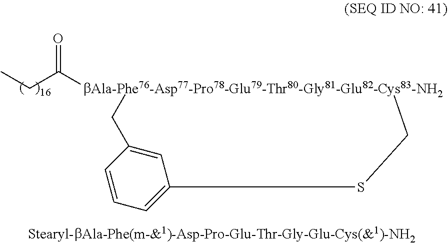

This 8 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. This analog was synthesized by incorporating the Fmoc-L-Phe(3-I)—OH in position 76 to allow the cyclization with Cys from position 83. The cleavage from the resin with global deprotection was carried out as described in section 2b. The cyclization was done by the Method E. Finally, the peptide crude was purified with equipment B and column 3 according to the Method I to afford a white solid with a purity of 99.9%.

This pure peptide was analyzed by analytical RP-HPLC with column 1 (gradient 40-100; t_{R}=6.4 min) and RP-HPLC-ESI-MS (calculated: 1230.6 (M); observed: 1231 [M+H]⁺).

### Example 42

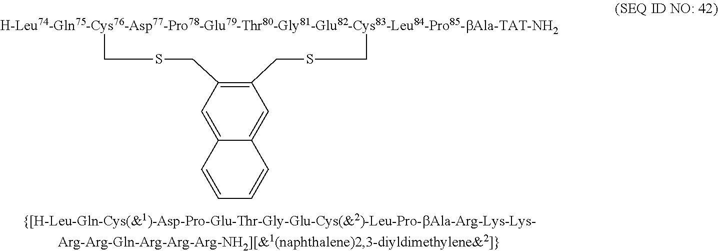

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid. The cleavage from the resin with global deprotection was carried out as described in section 2b. The cyclization through incorporation of 2,3-bis(bromomethyl)naphthalene linker between the cysteines was done the Method A. Finally, the peptide crude was purified with equipment B and column 3 according to the Method H to afford a white solid as a trifluoroacetate salt with a purity of 95.1%.

This pure peptide was analyzed by analytical RP-HPLC with column 1 (gradient 0-50; t_{R}=6.3 min) and RP-HPLC-ESI-MS (calculated: 2846.5 (M); observed: 712.4 [M+4H]⁺/4 and 750.1 [M+5H]⁺/5).

### Example 43

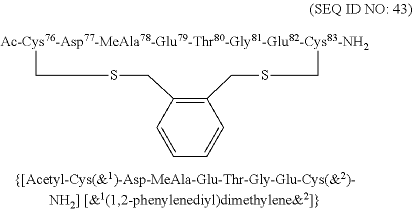

This 8 mer peptide analog was synthesized according to the general scheme 1. The linear sequence was obtained following the procedure described in section 1a for those C-terminal amide peptide sequences and using the Fmoc-Rink amide AM PS resin. The N-terminus was acetylated following the protocol detailed in section 1a. This analog contained an N-methyl-alanine in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment B and column 3 according to the Method G to afford a white solid with a purity of 99.9%.

This pure peptide was analyzed by analytical RP-HPLC with column 1 (gradient 10-60; t_{R}=2.9 min) and RP-HPLC-ESI-MS (calculated: 983.3 (M); observed: 985 [M+2H]⁺).

### Example 44

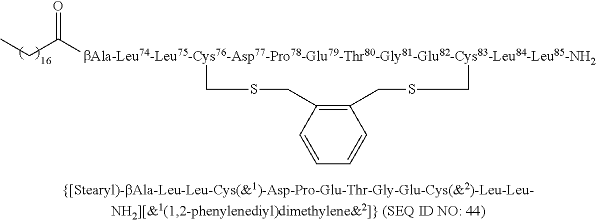

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and two leucines in position 75 and 85 instead of a glutamine and a proline, respectively. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 95.3%.

This pure peptide was analyzed by analytical RP-HPLC with column 1 (gradient 80-100; t_{R}=6.2 min) and RP-HPLC-ESI-MS (calculated: 1742.9 (M); observed: 872.4 [M+2H]⁺/2 and 582.0 [M+3H]⁺/3).

### Example 45

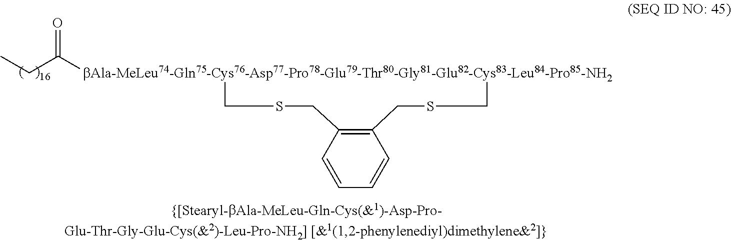

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and an N-methyl-leucine in position 74 instead of a leucine. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 96.3%.

This pure peptide was analyzed by analytical RP-HPLC with column 1 (gradient 80-100; t_{R}=4.3 min) and RP-HPLC-ESI-MS (calculated: 1755.9 (M); observed: 1756.1 [M+H]⁺ and 879.1 [M+2H]⁺/2).

### Example 46

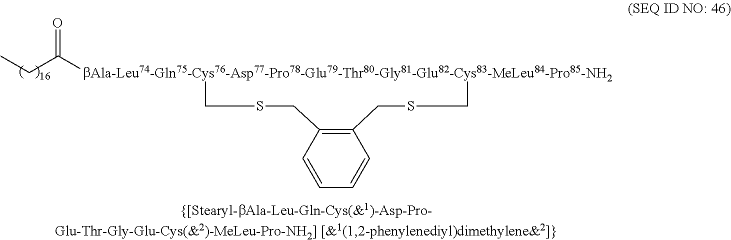

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and an N-methyl-leucine in position 84 instead of a leucine. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 99.5%.

This pure peptide was analyzed by analytical RP-HPLC with column 1 (gradient 70-90; t_{R}=5.2 min) and RP-HPLC-ESI-MS (calculated: 1755.9 (M); observed: 1756.0 [M+H]⁺ and 879.0 [M+2H]⁺/2).

### Example 47

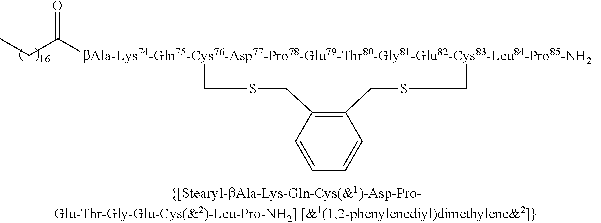

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and a lysine in position 74 instead of a leucine. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method J to afford a white solid as a trifluoroacetate salt with a purity of 98.7%.

This pure peptide was analyzed by analytical RP-HPLC with column 1 (gradient 50-70; t_{R}=6.9 min) and RP-HPLC-ESI-MS (calculated: 1756.9 (M); observed: 879.5 [M+2H]⁺/2 and 586.6 [M+3H]⁺/3).

### Example 48

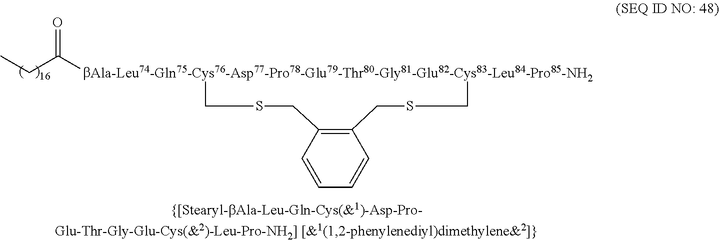

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method I to afford a white solid with a purity of 97.1%.

This pure peptide was analyzed by analytical RP-HPLC with column 1 (gradient 80-100; t_{R}=3.9 min) and RP-HPLC-ESI-MS (calculated: 1741.9 (M); observed: 1745 [M+3H]⁺).

### Example 49

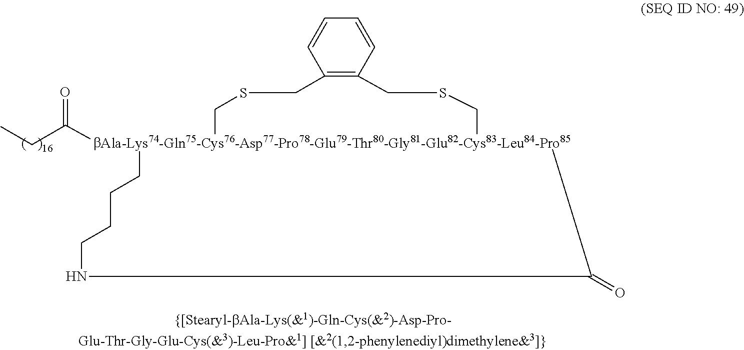

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid and a lysine in position 74 instead of a leucine. The lysine was introduced as a Fmoc-Lys(Alloc)-OH and the Alloc protecting group was removed following the methodology detailed in section 1a. The incorporation of the stearic acid was carried out on solid phase at the N-terminal position according to the protocol detailed in section 1 a. The cleavage from the resin without global deprotection was carried out as described in section 2a. The cyclization 1 (see scheme 2), to form the amide bond between the C-terminal and the side chain of lysine, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 3 according to the Method I to afford a white solid with a purity of 95.0%.

This pure peptide was analyzed by analytical RP-HPLC with column 1 (gradient 70-100; t_{R}=4.9 min) and RP-HPLC-ESI-MS (calculated: 1739.9 (M); observed: 1743 [M+3H]⁺).

### Example 50

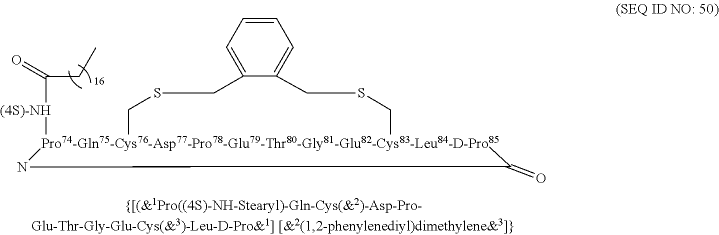

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 98.6%.

This pure peptide was analyzed by analytical RP-HPLC with column 1 (gradient 70-100; t_{R}=5.3 min) and RP-HPLC-ESI-MS (calculated: 1652.8 (M); observed: 1654.1 [M+H]⁺ and 827.0 [M+2H]⁺/2).

### Example 51

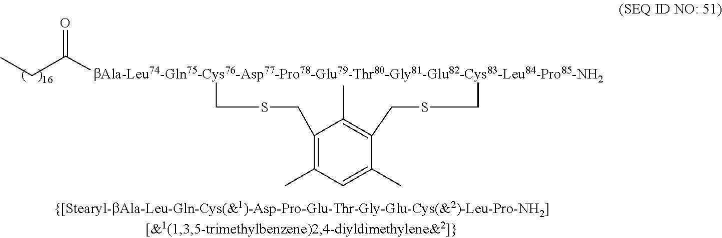

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 2,4-bis(chloromethyl)mesitylene linker between the cysteines was done by the Method B. Finally, the peptide crude was dissolved in DMSO and purified with equipment B and column 3 according to the Method I to afford a white solid with a purity of 94.5%.

This pure peptide was analyzed by analytical RP-HPLC with column 1 (gradient 70-100; t_{R}=4.9 min) and RP-HPLC-ESI-MS (calculated: 1784.0 (M); observed: 1788 [M+4H]⁺).

### Example 52

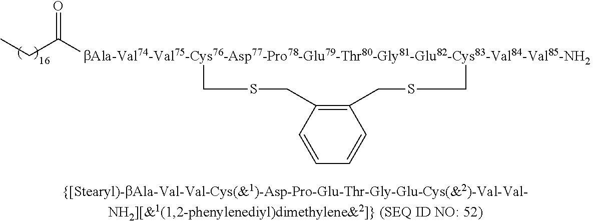

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM ChemMatrix resin (0.49 mmol/g). The incorporation of the stearic acid was carried out on solid phase at the N-terminal position following the protocol detailed in section 1a. This analog contained a proline in position 78 instead of a glutamic acid and four valines in positions 74, 75, 84 and 85 instead of a leucine, a glutamine, a leucine and a proline, respectively. The cleavage from the resin with global deprotection was carried out as described in section 2b. The cyclization through incorporation of 1,2-bis(bromomethyl)benzene linker between the cysteines was done by the Method B. Finally, the peptide crude was dissolved in DMSO-DMF (1:1) and purified with equipment B and column 3 according to the Method I to afford a white solid with a purity of 91.7%.

This pure peptide was analyzed by analytical RP-HPLC with column 1 (gradient 70-100; t_{R}=6.3 min) and RP-HPLC-ESI-MS (calculated: 1686.9 (M); observed: 844.6 [M+2H]⁺/2 and 563.5 [M+3H]⁺/3).

### Example 53

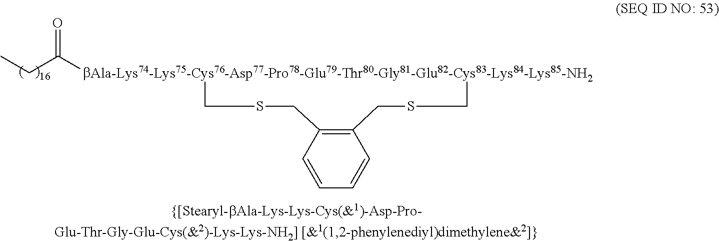

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and four lysines in position 74, 75, 84 and 85 instead of a leucine, a glutamine, a leucine and a proline, respectively. The cleavage from the resin with global deprotection was carried out as described in section 2b. The cyclization through incorporation of 1,2-bis(bromomethyl)benzene linker between the cysteines was done by the Method B. Finally, the peptide crude was purified with equipment B and column 3 according to the Method J to afford a white solid as a trifluoroacetate salt with a purity of 97.3%.

This pure peptide was analyzed by analytical RP-HPLC with column 1 (gradient 30-80; t_{R}=5.9 min) and RP-HPLC-ESI-MS (calculated: 1803.0 (M); observed: 902.3 [M+2H]⁺/2 and 602.2 [M+3H]⁺/3).

### Example 54

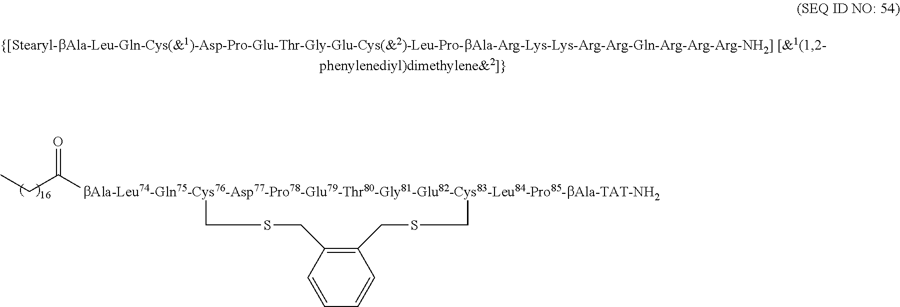

This 12 mer peptide analog conjugated to the CPP TAT and to the stearic fatty acid was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 1,2-bis(bromomethyl)benzene linker between the cysteines was done by the Method A. Finally, the peptide crude was dissolved in H₂O-ACN (4:1) and purified with equipment B and column 3 according to the Method H to afford a white solid as a trifluoroacetate salt with a purity of 95.3%.

This pure peptide was analyzed by analytical RP-HPLC with column 1 (gradient 45-55; t_{R}=4.7 min) and RP-HPLC-ESI-MS (calculated: 3133.8 (M); observed: 1047 [M+3H]⁺/3 and 786 [M+4H]⁺/4).

### Example 55

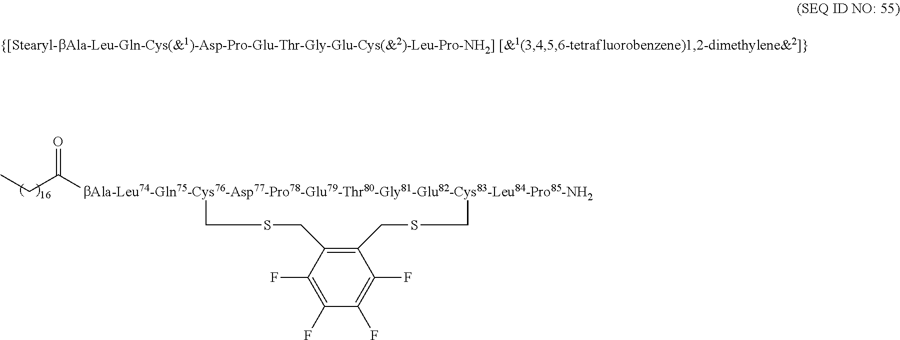

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid.

The cleavage from the resin with global deprotection was carried out as described in section 2b. The cyclization through incorporation of 1,2-bis(bromomethyl)-3,4,5,6-tetrafluorobenzene linker between the cysteines was done by decreasing the quantity of NH₄HCO₃ until 5 mM and by the Method B. Finally, the peptide crude was purified with equipment B and column 3 according to the Method I to afford a white solid with a purity of 91.7%.

This pure peptide was analyzed by analytical RP-HPLC with column 1 (gradient 75-80; t_{R}=6.4 min) and RP-HPLC-ESI-MS (calculated: 1813.9 (M); observed: 1817 [M+3H]⁺).

### Example 56

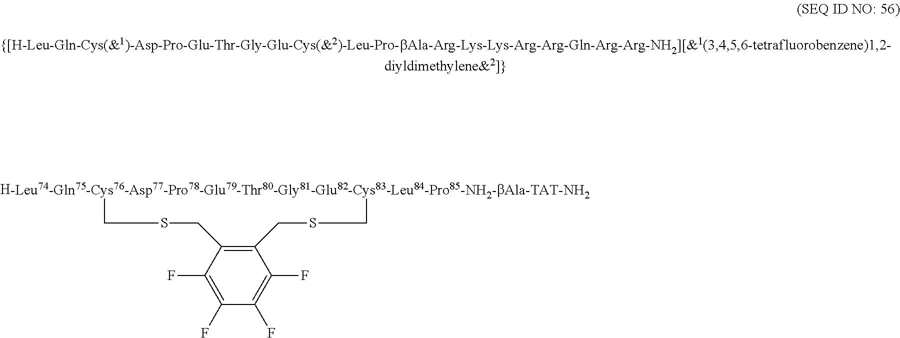

This 12 mer peptide analog conjugates to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid. The cleavage from the resin with global deprotection was carried out as described in section 2b. The cyclization through incorporation of 1,2-bis(bromomethyl)-3,4,5,6-tetrafluorobenzene linker between the cysteines was done by dissolving the peptide crude in H₂O and using two kind of bases, NH₄HCO₃ (10 mM) and DIEA (10 mM) following the Method A. Finally, the peptide crude was purified with equipment B and column 3 according to the Method H to afford a white solid as a trifluoroacetate salt with a purity of 95.5%.

This pure peptide was analyzed by analytical RP-HPLC with column 1 (gradient 20-40; t_{R}=3.8 min) and RP-HPLC-ESI-MS (calculated: 2868.5 (M); observed: 959 [M+3H]⁺/3 and 719 [M+4H]⁺/4).

### Example 57

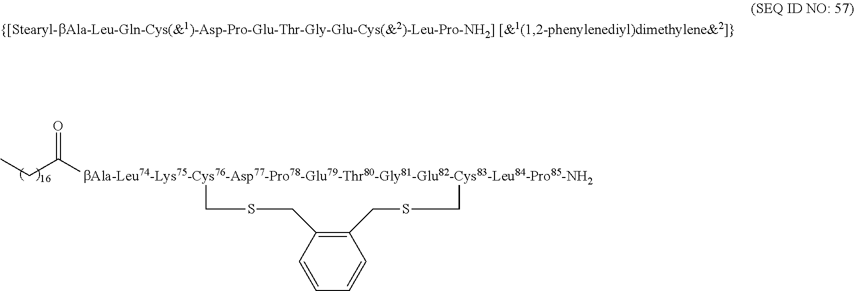

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and two lysines in position 74 and 75 instead of a leucine and a proline, respectively. The cleavage from the resin with global deprotection was carried out as described in section 2b. The cyclization through incorporation of 1,2-bis(bromomethyl)benzene linker between the cysteines was done by the Method B. Finally, the peptide crude was dissolved in H₂O and purified with equipment B and column 3 according to the Method H to afford a white solid as a trifluoroacetate salt with a purity of 95.0%.

This pure peptide was analyzed by analytical RP-HPLC with column 1 (gradient 50-70; t_{R}=5.2 min) and RP-HPLC-ESI-MS (calculated: 1756.9 (M); observed: 879.6 [M+2H]⁺/2 and 586.8 [M+3H]⁺/3).

### Example 58

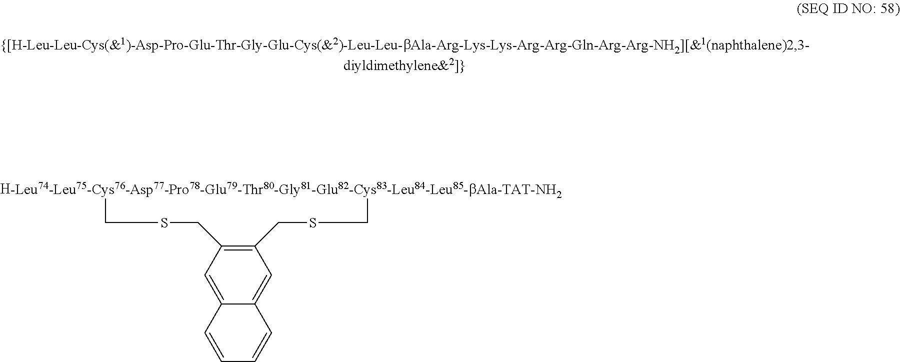

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid and two leucines in position 75 and 85 instead of a glutamine and a proline, respectively. The cleavage from the resin with global deprotection was carried out as described in section 2b. The cyclization through incorporation of 2,3-bis(bromomethyl)naphthalene linker between the cysteines was done by the Method A. Finally, the peptide crude was purified with equipment B and column 3 according to the Method H to afford a white solid as a trifluoroacetate salt with a purity of 96.0%.

This pure peptide was analyzed by analytical RP-HPLC with column 1 (gradient 10-50; t_{R}=5.8 min) and RP-HPLC-ESI-MS (calculated: 2848.6 (M); observed: 712.8 [M+4H]⁺/4 and 750.6 [M+5H]⁺/5).

### Example 59

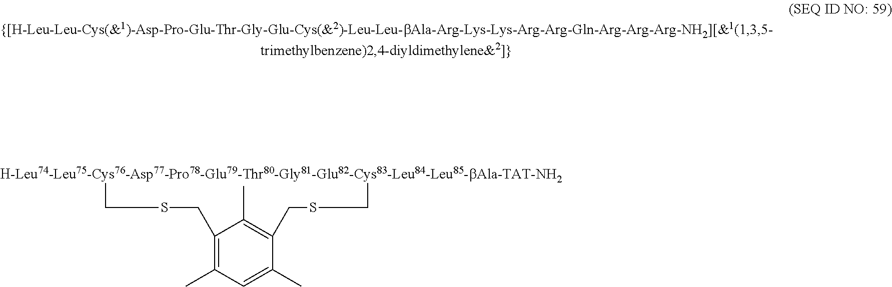

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid and two leucine in position 75 and 85 instead of a glutamine and a proline, respectively. The cleavage from the resin with global deprotection was carried out as described in section 2b. The cyclization through incorporation of 2,4-bis(chloromethyl)mesitylene linker between the cysteines was done by the Method A. Finally, the peptide crude was purified with equipment B and column 3 according to the Method H to afford a white solid as a trifluoroacetate salt with a purity of 97.0%.

This pure peptide was analyzed by analytical RP-HPLC with column 1 (gradient 20-60; t_{R}=3.8 min) and RP-HPLC-ESI-MS (calculated: 2839.6 (M); observed: 948.1 [M+3H]⁺/3 and 711.3 [M+4H]⁺/4).

### Example 60

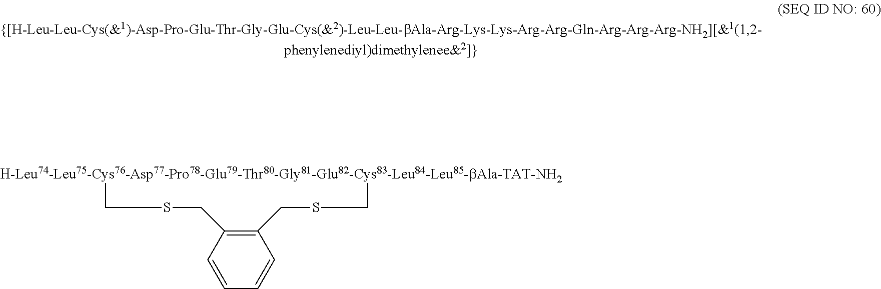

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase and stepwise until complete its nine amino acid sequence. This analog contained a proline in position 78 instead of a glutamic acid and two leucine in position 75 and 85 instead of a glutamine and a proline, respectively. The cleavage from the resin with global deprotection was carried out as described in section 2b. The cyclization through incorporation of 1,2-bis(bromomethyl)benzene linker between the cysteines was done by the Method A. Finally, the peptide crude was purified with equipment B and column 3 according to the Method H to afford a white solid as a trifluoroacetate salt with a purity of 96.0%.

This pure peptide was analyzed by analytical RP-HPLC with column 1 (gradient 20-60; t_{R}=3.3 min) and RP-HPLC-ESI-MS (calculated: 2797.5 (M); observed: 700.5 [M+4H]⁺/4 and 560.8 [M+5H]⁺/5).

### Example 61

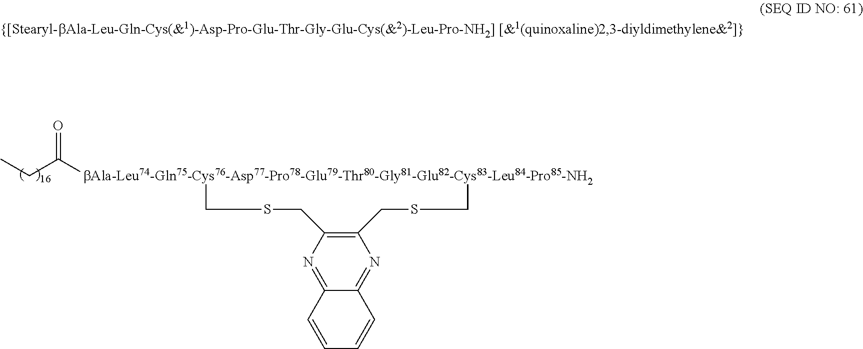

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 2,3-bis(bromomethyl)quinoxaline linker between the cysteines was done by the Method B. Finally, the peptide crude was purified with equipment B and column 3 according to the Method J to afford a white solid as a trifluoroacetate salt with a purity of 99.5%.

This pure peptide was analyzed by analytical RP-HPLC with column 1 (gradient 70-100; t_{R}=4.8 min) and RP-HPLC-ESI-MS (calculated: 1793.9 (M); observed: 898.3 [M+2H]⁺/2 and 599.2 [M+3H]⁺/3).

### Example 62

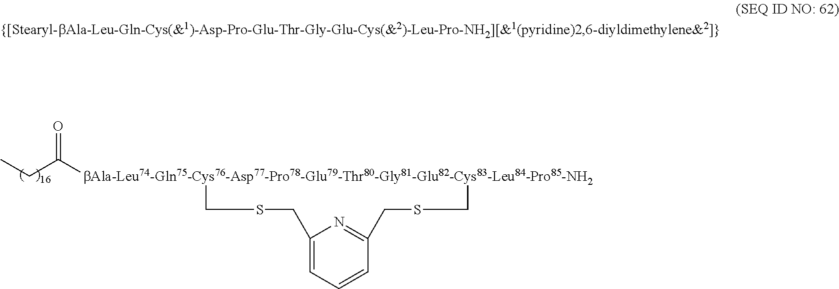

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 2,6-bis(bromomethyl)pyridine linker between the cysteines was done by the Method B. Finally, the peptide crude was purified with equipment B and column 3 according to the Method J to afford a white solid as a trifluoroacetate salt with a purity of 98.0%.

This pure peptide was analyzed by analytical RP-HPLC with column 1 (gradient 70-100; t_{R}=4.6 min) and RP-HPLC-ESI-MS (calculated: 1742.9 (M); observed: 872.7 [M+2H]⁺/2 and 582.0 [M+3H]⁺/3).

### Example 63

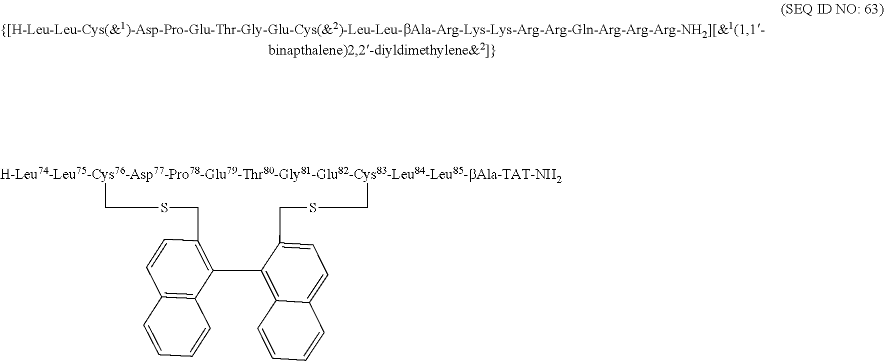

This 12 mer peptide analog conjugated to the CPP TAT was synthesized according to the general scheme 1. The linear sequence was obtained following the procedure described in section 1a for those C-terminal amide peptide sequences and using the Fmoc-Rink amide AM PS resin. The incorporation of the CPP TAT was carried out on solid phase stepwise until complete its nine amino acid sequence. This analog contained a proline in position 78 instead of a glutamic acid and two leucine in position 75 and 85 instead of a glutamine and a proline, respectively. The cleavage from the resin with global deprotection was carried out as described in section 2b. The cyclization through incorporation of (R)-2,2′-bis(bromomethyl)-1,1′-binaphthyl linker between the cysteines was done by the Method A. Finally, the peptide crude was purified with equipment B and column 3 according to the Method H to afford a white solid as a trifluoroacetate salt with a purity of 99.5%.

This pure peptide was analyzed by analytical RP-HPLC with column 1 (gradient 20-60; t_{R}=5.3 min) and RP-HPLC-ESI-MS (calculated: 2973.6 (M); observed: 992.2 [M+3H]⁺/3 and 744.7 [M+4H]⁺/4).

### Example 64

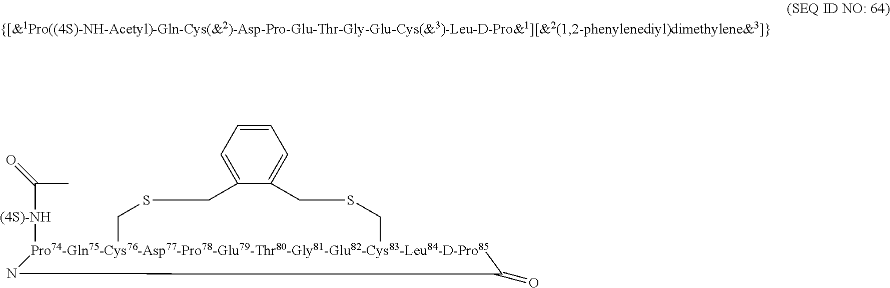

This 12 mer bicyclic peptide analog was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The deprotected amino from side chain of (4S)-aminoproline was acetylated on solid phase following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH—Ac)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the the N-, was done by the Method F, cyclization 1. The global deprotection was performed according to the protocol described in section 4. The cyclization 2 (see scheme 2), to form the incorporation of 1,2-bis(bromomethyl)benzene linker between the cysteines, was done by the Method F, cyclization 2a. Finally, the peptide crude was purified with equipment B and column 3 according to the Method G to afford a white solid with a purity of 99.0%.

This pure peptide was analyzed by analytical RP-HPLC with column 1 (gradient 20-60; t_{R}=3.8 min) and RP-HPLC-ESI-MS (calculated: 1428.6 (M); observed: 1430.0 [M+H]⁺ and 715.6 [M+2H]⁺/2).

### Example 65

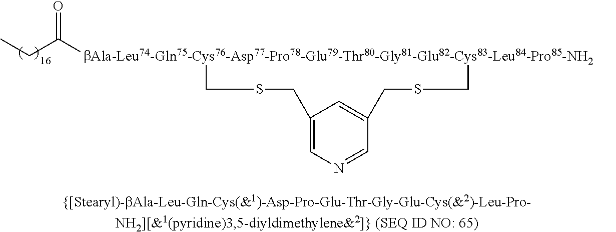

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid. The cleavage from the resin with global deprotection was carried out as described in section 2b. The cyclization through incorporation of 3,5-bis(bromomethyl)pyridine linker between the cysteines was done by the Method B. Finally, the peptide crude was purified with equipment B and column 3 according to the Method J to afford a white solid with a purity of 98.0%.

This pure peptide was analyzed by analytical RP-HPLC with column 1 (gradient 70-100; t_{R}=3.2 min) and RP-HPLC-ESI-MS (calculated: 1742.9 (M); observed: 1744.7 [M+H]⁺ and 872.6 [M+2H]⁺/2).

### Example 66

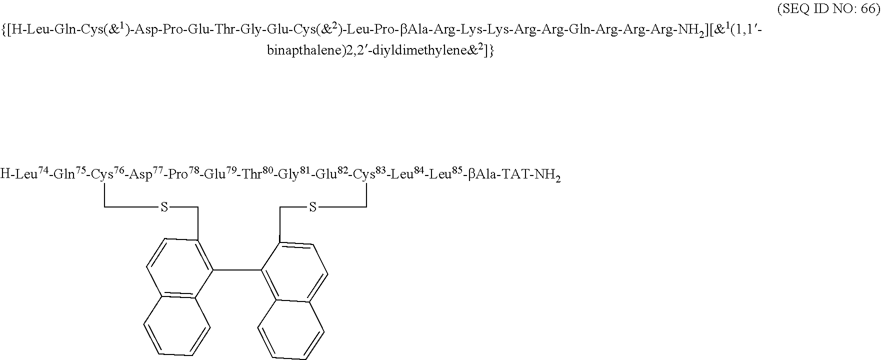

This 12 mer peptide analog conjugated to the CPP TAT was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.12 mmol/g by reducing the equivalents of the first Aa (Arg) coupled to the resin. The incorporation of the CPP TAT was carried out on solid phase and stepwise until completion of its nine amino acid sequence. This analog contained a proline in position 78 instead of a glutamic acid. The two cysteines were introduced as a Fmoc-L-Cys(Mmt)-OH and the Mmt protecting group was removed after the peptide elongation (before the cyclization) following the methodology detailed in section 3h. The cyclization on solid phase through incorporation of (R)-2,2′-bis(bromomethyl)-1,1′-binaphthyl linker between the cysteines was done by the Method H. The cleavage from the resin with global deprotection was carried out after the cyclization as described in section 3h. Finally, the peptide crude was purified with equipment B and column 3 according to the Method H to afford a white solid as a trifluoroacetate salt with a purity of 95.6%.

This pure peptide was analyzed by analytical RP-HPLC with column 1 (gradient 25-35; t_{R}=3.9 min) and RP-HPLC-ESI-MS (calculated: 2972.6 (M); observed: 744.3 [M+4H]⁺/4 and 595.8 [M+5H]⁺/5).

### Example 67

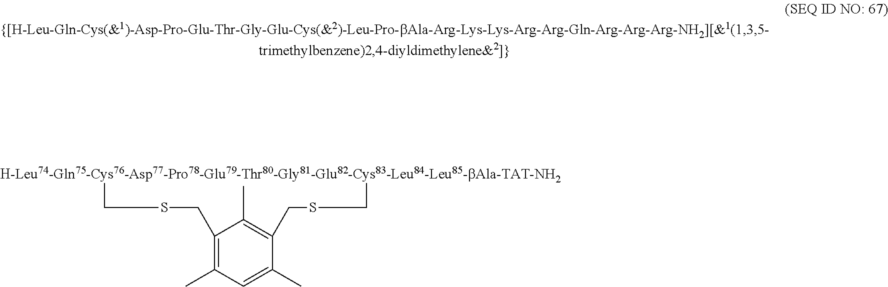

This 12 mer peptide analog conjugated to the CPP TAT was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.12 mmol/g by decreasing the equivalents of the first Aa (Arg) coupled to the resin. The incorporation of the CPP TAT was carried out on solid phase and stepwise until complete its nine amino acid sequence. This analog contained a proline in position 78 instead of a glutamic acid. The two cysteines were introduced as a Fmoc-L-Cys(Mmt)-OH and the Mmt protecting group was removed after the peptide elongation (before the cyclization) according to the methodology detailed in section 3h. The cyclization on solid phase through incorporation of 2,4-bis(chloromethyl)mesitylene linker between the cysteines was done by the Method H. The cleavage from the resin with global deprotection was carried out after the cyclization as described in section 3h. Finally, the peptide crude was purified with equipment B and column 3 according to the Method H to afford a white solid as a trifluoroacetate salt with a purity of 97.9%.

This pure peptide was analyzed by analytical RP-HPLC with column 1 (gradient 10-40; t_{R}=6.1 min) and RP-HPLC-ESI-MS (calculated: 2838.5 (M); observed: 710.8 [M+4H]⁺/4 and 569.2 [M+5H]⁺/5).

### Example 68

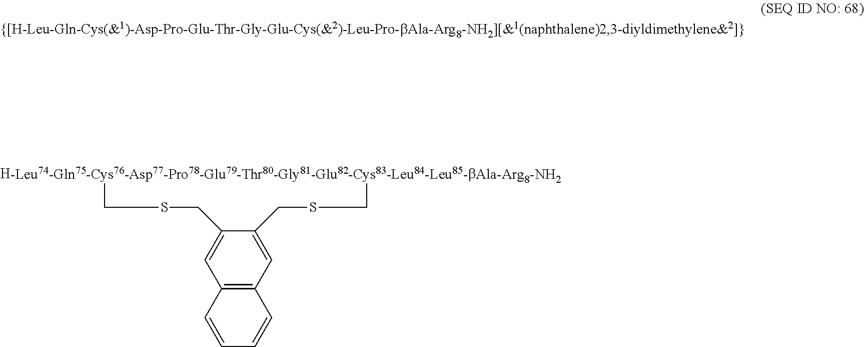

This 12 mer peptide analog conjugated to the CPP L-Arg₈ was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.40 mmol/g by decreasing the equivalents of the first Aa (Arg) coupled to the resin. The incorporation of the CPP L-Arg₈ was carried out on solid phase and stepwise until complete its nine amino acid sequence. This analog contained a proline in position 78 instead of a glutamic acid. The two cysteines were introduced as a Fmoc-L-Cys(Mmt)-OH and the Mmt protecting group was removed after the peptide elongation (before the cyclization) following the methodology detailed in section 3h.

The cyclization on solid phase through incorporation of 2,3-bis(bromomethyl)naphthalene linker between the cysteines was done by the Method H. The cleavage from the resin with global deprotection was carried out after the cyclization as described in section 3h. Finally, the peptide crude was purified with equipment B and column 3 according to the Method H to afford a white solid as a trifluoroacetate salt with a purity of 97.3%.

This pure peptide was analyzed by analytical RP-HPLC with column 1 (gradient 0-60; t_{R}=5.7 min) and RP-HPLC-ESI-MS (calculated: 2774.5 (M); observed: 1388.4 [M+2H]⁺/2 and 925.9 [M+3H]⁺/3).

### Example 69

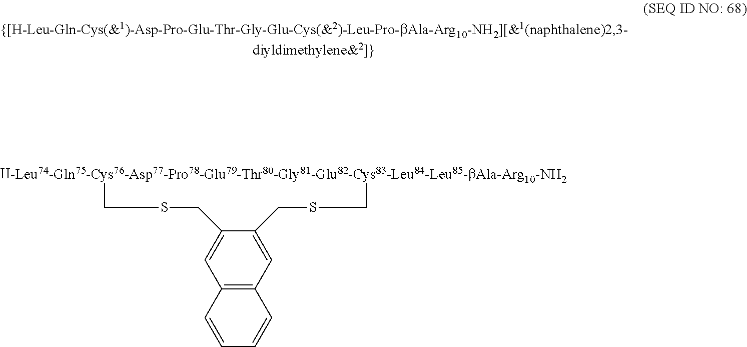

This 12 mer peptide analog conjugated to the CPP Poli-L-Arg₁₀ was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.30 mmol/g by reducing the equivalents of the first Aa (Arg) coupled to the resin. The incorporation of the CPP Poli-L-Arg₁₀ was carried out on solid phase and stepwise until complete its nine amino acid sequence. This analog contained a proline in position 78 instead of a glutamic acid. The two cysteines were introduced as a Fmoc-L-Cys(Mmt)-OH and the Mmt protecting group was removed after the peptide elongation (before the cyclization) following the methodology detailed in section 3h. The cyclization on solid phase through incorporation of 2,3-bis(bromomethyl)naphthalene linker between the cysteines was done by the Method H. The cleavage from the resin with global deprotection was carried out after the cyclization as described in section 3h. Finally, the peptide crude was purified with equipment B and column 3 according to the Method H to afford a white solid as a trifluoroacetate salt with a purity of 98.3%.

This pure peptide was analyzed by analytical RP-HPLC with column 1 (gradient 0-50; t_{R}=6.5 min) and RP-HPLC-ESI-MS (calculated: 3086.7 (M); observed: 773.2 [M+4H]⁺/4, 618.9 [M+5H]⁺/5 and 515.9 [M+6H]⁺/6).

### Example 70

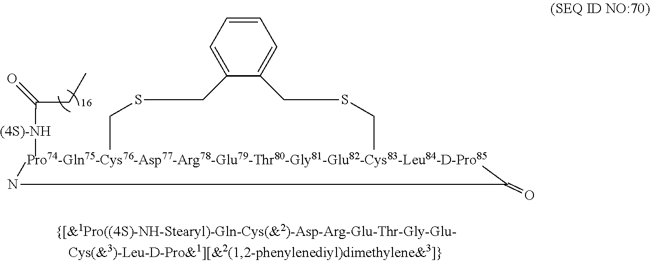

This 12 mer bicyclic peptide analog conjugated to the stearic fatty acid was synthesized according to the general scheme 2. The linear sequence was obtained following the procedure described in section 1b for those C-terminal acid peptide sequences. This analog contained an arginine in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Arg-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed following the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method J to afford a white solid with a purity of 95.1%.

This pure peptide was analyzed by analytical RP-HPLC with column 1 (gradient 70-100; t_{R}=5.3 min) and RP-HPLC-ESI-MS (calculated: 1711.8 (M); observed: 1712.8 [M+H]⁺, 857.2 [M+2H]⁺/2 and 572.0 [M+3H]⁺/3).

### Example 71

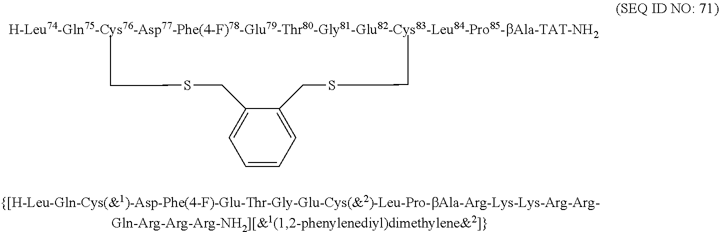

This 12 mer peptide analog conjugated to the CPP TAT was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.30 mmol/g by decreasing the equivalents of the first Aa (Arg) coupled to the resin. The incorporation of the CPP TAT was carried out on solid phase and stepwise until complete its nine amino acid sequence. This analog contained a 4-fluoro-phenylalanine in position 78 instead of a glutamic acid. The two cysteines were introduced as a Fmoc-L-Cys(Mmt)-OH and the Mmt protecting group was removed after the peptide elongation (before the cyclization) following the methodology detailed in section 3h. The cyclization on solid phase through incorporation of 1,2-bis(bromomethyl)benzene linker between the cysteines was done by the Method H. The cleavage from the resin with global deprotection was carried out after the cyclization as described in section 3h. Finally, the peptide crude was purified with equipment B and column 3 according to the Method H to afford a white solid as a trifluoroacetate salt with a purity of 90.2%.

This pure peptide was analyzed by analytical RP-HPLC with column 2 (gradient 10-25; t_{R}=12.7 min) and RP-HPLC-ESI-MS (calculated: 2864.5 (M); observed: 1433.6 [M+2H]⁺/2, 956.2 [M+3H]⁺/3, 717.4 [M+4H]⁺/4, 574.2 [M+5H]⁺/5).

### Example 72

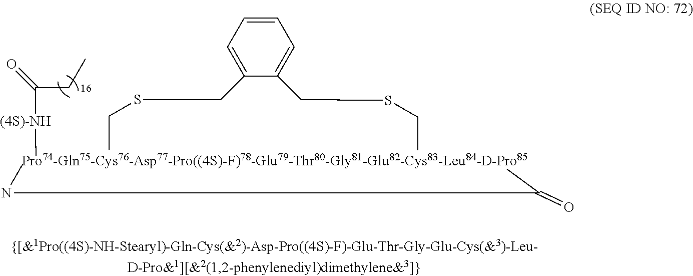

This 12 mer bicyclic peptide analog conjugated to the stearic fatty acid was synthesized according to the general scheme 2. The linear sequence was obtained following the procedure described in section 1b for those C-terminal acid peptide sequences. This analog contained a (4S)-fluoro-proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro((4S)F)-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed following the protocol described in section 4. The cyclization 2 (see scheme 2), through the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 91.7%.

This pure peptide was analyzed by analytical RP-HPLC with column 1 (gradient 70-100; t_{R}=5.2 min) and RP-HPLC-ESI-MS (calculated: 1670.8 (M); observed: 1694.1 [M+Na]⁺, 1671.2 [M+H]⁺ and 836.6 [M+2H]⁺/2).

### Example 73

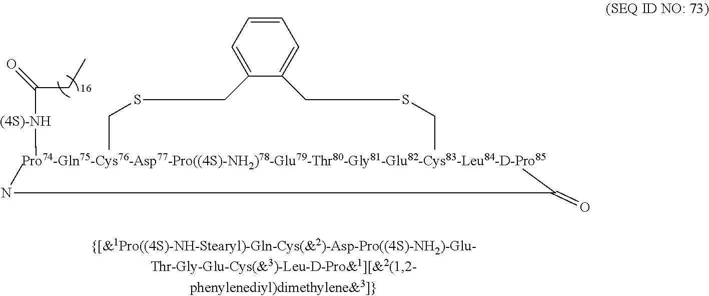

This 12 mer bicyclic peptide analog conjugated to the stearic fatty acid was synthesized according to the general scheme 2. The linear sequence was obtained according to the procedure described in section 1b for those C-terminal acid peptide sequences. This analog contained a (4S)-aminoproline in position 78 instead of a glutamic acid, a D-proline in position 85 instead of a L-proline and a (4S)-aminoproline in position 74 instead of a leucine. The (4S)-aminoproline in the position 78 was introduced as Fmoc-L-Pro(4-(S)—NHBoc)-OH. The (4S)-aminoproline 74 was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline 74 after the Alloc removal and following the protocol detailed in section 1 a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro((4S)—NHBoc)-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 98.0%.

This pure peptide was analyzed by analytical RP-HPLC with column 1 (gradient 70-100; t_{R}=3.7 min) and RP-HPLC-ESI-MS (calculated: 1667.8 (M); observed: 1668.8 [M+H]⁺).

### Example 74

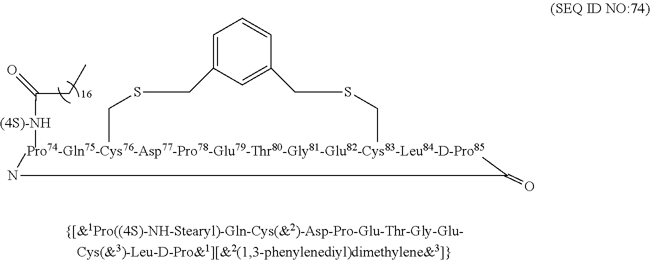

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed following the protocol described in section 4. The cyclization 2 (see scheme 2), to form the incorporation of 1,3-bis(bromomethyl)benzene linker between the cysteines, was done by the Method G, cyclization 2. Finally, the peptide crude was purified with equipment B and column 2 according to the Method I to afford a white solid with a purity of 97.6%.

This pure peptide was analyzed by analytical RP-HPLC with column 1 (gradient 70-100; t_{R}=5.3 min) and RP-HPLC-ESI-MS (calculated: 1652.8 (M); observed: 1654.6 [M+H]⁺ and 827.6 [M+2H]⁺/2).

### Example 75

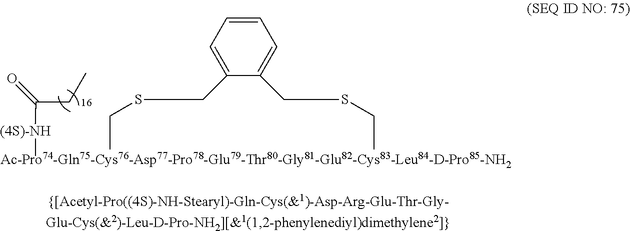

This 12 mer monocyclic peptide analog conjugated to the the stearic fatty acid was synthesized according to the general scheme 3 to perform the cyclization step on solid phase. The linear sequence was obtained following the procedure described in section 1a for those C-terminal amide peptide sequences and using the H-Rink amide AM ChemMatrix resin (0.47 mmol/g). In order to favor the cyclization on solid phase, the loading of the resin was decreased until 0.40 mmol/g by decreasing the equivalents of the first Aa (D-Pro) coupled to the resin. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The two cysteines were introduced as a Fmoc-L-Cys(Mmt)-OH and the Mmt protecting group was removed after the peptide elongation (before the cyclization) following the methodology detailed in section 3h. The N-terminus was acetylated following the protocol detailed in section 1a. The incorporation of the stearic acid was carried out on solid phase at the side chain of (4S)-aminoproline after the Alloc removal following the protocol detailed in section 1a. The cyclization on solid phase through incorporation of 1,2-bis(bromomethyl)benzene linker between the cysteines was done by the Method H. The cleavage from the resin with global deprotection was carried out after the cyclization as described in section 3h. Finally, the peptide crude was purified with equipment B and column 2 according to the Method I to afford a white solid with a purity of 98.3%.

This pure peptide was analyzed by analytical RP-HPLC with column 1 (gradient 60-100; t_{R}=5.2 min) and RP-HPLC-ESI-MS (calculated: 1711.8 (M); observed: 1713.9 [M+H]⁺ and 857.1 [M+2H]⁺/2).

### Example 76

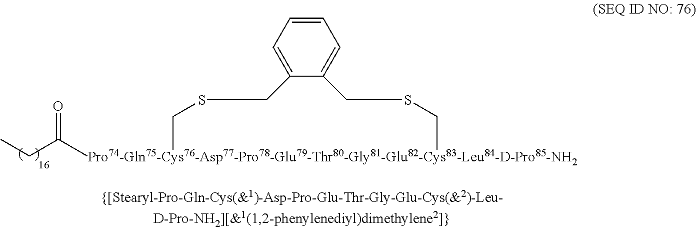

This 12 mer monocyclic peptide analog conjugated to the the stearic fatty acid was synthesized according to the general scheme 3 to perform the cyclization step on solid phase. The linear sequence was obtained according to the procedure described in section 1a for those C-terminal amide peptide sequences and using the H-Rink amide AM ChemMatrix resin (0.47 mmol/g). In order to favor the cyclization on solid phase, the loading of the resin was decreased until 0.40 mmol/g by decreasing the equivalents of the first Aa (D-Pro) coupled to the resin. This analog contained a proline in position 78 instead of a glutamic acid, a D-proline in position 85 instead of a L-proline and a proline in position 74 instead of a leucine. The two cysteines were introduced as a Fmoc-L-Cys(Mmt)-OH and the Mmt protecting group was removed after the peptide elongation (before the cyclization) according to the methodology detailed in section 3h. The incorporation of the stearic acid was carried out on solid phase at the N-terminal position following the protocol detailed in section 1a. The cyclization on solid phase through incorporation of 1,2-bis(bromomethyl)benzene linker between the cysteines was done by the Method H. The cleavage from the resin with global deprotection was carried out after the cyclization as described in section 3h. Finally, the peptide crude was purified with equipment B and column 2 according to the Method I to afford a white solid with a purity of 94.5%.

This pure peptide was analyzed by analytical RP-HPLC with column 1 (gradient 60-100; t_{R}=5.2 min) and RP-HPLC-ESI-MS (calculated: 1654.8 (M); observed: 1655.6 [M+H]⁺ and 828.7 [M+2H]⁺/2).

### Example 77

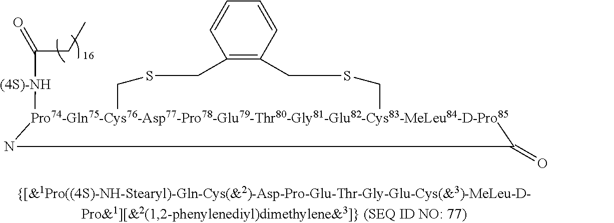

This 12 mer bicyclic peptide analog conjugated to the stearic fatty acid was synthesized according to the general scheme 2. The linear sequence was obtained according to the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine and an N-methyl-leucine in position 84 instead of a leucine. The (4S)-aminoproline was introduced as a Fmoc-L-Pro(4-(S)—NHAlloc)-OH and the Alloc protecting group was removed according to the methodology detailed in section 1a. The incorporation of the stearic acid was carried out on solid phase at the side chain of (4S)-aminoproline after the Alloc removal following the protocol detailed in section 1a. The cleavage from the resin without global deprotection was carried out as described in section 2a to afford the linear sequence H-Glu(OtBu)-Cys(Trt)-MeLeu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 92.2%.

This pure peptide was analyzed by analytical RP-HPLC with column 1 (gradient 70-100; t_{R}=1.2 min) and RP-HPLC-ESI-MS (calculated: 1666.8 (M); observed: 1689.6 [M+Na], and 1667.0 [M+H]⁺).

### Example 78

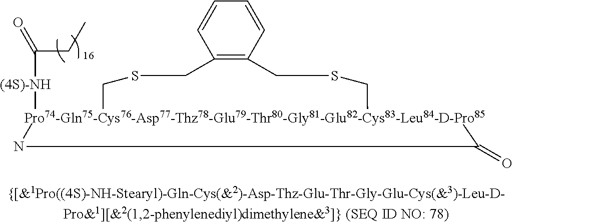

This 12 mer bicyclic peptide analog conjugated to the stearic fatty acid was synthesized according to the general scheme 2. The linear sequence was obtained following the procedure described in section 1b for those C-terminal acid peptide sequences. This analog contained a L-thioproline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the stearic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Thz-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,2-bis(bromomethyl)benzene linker between the cysteines, was done by the Method B. Finally, the peptide crude was purified with equipment B and column 2 according to the Method I to afford a white solid with a purity of 93.5%.

This pure peptide was analyzed by analytical RP-HPLC with column 3 (gradient 70-100; t_{R}=4.9 min) and RP-HPLC-ESI-MS (calculated: 1670.8 (M); observed: 1671.9 [M+H]+ and 836.6 [M+2H]+/2).

### Example 79

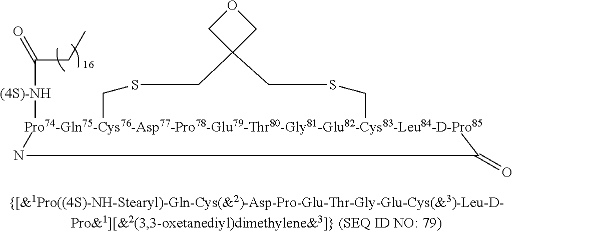

This 12 mer bicyclic peptide analog conjugated to the stearic fatty acid was synthesized according to the general scheme 2. The linear sequence was obtained following the procedure described in section 1b for those C-terminal acid peptide sequences. This analog contained a L-proline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the stearic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 3,3-bis(bromomethyl)oxetane linker between the cysteines, was done by the cyclization Method I (section 3i). Finally, the peptide crude was purified with equipment B and column 2 according to the Method I to afford a white solid with a purity of 93.4%. This pure peptide was analyzed by analytical RP-HPLC with column 3 (gradient 70-100; t_{R}=4.7 min) and RP-HPLC-ESI-MS (calculated: 1634.0 (M); observed: 1634.3 [M+H]+ and 817.7 [M+2H]+/2).

### Example 80

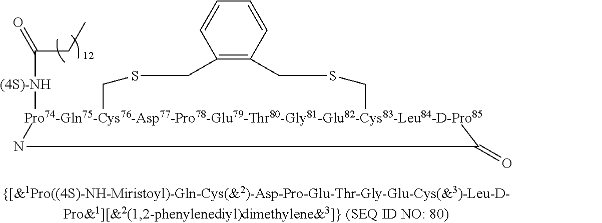

This 12 mer bicyclic peptide analog conjugated to the myristic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro(4-(S)—NHAlloc)-OH and the Alloc protecting group was removed following the methodology detailed in section 1a. The incorporation of the myristic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-myristyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,2-bis(bromomethyl)benzene linker between the cysteines, was done by the Method B. Finally, the peptide crude was purified with equipment B and column 2 according to the Method I to afford a white solid with a purity of 99.3%.

This pure peptide was analyzed by analytical RP-HPLC with column 3 (gradient 50-100; t_{R}=5.1 min) and RP-UPLC-ESI-MS (calculated: 1596.8 (M); observed: 1598.5 [M+H]+ and 799.8 [M+2H]+/2).

### Example 81

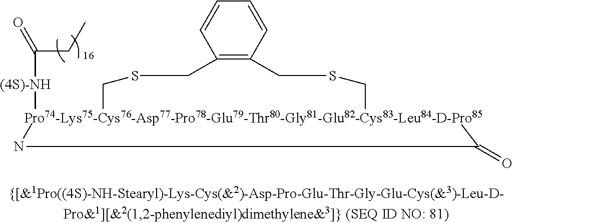

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline, a lysine in position 75 instead of a glutamine and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1 a. The cleavage from the resin without global deprotection was carried out as described in section 2a to afford the linear sequence H-Glu(OtBu)-Cys(Trt)-Leu-D-Pro-Pro((4S)—NH-Stearyl)-Lys(Boc)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid as a formate salt with a purity of 97.6%.

This pure peptide was analyzed by analytical RP-HPLC with column 3 (gradient 60-90; t_{R}=3.6 min) and RP-HPLC-ESI-MS (calculated: 1652.9 (M); observed: 1654.0 [M+H]+ and 827.8 [M+2H]2+/2).

### Example 82

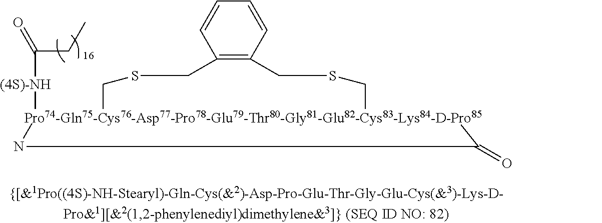

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline, a lysine in position 84 instead of a leucine and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1 a. The cleavage from the resin without global deprotection was carried out as described in section 2a to afford the linear sequence H-Glu(OtBu)-Cys(Trt)-Lys(Boc)-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid as a formate salt with a purity of 94.8%.

This pure peptide was analyzed by analytical RP-HPLC with column 3 (gradient 60-90; t_{R}=3.1 min) and RP-HPLC-ESI-MS (calculated: 1667.8 (M); observed: 1669.0 [M+H]+ and 835.1 [M+2H]2+/2).

### Example 83

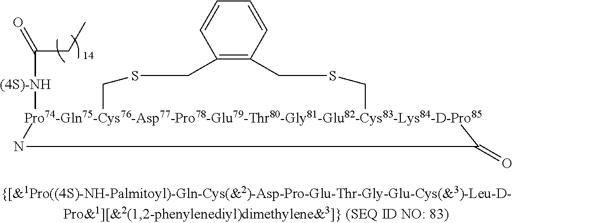

This 12 mer bicyclic peptide analog conjugated to the Palmitic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro(4-(S)—NHAlloc)-OH and the Alloc protecting group was removed following the methodology detailed in section 1a. The incorporation of the Palmitic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-palmitoyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,2-bis(bromomethyl)benzene linker between the cysteines, was done by the Method B. Finally, the peptide crude was purified with equipment B and column 2 according to the Method I to afford a white solid with a purity of 98.6%.

This pure peptide was analyzed by analytical RP-HPLC with column 3 (gradient 50-100; t_{R}=6.4 min) and RP-UPLC-ESI-MS (calculated: 1624.8 (M); observed: 1625.8 [M+H]+ and 813.7 [M+2H]+/2).

### Example 84

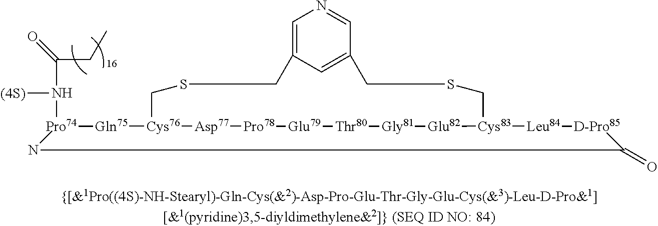

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1.

The global deprotection was performed according to the protocol described in section 4. The cyclization 2 (see scheme 2), to incorporate the 3,5-bis(chloromethyl)pyridine linker between the cysteines, was done by the Method G, cyclization 2. Finally, the peptide crude was purified with equipment B and column 2 according to the Method K to afford a white solid as a formate salt with a purity of 93.6%.

This pure peptide was analyzed by analytical RP-UPLC with column 1 (gradient 50-100; t_{R}=1.22 min) and RP-UPLC-ESI-MS (calculated: 1653.8 (M); observed: 1654.8 [M+H]+ and 828.2 [M+2H]2+/2).

### Example 85

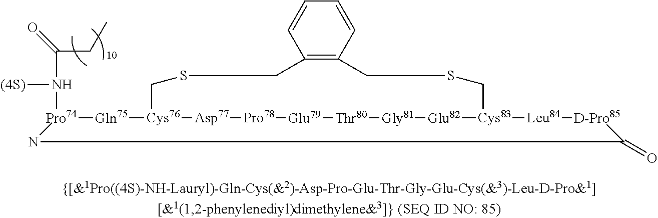

This 12 mer bicyclic peptide analog conjugated to the Lauric fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro(4-(S)—NHAlloc)-OH and the Alloc protecting group was removed following the methodology detailed in section 1a. The incorporation of the Lauric acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-lauryl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,2-bis(bromomethyl)benzene linker between the cysteines, was done by the Method B. Finally, the peptide crude was purified with equipment B and column 2 according to the Method I to afford a white solid with a purity of 99.6%.

This pure peptide was analyzed by analytical RP-HPLC with column 1 (gradient 40-70; t_{R}=6.5 min) and RP-UPLC-ESI-MS (calculated: 1568.7 (M); observed: 1569.7 [M+H]+ and 785.6 [M+2H]+/2).

### Example 86

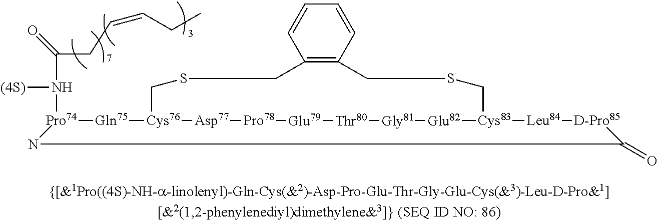

This 12 mer bicyclic peptide analog conjugated to the α-linolenic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the α-linolenic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-α-linolenyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4 with the corresponding modification for unsatured fatty acids. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method K to afford a white solid with a purity of 94.0%.

This pure peptide was analyzed by analytical RP-HPLC with column 3 (gradient 50-80; t_{R}=5.5 min) and RP-UPLC-ESI-MS (calculated: 1646.8 (M); observed: 1647.9 [M+H]+ and 824.5 [M+2H]2+/2).

### Example 87

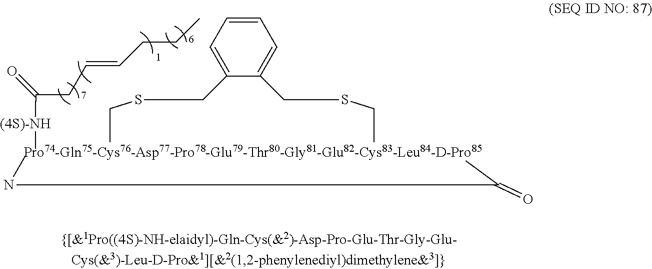

This 12 mer bicyclic peptide analog conjugated to the elaidic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the elaidic acid was carried out on solid phase at the side chain of (4S)-aminoproline after the Alloc removal and following the protocol detailed in section 1 a. The cleavage from the resin without global deprotection was carried out as described in section 2a to afford the linear sequence H-Glu(OtBu)-Cys(Trt)-Leu-D-Pro-Pro((4S)—NH-elaidyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4 with the corresponding modification for unsatured fatty acids. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method K to afford a white solid with a purity of 95.6%.

This pure peptide was analyzed by analytical RP-HPLC with column 3 (gradient 65-85; t_{R}=4.8 min) and RP-UPLC-ESI-MS (calculated: 1650.8 (M); observed: 1651.9 [M+H]+ and 826.5 [M+2H]2+/2).

### Example 88

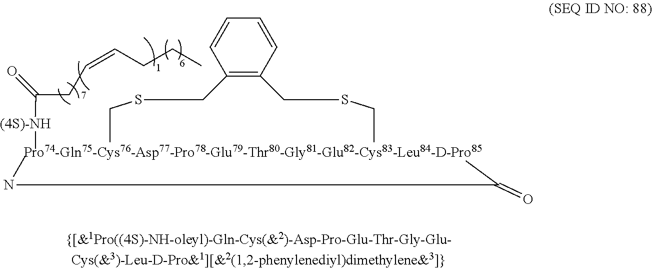

This 12 mer bicyclic peptide analog conjugated to the oleic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the oleic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-oleyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4 with the corresponding modification for unsatured fatty acids. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method K to afford a white solid with a purity of 96.1%.

This pure peptide was analyzed by analytical RP-HPLC with column 3 (gradient 70-100; t_{R}=4.7 min) and RP-UPLC-ESI-MS (calculated: 1650.8 (M); observed: 1651.4 [M+H]+ and 826.6 [M+2H]2+/2).

### Example 89

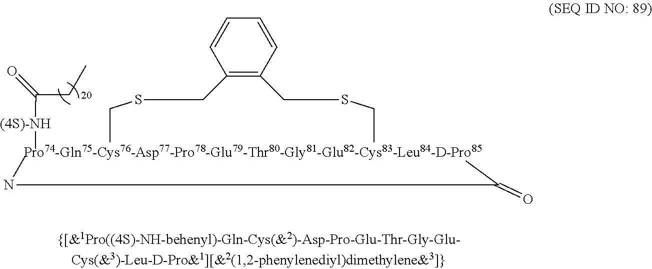

This 12 mer bicyclic peptide analog conjugated to the behenic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the behenic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-behenyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 4 according to the Method K to afford a white solid with a purity of 95.1%.

This pure peptide was analyzed by analytical RP-HPLC with column 4 (gradient 45-60; t_{R}=8.9 min) and RP-UPLC-ESI-MS (calculated: 1708.9 (M); observed: 855.4 [M+2H]2+/2).

### Example 90

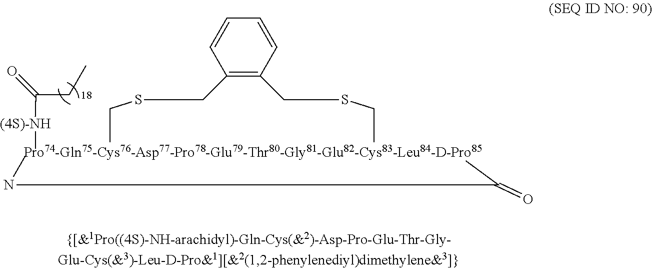

This 12 mer bicyclic peptide analog conjugated to the arachidic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the arachidic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-arachidyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 4 according to the Method K to afford a white solid with a purity of 95.1%.

This pure peptide was analyzed by analytical RP-HPLC with column 4 (gradient 45-55; t_{R}=7.9 min) and RP-UPLC-ESI-MS (calculated: 1680.9 (M); observed: 841.6 [M+2H]2+/2).

### Example 91

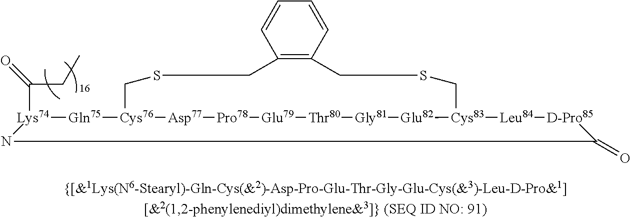

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline, and a lysine 74 instead of a leucine. The stearic fatty acid was appended from the lateral chain of the lysine in position 74, while the peptide was elongated through its Nα atom. Alloc removal from the lateral chain of lysine in position 74 was performed following the protocol detailed in section 1a. The cleavage from the resin without global deprotection was carried out as described in section 2a to afford the linear sequence H-Glu(OtBu)-Cys(Trt)-Leu-D-Pro-Lys(NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 97.0%.

This pure peptide was analyzed by analytical RP-HPLC with column 3 (gradient 70-100; t_{R}=6.0 min) and RP-UPLC-ESI-MS (calculated: 1668.9 (M); observed: 835.7 [M+2H]2+/2 and 846.6 [M+H+Na]2+/2).

### Example 92

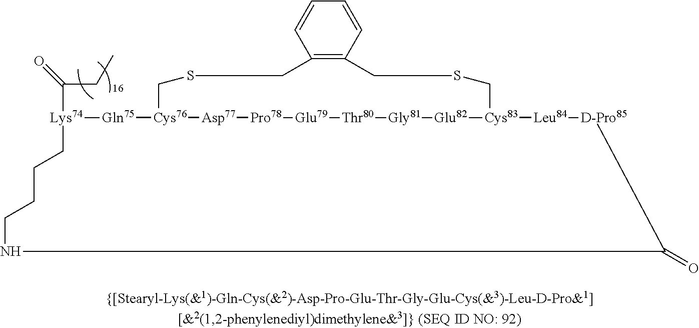

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline, and a lysine in position 74 instead of a leucine. The peptide was elongated through the lateral chain of the lysine in position 74, while the stearic fatty acid was appended from its Nα atom. Alloc removal from the lateral chain of lysine in position 74 was performed following the protocol detailed in section 1 a. The cleavage from the resin without global deprotection was carried out as described in section 2a to afford the linear sequence H-Glu(OtBu)-Cys(Trt)-Leu-D-Pro-Lys[Gln(Trt)-Cys(Trt)-Asp(OtBu)-Pro-Glu(OtBu)-Thr(tBu)-Gly-OH]-Stearyl)-. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 2 according to the Method I to afford a white solid with a purity of 98.3%.

This pure peptide was analyzed by analytical RP-HPLC with column 3 (gradient 70-100; t_{R}=6.0 min) and RP-UPLC-ESI-MS (calculated: 1668.9 (M); observed: 846.6 [M+H+Na]2+/2).

### Example 93

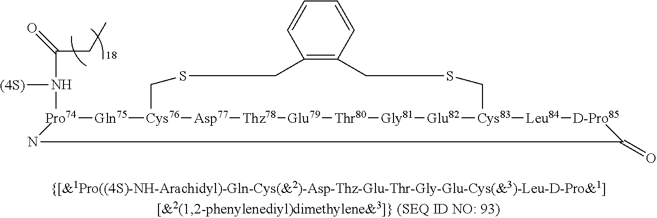

This 12 mer bicyclic peptide analog conjugated to the arachidyl fatty acid was synthesized according to the general scheme 2. The linear sequence was obtained following the procedure described in section 1b for those C-terminal acid peptide sequences. This analog contained a L-thioproline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the arachidic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Arachidyl)-Gln(Trt)-Cys(Trt)-Asp(OtBu)-Thz-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,2-bis(bromomethyl)benzene linker between the cysteines, was done by the Method B. Finally, the peptide crude was purified with equipment B and column 4 according to the Method I to afford a white solid with a purity of 95.1%.

This pure peptide was analyzed by analytical RP-HPLC with column 1 (gradient 80-100; t_{R}=5.9 min) and RP-HPLC-ESI-MS (calculated: 1698.8 (M); observed: 1699.5 [M+H]+ and 850.3 [M+2H]+/2).

### Example 94

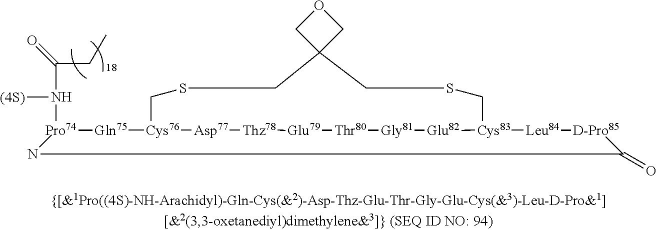

This 12 mer bicyclic peptide analog conjugated to the arachidyl fatty acid was synthesized according to the general scheme 2. The linear sequence was obtained following the procedure described in section 1b for those C-terminal acid peptide sequences. This analog contained a L-thioproline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the arachidic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Arachidyl)-Gln(Trt)-Cys(Trt)-Asp(OtBu)-Thz-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 3,3-bis(bromomethyl)oxetane linker between the cysteines, was done by the Method I. Finally, the peptide crude was purified with equipment B and column 4 according to the Method I to afford a white solid with a purity of 99.1%.

This pure peptide was analyzed by analytical RP-HPLC with column 1 (gradient 70-100; t_{R}=7.1 min) and RP-HPLC-ESI-MS (calculated: 1678.8 (M); observed: 1681.7 [M+H]+ and 840.6 [M+2H]+/2).

### Example 95

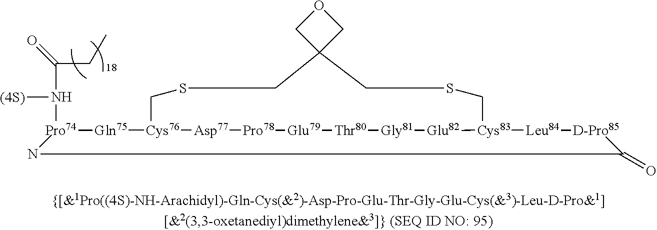

This 12 mer bicyclic peptide analog conjugated to the arachidic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the arachidic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-arachidyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 3,3-bis(bromomethyl)oxetane linker between the cysteines, was done by the Method G, cyclization 2. Finally, the peptide crude was purified with equipment B and column 4 according to the Method I to afford a white solid with a purity of 99.3%

This pure peptide was analyzed by analytical RP-UPLC with column 1 (gradient 70-100; t_{R}=0.76 min) and RP-UPLC-ESI-MS (calculated: 1662.0 (M); observed: 831.7 [M+2H]2+/2).

### Example 96

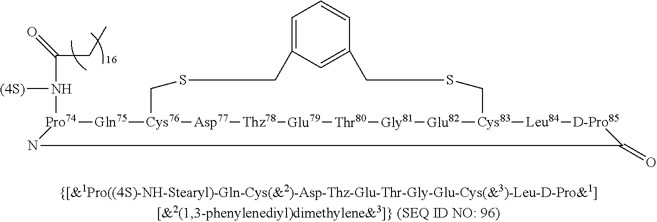

This 12 mer bicyclic peptide analog conjugated to the stearic fatty acid was synthesized according to the general scheme 2. The linear sequence was obtained following the procedure described in section 1b for those C-terminal acid peptide sequences. This analog contained a L-thioproline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the stearic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Thz-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 1,3-bis(bromomethyl)benzene linker between the cysteines, was done by the Method B. Finally, the peptide crude was purified with equipment B and column 2 according to the Method I to afford a white solid with a purity of 96.4%.

This pure peptide was analyzed by analytical RP-HPLC with column 1 (gradient 70-100; t_{R}=6.1 min) and RP-HPLC-ESI-MS (calculated: 1670.8 (M); observed: 1671.8 [M+H]+ and 836.4 [M+2H]+/2).

### Example 97

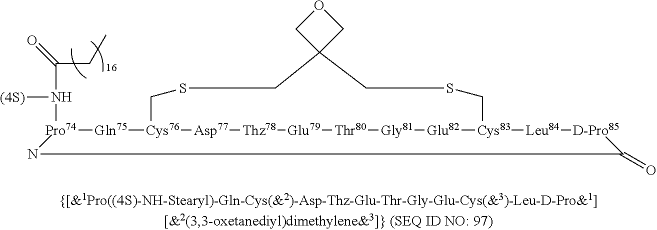

This 12 mer bicyclic peptide analog conjugated to the stearic fatty acid was synthesized according to the general scheme 2. The linear sequence was obtained following the procedure described in section 1b for those C-terminal acid peptide sequences. This analog contained a L-thioproline in position 78 instead of a glutamic acid, a D-proline in position 85 instead of a L-proline and a (4S)-amino-L-proline in position 74 instead of a leucine. The (4S)-amino-L-proline was introduced as a Fmoc-L-Pro((4S)—NHAlloc)-OH and the Alloc protecting group was removed following the methodology detailed in section 1a. The incorporation of the stearic acid was carried out on solid phase at the side chain of (4S)-amino-L-proline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Thz-Glu(OtBu)-Thr(tBu)-Gly-OH. The cyclization 1 (see scheme 2), to form the amide bond between the C-terminal and the N-terminal, was done by the Method G₂, cyclization 1. The global deprotection was performed according to the protocol described in section 4. The cyclization 2 (see scheme 2), to form the incorporation of 3,3-bis(bromomethyl)oxtane linker between the cysteines, was done by the Method I. Finally, the peptide crude was purified with equipment B and column 2 according to the Method I to afford a white solid with a purity of 98.8%.

This pure peptide was analyzed by analytical RP-HPLC with column 1 (gradient 70-100; t_{R}=5.2 min) and RP-HPLC-ESI-MS (calculated: 1650.8 (M); observed: 16251.5 [M+H]+ and 826.5 [M+2H]+/2).

### Example 98

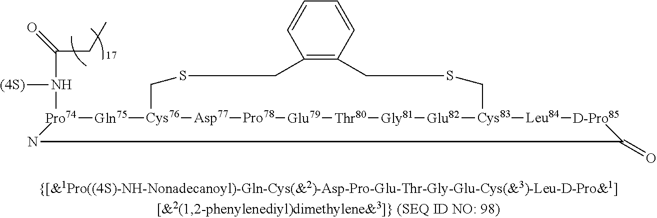

This 12 mer bicyclic peptide analog conjugated to the nonadecanoic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the nonadecanoic acid was carried out on solid phase at the side chain of (4S)-aminoproline after the Alloc removal and following the protocol detailed in section 1a. The cleavage from the resin without global deprotection was carried out as described in section 2a to afford the linear sequence H-Glu(OtBu)-Cys(Trt)-Leu-D-Pro-Pro((4S)—NH-nonadecanoyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of 1,2-bis(bromomethyl)benzene linker between the cysteines, was done by the Method G, cyclization 2. Finally, the peptide crude was purified with equipment B and column 4 according to the Method I to afford a white solid with a purity of 94.9%.

This pure peptide was analyzed by analytical RP-HPLC with column 1 (gradient 70-100; t_{R}=6.9 min) and RP-UPLC-ESI-MS (calculated: 1666.8 (M); observed: 834.5[M+2H]2+/2).

### Example 99

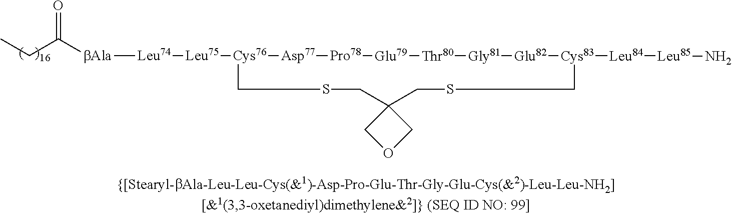

This 12 mer peptide analog conjugated to the stearic fatty acid was synthesized according to the general scheme 1. The linear sequence was obtained following the procedure described in section 1a for those C-terminal amide peptide sequences and using the Fmoc-Rink amide AM PS resin. The incorporation of the stearic acid was carried out on solid phase at the N-terminal position following the protocol detailed in section 1a. This analog contained a proline in position 78 instead of a glutamic acid and two leucines in position 75 and 85 instead of a glutamine and a proline, respectively. The cleavage from the resin with global deprotection was carried out as described in section 2b. The cyclization through incorporation of 3,3-bis(bromomethyl)oxetane linker between the cysteines was done by the Method B. Finally, the peptide crude was purified with equipment B and column 2 according to the Method K to afford a white solid with a purity of 95.1%.

This pure peptide was analyzed by analytical RP-UPLC with column 1 (gradient 70-100; t_{R}=0.82 min) and RP-UPLC-ESI-MS (calculated: 1723.0 (M); observed: 1746.4 [M+Na]+ and 1762.4 [M+K]+).

### Example 100

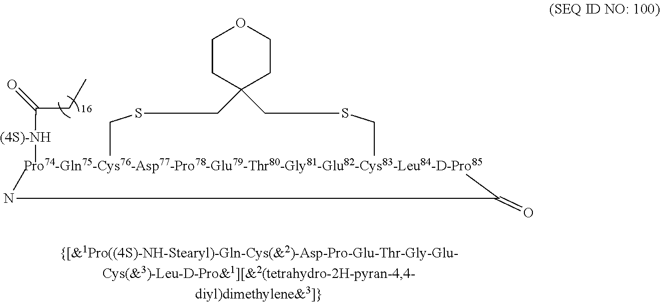

This 12 mer bicyclic peptide analog conjugated to the stearic fatty acid was synthesized according to the general scheme 2. The linear sequence was obtained following the procedure described in section 1b for those C-terminal acid peptide sequences. This analog contained a proline in position 78 instead of a glutamic acid, a D-proline in position 85 instead of a L-proline and a (4S)-aminoproline in position 74 instead of a leucine. The (4S)-aminoproline was introduced as a Fmoc-L-Pro(4-(S)—NHAlloc)-OH and the Alloc protecting group was removed following the methodology detailed in section 1a. The incorporation of the stearic acid was carried out on solid phase at the side chain of (4S)-aminoproline after the Alloc removal and following the protocol detailed in section 1 a. The cleavage from the resin without global deprotection was carried out as described in section 2a to afford the linear sequence H-Glu(OtBu)-Cys(Trt)-Leu-D-Pro-Pro((4S)—NH-Stearyl)-Gln(Trt)-Cys(Trt)-Asp(OtBu)-Pro-Glu(OtBu)-Thr(tBu)-Gly-OH. The cyclization 1 (see scheme 2), to form the amide bond between the C-terminal and the N-terminal, was done by the Method G, cyclization 1. The global deprotection was performed according to the protocol described in section 4. The cyclization 2 (see scheme 2), to form the incorporation of tetrahydro-2H-pyran-4,4-diyl)bis(methylene) linker between the cysteines, was done by the Method G, cyclization 2. Finally, the peptide crude was purified with equipment B and column 2 according to the Method I to afford a white solid as a formate salt with a purity of 100.0%.

This pure peptide was analyzed by analytical RP-HPLC with column 1 (gradient 70-100; t_{R}=4.48 min) and RP-HPLC-ESI-MS (calculated: 1662.04 (M); observed: 1662.7 [M+H]+ 1; 1680.1 [M+H₂O]+ land 831.6 [M+2H/2]+1).

Nrf2-Keap1 HTRF Binding Assay

A homogenous time-resolved FRET (TR-FRET) assay was used for identifying compounds that disrupt binding of ETGE peptide (QLQLDEETGEFL, Nrf2 high affinity domain) to Keap1-Maltose Binding Protein (MBP tag).

In a 384 well plate (ref.4513 Corning), 2 μl of test compound diluted in assay buffer (50 mM phosphate buffer pH 7, 0.1% BSA) were mixed with 4 μl of the Keap1-MBP (2.5 nM). The final DMSO concentration was 1%. After 10 minutes of compound preincubation, 4 μl of 10 nM ETGE-biotin was added to each well. After 30 minutes of incubation, 50 ng of streptavidin-d2 (donor fluorophore, ref.610SADLA, Cisbio) and 20 ng of anti-MBP-Eu³⁺ (acceptor fluorophore, ref.61MBPKAB, Cisbio) diluted with assay buffer+0.2M KF were added as detection reagents. After 2h 30 min, the fluorescence was measured on Envision (emission at 665 nm and excitation at 620 nm) in Envision instrument. When the Keap1-MBP and the ETGE peptide were bound, the energy transfer between the donor and acceptor fluorophores is measured as fluorescence from the acceptor fluorophore. A decrease in fluorescence indicates that the compound competes with the labeled ETGE peptide for binding to Keap-MBP.

For calculation, the data was normalized against DMSO and the positive control (the compound 2,2′-(naphthalene-1,4-diylbis(((4-methoxyphenyl)sulfonyl)azanediyl))diacetic acid at 10 μM described in J. Med. Chem. 2014, 57, 2736-2745 was used as positive control).

The IC₅₀ values are presented below. The values have been banded into grades. Grade A represents a value of less than 0.001 μM. Grade B represents a value of less than 0.1 but more than or equal to 0.001 μM. Grade C represents a value of less than 5 but more than or equal to 0.1 μM.

TABLE 3
Binding (IC₅₀) ETGE-Keap1 of final synthesized peptides

| Example | Binding (IC₅₀) ETGE-Keap1 |
|---|---|
| 1 | B |
| 2 | C |
| 3 | B |
| 4 | A |
| 5 | B |
| 6 | B |
| 7 | B |
| 8 | B |
| 9 | B |
| 10 | C |
| 11 | B |
| 12 | B |
| 13 | B |
| 14 | A |
| 15 | B |
| 16 | A |
| 17 | B |
| 18 | B |
| 19 | B |
| 20 | B |
| 21 | B |
| 22 | B |
| 23 | B |
| 24 | B |
| 25 | B |
| 26 | B |
| 27 | B |
| 28 | B |
| 29 | B |
| 30 | B |
| 31 | B |
| 32 | B |
| 33 | B |
| 34 | B |
| 35 | B |
| 36 | B |
| 37 | B |
| 38 | B |
| 39 | B |
| 40 | B |
| 41 | B |
| 42 | A |
| 43 | B |
| 44 | B |
| 45 | B |
| 46 | B |
| 47 | A |
| 48 | B |
| 49 | B |
| 50 | A |
| 51 | B |
| 52 | B |
| 53 | B |
| 54 | B |
| 55 | B |
| 56 | A |
| 57 | A |
| 58 | A |
| 59 | A |
| 60 | A |
| 61 | B |
| 62 | B |
| 63 | B |
| 64 | A |
| 65 | A |
| 67 | B |
| 68 | B |
| 69 | B |
| 70 | A |
| 71 | B |
| 72 | B |
| 73 | B |
| 74 | A |
| 75 | B |
| 76 | B |
| 77 | B |
| 78 | A |
| 79 | B |
| 80 | A |
| 81 | B |
| 82 | B |
| 83 | A |
| 84 | B |
| 85 | A |
| 86 | A |
| 87 | B |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | B |

The data demonstrate that the peptidic compounds of the invention have a high binding affinity for Keap1.

1. A peptidic compound of Formula (I)′, or a pharmaceutically acceptable salt, or solvate, or N-oxide, or stereoisomer thereof: 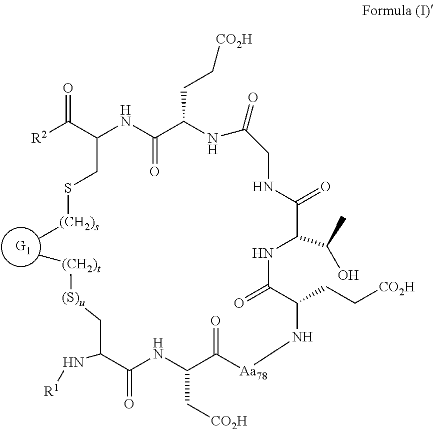
wherein:
R¹ is chosen from a hydrogen atom, a —CO(C₁-C₄ alkyl) group, a —CONH(C₁-C₄ alkyl) group, and a -Aa₇₅-Aa₇₄-[L₁]ₘ-[Tag₁]ₙ group;
R² is chosen from a —OH group, a —NH₂ group, and a -Aa₈₄-Aa₈₅-[L₂]ₚ[Tag₂]_{q} group;
Aa₇₄ is chosen from a direct bond, a leucine, a valine, a lysine, an arginine, a phenylalanine, a proline, and a N-acetyl-proline residue, wherein when Aa₇₄ is other than a direct bond: (a) Aa₇₄ is optionally linked to Aa₈₅; and/or (b) Aa₇₄ is optionally alkylated with a C₁-C₄ alkyl group on the N at the peptidic bond, and wherein when Aa₇₄ is a proline or a N-acetyl-proline residue it is unsubstituted or substituted by a —NH₂ group or a —NHC(O)CH₃ group;
Aa₇₅ is chosen from a direct bond, a glutamine, a leucine, a lysine, a valine, a phenylalanine, and an arginine residue, wherein when Aa₇₅ is other than a direct bond Aa₇₅ is optionally alkylated with a C₁-C₄ alkyl group on the N at the peptidic bond;
m and n are independently represent an integer selected from 0 and 1, wherein when m and n are 0 and Aa₇₄ is not linked to Aa₈₅, the or each amino-terminal group of R¹ is a —NH₂ group or a —NHC(O)CH₃ group and provided that when m and n are 0, Aa₇₄ and Aa₇₅ cannot simultaneously be a direct bond;
when m is 1 and n is 1, L₁ is a —C(O)—(CH₂)₍₁₋₄₎—NH— group, and when m is 1 and n is 0, L₁ is a —C(O)—(CH₂)₍₁₋₄₎—NH₂ group;
Tag₁ is a —C(O)—(CH₂)ᵣ—CH₃ group or a —C(O)—(CH₂)₇—(CH═CH—CH₂)₁₋₃—(CH₂)₀₋₆—CH₃ group, wherein when Aa₇₄ represents a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetyl-proline residue;
r is an integer selected from 6 to 24;
Aa₈₄ is chosen from a direct bond, a leucine, a valine, a lysine, and an arginine residue, wherein, when Aa₈₄ is other than a direct bond, Aa₈₄ is optionally alkylated with a C₁-C₄ alkyl group on the N at the peptidic bond;
Aa₈₅ is chosen from a direct bond, a proline, a leucine, a valine, a lysine, an arginine, and a D-proline residue, wherein when Aa₈₅ is other than a direct bond: (a) Aa₈₅ is optionally linked to Aa₇₄; and/or (b) Aa₈₅ is optionally alkylated with a C₁-C₄ alkyl group on the N at the peptidic bond;
p and q each are independently an integer selected from 0 and 1, wherein, when p and q are 0 and Aa₇₄ is not linked to Aa₈₅, the or each carboxy-terminal group of R² is a —COOH group or a —CONH₂ group and provided that when p and q each are 0, Aa₈₄ and Aa₈₅ cannot simultaneously be a direct bond;
when p is 1 and q is 1, L₂ is a —NH—(CH₂)₍₁₋₄₎—CO— group, and when p is 1 and q is 0, L₂ is a —NH—(CH₂)₍₁₋₄₎—COOH or a —NH—(CH₂)₍₁₋₄₎—CONH₂ group;
Tag₂ is a peptide containing from 6 to 20 amino acids wherein at least three of these amino acids are selected from the group consisting of lysine and arginine, wherein the or each carboxy-terminal group of Tag₂ is a —COOH group or a —CONH₂ group;
s is 0 or 1;
t is 0 or 1;
u is 0 or 1;
Aa₇₈ is chosen from a proline, a L-thioproline, an alanine, a phenylalanine, an arginine, and a glutamic acid residue, wherein the proline, L-thioproline, alanine, phenylalanine, arginine or glutamic acid residue is optionally substituted by one, two or three substituents selected from a halogen atom and amino group and wherein Aa₇₈ is optionally alkylated with a C₁-C₄ alkyl group on the N at the peptidic bond; and
G₁ is a C₆₋₂₀ aryl group, wherein the C₆₋₂₀ aryl group is selected from a phenyl group, a naphthyl group, a biphenyl group, and a binaphthyl group; or a 6-10 membered heteroaryl group selected from a pyridine group, an indolyl group and a quinoxaline group; wherein the aryl and heteroaryl groups are optionally substituted by one or more substituents selected from a C₁-C₄ alkyl group and a halogen atom; or a 4-6 membered saturated heterocyclyl group containing one oxygen atom selected from an oxetanyl group, a tetrahydrofuranyl group, and a tetrahydro-2H-pyranyl group.

2. The peptidic compound according to claim 1, wherein the peptidic compound is a compound of Formula (I), or a pharmaceutically acceptable salt, or solvate, or N-oxide, or stereoisomer thereof: 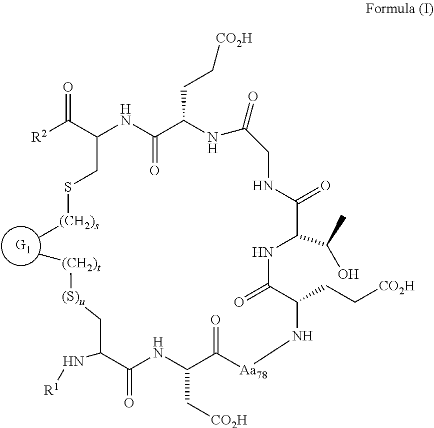
wherein:
R¹ is chosen from a hydrogen atom, a —CO(C₁-C₄ alkyl) group, a —CONH(C₁-C₄ alkyl) group, and a -Aa₇₅-Aa₇₄-[L₁]ₘ[Tag₁]ₙ group;
R² is chosen from a —OH group, a —NH₂ group, and a -Aa₈₄-Aa₈₅-[L₂]ₚ[Tag₂]_{q} group;
Aa₇₄ is chosen from a direct bond, a leucine, a valine, a lysine, an arginine, a phenylalanine, a proline, and a N-acetyl-proline residue, wherein when Aa₇₄ is other than a direct bond: (a) Aa₇₄ is optionally linked to Aa₈₅; and/or (b) Aa₇₄ is optionally alkylated with a C₁-C₄ alkyl group on the N at the peptidic bond, and wherein when Aa₇₄ is a proline or a N-acetyl-proline residue it is unsubstituted or substituted by a —NH₂ group or a —NHC(O)CH₃ group;
Aa₇₅ is chosen from a direct bond, a glutamine, a leucine, a lysine, a valine, a phenylalanine, and an arginine residue, wherein when Aa₇₅ is other than a direct bond Aa₇₅ is optionally alkylated with a C₁-C₄ alkyl group on the N at the peptidic bond;
m and n each are independently an integer selected from 0 and 1, wherein when m and n are 0 and Aa₇₄ is not linked to Aa₈₅, the or each amino-terminal group of R¹ is a —NH₂ group or a —NHC(O)CH₃ group and provided that when m and n are 0, Aa₇₄ and Aa₇₅ cannot simultaneously be a direct bond;
when m is 1 and n is 1, L₁ is a —C(O)—(CH₂)₍₁₋₄₎—NH— group, and when m is 1 and n is 0, L₁ is a —C(O)—(CH₂)₍₁₋₄₎—NH₂ group;
Tag₁ is a —C(O)—(CH₂)ᵣ—CH₃ group, wherein when Aa₇₄ is a 4-aminoproline or a 4-amino-N-acetyl-proline residue and m is 0, the Tag₁ group is linked to Aa₇₄ through the 4-amino substituent of the 4-aminoproline residue or through the 4-amino substituent of the 4-amino-N-acetyl-proline residue;
r is an integer selected from 6 to 18;
Aa₈₄ is chosen from a direct bond, a leucine, a valine, a lysine, and an arginine residue, wherein, when Aa₈₄ is other than a direct bond, Aa₈₄ is optionally alkylated with a C₁-C₄ alkyl group on the N at the peptidic bond;
Aa₈₅ is chosen from a direct bond, a proline, a leucine, a valine, a lysine, an arginine, and a D-proline residue, wherein when Aa₈₅ is other than a direct bond: (a) Aa₈₅ is optionally linked to Aa₇₄; and/or (b) Aa₈₅ is optionally alkylated with a C₁-C₄ alkyl group on the N at the peptidic bond;
p and q are independently an integer selected from 0 and 1, wherein, when p and q are 0 and Aa₇₄ is not linked to Aa₈₅, the or each carboxy-terminal group of R² is a —COOH group or a —CONH₂ group and provided that when p and q are 0, Aa₈₄ and Aa₈₅ cannot simultaneously be a direct bond;
when p is 1 and q is 1, L₂ is a —NH—(CH₂)₍₁₋₄₎—CO— group, and when p is 1 and q is 0, L₂ is a —NH—(CH₂)₍₁₋₄₎—COOH or a —NH—(CH₂)₍₁₋₄₎—CONH₂ group;
Tag₂ is a peptide containing from 6 to 20 amino acids wherein at least three of these amino acids are selected from the group consisting of lysine and arginine, wherein the or each carboxy-terminal group of Tag₂ is a —COOH group or a —CONH₂ group;
s is 0 or 1;
t is 0 or 1;
u is 0 or 1;
Aa₇₈ is chosen from a proline, an alanine, a phenylalanine, an arginine, and a glutamic acid residue, wherein the proline, alanine, phenylalanine, arginine or glutamic acid residue is optionally substituted by one, two or three substituents selected from a halogen atom and amino group and wherein Aa₇₈ is optionally alkylated with a C₁-C₄ alkyl group on the N at the peptidic bond; and
G₁ is a C₆₋₂₀ aryl group, wherein the C₆₋₂₀ aryl group is selected from a phenyl group, a naphthyl group, a biphenyl group and a binaphthyl group; or a 6-10 membered heteroaryl group selected from a pyridine group, an indolyl group and a quinoxaline group; wherein the aryl and heteroaryl groups are optionally substituted by one or more substituents selected from a C₁-C₄ alkyl group and a halogen atom.

3. The peptidic compound according to claim 1, wherein Aa₇₄ is chosen from a direct bond, a leucine, a valine, a lysine, a proline, a 4-aminoproline, and a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond.

4. The peptidic compound according to claim 1, wherein Aa₇₅ is chosen from a direct bond, a glutamine, a leucine, a lysine, and a valine residue.

5. The peptidic compound according to claim 1, wherein, when m is 1 and n is 1, L₁ is a —C(O)—(CH₂)₂-NH— group.

6. The peptidic compound according to claim 1, wherein Tag, is chosen from a —C(O)—(CH₂)ᵣ—CH₃ group, a —C(O)—(CH₂)₇-((E-CH═CH)—CH₂)₁—(CH₂)₈—CH₃ group, a —C(O)—(CH₂)₇—((Z—CH═CH)—CH₂)₁—(CH₂)₈—CH₃ group, and a —C(O)—(CH₂)₇—((Z—CH═CH)—CH₂)₃—CH₃ group, wherein when Aa₇₄ is a 4-aminoproline residue and m is 0, the Tag₁ group is linked to Aa₇₄ through the 4-amino substituent of the 4-aminoproline residue; and r is 6 to 20.

7. The peptidic compound according to claim 6, wherein Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue; and r is 6 to 20.

8. The peptidic compound according to claim 1, wherein in -Aa₇₅-Aa₇₄-[L₁]ₘ-[Tag₁]ₙ:
Aa₇₄ is chosen from a direct bond, a leucine, a valine, a lysine, a proline, a 4-aminoproline, and a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond (i.e. the leucine residue is optionally N methylated at the peptidic bond);
Aa₇₅ is chosen from a direct bond, a glutamine, a leucine, a lysine, and a valine residue;
(i) m is 0 and n is 0; or (ii) m is 0 and n is 1; or (iii) m is 1 and n is 1, wherein when m and n are 0, and Aa₇₄ is not linked to Aa₈₅, the or each amino-terminal group of R¹ is a —NH₂ group and provided that when m and n are 0, Aa₇₄ and Aa₇₅ cannot simultaneously be a direct bond;
L₁ is a —C(O)—(CH₂)₂—NH— group;
Tag₁ is chosen from a —C(O)—(CH₂)ᵣ—CH₃ group, a —C(O)—(CH₂)₇-((E-CH═CH)—CH₂)₁—(CH₂)₆—CH₃ group, a —C(O)—(CH₂)₇—((Z—CH═CH)—CH₂)₁—(CH₂)₆—CH₃ group, and a —C(O)—(CH₂)₇—((Z—CH═CH)—CH₂)₃—CH₃ group, wherein when Aa₇₄ is a 4-aminoproline residue and m is 0, the Tag₁ group is linked to Aa₇₄ through the 4-amino substituent of the 4-aminoproline residue; and
r is 6 to 20.

9. The peptidic compound according to claim 8, wherein in -Aa₇₅-Aa₇₄-[L₁]ₘ-[Tag₁]ₙ:
Aa₇₄ is chosen from a direct bond, a leucine, a valine, a lysine, a proline, a 4-aminoproline, and a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond;
Aa₇₅ is chosen from a direct bond, a glutamine, a leucine, a lysine, and a valine residue;
(i) m is 0 and n is 0; or (ii) m is 0 and n is 1; or (iii) m is 1 and n is 1, wherein when m and n are 0, and Aa₇₄ is not linked to Aa₈₅, the or each amino-terminal group of R¹ is a —NH₂ group and provided that when m and n are 0, Aa₇₄ and Aa₇₅ cannot simultaneously be a direct bond;
L₁ is a —C(O)—(CH₂)₂—NH— group; and
Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue; and
r is 6 to 20.

10. The peptidic compound according to claim 1, wherein Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue; and r is 6 or 16.

11. The peptidic compound according to claim 1, wherein in -Aa₇₅-Aa₇₄-[L₁]ₘ-[Tag₁]ₙ:
Aa₇₄ is chosen from a direct bond, a leucine, a valine, a lysine, a proline, a 4-aminoproline, and a 4-acetaminoproline residue, wherein (a) when Aa₇₄ is other than a direct bond it is optionally linked to Aa₈₅; and/or (b) when Aa₇₄ is leucine, the leucine residue is optionally alkylated with a methyl group on the N at the peptidic bond;
Aa₇₅ is chosen from a direct bond, a glutamine, a leucine, a lysine, and a valine residue;
(i) m is 0 and n is 0; or (ii) m is 0 and n is 1; or (iii) m is 1 and n is 1, wherein when m and n are 0, and Aa₇₄ is not linked to Aa₈₅, the or each amino-terminal group of R¹ is a —NH₂ group and provided that when m and n are 0, Aa₇₄ and Aa₇₅ cannot simultaneously be a direct bond;
L₁ is a —C(O)—(CH₂)₂—NH— group;
Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue; and
r is 6 or 16.

12. The peptidic compound according to claim 1, wherein R¹ is a —COCH₃ group or a -Aa₇₅-Aa₇₄-[L₁]ₘ-[Tag₁]ₙ group.

13. The peptidic compound according to claim 1, wherein Aa₈₄ is chosen from a direct bond, a leucine, a valine, and a lysine residue, wherein, when Aa₈₄ is a leucine residue, Aa₈₄ is optionally alkylated with a methyl group on the N at the peptidic bond.

14. The peptidic compound according to claim 1, wherein Aa₈₅ is chosen from a direct bond, a proline, a leucine, a valine, a lysine, and a D-proline residue, wherein when Aa₈₅ is other than a direct bond it is optionally linked to Aa₇₄.

15. The peptidic compound according to claim 1, wherein p is 1, and q is 1, and L₂ is a —NH—(CH₂)₂—CO— group.

16. The peptidic compound according to claim 1, wherein Tag₂ is a cell penetrating peptide containing from 8 to 11 amino acids, wherein at least three of these amino acids are selected from the group consisting of lysine and arginine and wherein the or each carboxy-terminal group of Tag₂ is a —CONH₂ group.

17. The peptidic compound according to claim 1, wherein in -Aa₈₄-Aa₈₅-[L₂]ₚ[Tag₂]_{q}:
Aa₈₄ is chosen from a direct bond, a leucine, a valine, and a lysine residue, wherein, when Aa₈₄ is a leucine residue, Aa₈₄ is optionally alkylated with a methyl group on the N at the peptidic bond;
Aa₈₅ is chosen from a direct bond, a proline, a leucine, a valine, a lysine, and a D-proline residue, wherein when Aa₈₅ is other than a direct bond it is optionally linked to Aa₇₄;
(i) p is 0 and q is 0; or (ii) p is 1 and q is 1, wherein, when p and q are 0 and Aa₇₄ is not linked to Aa₈₅, the or each carboxy-terminal group of R² is a —COOH group or a —CONH₂ group and provided that when p and q are 0, Aa₈₄ and Aa₈₅ cannot simultaneously be a direct bond;
L₂ is a —NH—(CH₂)₂—CO— group; and
Tag₂ is a peptide containing from 8 to 11 amino acids, wherein at least three of these amino acids are selected from the group consisting of lysine and arginine and the or each carboxy-terminal group of Tag₂ is a —CONH₂ group.

18. The peptidic compound according to claim 1, wherein R² is a —NH₂ group or a -Aa₈₄-Aa₈₅-[L₂]ₚ-[Tag₂]_{q} group.

19. The peptidic compound according to claim 1,
wherein:
R¹ is selected from:
COCH₃;
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₂₀—CH₃)—;
-Gln-Pro((4S)—NH—CO—(CH₂)₁₈—CH₃)—;
-Gln-Pro((4S)—NH—CO—(CH₂)₁₇—CH₃)—;
-Gln-Pro((4S)—NH—CO—(CH₂)₁₈—CH₃)—;
-Gln-Pro((4S)—NH—CO—(CH₂)₁₄—CH₃)—;
-Gln-Pro((4S)—NH—CO—(CH₂)₁₂—CH₃)—;
-Gln-Pro((4S)—NH—CO—(CH₂)₁₀—CH₃)—;
-Gln-Pro((4S)—NH—CO—(CH₂)₇-((E-CH═CH)—CH₂)₁—(CH₂)₆—CH₃)—;
-Gln-Pro((4S)—NH—CO—(CH₂)₇—((Z—CH═CH)—CH₂)₁—(CH₂)₆—CH₃)—;
-Gln-Pro((4S)—NH—CO—(CH₂)₇—((Z—CH═CH)—CH₂)₃—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-Lys(M-CO—(CH₂)₁₆—CH₃)—;
-Gln-Lys(-CO—(CH₂)₁₆—CH₃)—;
-Gln-AcPro((4S)—NH—CO—(CH₂)₁₆—CH₃)_{;}
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃;
-Lys-Pro((4S)—NH—CO—(CH₂)₁₆—CH₃)—; and
-Val-Val-CO—(CH₂)₂—NH—CO—(CH₂)₁₆—CH₃;
R² is selected from:
-NH₂;
-NH-(CH₂)2-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
-Lys-D-Pro-;
and
-Val-Val-NH₂;
either (i) s, t, and u are each 0; or (ii) s, t, and u are each 1;
Aa₇₈ is chosen from an unsubstituted alanine, arginine, glutamic acid or L-thioproline residue, or a proline or a phenylalanine residue optionally substituted by one substituent selected from a fluorine atom and amino group, and wherein Aa₇₈ is optionally alkylated with a methyl group on the N at the peptidic bond; and
G₁ is an unsubstituted 6-10 membered heteroaryl group, wherein the unsubstituted 6-10 membered heteroaryl group is selected from a pyridine group, an indolyl group, and a quinoxaline group; or a C₆₋₂₀ aryl group selected from a phenyl group, a naphthyl group, a biphenyl group and a binaphthyl group, which aryl group is optionally substituted by three or four substituents selected from a methyl group and a halogen atom; or a 4-6 membered saturated heterocyclyl group containing one oxygen atom selected from an oxetanyl group group and a tetrahydro-2H-pyranyl group.

20. The peptidic compound according to claim 1, wherein:
R¹ is selected from:
—COCH₃;
—CO—(CH₂)₂—NH—CO—(CH₂)₁₆—CH₃;
—CO—(CH₂)₂—NH—CO—(CH₂)₆—CH₃;
-Gln-Pro((4S)—NH—CO—(CH₂)₁₆—CH₃)—;
-Gln-Pro((4S)—NHC(O)CH₃)—;
-Gln-Leu-H;
-Gln-Leu-;
-Gln-Leu-CO—(CH₂)₂—NH—CO—(CH₂)₁₆—CH₃;
-Gln-Leu-CO—(CH₂)₂—NH—CO—(CH₂)₆—CH₃;
-Gln-MeLeu-CO—(CH₂)₂—NH—CO—(CH₂)₁₆—CH₃;
-Gln-Lys-CO—(CH₂)₂—NH—CO—(CH₂)₁₆—CH₃;
-Gln-Lys(-CO—(CH₂)₂—NH—CO—(CH₂)₁₆—CH₃)—;
-Gln-AcPro((4S)—NH—CO—(CH₂)₁₆—CH₃)_{;}
-Gln-Pro-CO—(CH₂)₁₆—CH₃;
-Leu-Leu-CO—(CH₂)₂—NH—CO—(CH₂)₁₆—CH₃;
-Leu-Leu-H;
-Lys-Lys-CO—(CH₂)₂—NH—CO—(CH₂)₁₆—CH₃; and
-Val-Val-CO—(CH₂)₂—NH—CO—(CH₂)₁₆—CH₃;
R² is selected from:
-NH₂;
-NH-(CH₂)₂-CO-Arg-Lys-Lys-Arg-Arg-Gln-Arg-Arg-Arg-
NH₂;
-Leu-D-Pro-;
-Leu-D-Pro-NH₂;
-Leu-Leu-NH-(CH₂)₂-CO-Arg-Lys-Lys-Arg-Arg-Gln-Arg-
Arg-Arg-NH₂;
-Leu-Leu-NH₂;
-Leu-Pro-OH;
-Leu-Pro-NH₂;
-Leu-Pro-;
-Leu-Pro-NH-(CH₂)₂-CO-Arg-Arg-Arg-Arg-Arg-Arg-Arg-
Arg-Arg-Arg-NH₂;
-Leu-Pro-NH-(CH₂)₂-CO-Arg-Arg-Arg-Arg-Arg-Arg-Arg-
Arg-NH₂;
-Leu-Pro-NH-(CH₂)₂-CO-Arg-Lys-Lys-Arg-Arg-Gln-Arg-
Arg-Arg-NH₂;
-Leu-Pro-NH-(CH₂)₂-CO-Tyr-Ala-Arg-Ala-Ala-Ala-Arg-
Gln-Ala-Arg-Ala-NH₂;
-MeLeu-D-Pro-;
-MeLeu-Pro-NH₂;
-Lys-Lys-NH₂;
and
-Val-Val-NH₂;
either (i) s, t, and u are each 0; or (ii) s, t, and u are each 1;
Aa₇₈ is chosen from an unsubstituted alanine, arginine or glutamic acid residue, or a proline or a phenylalanine residue optionally substituted by one substituent selected from a fluorine atom and amino group, and wherein Aa₇₈ is optionally alkylated with a methyl group on the N at the peptidic bond; and
G₁ is an unsubstituted 6-10 membered heteroaryl group, wherein the unsubstituted 6-10 membered heteroaryl group is selected from a pyridine group, an indolyl group, and a quinoxaline group; or a C₆₋₂₀ aryl group selected from a phenyl group, a naphthyl group, a biphenyl group, and a binaphthyl group, which aryl group is optionally substituted by three or four substituents selected from a methyl group and a halogen atom.

21. The peptidic compound according to claim 1, wherein the peptidic compound is a compound of Formula (IA)′, or a pharmaceutically acceptable salt, or solvate, or N-oxide, or stereoisomer thereof: 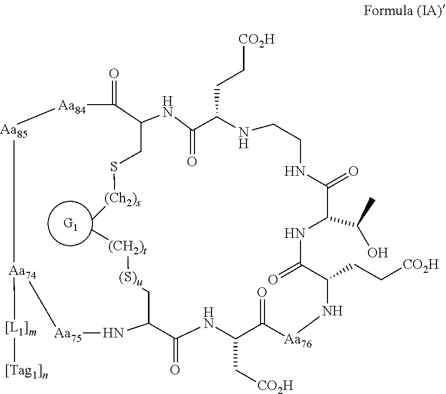
wherein:
Aa₇₄ is chosen from a leucine, a lysine, a 4-aminoproline, and a 4-acetaminoproline residue;
Aa₇₅ is a glutamine or a lysine residue;
m and n are independently an integer selected from 0 and 1;
when m is 1 and n is 1, L₁ is a —C(O)—(CH₂)₍₁₋₄₎—NH— group, and when m is 1 and n is 0, L₁ is a —C(O)—(CH₂)₍₁₋₄₎—NH₂ group;
Tag₁ is chosen from a —C(O)—(CH₂)ᵣ—CH₃ group, a —C(O)—(CH₂)₇-((E-CH═CH)—CH₂)₁—(CH₂)₈—CH₃ group, a —C(O)—(CH₂)₇—((Z—CH═CH)—CH₂)₁—(CH₂)₆—CH₃ group, and a —C(O)—(CH₂)₇—((Z—CH═CH)—CH₂)₃—CH₃ group, wherein when Aa₇₄ is a 4-aminoproline residue and m is 0, the Tag₁ group is linked to Aa₇₄ through the 4-amino substituent of the 4-aminoproline residue;
r is an integer selected from 6 to 20;
Aa₈₄ is a leucine residue or a lysine residue, wherein the leucine residue is optionally N-methylated at the peptidic bond;
Aa₈₅ is a proline or a D-proline residue;
s is 0 or 1;
t is 0 or 1;
u is 0 or 1;
Aa₇₈ is chosen from a proline, a L-thioproline, an alanine, an arginine, and a glutamic acid residue, wherein the proline, L-thioproline, alanine, arginine or glutamic acid residue is optionally substituted by one or two substituents selected from a halogen atom and amino group; and
G₁ is chosen from a phenyl group, a pyridine group, or an indolyl group; wherein the phenyl, pyridine and indolyl groups are optionally substituted by one, two, three or four substituents selected from a C₁-C₄ alkyl group and a halogen atom; or a 4-6 membered saturated heterocyclyl group containing one oxygen atom selected from an oxetanyl group and a tetrahydro-2H-pyranyl group.

22. The peptidic compound according to claim 21, wherein:
Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue; and
r is an integer selected from 6 to 20.

23. The peptidic compound according to claim 1, wherein the peptidic compound is a compound of Formula (IA), or a pharmaceutically acceptable salt, or solvate, or N-oxide, or stereoisomer thereof: 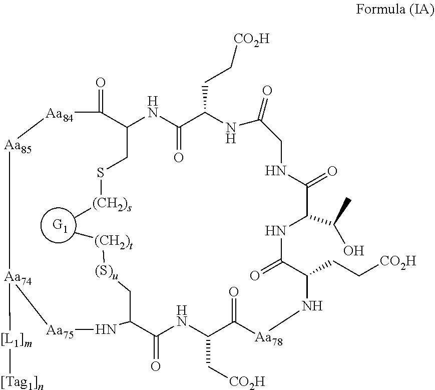
wherein:
Aa₇₄ is chosen from a leucine, a lysine, a 4-aminoproline, and a 4-acetaminoproline residue;
Aa₇₅ is a glutamine residue;
m and n are independently an integer selected from 0 and 1;
when m is 1 and n is 1, L₁ is a —C(O)—(CH₂)₍₁₋₄₎—NH— group, and 1;
when m is 1 and n is 0, L₁ is a —C(O)—(CH₂)₍₁₋₄₎—NH₂ group;
Tag₁ is a —C(O)—(CH₂)ᵣ—CH₃ group, wherein when Aa₇₄ is a 4-aminoproline residue and m is 0, the Tag₁ group is linked to Aa₇₄ through the 4-amino substituent of the 4-aminoproline residue;
r is an integer selected from 6 to 18;
Aa₈₄ is a leucine residue, wherein the leucine residue is optionally N-methylated at the peptidic bond;
Aa₈₅ is a proline or a D-proline residue;
s is 0 or 1;
t is 0 or 1;
u is 0 or 1;
Aa₇₈ is chosen from a proline, an alanine, an arginine, and a glutamic acid residue, wherein the proline, alanine, arginine or glutamic acid residue is optionally substituted by one or two substituents selected from a halogen atom and amino group; and
G₁ is a phenyl group or an indolyl group; wherein the phenyl and indolyl groups are optionally substituted by one, two, three or four substituents selected from a C₁-C₄ alkyl group and a halogen atom.

24. The peptidic compound according to claim 1, wherein the peptidic compound has a sequence selected from:
(SEQ ID NO: 1)
H-Leu-Gln-Trp(indol-2-yl-&¹)-Asp-Glu-Glu-Thr-Gly-Glu-Cys(&¹)-Leu-Pro-OH;
(SEQ ID NO: 2)
{[H-Leu-Gln-Cys(&¹)-Asp-Glu-Glu-Thr-Gly-Glu-Cys(&²)-Leu-Pro-OH][&¹(1,4-
phenylenediyl)dimethylene&²]};
(SEQ ID NO: 3)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]};
(SEQ ID NO: 4)
{[&¹Leu-Gln-Cys(&²)-Asp-Glu-Glu-Thr-Gly-Glu-Cys(&³)-Leu-Pro&¹][&²(1,3-
phenylenediyl)dimethylene&³]};
(SEQ ID NO: 5)
{[&¹Leu-Gln-Cys(&²)-Asp-Glu-Glu-Thr-Gly-Glu-Cys(&³)-Leu-Pro&¹][&²(1,4-
phenylenediyl)dimethylene&³]};
(SEQ ID NO: 6)
{[Acetyl-Cys(&¹)-Asp-Glu-Glu-Thr-Gly-Glu-Cys(&²)-NH₂][&¹(1,3-
phenylenediyl)dimethylene&²]};
(SEQ ID NO: 7)
Acetyl-Trp(indol-2-yl-&¹)-Asp-Glu-Glu-Thr-Gly-Glu-Cys(&¹)-NH₂;
(SEQ ID NO: 8)
H-Leu-Gln-Trp(indol-2-yl-&¹)-Asp-Glu-Glu-Thr-Gly-Glu-Cys(&¹)-Leu-Pro-βAla-Arg-
Lys-Lys-Arg-Arg-Gln-Arg-Arg-Arg-NH₂;
(SEQ ID NO: 9)
{[H-Leu-Gln-Cys(&¹)-Asp-Glu-Glu-Thr-Gly-Glu-Cys(&²)-Leu-Pro-βAla-Arg-Lys-
Lys-Arg-Arg-Gln-Arg-Arg-Arg-NH₂][&¹(1,3-phenylenediyl)dimethylene&²]};
(SEQ ID NO: 10)
Acetyl-Phe(p-&¹)-Asp-Glu-Glu-Thr-Gly-Glu-Cys(&¹)-NH₂;
(SEQ ID NO: 11)
&¹Leu-Gln-Trp(indol-2-yl-&²)-Asp-Glu-Glu-Thr-Gly-Glu-Cys(&²)-Leu-Pro&¹;
(SEQ ID NO: 12)
{[Acetyl-Cys(&¹)-Asp-Glu-Glu-Thr-Gly-Glu-Cys(&²)-NH₂][&¹(1,2-
phenylenediyl)dimethylene&²]};
(SEQ ID NO: 13)
{[H-Leu-Gln-Cys(&¹)-Asp-Glu-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]};
(SEQ ID NO: 14)
{[H-Leu-Gln-Cys(&¹)-Asp-Ala-Glu-Thr-Gly-Glu-Cys(&²)-Leu-Pro-OH][&¹(1,3-
phenylenediyl)dimethylene&²]};
(SEQ ID NO: 15)
{[H-Leu-Gln-Cys(&¹)-Asp-Ala-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]};
(SEQ ID NO: 16)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,3-phenylenediyl)dimethylene&²]};
(SEQ ID NO: 17)
{[&¹Leu-Gln-Cys(&²)-Asp-Ala-Glu-Thr-Gly-Glu-Cys(&³)-Leu-Pro&¹][&²(1,3-
phenylenediyl)dimethylene&³]};
(SEQ ID NO: 18)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 19)
{[H-Leu-Gln-Cys(&¹)-Asp-Ala-Glu-Thr-Gly-Glu-Cys(&²)-Leu-Pro-OH][&¹(1,2-
phenylenediyl)dimethylene&²]};
(SEQ ID NO: 20)
{[Acetyl-Cys(&¹)-Asp-Ala-Glu-Thr-Gly-Glu-Cys(&²)-NH₂][&¹(1,2-
phenylenediyl)dimethylene&²]};
(SEQ ID NO: 21)
{[Acetyl-Cys(&¹)-Asp-Ala-Glu-Thr-Gly-Glu-Cys(&²)-βAla-Arg-Lys-Lys-Arg-Arg-
Gln-Arg-Arg-Arg-NH₂][&¹(1,2-phenylenediyl)dimethylene&²]};
(SEQ ID NO: 22)
Acetyl-Phe(m-&¹)-Asp-Glu-Glu-Thr-Gly-Glu-Cys(&¹)-NH₂;
(SEQ ID NO: 23)
Acetyl-Phe(o-&¹)-Asp-Glu-Glu-Thr-Gly-Glu-Cys(&¹)-NH₂;
(SEQ ID NO: 24)
{[Acetyl-Cys(&¹)-Asp-Glu-Glu-Thr-Gly-Glu-Cys(&²)-NH₂][&¹(1,1′-biphenyl)2,2′-
diyldimethylene&²]};
(SEQ ID NO: 25)
{[Acetyl-Cys(&¹)-Asp-Glu-Glu-Thr-Gly-Glu-Cys(&²)-NH₂][&¹(1,1′-binaphthalene)2,2′-
diyldimethylene&²]};
(SEQ ID NO: 26)
{[Acetyl-Cys(&¹)-Asp-Glu-Glu-Thr-Gly-Glu-Cys(&²)-NH₂][&¹(quinoxaline)2,3-
diyldimethylene&²]};
(SEQ ID NO: 27)
{[Acetyl-Cys(&¹)-Asp-Glu-Glu-Thr-Gly-Glu-Cys(&²)-NH₂][&¹(naphthalene)2,3-
diyldimethylene&²]};
(SEQ ID NO: 28)
{[Stearyl-βAla-Cys(&¹)-Asp-Ala-Glu-Thr-Gly-Glu-Cys(&²)-NH₂][&¹(1,2-
phenylenediyl)dimethylene&²]};
(SEQ ID NO: 29)
{[Stearyl-Ala-Leu-Gln-Cys(&¹)-Asp-Ala-G1u-Thr-Gly-Glu-Cys(&²)-Leu-Pro-OH][&¹(1,2-
phenylenediyl)dimethylene&²]};
(SEQ ID NO: 30)
{[H-Leu-Gln-Cys(&¹)-Asp-Ala-Glu-Thr-Gly-Glu-Cys(&²)-Leu-Pro-βAla-Arg-Lys-Lys-Arg-
Arg-Gln-Arg-Arg-Arg-NH₂][&¹(naphtalene)2,3-diyldimethylene&²]};
(SEQ ID NO: 31)
{[Stearyl-βAla-Cys(&¹)-Asp-Pro-Glu-Thr-Gly-Glu-Cys(&²)-NH₂][&¹(1,2-
phenylenediyl)dimethylene&²]};
(SEQ ID NO: 32)
{[Stearyl-βAla-Cys(&¹)-Asp-Pro-Glu-Thr-Gly-Glu-Cys(&²)-NH₂][&¹(naphthalene)2,3-
diyldimethylene&²]};
(SEQ ID NO: 33)
{[Stearyl-βAla-Cys(&¹)-Asp-Pro-Glu-Thr-Gly-Glu-Cys(&²)-NH₂][&¹(1,3-
phenylenediyl)dimethylene&²]};
(SEQ ID NO: 34)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 35)
{[Stearyl-βAla-Leu-Gln-Cys(&¹)-Asp-Pro-Glu-Thr-Gly-Glu-Cys(&²)-Leu-Pro-OH][&¹(1,2-
phenylenediyl)dimethylene&²]};
(SEQ ID NO: 36)
{[Stearyl-βAla-Leu-Gln-Cys(&¹)-Asp-Pro-Glu-Thr-Gly-Glu-Cys(&²)-Leu-Pro-OH]
[&¹(naphthalene)2,3-diyldimethylene&²]};
(SEQ ID NO: 37)
{[Stearyl-βAla-Leu-Gln-Cys(&¹)-Asp-Pro-Glu-Thr-Gly-Glu-Cys(&²)-Leu-Pro-OH][&¹(1,3-
phenylenediyl)dimethylene&²]};
(SEQ ID NO: 38)
{[Stearyl-βAla-Leu-Gln-Cys(&¹)-Asp-Ala-Glu-Thr-Gly-Glu-Cys(&²)-Leu-Pro-OH]
[&¹(naphthalene)2,3-diyldimethylene&²]};
(SEQ ID NO: 39)
{[Stearyl-βAla-Leu-Gln-Cys(&¹)-Asp-Ala-Glu-Thr-Gly-Glu-Cys(&²)-Leu-Pro-OH][&¹(1,3-
phenylenediyl)dimethylene&²]};
(SEQ ID NO: 40)
Stearyl-βAla-Leu-Gln-Phe(m-&¹)-Asp-Pro-Glu-Thr-Gly-Glu-Cys(&¹)-Leu-Pro-OH;
(SEQ ID NO: 41)
Stearyl-βAla-Phe(m-&¹)-Asp-Pro-Glu-Thr-Gly-Glu-Cys(&¹)-NH₂;
(SEQ ID NO: 42)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(naphthalene)2,3-diyldimethylene&²]};
(SEQ ID NO: 43)
{[Acetyl-Cys(&¹)-Asp-MeAla-Glu-Thr-Gly-Glu-Cys(&²)-NH₂][&¹(1,2-
phenylenediyl)dimethylene&²]};
(SEQ ID NO: 44)
{[Stearyl-βAla-Leu-Leu-Cys(&¹)-Asp-Pro-Glu-Thr-Gly-Glu-Cys(&²)-Leu-Leu-
NH₂][&¹(1,2-phenylenediyl)dimethylene&²]};
(SEQ ID NO: 45)
{[Stearyl-βAla-MeLeu-Gln-Cys(&¹)-Asp-Pro-Glu-Thr-Gly-Glu-Cys(&²)-Leu-Pro-
NH₂][&¹(1,2-phenylenediyl)dimethylene&²]};
(SEQ ID NO: 46)
{[Stearyl-Ala-Leu-Gln-Cys(&¹)-Asp-Pro-Glu-Thr-Gly-Glu-Cys(&²)-MeLeu-Pro-
NH₂][&¹(1,2-phenylenediyl)dimethylene&²]};
(SEQ ID NO: 47)
{[Stearyl-βAla-Lys-Gln-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 48)
{[Stearyl-βAla-Leu-Gln-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 49)
{[Stearyl-βAla-Lys(&¹)-Gln-Cys(&²)-Asp-Pro-Glu-Thr-Gly-Glu-Cys(&³)-Leu-
Pro&¹][&²(1,2-phenylenediyl)dimethylene&³]};
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 51)
{[Stearyl-Ala-Leu-Gln-Cys(&¹)-Asp-Pro-Glu-Thr-Gly-Glu-Cys(&²)-Leu-Pro-
NH₂][&¹(1,3,5-nimethylbenzene)2,4-diyldimethylene&²]};
(SEQ ID NO: 52)
{[Stearyl-βAla-Val-Val-Cys(&¹)-Asp-Pro-Glu-Thr-Gly-Glu-Cys(&²)-Val-Val-NH₂][&¹(1,2-
phenylenediyl)dimethylene&²]};
(SEQ ID NO: 53)
{[Stearyl-βAla-Lys-Lys-Cys(&¹)-Asp-Pro-Glu-Thr-Gly-Glu-Cys(&²)-Lys-Lys-NH₂][&¹(1,2-
phenylenediyl)dimethylene&²]};
(SEQ ID NO: 54)
{[Stearyl-βAla-Leu-Gln-Cys(&¹)-Asp-Pro-Glu-Thr-Gly-Glu-Cys(&²)-Leu-Pro-βAla-Arg-
Lys-Lys-Arg-Arg-Gln-Arg-Arg-Arg-NH₂][&¹(1,2-phenylenediyl)dimethylene&²]};
(SEQ ID NO: 55)
{[Stearyl-Ala-Leu-Gln-Cys(&¹)-Asp-Pro-Glu-Thr-Gly-Glu-Cys(&²)-Leu-Pro-
NH₂][&¹(3,4,5,6-tenafluorobenzene)1,2-diyldimethylene&²]};
(SEQ ID NO: 56)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(3,4,5,6-tetrafluorobenzene)1,2-diyldimethylene&²]};
(SEQ ID NO: 57)
{[Stearyl-βAla-Lys-Lys-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 58)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(naphthalene)2,3-diyldimethylene&²]};
(SEQ ID NO: 59)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,3,5-trimethylbenzene)2,4-diyldimethylene&²]};
(SEQ ID NO: 60)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,2-phenylenediyl)dimethylene&²]};
(SEQ ID NO: 61)
{[Stearyl-βAla-Leu-Gln-Cys(&¹)-Asp-Pro-Glu-Thr-Gly-Glu-Cys(&²)-Leu-Pro-
NH₂][&¹(quinoxaline)2,3-diyldimethylene&²]};
(SEQ ID NO: 62)
{[Stearyl-βAla-Leu-Gln-Cys(&¹)-Asp-Pro-Glu-Thr-Gly-Glu-Cys(&²)-Leu-Pro-
NH₂][&¹(pyridine)2,6-diyldimethylene&²]};
(SEQ ID NO: 63)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,1′-binaphthalene)2,2′-diyldimethylene&²]};
(SEQ ID NO: 64)
{[&¹Pro((4S)-NH-Acetyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 65)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]};
(SEQ ID NO: 66)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,1′-binaphthalene)2,2′-diyldimethylene&²]};
(SEQ ID NO: 67)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,3,5-trimethylbenzene)2,4-diyldimethylene&²]};
(SEQ ID NO: 68)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]};
(SEQ ID NO: 69)
{[H-Leu-Gln-Cys(&¹)-Asp-Pro-Glu-Thr-Gly-Glu-Cys(&²)-Leu-Pro-βAla-Arg₁₀-
NH₂][&¹(naphthalene)2,3-diyldimethylene&²]};
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&²]};
(SEQ ID NO: 71)
{[H-Leu-Gln-Cys(&¹)-Asp-Phe(4-F)-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 72)
{[&¹Pro((4S)-NH-Stearyl)-Gln-Cys(&²)-Asp-Pro((4S)-F)-Glu-Thr-Gly-Glu-Cys(&³)-Leu-D-
Pro&¹][&²(1,2-phenylenediyl)dimethylene&²]};
(SEQ ID NO: 73)
{[&¹Pro((4S)-NH-Stearyl)-Gln-Cys(&²)-Asp-Pro((4S)-NH₂)-Glu-Thr-Gly-Glu-Cys(&³)-Leu-
D-Pro&¹][&²(1,2-phenylenediyl)dimethylene&³]};
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]};
(SEQ ID NO: 75)
{[Acetyl-Pro((4S)-NH-Stearyl)-Gln-Cys(&¹)-Asp-Pro-Glu-Thr-Gly-Glu-Cys(&²)-Leu-D-
Pro-NH₂][&¹(1,2-phenylenediyl)dimethylene²]};
(SEQ ID NO: 76)
{[Stearyl-Pro-Gln-Cys(&¹)-Asp-Pro-Glu-Thr-Gly-Glu-Cys(&²)-Leu-D-Pro-NH₂][&¹(1,2-
phenylenediyl)dimethylene²]};
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&³]};
(SEQ ID NO: 78)
{[&¹Pro((4S)-NH-Stearyl)-Gln-Cys(&²)-Asp-Thz-Glu-Thr-Gly-Glu-Cys(&³)-Leu-D-
Pro&¹][&²(1,2-phenylenediyl)dimethylene&³]};
(SEQ ID NO: 79)
{[&¹Pro((4S)-NH-Stearyl)-Gln-Cys(&²)-Asp-Pro-Glu-Thr-Gly-Glu-Cys(&³)-Leu-D-
Pro&¹][&²(3,3-oxetanediyl)dimethylene&³]};
(SEQ ID NO: 80)
{[&¹Pro((4S)-NH-Miristoyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 81)
{[&¹Pro((4S)-NH-Stearyl)-Lys-Cys(&²)-Asp-Pro-Glu-Thr-Gly-Glu-Cys(&³)-Leu-D-
Pro&¹][&²(1,2-phenylenediy1)dimethylene&³]};
(SEQ ID NO: 82)
{[&¹Pro((4S)-NH-Stearyl)-Gln-Cys(&²)-Asp-Pro-Glu-Thr-Gly-Glu-Cys(&³)-Lys-D-
Pro&¹][&²(1,2-phenylenediyl)dimethylene&³]};
(SEQ ID NO: 83)
{[&¹Pro((4S)-NH-Palmitoyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 84)
{[&¹Pro((4S)-NH-Stearyl)-Gln-Cys(&²)-Asp-Pro-Glu-Thr-Gly-Glu-Cys(&³)-Leu-D-
Pro&¹][&¹(pyridine)3,5-diyldimethylene&²]};
(SEQ ID NO: 85)
{[&¹Pro((4S)-NH-Lauryl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 86)
{[&¹Pro((4S)-NH-α-linolenyl)-Gln-Cys(&²)-Asp-Pro-Glu-Thr-Gly-Glu-Cys(&³)-Leu-
D-Pro&¹][&²(1,2-phenylenediyl)dimethylene&³]};
(SEQ ID NO: 87)
{[&¹Pro((4S)-NH-elaidyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 88)
{[&¹Pro((4S)-NH-oleyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 89)
{[&¹Pro((4S)-NH-behenyl)-Gln-Cys(&²)-Asp-Pro-Glu-Thr-Gly-Glu-Cys(&3)-Leu-D-
Pro&¹][&²(1,2-phenylenediyl)dimethylene&³]};
(SEQ ID NO: 90)
{[&¹Pro((4S)-NH-arachidyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 91)
{[&¹Lys(N⁶-Stearyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 92)
{[Stearyl-Lys(&¹)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 93)
{[&¹Pro((4S)-NH-Arachidyl)-Gln-Cys(&²)-Asp-Thz-Glu-Thr-Gly-Glu-Cys(&³)-Leu-D-
Pro&¹][&²(1,2-phenylenediyl)dimethylene&³]};
(SEQ ID NO: 94)
{[&¹Pro((4S)-NH-Arachidyl)-Gln-Cys(&²)-Asp-Thz-Glu-Thr-Gly-Glu-Cys(&³)-Leu-D-
Pro&¹][&²(3,3-oxetanediyl)dimethylene&³]};
(SEQ ID NO: 95)
{[&¹Pro((4S)-NH-Arachidyl)-Gln-Cys(&²)-Asp-Pro-Glu-Thr-Gly-Glu-Cys(&³)-Leu-D-
Pro&¹][&²(3,3-oxetanediyl)dimethylene&³]};
(SEQ ID NO: 96)
{[&¹Pro((4S)-NH-Stearyl)-Gln-Cys(&²)-Asp-Thz-Glu-Thr-Gly-Glu-Cys(&³)-Leu-D-
Pro&¹][&²(1,3-phenylenediyl)dimethylene&³]};
(SEQ ID NO: 97)
{[&¹Pro((4S)-NH-Stearyl)-Gln-Cys(&²)-Asp-Thz-Glu-Thr-Gly-Glu-Cys(&³)-Leu-D-
Pro&¹][&²(3,3-oxetanediyl)dimethylene&³]};
(SEQ ID NO: 98)
{[&¹Pro((4S)-NH-Nonadecanoyl)-Gln-Cys(&²)-Asp-Pro-Glu-Thr-Gly-Glu-Cys(&³)-
Leu-D-Pro&¹][&²(1,2-phenylenediyl)dimethylene&³]};
(SEQ ID NO: 99)
{[Stearyl-βAla-Leu-Leu-Cys(&¹)-Asp-Pro-Glu-Thr-Gly-Glu-Cys(&²)-Leu-Leu-
NH₂][&¹(3,3-oxetanediyl)dimethylene&²]};
(SEQ ID NO: 100)
{[&¹Pro((4S)-NH-Stearyl)-Gln-Cys(&²)-Asp-Pro-Glu-Thr-Gly-Glu-Cys(&³)-Leu-D-
Pro&¹][&²(tetrahydro-2H-pyran-4,4-diyl)dimethylene&³]};
or a pharmaceutically acceptable salt, or solvate, or N-oxide, or stereoisomer thereof.

25. The peptidic compound according to claim 1, wherein the peptidic compound has a sequence selected from:
(SEQ ID NO: 1)
H-Leu-Gln-Trp(indol-2-yl-&¹)-Asp-Glu-Glu-Thr-Gly-Glu-Cys(&¹)-Leu-Pro-OH;
(SEQ ID NO: 2)
{[H-Leu-Gln-Cys(&¹)-Asp-Glu-Glu-Thr-Gly-Glu-Cys(&²)-Leu-Pro-OH][&¹(1,4-
phenylenediyl)dimethylene&²]};
(SEQ ID NO: 3)
{[H-Leu-Gln-Cys(&¹)-Asp-Glu-Glu-Thr-Gly-Glu-Cys(&²)-Leu-Pro-OH][&¹(1,3-
phenylenediyl)dimethylene&²]};
(SEQ ID NO: 4)
{[&¹Leu-Gln-Cys(&²)-Asp-Glu-Glu-Thr-Gly-Glu-Cys(&³)-Leu-Pro&¹][&²(1,3-
phenylenediyl)dimethylene&³]};
(SEQ ID NO: 5)
{[&¹Leu-Gln-Cys(&²)-Asp-Glu-Glu-Thr-Gly-Glu-Cys(&³)-Leu-Pro&¹][&²(1,4-
phenylenediyl)dimethylene&³]};
(SEQ ID NO: 6)
{[Acetyl-Cys(&¹)-Asp-Glu-Glu-Thr-Gly-Glu-Cys(&²)-NH₂][&¹(1,3-
phenylenediyl)dimethylene&²]};
(SEQ ID NO: 7)
Acetyl-Trp(indol-2-yl-&¹)-Asp-Glu-Glu-Thr-Gly-Glu-Cys(&¹)-NH₂;
(SEQ ID NO: 8)
H-Leu-Gln-Trp(indol-2-yl-&¹)-Asp-Glu-Glu-Thr-Gly-Glu-Cys(&¹)-Leu-Pro-βAla-Arg-
Lys-Lys-Arg-Arg-Gln-Arg-Arg-Arg-NH₂;
(SEQ ID NO: 9)
{[H-Leu-Gln-Cys(&¹)-Asp-Glu-Glu-Thr-Gly-Glu-Cys(&²)-Leu-Pro-βAla-Arg-Lys-
Lys-Arg-Arg-Gln-Arg-Arg-Arg-NH₂][&¹(1,3-phenylenediyl)dimethylene&²]};
(SEQ ID NO: 10)
Acetyl-Phe(p-&¹)-Asp-Glu-Glu-Thr-Gly-Glu-Cys(&¹)-NH₂;
(SEQ ID NO: 11)
&¹Leu-Gln-Trp(indol-2-yl-&²)-Asp-Glu-Glu-Thr-Gly-Glu-Cys(&²)-Leu-Pro&¹;
(SEQ ID NO: 12)
{[Acetyl-Cys(&¹)-Asp-Glu-Glu-Thr-Gly-Glu-Cys(&²)-NH₂][&¹(1,2-
phenylenediyl)dimethylene&²]};
(SEQ ID NO: 13)
{[H-Leu-Gln-Cys(&¹)-Asp-Glu-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]};
(SEQ ID NO: 14)
{[H-Leu-Gln-Cys(&¹)-Asp-Ala-Glu-Thr-Gly-Glu-Cys(&²)-Leu-Pro-OH][&¹(1,3-
phenylenediyl)dimethylene&²]};
(SEQ ID NO: 15)
{[H-Leu-Gln-Cys(&¹)-Asp-Ala-Glu-Thr-Gly-Glu-Cys(&²)-Leu-Pro-βAla-Tyr-Ala-Arg-
Ala-Ala-Ala-Arg-Gln-Ala-Arg-Ala-NH₂][&¹(1,3-phenylenediyl)dimethylene&²]};
(SEQ ID NO: 16)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,3-phenylenediyl)dimethylene&²]};
(SEQ ID NO: 17)
{[&¹Leu-Gln-Cys(&²)-Asp-Ala-Glu-Thr-Gly-Glu-Cys(&³)-Leu-Pro&¹][&²(1,3-
phenylenediyl)dimethylene&³]};
(SEQ ID NO: 18)
{[H-Leu-Gln-Cys(&¹)-Asp-Ala-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 19)
{[H-Leu-Gln-Cys(&¹)-Asp-Ala-Glu-Thr-Gly-Glu-Cys(&²)-Leu-Pro-OH][&¹(1,2-
phenylenediyl)dimethylene&²]};
(SEQ ID NO: 20)
{[Acetyl-Cys(&¹)-Asp-Ala-Glu-Thr-Gly-Glu-Cys(&²)-NH₂][&¹(1,2-
phenylenediyl)dimethylene&²]};
(SEQ ID NO: 21)
{[Acetyl-Cys(&¹)-Asp-Ala-Glu-Thr-Gly-Glu-Cys(&²)-βAla-Arg-Lys-Lys-Arg-Arg-
Gln-Arg-Arg-Arg-NH₂][&¹(1,2-phenylenediyl)dimethylene&²]};
(SEQ ID NO: 22)
Acetyl-Phe(m-&¹)-Asp-Glu-Glu-Thr-Gly-Glu-Cys(&¹)-NH₂;
(SEQ ID NO: 23)
Acetyl-Phe(o-&¹)-Asp-Glu-Glu-Thr-Gly-Glu-Cys(&¹)-NH₂;
(SEQ ID NO: 24)
{[Acetyl-Cys(&¹)-Asp-Glu-Glu-Thr-Gly-Glu-Cys(&²)-NH₂][&¹(1,1′-biphenyl)2,2′-
diyldimethylene&²]};
(SEQ ID NO: 25)
{[Acetyl-Cys(&¹)-Asp-Glu-Glu-Thr-Gly-Glu-Cys(&²)-NH₂][&¹(1,1′-binaphthalene)2,2′-
diyldimethylene&²]};
(SEQ ID NO: 26)
{[Acetyl-Cys(&¹)-Asp-Glu-Glu-Thr-Gly-Glu-Cys(&²)-NH₂][&¹(quinoxaline)2,3-
diyldimethylene&²]};
(SEQ ID NO: 27)
{[Acetyl-Cys(&¹)-Asp-Glu-Glu-Thr-Gly-Glu-Cys(&²)-NH₂][&¹(naphthalene)2,3-
diyldimethylene&²]};
(SEQ ID NO: 28)
{[Stearyl-βAla-Cys(&¹)-Asp-Ala-Glu-Thr-Gly-Glu-Cys(&²)-NH₂][&¹(1,2-
phenylenediyl)dimethylene&²]};
(SEQ ID NO: 29)
{[Stearyl-Ala-Leu-Gln-Cys(&¹)-Asp-Ala-G1u-Thr-Gly-Glu-Cys(&²)-Leu-Pro-OH][&¹(1,2-
phenylenediyl)dimethylene&²]};
(SEQ ID NO: 30)
{[H-Leu-Gln-Cys(&¹)-Asp-Ala-Glu-Thr-Gly-Glu-Cys(&²)-Leu-Pro-βAla-Arg-Lys-Lys-Arg-
Arg-Gln-Arg-Arg-Arg-NH₂][&¹(naphtalene)2,3-diyldimethylene&²]};
(SEQ ID NO: 31)
{[Stearyl-βAla-Cys(&¹)-Asp-Pro-Glu-Thr-Gly-Glu-Cys(&²)-NH₂][&¹(1,2-
phenylenediyl)dimethylene&²]};
(SEQ ID NO: 32)
{[Stearyl-βAla-Cys(&¹)-Asp-Pro-Glu-Thr-Gly-Glu-Cys(&²)-NH₂][&¹(naphthalene)2,3-
diyldimethylene&²]};
(SEQ ID NO: 33)
{[Stearyl-βAla-Cys(&¹)-Asp-Pro-Glu-Thr-Gly-Glu-Cys(&²)-NH₂][&¹(1,3-
phenylenediyl)dimethylene&²]};
(SEQ ID NO: 34)
{[H-Leu-Gln-Cys(&¹)-Asp-Pro-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 35)
{[Stearyl-βAla-Leu-Gln-Cys(&¹)-Asp-Pro-Glu-Thr-Gly-Glu-Cys(&²)-Leu-Pro-OH][&¹(1,2-
phenylenediyl)dimethylene&²]};
(SEQ ID NO: 36)
{[Stearyl-βAla-Leu-Gln-Cys(&¹)-Asp-Pro-Glu-Thr-Gly-Glu-Cys(&²)-Leu-Pro-OH]
[&¹(naphthalene)2,3-diyldimethylene&²]};
(SEQ ID NO: 37)
{[Stearyl-βAla-Leu-Gln-Cys(&¹)-Asp-Pro-Glu-Thr-Gly-Glu-Cys(&²)-Leu-Pro-OH][&¹(1,3-
phenylenediyl)dimethylene&²]};
(SEQ ID NO: 38)
{[Stearyl-βAla-Leu-Gln-Cys(&¹)-Asp-Ala-Glu-Thr-Gly-Glu-Cys(&²)-Leu-Pro-OH]
[&¹(naphthalene)2,3-diyldimethylene&²]};
(SEQ ID NO: 39)
{[Stearyl-βAla-Leu-Gln-Cys(&¹)-Asp-Ala-Glu-Thr-Gly-Glu-Cys(&²)-Leu-Pro-OH][&¹(1,3-
phenylenediyl)dimethylene&²]};
(SEQ ID NO: 40)
Stearyl-βAla-Leu-Gln-Phe(m-&¹)-Asp-Pro-Glu-Thr-Gly-Glu-Cys(&¹)-Leu-Pro-OH;
(SEQ ID NO: 41)
Stearyl-βAla-Phe(m-&¹)-Asp-Pro-Glu-Thr-Gly-Glu-Cys(&¹)-NH₂;
(SEQ ID NO: 42)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(naphthalene)2,3-diyldimethylene&²]};
(SEQ ID NO: 43)
{[Acetyl-Cys(&¹)-Asp-MeAla-Glu-Thr-Gly-Glu-Cys(&²)-NH₂][&¹(1,2-
phenylenediyl)dimethylene&²]};
(SEQ ID NO: 44)
{[Stearyl-βAla-Leu-Leu-Cys(&¹)-Asp-Pro-Glu-Thr-Gly-Glu-Cys(&²)-Leu-Leu-
NH₂][&¹(1,2-phenylenediyl)dimethylene&²]};
(SEQ ID NO: 45)
{[Stearyl-βAla-MeLeu-Gln-Cys(&¹)-Asp-Pro-Glu-Thr-Gly-Glu-Cys(&²)-Leu-Pro-
NH₂][&¹(1,2-phenylenediyl)dimethylene&²]};
(SEQ ID NO: 46)
{[Stearyl-Ala-Leu-Gln-Cys(&¹)-Asp-Pro-Glu-Thr-Gly-Glu-Cys(&²)-MeLeu-Pro-
NH₂][&¹(1,2-phenylenediyl)dimethylene&²]};
(SEQ ID NO: 47)
{[Stearyl-βAla-Lys-Gln-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 48)
{[Stearyl-βAla-Leu-Gln-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 49)
{[Stearyl-βAla-Lys(&¹)-Gln-Cys(&²)-Asp-Pro-Glu-Thr-Gly-Glu-Cys(&³)-Leu-
Pro&¹][&²(1,2-phenylenediyl)dimethylene&³]};
(SEQ ID NO: 50)
{[&¹Pro((4S)-NH-Stearyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 51)
{[Stearyl-Ala-Leu-Gln-Cys(&¹)-Asp-Pro-Glu-Thr-Gly-Glu-Cys(&²)-Leu-Pro-
NH₂][&¹(1,3,5-nimethylbenzene)2,4-diyldimethylene&²]};
(SEQ ID NO: 52)
{[Stearyl-βAla-Val-Val-Cys(&¹)-Asp-Pro-Glu-Thr-Gly-Glu-Cys(&²)-Val-Val-NH₂][&¹(1,2-
phenylenediyl)dimethylene&²]};
(SEQ ID NO: 53)
{[Stearyl-βAla-Lys-Lys-Cys(&¹)-Asp-Pro-Glu-Thr-Gly-Glu-Cys(&²)-Lys-Lys-NH₂][&¹(1,2-
phenylenediyl)dimethylene&²]};
(SEQ ID NO: 54)
{[Stearyl-βAla-Leu-Gln-Cys(&¹)-Asp-Pro-Glu-Thr-Gly-Glu-Cys(&²)-Leu-Pro-βAla-Arg-
Lys-Lys-Arg-Arg-Gln-Arg-Arg-Arg-NH₂][&¹(1,2-phenylenediyl)dimethylene&²]};
(SEQ ID NO: 55)
{[Stearyl-Ala-Leu-Gln-Cys(&¹)-Asp-Pro-Glu-Thr-Gly-Glu-Cys(&²)-Leu-Pro-
NH₂][&¹(3,4,5,6-tenafluorobenzene)1,2-diyldimethylene&²]};
(SEQ ID NO: 56)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(3,4,5,6-tetrafluorobenzene)1,2-diyldimethylene&²]};
(SEQ ID NO: 57)
{[Stearyl-βAla-Lys-Lys-Cys(&¹)-Asp-Pro-Glu-Thr-Gly-Glu-Cys(&²)-Leu-Pro-NH₂][&¹(1,2-
phenylenediyl)dimethylene&²]};
(SEQ ID NO: 58)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(naphthalene)2,3-diyldimethylene&²]};
(SEQ ID NO: 59)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,3,5-trimethylbenzene)2,4-diyldimethylene&²]};
(SEQ ID NO: 60)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,2-phenylenediyl)dimethylene&²]};
(SEQ ID NO: 61)
{[Stearyl-βAla-Leu-Gln-Cys(&¹)-Asp-Pro-Glu-Thr-Gly-Glu-Cys(&²)-Leu-Pro-
NH₂][&¹(quinoxaline)2,3-diyldimethylene&²]};
(SEQ ID NO: 62)
{[Stearyl-βAla-Leu-Gln-Cys(&¹)-Asp-Pro-Glu-Thr-Gly-Glu-Cys(&²)-Leu-Pro-
NH₂][&¹(pyridine)2,6-diyldimethylene&²]};
(SEQ ID NO: 63)
{[H-Leu-Leu-Cys(&¹)-Asp-Pro-Glu-Thr-Gly-Glu-Cys(&²)-Leu-Leu-βAla-Arg-Lys-Lys-Arg-
Arg-Gln-Arg-Arg-Arg-NH₂][&¹(1,1′-binaphthalene)2,2′-diyldimethylene&²]};
(SEQ ID NO: 64)
{[&¹Pro((4S)-NH-Acetyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 65)
{[Stearyl-βAla-Leu-Gln-Cys(&¹)-Asp-Pro-Glu-Thr-Gly-Glu-Cys(&²)-Leu-Pro-
NH₂][&¹(pyridine)3,5-diyldimethylene&²]};
(SEQ ID NO: 66)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,1′-binaphthalene)2,2′-diyldimethylene&²]};
(SEQ ID NO: 67)
{[H-Leu-Gln-Cys(&¹)-Asp-Pro-Glu-Thr-Gly-Glu-Cys(&²)-Leu-Pro-βAla-Arg-Lys-Lys-Arg-
Arg-Gln-Arg-Arg-Arg-NH₂][&¹(1,3,5-trimethylbenzene)2,4-diyldimethylene&²]};
(SEQ ID NO: 68)
{[H-Leu-Gln-Cys(&¹)-Asp-Pro-Glu-Thr-Gly-Glu-Cys(&²)-Leu-Pro-βAla-Arg₈-
NH₂][&¹(naphthalene)2,3-diyldimethylene&²]};
(SEQ ID NO: 69)
{[H-Leu-Gln-Cys(&¹)-Asp-Pro-Glu-Thr-Gly-Glu-Cys(&²)-Leu-Pro-βAla-Arg₁₀-
NH₂][&¹(naphthalene)2,3-diyldimethylene&²]};
(SEQ ID NO: 70)
{[&¹Pro((4S)-NH-Stearyl)-Gln-Cys(&²)-Asp-Arg-Glu-Thr-Gly-Glu-Cys(&³)-Leu-D-
Pro&¹][&²(1,2-phenylenediyl)dimethylene&²]};
(SEQ ID NO: 71)
{[H-Leu-Gln-Cys(&¹)-Asp-Phe(4-F)-Glu-Thr-Gly-Glu-Cys(&²)-Leu-Pro-βAla-Arg-Lys-Lys-
Arg-Arg-Gln-Arg-Arg-Arg-NH₂][&¹(1,2-phenylenediyl)dimethylene&²]};
(SEQ ID NO: 72)
{[&¹Pro((4S)-NH-Stearyl)-Gln-Cys(&²)-Asp-Pro((4S)-F)-Glu-Thr-Gly-Glu-Cys(&³)-Leu-D-
Pro&¹][&²(1,2-phenylenediyl)dimethylene&²]};
(SEQ ID NO: 73)
{[&¹Pro((4S)-NH-Stearyl)-Gln-Cys(&²)-Asp-Pro((4S)-NH₂)-Glu-Thr-Gly-Glu-Cys(&³)-Leu-
D-Pro&¹][&²(1,2-phenylenediyl)dimethylene&³]};
(SEQ ID NO: 74)
{[&¹Pro((4S)-NH-Stearyl)-Gln-Cys(&²)-Asp-Pro-Glu-Thr-Gly-Glu-Cys(&³)-Leu-D-
Pro&¹][&²(1,3-phenylenediyl)dimethylene&³]};
(SEQ ID NO: 75)
{[Acetyl-Pro((4S)-NH-Stearyl)-Gln-Cys(&¹)-Asp-Pro-Glu-Thr-Gly-Glu-Cys(&²)-Leu-D-
Pro-NH₂][&¹(1,2-phenylenediyl)dimethylene²]};
(SEQ ID NO: 76)
{[Stearyl-Pro-Gln-Cys(&¹)-Asp-Pro-Glu-Thr-Gly-Glu-Cys(&²)-Leu-D-Pro-NH₂][&¹(1,2-
phenylenediyl)dimethylene²]};
(SEQ ID NO: 77)
{[&¹Pro((4S)-NH-Stearyl)-Gln-Cys(&²)-Asp-Pro-Glu-Thr-Gly-Glu-Cys(&³)-MeLeu-D-
Pro&¹][&²(1,2-phenylenediyl)dimethylene&³]};
(SEQ ID NO: 78)
{[&¹Pro((4S)-NH-Stearyl)-Gln-Cys(&²)-Asp-Thz-Glu-Thr-Gly-Glu-Cys(&³)-Leu-D-
Pro&¹][&²(1,2-phenylenediyl)dimethylene&³]};
(SEQ ID NO: 79)
{[&¹Pro((4S)-NH-Stearyl)-Gln-Cys(&²)-Asp-Pro-Glu-Thr-Gly-Glu-Cys(&³)-Leu-D-
Pro&¹][&²(3,3-oxetanediyl)dimethylene&³]};
(SEQ ID NO: 80)
{[&¹Pro((4S)-NH-Miristoyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 81)
{[&¹Pro((4S)-NH-Stearyl)-Lys-Cys(&²)-Asp-Pro-Glu-Thr-Gly-Glu-Cys(&³)-Leu-D-
Pro&¹][&²(1,2-phenylenediy1)dimethylene&³]};
(SEQ ID NO: 82)
{[&¹Pro((4S)-NH-Stearyl)-Gln-Cys(&²)-Asp-Pro-Glu-Thr-Gly-Glu-Cys(&³)-Lys-D-
Pro&¹][&²(1,2-phenylenediyl)dimethylene&³]};
(SEQ ID NO: 83)
{[&¹Pro((4S)-NH-Palmitoyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 84)
{[&¹Pro((4S)-NH-Stearyl)-Gln-Cys(&²)-Asp-Pro-Glu-Thr-Gly-Glu-Cys(&³)-Leu-D-
Pro&¹][&¹(pyridine)3,5-diyldimethylene&²]};
(SEQ ID NO: 85)
{[&¹Pro((4S)-NH-Lauryl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 86)
{[&¹Pro((4S)-NH-α-linolenyl)-Gln-Cys(&²)-Asp-Pro-Glu-Thr-Gly-Glu-Cys(&³)-Leu-
D-Pro&¹][&²(1,2-phenylenediyl)dimethylene&³]};
(SEQ ID NO: 87)
{[&¹Pro((4S)-NH-elaidyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 88)
{[&¹Pro((4S)-NH-oleyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 89)
{[&¹Pro((4S)-NH-behenyl)-Gln-Cys(&²)-Asp-Pro-Glu-Thr-Gly-Glu-Cys(&3)-Leu-D-
Pro&¹][&²(1,2-phenylenediyl)dimethylene&³]};
(SEQ ID NO: 90)
{[&¹Pro((4S)-NH-arachidyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 91)
{[&¹Lys(N⁶-Stearyl)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 92)
{[Stearyl-Lys(&¹)-Gln-Cys(&²)-Asp-Pro-Glu-Thr-Gly-Glu-Cys(&³)-Leu-D-
Pro&¹][&²(1,2-phenylenediyl)dimethylene&³]};
(SEQ ID NO: 93)
{[&¹Pro((4S)-NH-Arachidyl)-Gln-Cys(&²)-Asp-Thz-Glu-Thr-Gly-Glu-Cys(&³)-Leu-D-
Pro&¹][&²(1,2-phenylenediyl)dimethylene&³]};
(SEQ ID NO: 94)
{[&¹Pro((4S)-NH-Arachidyl)-Gln-Cys(&²)-Asp-Thz-Glu-Thr-Gly-Glu-Cys(&³)-Leu-D-
Pro&¹][&²(3,3-oxetanediyl)dimethylene&³]};
(SEQ ID NO: 95)
{[&¹Pro((4S)-NH-Arachidyl)-Gln-Cys(&²)-Asp-Pro-Glu-Thr-Gly-Glu-Cys(&³)-Leu-D-
Pro&¹][&²(3,3-oxetanediyl)dimethylene&³]};
(SEQ ID NO: 96)
{[&¹Pro((4S)-NH-Stearyl)-Gln-Cys(&²)-Asp-Thz-Glu-Thr-Gly-Glu-Cys(&³)-Leu-D-
Pro&¹][&²(1,3-phenylenediyl)dimethylene&³]};
(SEQ ID NO: 97)
{[&¹Pro((4S)-NH-Stearyl)-Gln-Cys(&²)-Asp-Thz-Glu-Thr-Gly-Glu-Cys(&³)-Leu-D-
Pro&¹][&²(3,3-oxetanediyl)dimethylene&³]};
(SEQ ID NO: 98)
{[&¹Pro((4S)-NH-Nonadecanoyl)-Gln-Cys(&²)-Asp-Pro-Glu-Thr-Gly-Glu-Cys(&³)-
Leu-D-Pro&¹][&²(1,2-phenylenediyl)dimethylene&³]};
(SEQ ID NO: 99)
{[Stearyl-βAla-Leu-Leu-Cys(&¹)-Asp-Pro-Glu-Thr-Gly-Glu-Cys(&²)-Leu-Leu-
NH₂][&¹(3,3-oxetanediyl)dimethylene&²]};
(SEQ ID NO: 100)
{[&¹Pro((4S)-NH-Stearyl)-Gln-Cys(&²)-Asp-Pro-Glu-Thr-Gly-Glu-Cys(&³)-Leu-D-
Pro&¹][&²(tetrahydro-2H-pyran-4,4-diyl)dimethylene&³]};
or a pharmaceutically acceptable salt, or solvate, or N-oxide, or stereoisomer thereof.

26. A pharmaceutical composition comprising a peptidic compound according to claim 1 together with one or more pharmaceutically acceptable carriers and/or excipients.

27. (canceled)

28. A method for treating a subject afflicted with a pathological condition or disease associated with the activation of the Nrf2 pathway, the method comprising administering to the subject an effective amount of a compound according to claim 1 or a pharmaceutical composition according to claim 26.

29. The method according to claim 28, wherein the pathological condition or disease is selected from Parkinson's disease, depression, Alzheimer's disease, atherosclerosis, heart failure, myocardial infarction, diabetes, cancer, COPD, COPD exacerbations, acute lung injury, radiation-induced dermatitis, chemical induced dermatitis, contact induced dermatitis, Epidermolysis bullosa simplex, Pachyonychia congenital, Hailey-Hailey, vitiligo, photoaging, and photodamaged skin.

30. (canceled)

31. (canceled)


SEQUENCE LISTING (123 sequences)

SEQ ID NO: 1 - protein, 12 aa, Artificial Sequence
  peptidic sequences of the compounds of formula (I)
  BINDING (3)..(3): binding site for cyclization
  BINDING (10)..(10): binding site for cyclization
LQWDEETGECLP

SEQ ID NO: 2 - protein, 12 aa, Artificial Sequence
  peptidic sequences of the compounds of formula (I)
  BINDING (3)..(3): binding site for cyclization
  BINDING (10)..(10): binding site for cyclization
LQCDEETGECLP

SEQ ID NO: 3 - protein, 12 aa, Artificial Sequence
  peptidic sequences of the compounds of formula (I)
  BINDING (3)..(3): binding site for cyclization
  BINDING (10)..(10): binding site for cyclization
LQCDEETGECLP

SEQ ID NO: 4 - protein, 12 aa, Artificial
  peptidic sequences of the compounds of formula (I)
  BINDING (1)..(1): binding site for cyclization
  BINDING (3)..(3): binding site for cyclization
  BINDING (10)..(10): binding site for cyclization
  BINDING (12)..(12): binding site for cyclization
LQCDEETGECLP

SEQ ID NO: 5 - protein, 12 aa, Artificial
  peptidic sequences of the compounds of formula (I)
  BINDING (1)..(1): binding site for cyclization
  BINDING (3)..(3): binding site for cyclization
  BINDING (10)..(10): binding site for cyclization
  BINDING (12)..(12): binding site for cyclization
LQCDEETGECLP

SEQ ID NO: 6 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 7 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
WDEETGEC

SEQ ID NO: 8 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQWDEETGECLPXRKKRRQRRR

SEQ ID NO: 9 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDEETGECLPXRKKRRQRRR

SEQ ID NO: 10 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
FDEETGEC

SEQ ID NO: 11 - protein, 12 aa, Artificial Sequence
  peptidic sequences of the compounds of formula (I)
  BINDING (1)..(1): binding site for cyclization
  BINDING (3)..(3): binding site for cyclization
  BINDING (10)..(10): binding site for cyclization
  BINDING (12)..(12): binding site for cyclization
LQWDEETGECLP

SEQ ID NO: 12 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 13 - protein, 24 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (24)..(24): AMIDATION
LQCDEETGECLPXYARAAARQARA

SEQ ID NO: 14 - protein, 12 aa, Artificial Sequence
  peptidic sequences of the compounds of formula (I)
  BINDING (3)..(3): binding site for cyclization
  BINDING (10)..(10): binding site for cyclization
LQCDAETGECLP

SEQ ID NO: 15 - protein, 24 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (24)..(24): AMIDATION
LQCDAETGECLPXYARAAARQARA

SEQ ID NO: 16 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDAETGECLPXRKKRRQRRR

SEQ ID NO: 17 - protein, 12 aa, Artificial Sequence
  peptidic sequences of the compounds of formula (I)
  BINDING (1)..(1): binding site for cyclization
  BINDING (3)..(3): binding site for cyclization
  BINDING (10)..(10): binding site for cyclization
  BINDING (12)..(12): binding site for cyclization
LQCDAETGECLP

SEQ ID NO: 18 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDAETGECLPXRKKRRQRRR

SEQ ID NO: 19 - protein, 12 aa, Artificial Sequence
  peptidic sequences of the compounds of formula (I)
  BINDING (3)..(3): binding site for cyclization
  BINDING (10)..(10): binding site for cyclization
LQCDAETGECLP

SEQ ID NO: 20 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDAETGEC

SEQ ID NO: 21 - protein, 18 aa, Artificial Sequence
  peptidic sequences of the compounds of formula (I)
  BINDING (1)..(1): binding site for cyclization
  MOD_RES (1)..(1): ACETYLATION
  BINDING (8)..(8): binding site for cyclization
  MOD_RES (9)..(9): bAla
  MOD_RES (18)..(18): AMIDATION
CDAETGECXRKKRRQRRR

SEQ ID NO: 22 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
FDEETGEC

SEQ ID NO: 23 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
FDEETGEC

SEQ ID NO: 24 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 25 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 26 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 27 - protein, 8 aa, Artificial Sequence
  peptidic sequences of the compounds of formula (I)
  BINDING (1)..(1): binding site for cyclization
  MOD_RES (1)..(1): ACETYLATION
  MOD_RES (8)..(8): AMIDATION
  BINDING (8)..(8): binding site for cyclization
CDEETGEC

SEQ ID NO: 28 - protein, 9 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  MOD_RES (9)..(9): AMIDATION
  BINDING (9)..(9): binding site for cyclization
XCDAETGEC

SEQ ID NO: 29 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDAETGECLP

SEQ ID NO: 30 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDAETGECLPXRKKRRQRRR

SEQ ID NO: 31 - protein, 9 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  MOD_RES (9)..(9): AMIDATION
  BINDING (9)..(9): binding site for cyclization
XCDPETGEC

SEQ ID NO: 32 - protein, 9 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  MOD_RES (9)..(9): AMIDATION
  BINDING (9)..(9): binding site for cyclization
XCDPETGEC

SEQ ID NO: 33 - protein, 9 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  MOD_RES (9)..(9): AMIDATION
  BINDING (9)..(9): binding site for cyclization
XCDPETGEC

SEQ ID NO: 34 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDPETGECLPXRKKRRQRRR

SEQ ID NO: 35 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDPETGECLP

SEQ ID NO: 36 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDPETGECLP

SEQ ID NO: 37 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDPETGECLP

SEQ ID NO: 38 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDAETGECLP

SEQ ID NO: 39 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQCDAETGECLP

SEQ ID NO: 40 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
XLQFDPETGECLP

SEQ ID NO: 41 - protein, 9 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  MOD_RES (9)..(9): AMIDATION
  BINDING (9)..(9): binding site for cyclization
XFDPETGEC

SEQ ID NO: 42 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDPETGECLPXRKKRRQRRR

SEQ ID NO: 43 - protein, 8 aa, Artificial Sequence
  peptidic sequences of the compounds of formula (I)
  MOD_RES (1)..(1): ACETYLATION
  BINDING (1)..(1): binding site for cyclization
  MOD_RES (3)..(3): MeAla
  MOD_RES (8)..(8): AMIDATION
  BINDING (8)..(8): binding site for cyclization
CDAETGEC

SEQ ID NO: 44 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLLCDPETGECLL

SEQ ID NO: 45 - protein, 13 aa, Artificial Sequence
  peptidic sequences of the compounds of formula (I)
  LIPID (1)..(1): stearyl
  MOD_RES (1)..(1): bAla
  MOD_RES (2)..(2): MeLeu
  BINDING (4)..(4): binding site for cyclization
  BINDING (11)..(11): binding site for cyclization
  MOD_RES (13)..(13): AMIDATION
XLQCDPETGECLP

SEQ ID NO: 46 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (12)..(12): MeLeu
  MOD_RES (13)..(13): AMIDATION
XLQCDPETGECLP

SEQ ID NO: 47 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XKQCDPETGECLP

SEQ ID NO: 48 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 49 - protein, 13 aa, Artificial Sequence
  peptidic sequences of the compounds of formula (I)
  LIPID (1)..(1): stearyl
  MOD_RES (1)..(1): bAla
  BINDING (2)..(2): binding site for cyclization
  BINDING (4)..(4): binding site for cyclization
  BINDING (11)..(11): binding site for cyclization
  BINDING (13)..(13): binding site for cyclization
XKQCDPETGECLP

SEQ ID NO: 50 - protein, 12 aa, Artificial Sequence
  peptidic sequences of the compounds of formula (I)
  LIPID (1)..(1): stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 51 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 52 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XVVCDPETGECVV

SEQ ID NO: 53 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XKKCDPETGECKK

SEQ ID NO: 54 - protein, 23 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (14)..(14): bAla
  MOD_RES (23)..(23): AMIDATION
XLQCDPETGECLPXRKKRRQRRR

SEQ ID NO: 55 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 56 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDPETGECLPXRKKRRQRRR

SEQ ID NO: 57 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  BINDING (13)..(13): AMIDATION
XKKCDPETGECLP

SEQ ID NO: 58 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LLCDPETGECLLXRKKRRQRRR

SEQ ID NO: 59 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LLCDPETGECLLXRKKRRQRRR

SEQ ID NO: 60 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LLCDPETGECLLXRKKRRQRRR

SEQ ID NO: 61 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 62 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 63 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LLCDPETGECLLXRKKRRQRRR

SEQ ID NO: 64 - protein, 12 aa, Artificial Sequence
  peptidic sequences of the compounds of formula (I)
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 65 - protein, 13 aa, Artificial Sequence
  peptidic sequences of the compounds of formula (I)
  LIPID (1)..(1): stearyl
  MOD_RES (1)..(1): bAla
  BINDING (4)..(4): binding site for cyclization
  BINDING (11)..(11): binding site for cyclization
  MOD_RES (13)..(13): AMIDATION
XLQCDPETGECLP

SEQ ID NO: 66 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDPETGECLPXRKKRRQRRR

SEQ ID NO: 67 - protein, 22 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (22)..(22): AMIDATION
LQCDPETGECLPXRKKRRQRRR

SEQ ID NO: 68 - protein, 21 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (21)..(21): AMIDATION
LQCDPETGECLPXRRRRRRRR

SEQ ID NO: 69 - protein, 23 aa, Artificial Sequence
  peptidic sequences of the compounds of formula (I)
  BINDING (3)..(3): binding site for cyclization
  BINDING (10)..(10): binding site for cyclization
  MOD_RES (13)..(13): bAla
  MOD_RES (23)..(23): AMIDATION
LQCDPETGECLPXRRRRRRRRRR

SEQ ID NO: 70 - protein, 12 aa, Artificial Sequence
  peptidic sequences of the compounds of formula (I)
  MOD_RES (1)..(1): (4S)-aminoproline
  LIPID (1)..(1): Stearyl
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDRETGECLP

SEQ ID NO: 71 - protein, 22 aa, Artificial Sequence
  peptidic sequences of the compounds of formula (I)
  BINDING (3)..(3): binding site for cyclization
  MOD_RES (5)..(5): Phe(4-F)
  BINDING (10)..(10): binding site for cyclization
  MOD_RES (13)..(13): bAla
  MOD_RES (22)..(22): AMIDATION
LQCDFETGECLPXRKKRRQRRR

SEQ ID NO: 72 - protein, 12 aa, Artificial Sequence
  peptidic sequences of the compounds of formula (I)
  MOD_RES (1)..(1): (4S)-aminoproline
  LIPID (1)..(1): Stearyl
  BINDING (1)..(1): binding site for cyclization
  BINDING (3)..(3): binding site for cyclization
  MOD_RES (5)..(5): Pro((4S)-F)
  BINDING (10)..(10): binding site for cyclization
  MOD_RES (12)..(12): D-Pro
  BINDING (12)..(12): binding site for cyclization
PQCDPETGECLP

SEQ ID NO: 73 - protein, 12 aa, Artificial Sequence
  peptidic sequences of the compounds of formula (I)
  MOD_RES (1)..(1): (4S)-aminoproline
  LIPID (1)..(1): Stearyl
  BINDING (1)..(1): binding site for cyclization
  BINDING (3)..(3): binding site for cyclization
  MOD_RES (5)..(5): Pro((4S)-NH2)
  BINDING (10)..(10): binding site for cyclization
  MOD_RES (12)..(12): D-Pro
  BINDING (12)..(12): binding site for cyclization
PQCDPETGECLP

SEQ ID NO: 74 - protein, 12 aa, Artificial Sequence
  peptidic sequences of the compounds of formula (I)
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 75 - protein, 12 aa, Artificial Sequence
  peptidic sequences of the compounds of formula (I)
  LIPID (1)..(1): Stearyl
  MOD_RES (1)..(1): ACETYLATION
  MOD_RES (1)..(1): (4S)-aminoproline
  BINDING (3)..(3): binding site for cyclization
  BINDING (10)..(10): binding site for cyclization
  MOD_RES (12)..(12): D-Pro
PQCDPETGECLP

SEQ ID NO: 76 - protein, 12 aa, Artificial Sequence
  peptidic sequences of the compounds of formula (I)
  LIPID (1)..(1): stearyl
  BINDING (3)..(3): binding site for cyclization
  BINDING (10)..(10): binding site for cyclization
  MOD_RES (12)..(12): D-Pro
  MOD_RES (12)..(12): AMIDATION
PQCDPETGECLP

SEQ ID NO: 77 - protein, 12 aa, Artificial Sequence
  peptidic sequences of the compounds of formula (I)
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (11)..(11): MeLeu
  MOD_RES (12)..(12): D-Pro
  BINDING (12)..(12): binding site for cyclization
PQCDPETGECLP

SEQ ID NO: 78 - protein, 12 aa, Artificial Sequence
  peptidic sequences of the compounds of formula (I)′
  LIPID (1)..(1): Stearyl
  BINDING (1)..(1): binding site for cyclization
  MOD_RES (1)..(1): (4S)-aminoproline
  BINDING (3)..(3): binding site for cyclization
  MOD_RES (5)..(5): L-thioproline
  BINDING (10)..(10): binding site for cyclization
  MOD_RES (12)..(12): D-Pro
  BINDING (12)..(12): binding site for cyclization
PQCDPETGECLP

SEQ ID NO: 79 - protein, 12 aa, Artificial Sequence
  peptidic sequences of the compounds of formula (I)′
  LIPID (1)..(1): Stearyl
  BINDING (1)..(1): binding site for cyclization
  MOD_RES (1)..(1): (4S)-aminoproline
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 80 - protein, 12 aa, Artificial Sequence
  peptidic sequences of the compounds of formula (I)′
  LIPID (1)..(1): Miristoyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): D-Pro
  MOD_RES (12)..(12): binding site for cyclization
PQCDPETGECLP

SEQ ID NO: 81 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PKCDPETGECLP

SEQ ID NO: 82 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECKP

SEQ ID NO: 83 - protein, 12 aa, Artificial Sequence
  peptidic sequences of the compounds of formula (I)′
  LIPID (1)..(1): Palmitoyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 84 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 85 - protein, 12 aa, Artificial Sequence
  peptidic sequences of the compounds of formula (I)′
  LIPID (1)..(1): Lauryl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 86 - protein, 12 aa, Artificial Sequence
  peptidic sequences of the compounds of formula (I)′
  LIPID (1)..(1): alpha-linolenyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 87 - protein, 12 aa, Artificial Sequence
  peptidic sequences of the compounds of formula (I)′
  LIPID (1)..(1): Elaidyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 88 - protein, 12 aa, Artificial Sequence
  peptidic sequences of the compounds of formula (I)′
  LIPID (1)..(1): Oleyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 89 - protein, 12 aa, Artificial Sequence
  peptidic sequences of the compounds of formula (I)′
  LIPID (1)..(1): Behenyl
  MOD_RES (1)..(1): (4S)-aminoproline
  MOD_RES (1)..(1): binding site for cyclization
  MOD_RES (3)..(3): binding site for cyclization
  MOD_RES (10)..(10): binding site for cyclization
  MOD_RES (12)..(12): binding site for cyclization
  MOD_RES (12)..(12): D-Pro
PQCDPETGECLP

SEQ ID NO: 90 - protein, 12 aa, Artificial Sequence
  peptidic sequences of the compounds of formula (I)′
  LIPID (1)..(1): Arachidyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
PQCDPETGECLP

SEQ ID NO: 91 - protein, 12 aa, Artificial Sequence
  peptidic sequences of the compounds of formula (I)′
  LIPID (1)..(1): Stearyl (appended to lateral chain)
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
KQCDPETGECLP

SEQ ID NO: 92 - protein, 12 aa, Artificial Sequence
  peptidic sequences of the compounds of formula (I)′
  LIPID (1)..(1): Stearyl (appended to N-alpha atom)
  BINDING (1)..(1): binding site for cyclization
  BINDING (3)..(3): binding site for cyclization
  BINDING (10)..(10): binding site for cyclization
  BINDING (12)..(12): binding site for cyclization
  MOD_RES (12)..(12): D-Pro
KQCDPETGECLP

SEQ ID NO: 93 - protein, 12 aa, Artificial Sequence
  peptidic sequences of the compounds of formula (I)′
  LIPID (1)..(1): Arachidyl
  MOD_RES (1)..(1): (4S)-aminoproline
  BINDING (1)..(1): binding site for cyclization
  BINDING (3)..(3): binding site for cyclization
  MOD_RES (5)..(5): L-thioproline
  BINDING (10)..(10): binding site for cyclization
  BINDING (12)..(12): binding site for cyclization
  MOD_RES (12)..(12): D-Pro
PQCDPETGECLP

SEQ ID NO: 94 - protein, 12 aa, Artificial Sequence
  peptidic sequences of the compounds of formula (I)′
  LIPID (1)..(1): Arachidyl
  MOD_RES (1)..(1): (4S)-aminoproline
  BINDING (1)..(1): binding site for cyclization
  BINDING (3)..(3): binding site for cyclization
  MOD_RES (5)..(5): L-thioproline
  BINDING (10)..(10): binding site for cyclization
  MOD_RES (12)..(12): D-Pro
  BINDING (12)..(12): binding site for cyclization
PQCDPETGECLP

SEQ ID NO: 95 - protein, 12 aa, Artificial Sequence
  peptidic sequences of the compounds of formula (I)′
  LIPID (1)..(1): Arachidyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 96 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  MOD_RES (5)..(5): L-thioproline
  BINDING (10)..(10): binding site for cyclization
  MOD_RES (12)..(12): D-Pro
  BINDING (12)..(12): binding site for cyclization
PQCDPETGECLP

SEQ ID NO: 97 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  MOD_RES (5)..(5): L-thioproline
  BINDING (10)..(10): binding site for cyclization
  MOD_RES (12)..(12): D-Pro
  BINDING (12)..(12): binding site for cyclization
PQCDPETGECLP

SEQ ID NO: 98 - protein, 12 aa, Artificial Sequence
  peptidic sequences of the compounds of formula (I)′
  LIPID (1)..(1): Nonadecanoyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 99 - protein, 13 aa, Artificial Sequence
  peptidic sequences of the compounds of formula (I)′
  LIPID (1)..(1): Stearyl
  MOD_RES (1)..(1): bAla
  BINDING (4)..(4): binding site for cyclization
  BINDING (11)..(11): binding site for cyclization
  MOD_RES (13)..(13): Amidation
XLLCDPETGECLL

SEQ ID NO: 100 - protein, 12 aa, Artificial Sequence
  peptidic sequences of the compounds of formula (I)′
  LIPID (1)..(1): Stearyl
  MOD_RES (1)..(1): (4S)-aminoproline
  BINDING (1)..(1): binding site for cyclization
  BINDING (3)..(3): binding site for cyclization
  BINDING (10)..(10): binding site for cyclization
  MOD_RES (12)..(12): D-Pro
  BINDING (12)..(12): binding site for cyclization
PQCDPETGECLP

SEQ ID NO: 101 - protein, 4 aa, Artificial Sequence
  sequence motif of Nrf2
ETGE

SEQ ID NO: 102 - protein, 6 aa, Artificial Sequence
  high affinity sequence domain between Nrf2 and the kelch domain of Keap1
DEETGE

SEQ ID NO: 103 - protein, 6 aa, Artificial Sequence
  motif containing a beta turn region stablilized by aspartate and threonine residues
  MOD_RES (2)..(2): Xaa can be any naturally occurring amino acid
DXETGE

SEQ ID NO: 104 - protein, 11 aa, Artificial Sequence
  cell penetrating peptide
YGRKKRRQRRR

SEQ ID NO: 105 - protein, 12 aa, Artificial Sequence
  cell penetrating peptide
GRKKRRQRRRPQ

SEQ ID NO: 106 - protein, 8 aa, Artificial Sequence
  cell penetrating peptide
RRRRRRRR

SEQ ID NO: 107 - protein, 16 aa, Artificial Sequence
  cell penetrating peptide
RQIKIWFQNRRMKWKK

SEQ ID NO: 108 - protein, 15 aa, Artificial Sequence
  cell penetrating peptide
  MOD_RES (15)..(15): AMIDATION
KFHTFPQTAIGVGAP

SEQ ID NO: 109 - protein, 18 aa, Artificial Sequence
  cell penetrating peptide
KLALKLALKALKAALKLA

SEQ ID NO: 110 - protein, 15 aa, Artificial Sequence
  cell penetrating peptide
RQIKWFQNRRMKWKK

SEQ ID NO: 111 - protein, 18 aa, Artificial Sequence
  cell penetrating peptide
RGGRLSYSRRRFSTSTGR

SEQ ID NO: 112 - protein, 10 aa, Artificial Sequence
  cell penetrating peptide
RRLSYSRRRF

SEQ ID NO: 113 - protein, 12 aa, Artificial Sequence
  cell penetrating peptide
PIRRRKKLRRLK

SEQ ID NO: 114 - protein, 12 aa, Artificial Sequence
  cell penetrating peptide
RRQRRTSKLMKR

SEQ ID NO: 115 - protein, 15 aa, Artificial Sequence
  cell penetrating peptide
RRRRNRTRRNRRRVR

SEQ ID NO: 116 - protein, 19 aa, Artificial Sequence
  cell penetrating peptide
KMTRAQRRAAARRNRWTAR

SEQ ID NO: 117 - protein, 13 aa, Artificial Sequence
  cell penetrating peptide
TRRQRTRRARRNR

SEQ ID NO: 118 - protein, 13 aa, Artificial Sequence
  cell penetrating peptide
GRRRRRRRRRPPQ

SEQ ID NO: 119 - protein, 17 aa, Artificial Sequence
  cell penetrating peptide
KLALKLALKLALALKLA

SEQ ID NO: 120 - protein, 10 aa, Artificial Sequence
  Tag2 sequence
  MOD_RES (10)..(10): AMIDATION
RRRRRRRRRR

SEQ ID NO: 121 - protein, 8 aa, Artificial Sequence
  Tag2 sequence
  MOD_RES (8)..(8): AMIDATION
RRRRRRRR

SEQ ID NO: 122 - protein, 9 aa, Artificial Sequence
  Tag2 sequence
  MOD_RES (9)..(9): AMIDATION
RKKRRQRRR

SEQ ID NO: 123 - protein, 11 aa, Artificial Sequence
  Tag2 sequence
  MOD_RES (11)..(11): AMIDATION
YARAAARQARA